(19) United States
(12) Patent Application Publication
(10) Pub. No.: US 2025/0383357 A1
(43) Pub. Date: Dec. 18, 2025

(54) NOVEL FLUORESCENT DYES AND POLYMERS FROM DIHYDROPHENANTHRENE DERIVATIVES

(71) Applicant: Beckman Coulter, Inc., Brea, CA (US)

(72) Inventor: Arunkumar Easwaran, Miami, FL (US)

(21) Appl. No.: 18/877,833

(22) PCT Filed: Jun. 30, 2023
(86) PCT No.: PCT/US2023/069546

§ 371 Date: Dec. 20, 2024

Related U.S. Application Data
(60) Provisional application No. 63/357,980, filed on Jul. 1, 2022.
(60) Provisional application No. 63/490,921, filed on Mar. 17, 2023.

(51) Int. Cl. G01N 33/58; C09B 23/10; C09B 62/36; C09K 11/06

(52) U.S. Cl. CPC G01N 33/582; C09B 23/107; C09B 62/365; C09K 11/06; C09K 2211/1018

(57) ABSTRACT

The present disclosure provides novel dihydrophenanthrene (DHP)-cyanine and DHP-squaraine fluorescent compounds and water-soluble polymers thereof. The DHP-cyanine and DHP-squaraine fluorescent compounds and polymers can be excited using UV, violet, blue, yellow, green, red, or NIR wavelengths. The fluorescent dyes may be conjugated to antibodies for detection of target analytes in biological samples and are suitable for use in flow cytometry analyses.

This application is being filed on Jun. 30, 2023, as a PCT International Patent application and claims the benefit of and priority to U.S. Provisional patent application Ser. No. 63/357,980, filed Jul. 1, 2022, and U.S. Provisional patent application Ser. No. 63/490,921, filed Mar. 17, 2023, the entire disclosures of which are incorporated by reference herein in their entirety.

### BACKGROUND OF THE INVENTION

There is an increasing demand for a variety of fluorescent dyes for use in current flow cytometers as well as in spectral flow instruments. Water soluble fluorescent compounds and their conjugates can be used in a variety of biological applications by generating signals which can be monitored in real time and provide simple and rapid methods for the detection of biological targets and events, e.g., in diagnostic kits, in microscopy, in cytometry, or in drug screening.

Molecular recognition involves the specific binding of two molecules. Molecules which have binding specificity for a target biomolecule find use in a variety of research and diagnostic applications, such as the labelling and separation of analytes, flow cytometry, in situ hybridization, enzyme-linked immunosorbent assays (ELISAs), western blot analysis, magnetic cell separations and chromatography. Target biomolecules may be detected by labelling with a fluorescent dye.

Current dihydrophenanthrene based polymer dyes have strong excitation in the ultraviolet and violet regions of 350-450 nm. U.S. Pat. No. 11,208,527 describes water soluble dihydrophenanthrene (DHP) based fluorescent polymer dyes, for example, exhibiting excitation maxima between 395-415 nm with emitted light between about 415-475 nm. U.S. Pat. No. 11,584,825 describes water soluble DHP based violet excitable polymers and tandem dyes.

Demands have increased for multicolor panels for both conventional and spectral flow cytometry that require additional fluorescent dyes excitable with other lasers (e.g., 488, 563, 638, and 808 nm). Parameters considered by a user in choosing a fluorescent dye may include excitation wavelength maximum, the emission wavelength maximum, brightness of the dye, and the fluorescence lifetime. Brightness of a dye is an overall contribution from the extinction coefficient (ε, measure of the amount of light absorbed at a particular wavelength) and fluorescence quantum yield (Φ, measure of the light emitted in the form of radiation from its singlet excited state).

Attempts to shift the excitation range further to the red region by incorporating modifier unit monomers into existing DHP violet-excitable polymer backbones resulted in retention of original absorption of the polymer along with the absorption from the acceptor dye. Direct excitation of the resulting DHP-modifier unit polymer dyes resulted in strong UV and violet emission from the core. Attempts to design red shifted core polymers using other aromatic cyclic molecules as monomers encountered synthetic challenges. Therefore, more careful design was required for shifting the excitation maxima to blue, green, and red regions of the spectrum and beyond.

Organic fluorescent dyes and water-soluble polymer dyes that can be excited using UV, violet, blue, yellow, green, red, and near infrared (NIR) wavelengths are desirable.

### SUMMARY OF THE INVENTION

The present disclosure generally provides dihydrophenanthrene (DHP)-cyanine (Cy) and DHP-squaraine compounds and polymer dyes, water-soluble DHP-cyanine and DHP-squaraine compounds and water-soluble fluorescent polymers conjugated to a specific binding partner, their complexes, and methods for detecting analytes in a sample using the complexes comprising the water-soluble DHP-Cy and DHP-squaraine fluorescent compounds or polymers conjugated to a binding partner. Tandem dyes are also provided comprising the DHP-Cy and DHP-squaraine fluorescent compounds, polymers, or labeled specific binding partners according to the disclosure. The DHP-Cy and DHP-squaraine compounds, polymers, labeled specific binding partners, and tandem dyes according to the present disclosure are useful in biological applications, including for the detection of target analytes and use in diagnostic kits, etc. The kits may comprise a DHP-Cy compound, a DHP-squaraine compound, polymer, labeled specific binding partner, and/or tandem dye according to the present disclosure, optionally having a conjugation tag.

The disclosure provides a fluorescent compound comprising a structure according to Formula (I):

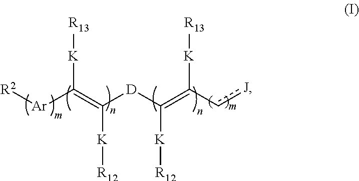

wherein
D is an aryl group, heteroaryl group,

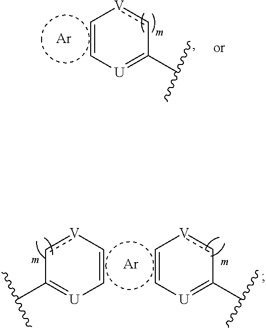

J is an aryl group, a heteroaryl group, or

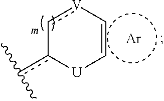

and at least one of D or J is independently selected from the group consisting of

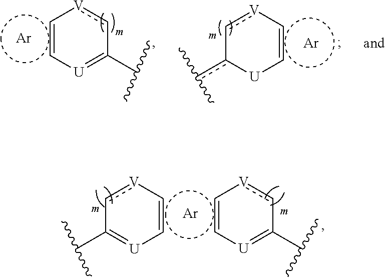

wherein
optional Ar and optional

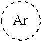

are each independently selected from the group consisting of a substituted or unsubstituted benzene, benzene derivative, monocyclic aryl group, polycyclic aryl group, monocyclic heteroaryl group, and polycyclic heteroaryl group, with at least one

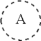

of D or J being present and comprising

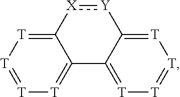

or a derivative thereof comprising an additional aryl or heteroaryl group fused at any available

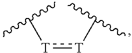

wherein
   each T is independently selected from the group consisting of C, C(R¹), N, P, O, S, and Si(R¹);
   each U is independently selected from the group consisting of NR¹⁰, O, P, and S;
   each V is independently selected from the group consisting of NR¹¹, CR¹¹, C(R¹¹)₂, S, SO₂, O, and Si(R¹¹)₂;
   each V′ is independently SO₂, SO, S, NR¹¹, CR¹¹, C(R¹¹)₂, O, Si(R¹¹)₂, >C═O, >Se═O, —CH═CH—, or —N═CH—;
   each X is independently CR¹R² or SiR¹R²;
   each Y is independently CR⁸R⁹ or SiR⁸R⁹;
   wherein each R¹, R², R⁸, and R⁹ is independently selected from the group consisting of a water-solubilizing moiety, a linker moiety, a linked E, hydrogen, hydroxy, halogen, alkoxy, alkyl, alkenyl, alkynyl, cycloalkyl, haloalkyl, aryl, heteroaryl, (hetero)aryloxy, (hetero)arylamino, sulfonamide-PEG, phosphoramide-PEG, ammonium alkyl salt, ammonium alkyloxy salt, carbonyl, acyl, ammonium oligoether salt, sulfonate alkyl salt, sulfonate alkoxy salt, sulfonate oligoether salt, sulfonamido oligoether, sulfonamide, sulfinamide, phosphonamidate, phosphinamide, alkoxy sulfonamide PEG, alkoxy sulfonate, alkyl sulfonate, alkyl sulfonate salt,

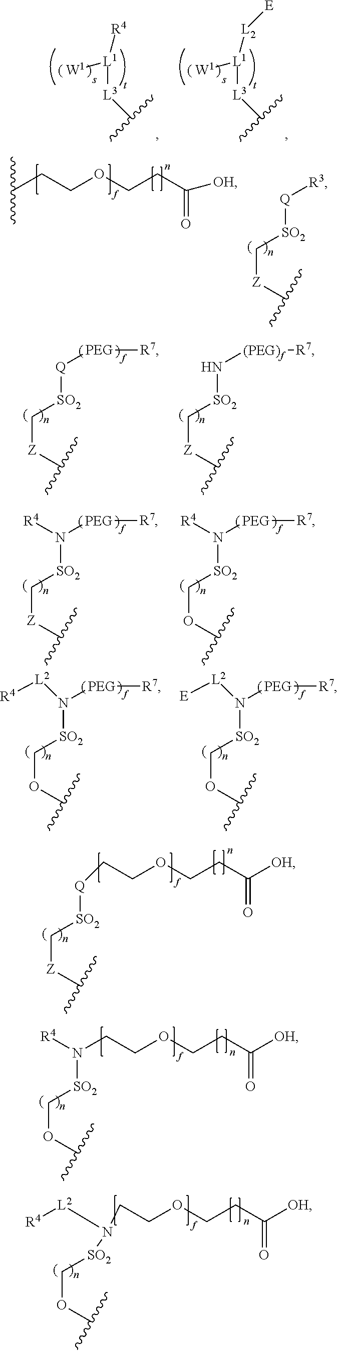

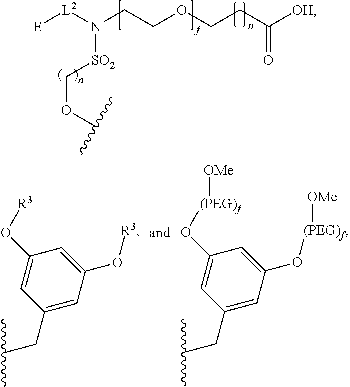

optionally wherein R¹ and R⁸ together form an unsubstituted or substituted cycloalkyl, cycloalkenyl, heterocycloalkyl, heterocycloalkenyl, cycloalkoxy, aryl, or heteroaryl having 3 to 9 ring members;
each R³ is independently selected from the group consisting of H, alkyl, alkene, alkyne, cycloalkyl, haloalkyl, alkoxy, (hetero)aryloxy, aryl, (hetero)arylamino, a water-solubilizing moiety, a chromophore, functional moiety, binding partner, and a PEG group;
each Q is independently a bond, NH, NR⁴, C₁-C₁₂ alkylene, CHR⁴, and CH₂CH₂, CHR⁴, O, NR⁴, or NH;
each Z is independently CH₂, CHR⁴, O, NR⁴, or NH;
each W¹ is independently a water-solubilizing moiety;
L¹, L², and L³ are each independently selected linker moieties;
each E is independently selected from the group consisting of a chromophore, a functional moiety, a substrate, and a binding partner;
each R⁴ is independently selected from the group consisting of H, alkyl, PEG, a water-solubilizing moiety, a linker moiety, a chromophore, functional group, a conjugation tag, carboxylic amine, amine, carbamate, carboxylic acid, carboxylate ester, maleimide, activated ester, N-hydroxysuccinimidyl, hydrazine, hydrazide, hydrazone, azide, alkyne, aldehyde, and thiol, or protected groups thereof;
each R⁷ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester, and C₁-C₁₂ alkoxy;
each R¹⁰ is independently selected from the group consisting of hydrogen, a linker moiety, a linked reactive group, a linked ionic group, a linked chromophore, a linked water-solubilizing moiety, a water-solubilizing moiety, a chromophore, a binding partner, a linked binding partner;
each R¹¹ is independently selected from the group consisting of hydrogen, a linker moiety, a linked reactive group, a linked ionic group, a linked chromophore, a linked water-solubilizing moiety, a water-solubilizing moiety, a chromophore, a binding partner, and a linked binding partner;
each R¹² and R¹³ is independently selected from the group consisting of hydrogen, C₁-C₆ alkyl, C₁-C₆ alkyl, C₃-C₁₀ cycloalkyl, substituted or unsubstituted aryl, and substituted or unsubstituted heteroaryl; or R¹² and R¹³ together form an unsubstituted or substituted unsaturated cycloalkyl group having 3 to 8 ring members or substituted or unsubstituted heterocycloalkyl having 3 to 8 ring members optionally substituted with 0;
each K is independently a covalent bond, O, S, P or CH₂
each f is independently an integer from 0 to 50.
each m is independently 0 or 1;
each n is independently 0, 1, 2, 3, or 4
each s is independently 1 or 2; and
each t is independently 0, 1, 2, or 3.

The disclosure provides a fluorescent compound comprising a structure according to Formula (II):

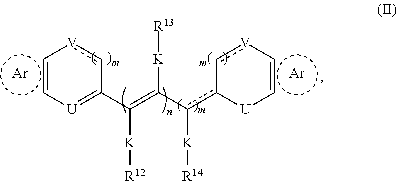

wherein
   each

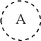

is independently selected from the group consisting of a substituted or unsubstituted benzene, benzene derivative, monocyclic aryl group, polycyclic aryl group, monocyclic heteroaryl group, and polycyclic heteroaryl group, wherein at least one

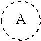

comprises

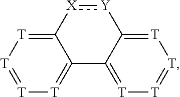

or a derivative thereof, wherein the derivative optionally comprises an additional aryl or heteroaryl group fused at any available

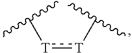

wherein
each V′ is independently SO₂, SO, S, NR¹¹, CR¹¹, C(R¹¹)₂, O, Si(R¹¹)₂, >C═O, >Se═O, —CH═CH—, or —N═CH—;
each T is independently selected from the group consisting of C, C(R¹), N, P, O, S, and Si(R¹);
each U is independently selected from the group consisting of NR¹⁰, O, P, Se, Te, and S;
each V is independently selected from the group consisting of NR¹¹, CR¹¹, C(R¹¹)₂, S, SO₂, O, Se, Te, and Si(R¹¹)₂;
each X is independently CR¹R² or SiR¹R²;
each Y is independently CR⁸R⁹ or SiR⁸R⁹;
wherein
each R¹, R², R⁸, and R⁹ is independently selected from the group consisting of a water-solubilizing moiety, a linked water-solubilizing moiety, a linker moiety, a linked E, a reactive group, a linked reactive group, binding partner, linked binding partner, a functional group, hydrogen, hydroxy, halogen, alkoxy, alkyl, alkenyl, alkynyl, cycloalkyl, haloalkyl, aryl, heteroaryl, (hetero)aryloxy, (hetero)arylamino, sulfonamide-PEG, phosphoramide-PEG, ammonium alkyl salt, ammonium alkyloxy salt, ammonium oligoether salt, carbonyl, acyl, sulfonate alkyl salt, sulfonate alkoxy salt, sulfonate oligoether salt, sulfonamido oligoether, sulfonamide, sulfinamide, phosphonamidate, phosphinamide [symbol] onamide PEG, alkoxy sulfonate, alkyl sulfonate, alkyl sulfonate salt,

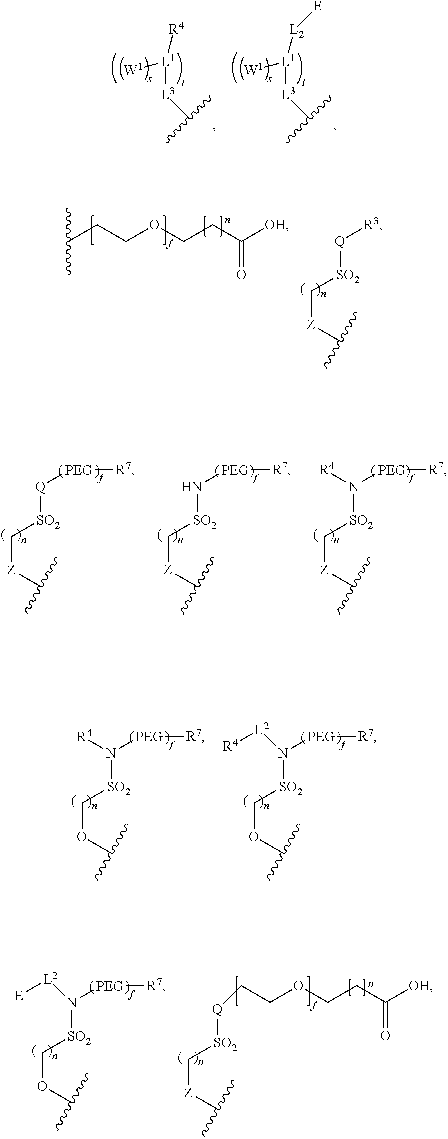

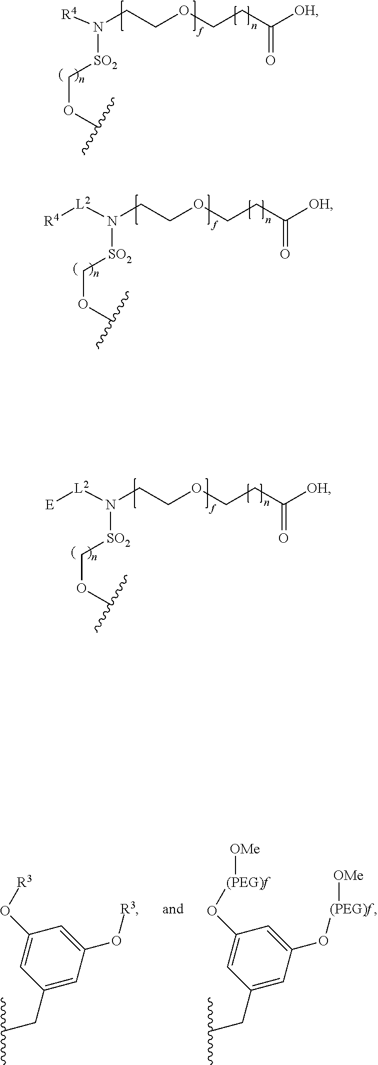

optionally wherein R¹ and R⁸ together form an unsubstituted or substituted cycloalkyl, cycloalkenyl, heterocycloalkyl, heterocycloalkenyl, cycloalkoxy, aryl, or heteroaryl having 3 to 9 ring members;
each R³ is independently selected from the group consisting of H, alkyl, alkene, alkyne, cycloalkyl, haloalkyl, alkoxy, (hetero)aryloxy, aryl, (hetero)arylamino, a water-solubilizing moiety, a chromophore, functional moiety, binding partner, and a PEG group;
each Q is independently a bond, NH, NR⁴, C₁-C₁₂ alkylene, CHR⁴, or CH₂;
each Z is independently CH₂, CHR⁴, O, NR⁴, or NH;
each W¹ is independently a water-solubilizing moiety;
L¹, L², and L³ are each independently selected linker moieties;
each E is independently selected from the group consisting of a chromophore, a functional moiety, a substrate, and a binding partner;
each R⁴ is independently selected from the group consisting of H, alkyl, PEG, a water-solubilizing moiety, a linker moiety, a chromophore, a functional group, carboxylic amine, amine, carbamate, carboxylic acid, carboxylate ester, maleimide, activated ester, N-hydroxysuccinimidyl, hydrazine, hydrazide, hydrazone, azide, alkyne, alkene, tetrazine, aldehyde, and thiol, or protected groups thereof;
each R⁷ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester, and C₁-C₁₂ alkoxy;
each R¹⁰ is independently selected from the group consisting of hydrogen, a linker moiety, a linked reactive group, a linked ionic group, a linked chromophore, a linked water-solubilizing moiety, a water-solubilizing moiety, a chromophore, a binding partner, a linked binding partner;
each R¹¹ is independently selected from the group consisting of hydrogen, a linker moiety, a linked reactive group, a linked ionic group, a linked chromophore, a linked water-solubilizing moiety, a chromophore, a binding partner, and a linked binding partner;
each R¹² and R¹³ is independently selected from the group consisting of hydrogen, halogen, substituted or unsubstituted C₁-C₆ alkyl, substituted or unsubstituted C₁-C₆ heteroalkyl, substituted or unsubstituted C₁-C₆ alkene, substituted or unsubstituted C₃-C₁₀ cycloalkyl, substituted or unsubstituted heterocycloalkyl, substituted or unsubstituted aryl, and substituted or unsubstituted heteroaryl; or R¹² and R¹³ together form an unsubstituted or substituted unsaturated cycloalkyl group having 3 to 8 ring members or substituted or unsubstituted heterocycloalkyl having 3 to 8 ring members optionally substituted with O;
each K is independently a covalent bond, O, S, P, NR¹, Se, Te, CR¹R², or CH₂;
each f is independently an integer from 0 to 50;
each m is independently 0 or 1;
each n is independently 0, 1, 2, 3, or 4;
each s is independently 1 or 2; and
each t is independently 0, 1, 2, or 3.

The derivative of

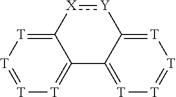

comprising an additional aryl or heteroaryl group fused at any available

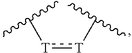

may optionally comprise,

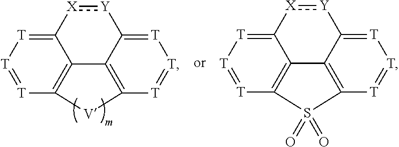

wherein V′ is SO₂, SO, S, NR¹¹, CR¹¹, C(R¹¹)₂, O, Si(R¹¹)₂, >C═O, >Se═O, —CH═CH—, or —N═CH—, and T, V, X, Y, and m are as defined herein above.

The present disclosure provides DHP-cyanine and DHP-squaraine compounds selected from the group consisting of Formulas (IIa), (IIb), (IIc), (IId), (IIe), (IIf), (IIg), (IIh), (IIi), (IIj), (Ik), (IIl), (IIm), (IIn), (IIo), (IIp), (IIq), (IIr), (IIs), (IIt), (IIu), (IIv), (IIw), (IIx), (IIy), and (IIz):

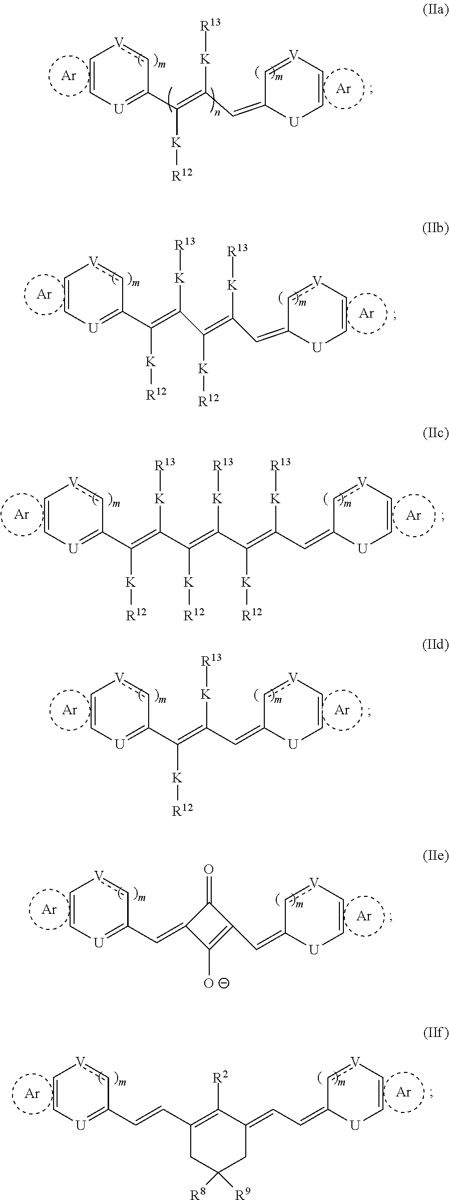

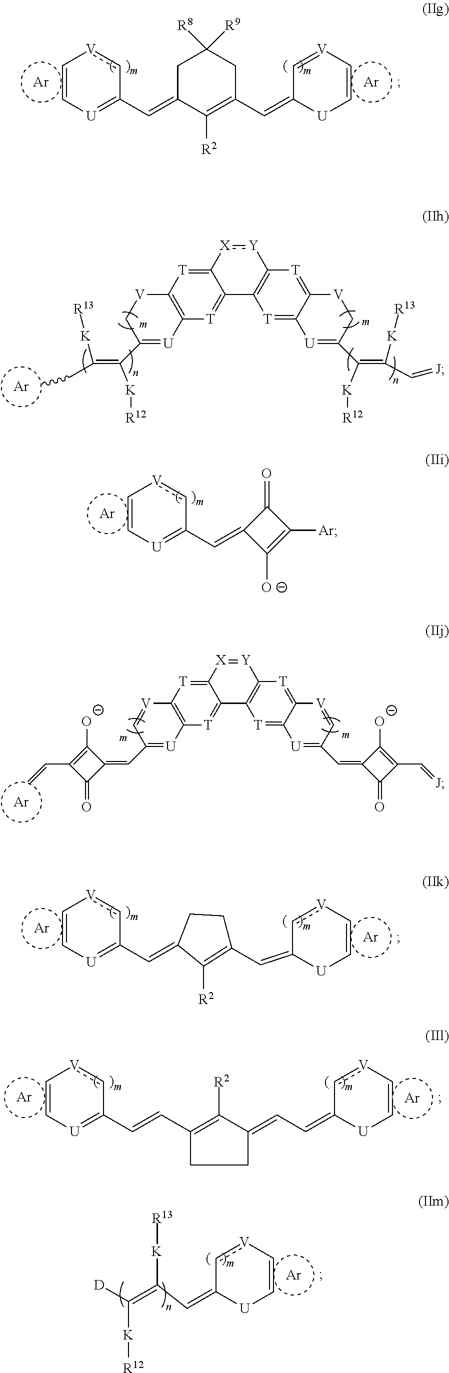

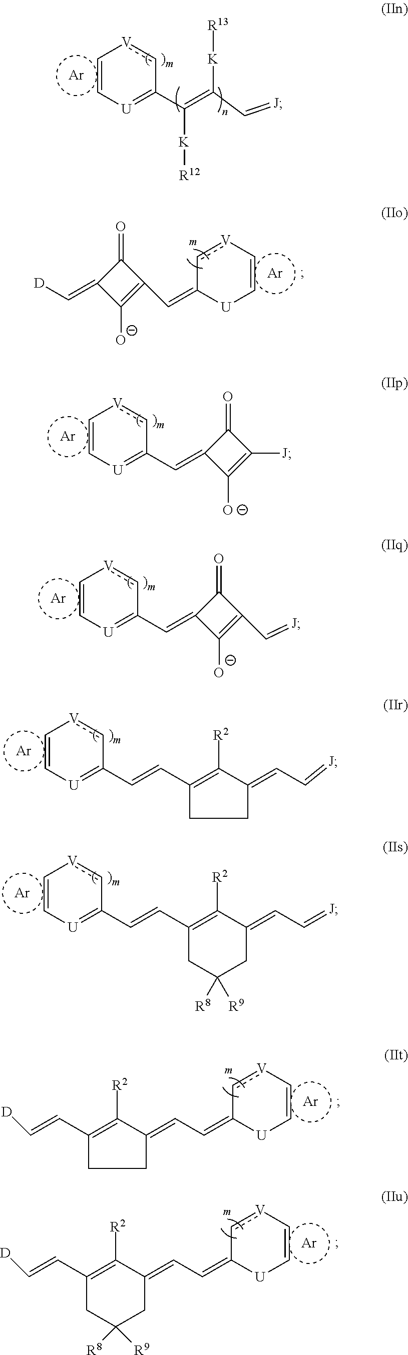

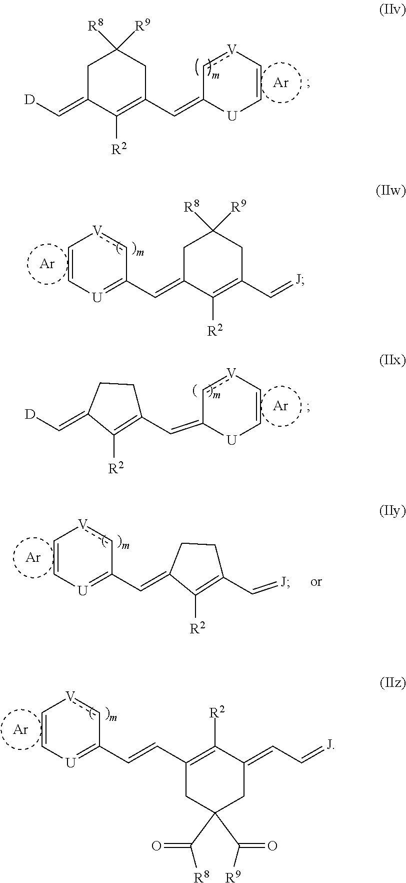

The disclosure provides DHP-cyanine and DHP-squaraine compounds selected from the group consisting of Formulas (I [symbol] IIc), (IIId), (IIIe), (IIIf), (IIIg), (IIIh), (IIIi), (IIIj), (IIIk), (IIIl), (IIIm), (IIIn), (IIIo), (IIIp), (IIIq), (IIIr), (IIIs), (IIIt), (IIIu), (IIIv), (IIIw), (IIIx), (IIIy), (IIIz), (IIIaa), (IIIbb), (IIIcc), (IIIdd), and (IIIee):

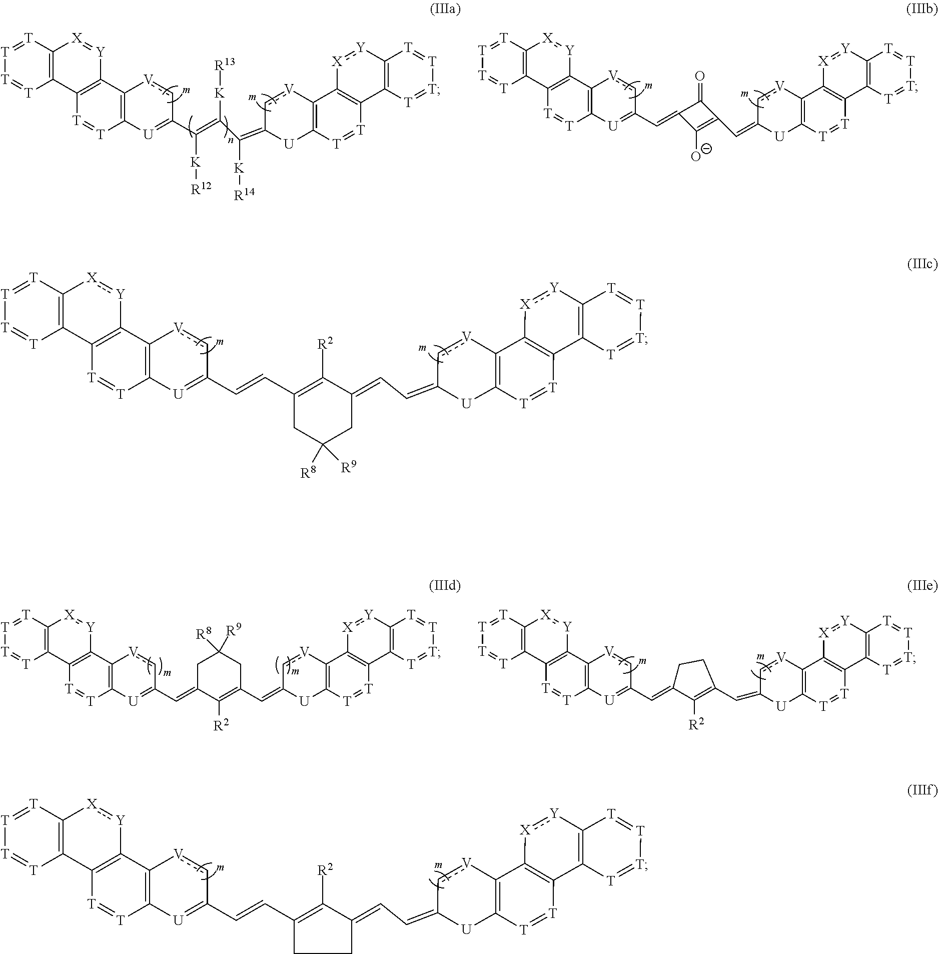

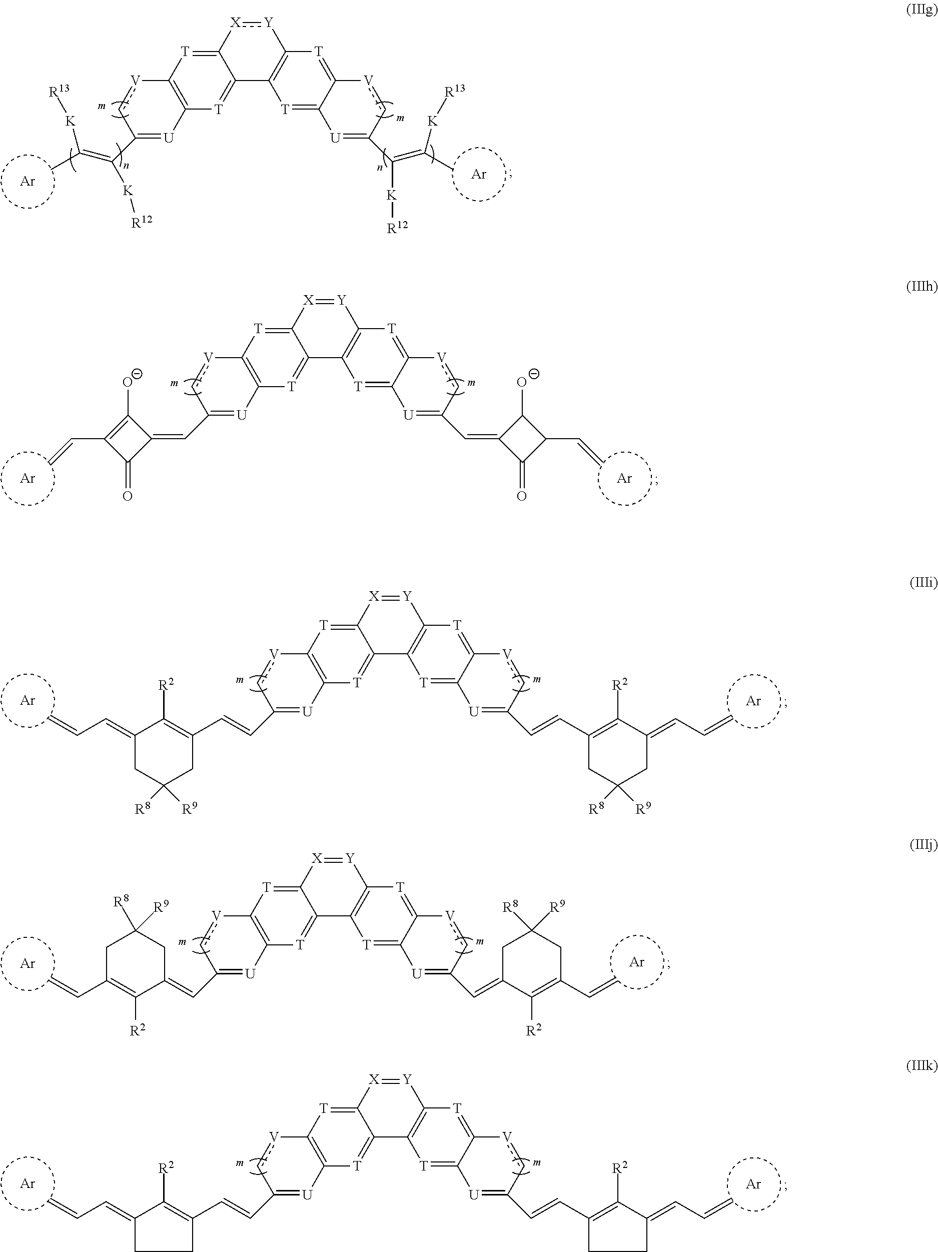

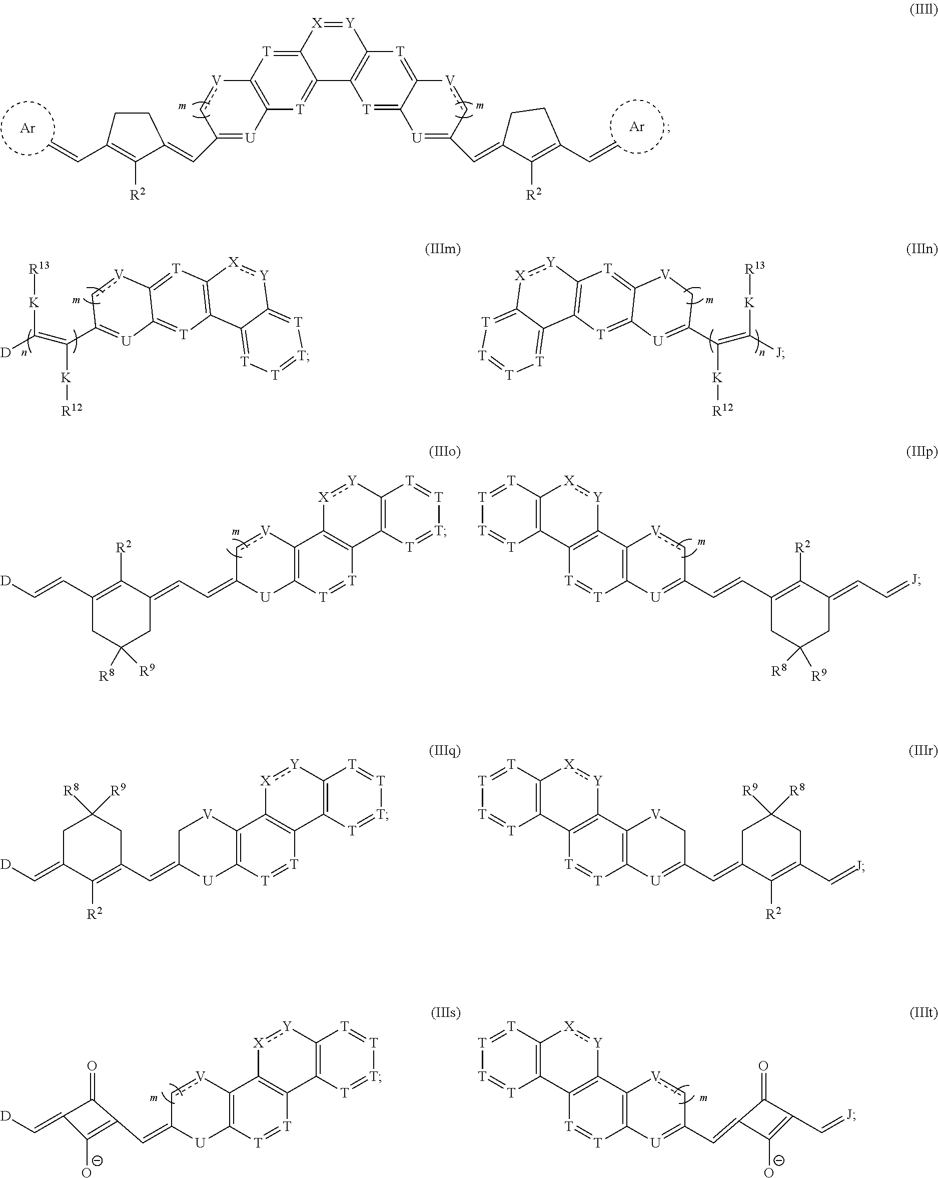

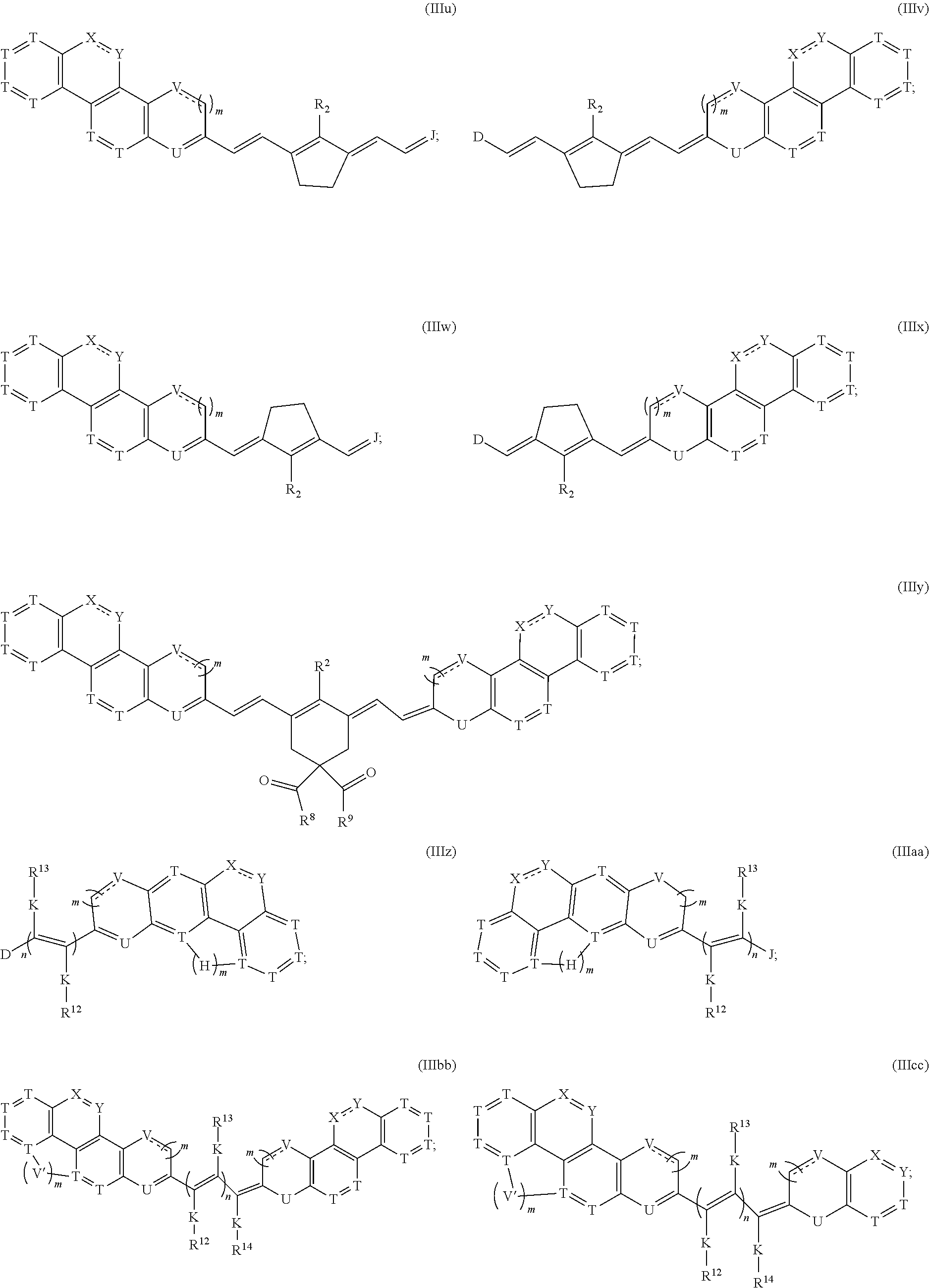

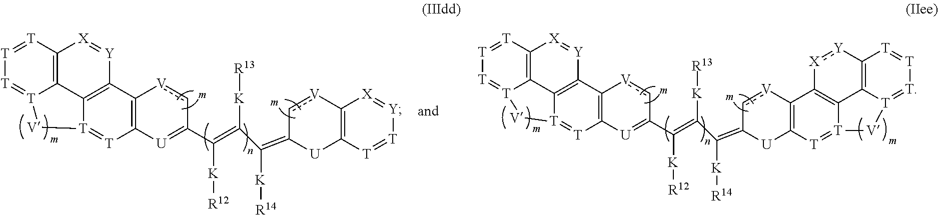

In some embodiments, the

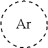

groups in both D and J comprise the same or different

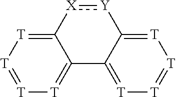

group or derivative thereof.

In some embodiments, the

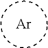

groups in both D and J comprise the same

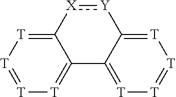

group or derivative thereof.

In some embodiments, the

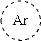

groups in both D and J comprise different

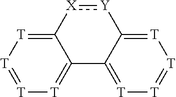

groups or derivatives thereof.

In some embodiments, D is

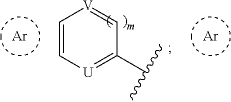

### of D is

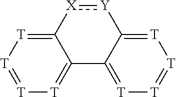

or a derivative thereof; and J is an aryl, heteroaryl or

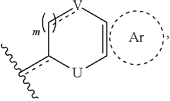

wherein

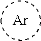

of J is an unsubstituted or substituted benzene, benzene derivative comprising fused monocyclic aryl, polycyclic aryl, monocyclic heteroaryl, or polycyclic heteroaryl group.

In some embodiments, J is

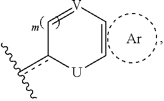

wherein

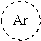

of J is an unsubstituted or substituted benzene or unsubstituted or substituted naphthalene; U is N; and V is N, O, or S.

In some embodiments, J is an unsubstituted or substituted quinoline, benzoxazole, benzothiazole, benzimidazole, indole, benzindole, pyridinium, benzopyrylium, thiopyrylium, fluorene, 9H-fluorene, phenanthrene, dihydrophenanthrene, 9,10-dihydrophenanthrene, 6,8-dihydro-5H-naphtho[2,1-f]indole, 4,5-dihydro-3H-naphtho[2,1-e]indole, 6,7-dihydro-3H-naphtho[2,1-g]indole, 5,6-dihydrophenanthro[3,2-d]thiazole, 4,5-dihydrophenanthro[2,1-d]thiazole, 6,7-dihydrophenanthro[4,3-d]thiazole, 5,6-dihydrophenanthro[3,2-d]oxazole, 4,5-dihydrophenanthro[2,1-d]oxazole, 6,7-dihydrophenanthro[4,3-d]oxazole, 5,6-dihydronaphtho[2,1-g]quinoline, 7,8-dihydronaphtho[2,1-h]quinoline, 5,6-dihydronaphtho[2,1-f]quinoline, 5,6-dihydro-8λ2-phenanthro[3,2-d]imidazole, 4,5-dihydro-3λ2-phenanthro[2,1-d]imidazole, 6,7-dihydro-3λ2-phenanthro[4,3-d]imidazole, 5,6-dihydronaphtho[1,2-g]quinoxaline, 5,6-dihydronaphtho[2,1-f]quinoxaline, 7,8-dihydronaphtho[1,2-f]quinoxaline, phenyl, 1H-benzo[e]indol-3-ium, 1H-benzo[e]indole, and 9,10-dihydro-8H-thieno[2′,3′,4′,5′:4,5]phenanthro[2,1-b]pyrrole 4,4-dioxide.

In some embodiments, D is aryl, heteroaryl or

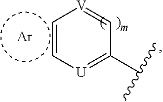

wherein

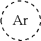

of D is a substituted or unsubstituted benzene, benzene derivative, monocyclic aryl group, polycyclic aryl group, monocyclic heteroaryl group and polycyclic heteroaryl group; and J is

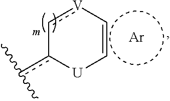

wherein

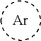

### of J is

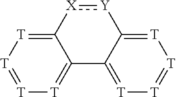

or a derivative thereof.

In some embodiments, D is

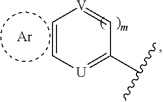

wherein

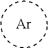

of D is an unsubstituted or substituted benzene or unsubstituted or substituted naphthalene; U is N; and V is N, O, or S.

In some embodiments, D is selected from the group consisting of unsubstituted or substituted quinoline, benzoxazole, benzthiazole, benzimidazole, indole, benzindole, pyridinium, benzopyrylium, thiopyrylium, fluorene, 9H-fluorene, phenanthrene, dihydrophenanthrene, 9,10-dihydrophenanthrene, 6,8-dihydro-5H-naphtho[2,1-f]indole, 4,5-dihydro-3H-naphtho[2,1-e]indole, 6,7-dihydro-3H-naphtho[2,1-g]indole, 5,6-dihydrophenanthro[3,2-d]thiazole, 4,5-dihydrophenanthro[2,1-d]thiazole, 6,7-dihydrophenanthro[4,3-d]thiazole, 5,6-dihydrophenanthro[3,2-d]oxazole, 4,5-dihydrophenanthro[2,1-d]oxazole, 6,7-dihydrophenanthro[4,3-d]oxazole, 5,6-dihydronaphtho[2,1-g]quinoline, 7,8-dihydronaphtho[2,1-h]quinoline, 5,6-dihydronaphtho[2,1-f]quinoline, 5,6-dihydro-8λ2-phenanthro[3,2-d]imidazole, 4,5-dihydro-3λ2-phenanthro[2,1-d]imidazole, 6,7-dihydro-3λ2-phenanthro[4,3-d]imidazole, 5,6-dihydronaphtho[1,2-g]quinoxaline, 5,6-dihydronaphtho[2,1-f]quinoxaline, 7,8-dihydronaphtho[1,2-f]quinoxaline, phenyl, 3,5,6,8-tetrahydrophenanthro[2,3-e:7,6-e′]diindole, 5,6-dihydropentapheno[3,4-d:10,9-d′]bis(oxazole), 5,6-dihydropentapheno[3,4-d:10,9-d′]bis(thiazole), 6,7-dihydrophenanthro[2,3-f:7,6-f]diquinoline, 5,6-dihydropentapheno[3,2,1-cd:10,11,12-c′d′]diindole, 2,6,7,11-tetrahydrobenzo[1,2-g:4,3-g′]dichromene, 2,6,7,11-tetrahydrobenzo[1,2-g:4,3-g′]bis(thiochromene), and 9,10-dihydro-8H-thieno[2′,3′,4′,5′:4,5]phenanthro[2,1-b]pyrrole 4,4-dioxide.

In some embodiments,
D is

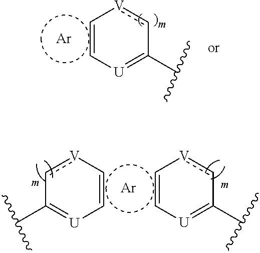

or J is

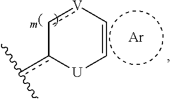

wherein at least one

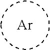

is a

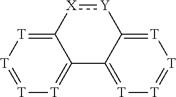

group or derivative thereof, which is fused to

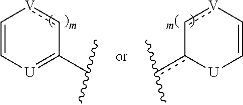

at any available

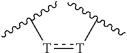

position on the

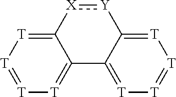

group or derivative thereof. The derivative of

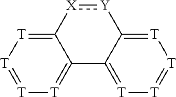

can comprise an additional aryl or heteroaryl group fused at any available

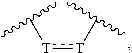

optionally

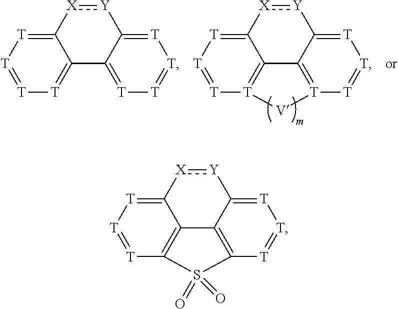

wherein V′ is SO₂, SO, S, NR¹¹, CR¹¹, C(R¹¹)₂, O, Si(R¹¹)₂, >C═O, >Se═O, —CH═CH—, or —N═CH—, and T, V, X, Y, and m are as defined herein above.

In some embodiments, D is

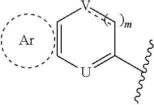

and the

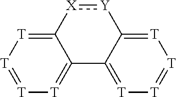

group or derivative thereof is fused to

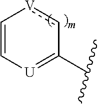

at any available

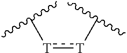

position on the

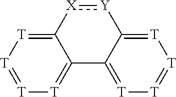

group or derivative thereof.

In some embodiments, D is selected from the group consisting of:

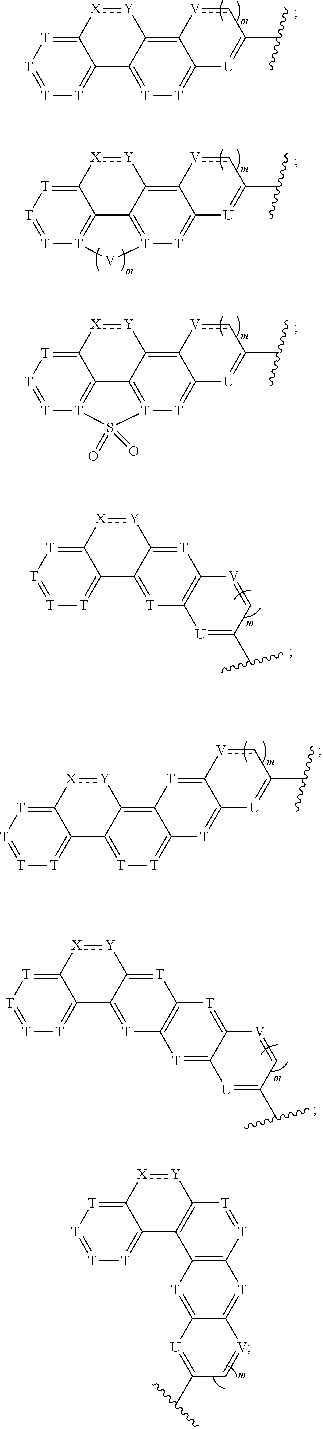

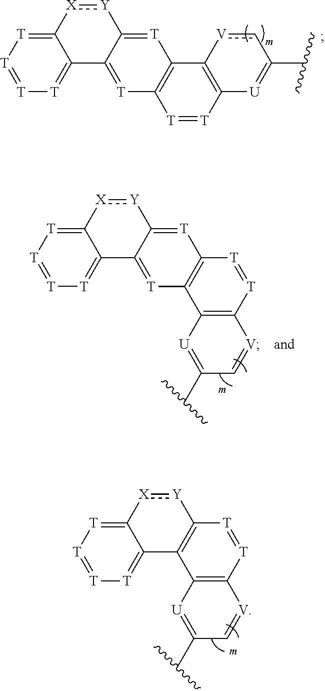

In some embodiments, D is

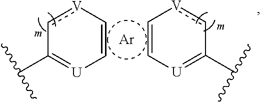

wherein the

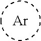

### of D is a

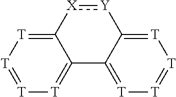

group or derivative thereof, which is fused to

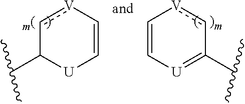

at any available

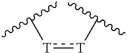

positions on the

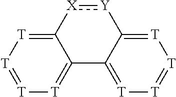

group or derivative thereof.

In some embodiments, J is

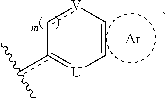

wherein the

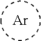

group is a

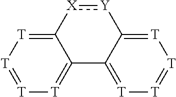

group or derivative thereof that is fused to

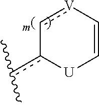

at any available

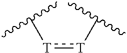

position on the

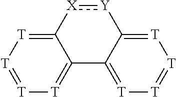

group or derivative thereof. The derivative of

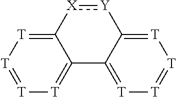

can comprise an additional aryl or heteroaryl group fused at any available

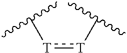

optionally

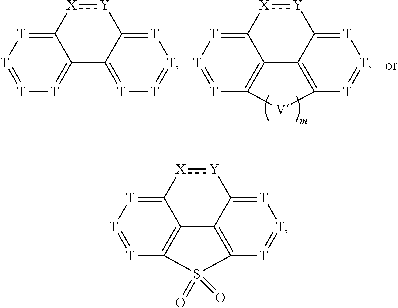

wherein V′ is SO₂, SO, S, NR¹¹, CR¹¹, C(R¹¹)₂, O, Si(R¹¹)₂, >C═O, >Se═O, —CH═CH—, or —N═CH—, and T, V, X, Y, and m are as defined herein above.

In some embodiments, J is selected from the group consisting of

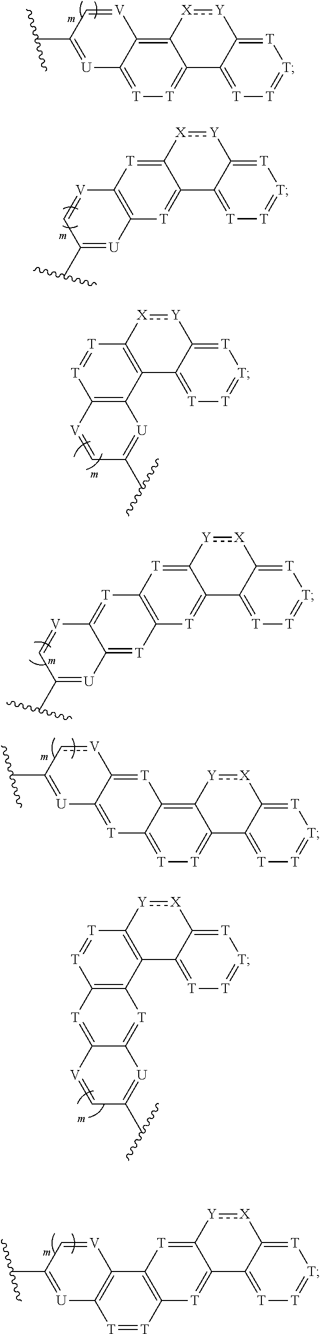

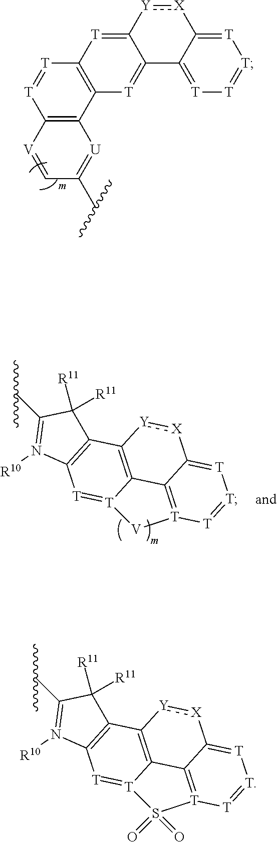

In some embodiments,
the at least one

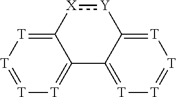

group or derivative thereof is selected from the group consisting of

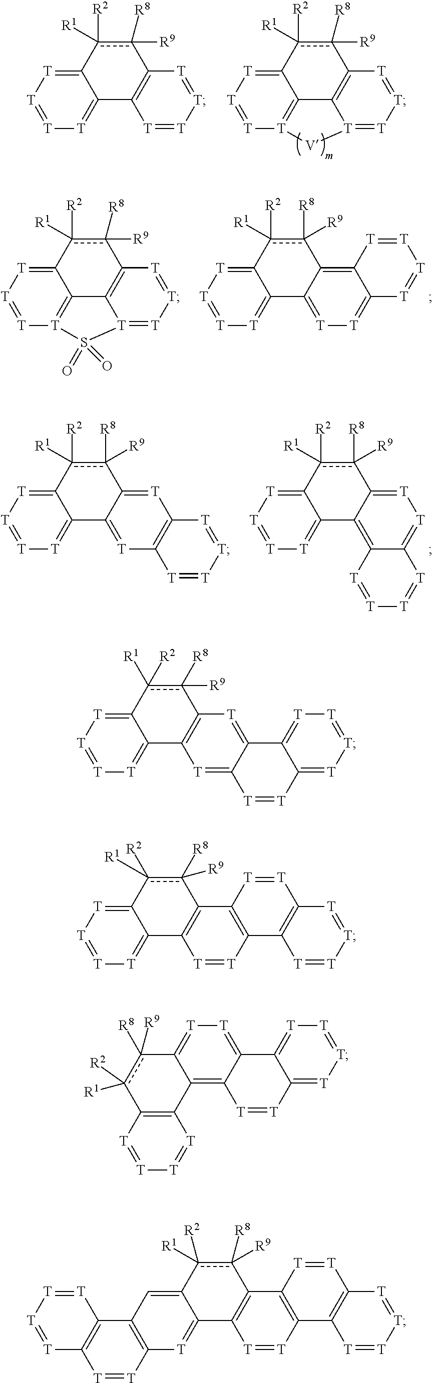

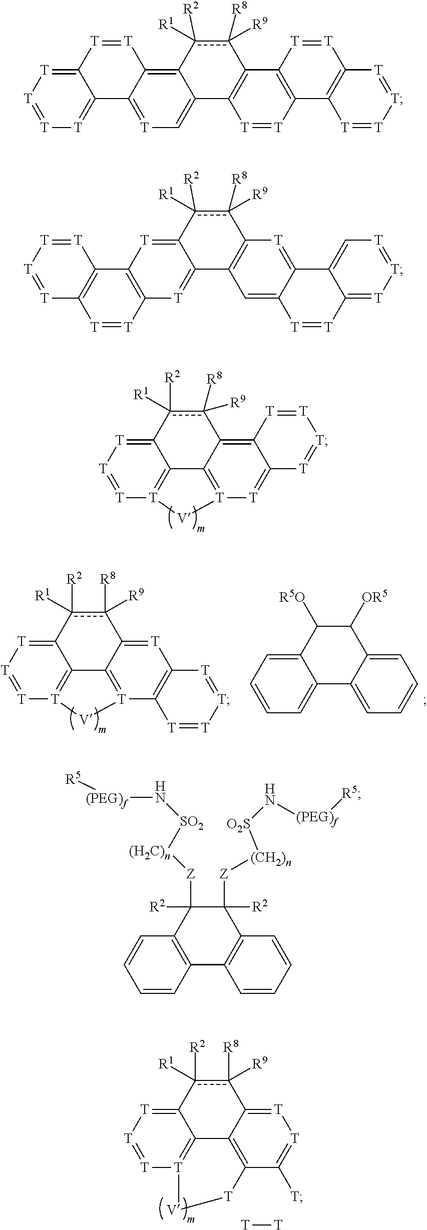

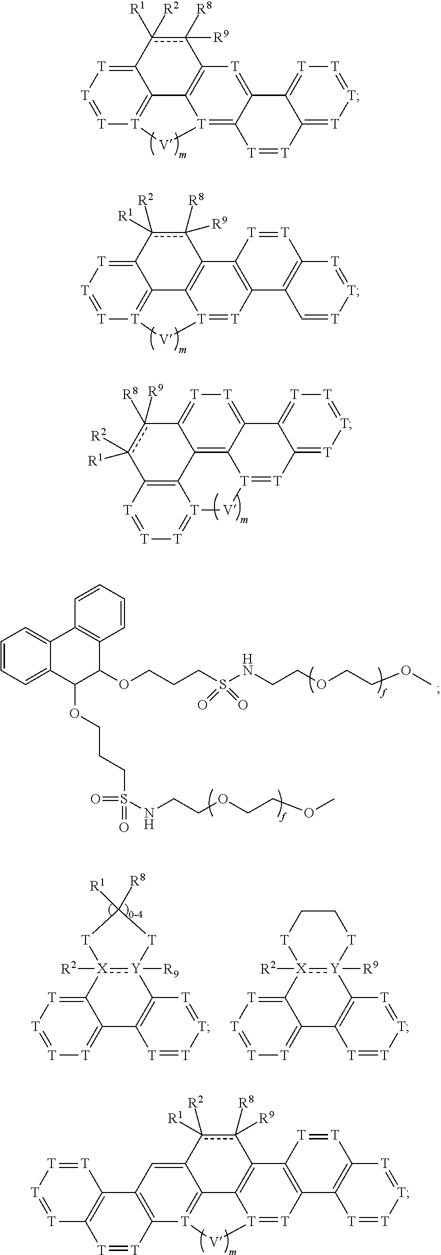

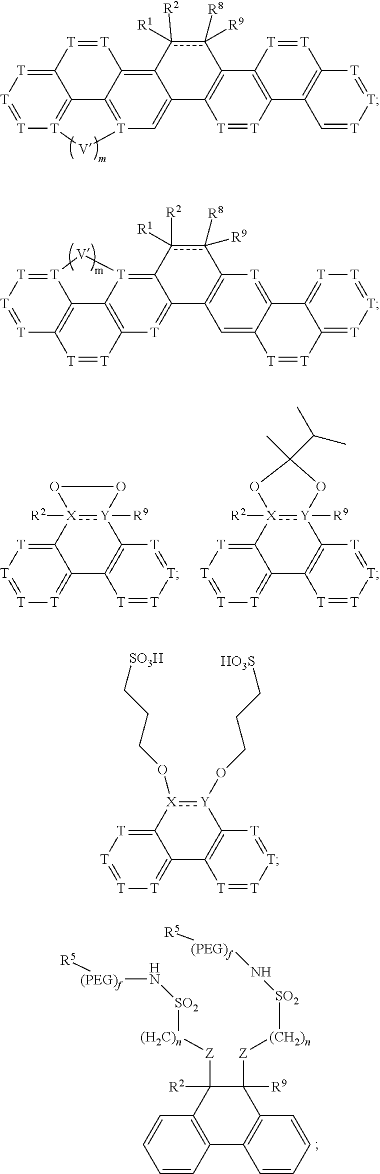

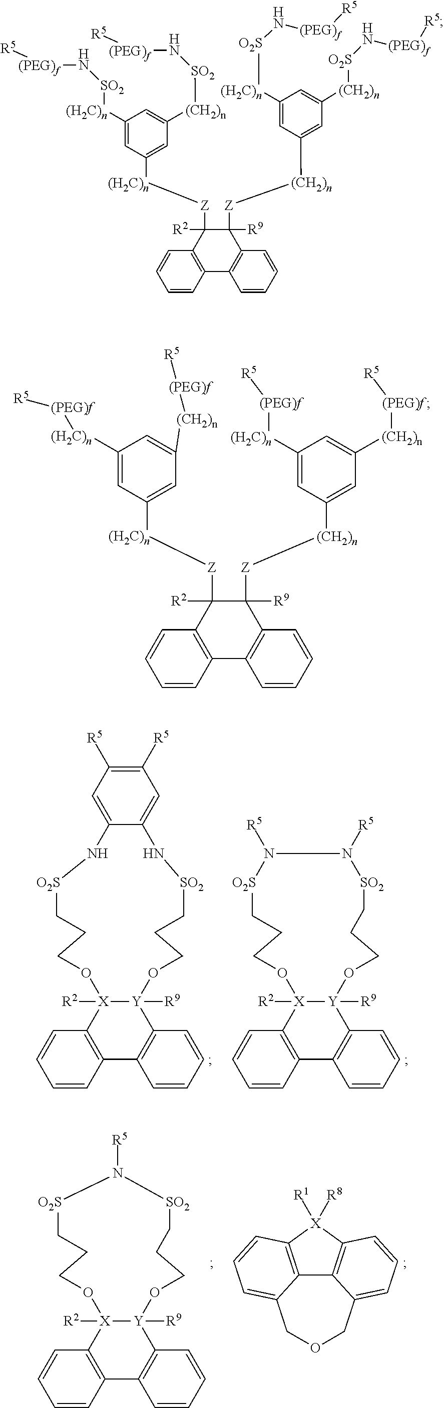

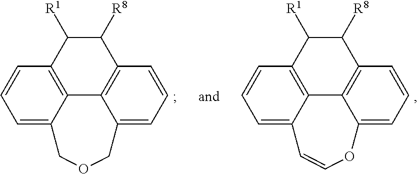

wherein each R⁵ is independently selected from the group consisting of halogen, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, a C₂-C₁₈ (hetero)aryl group, C₂-C₁₈ (hetero)aryloxy, C₂-C₁₈ (hetero)arylamino, carboxylic acid, carboxylate ester, (CH₂)_{x′}(OCH₂—CH₂)_{y′}OCH₃, and (CH₂)_{x′}(OCH₂—CH₂)_{y′}OCF₃, where each x′ is independently an integer from 0-20, and each y′ is independently an integer from 0-50.

In some embodiments, D is selected from the group consisting of

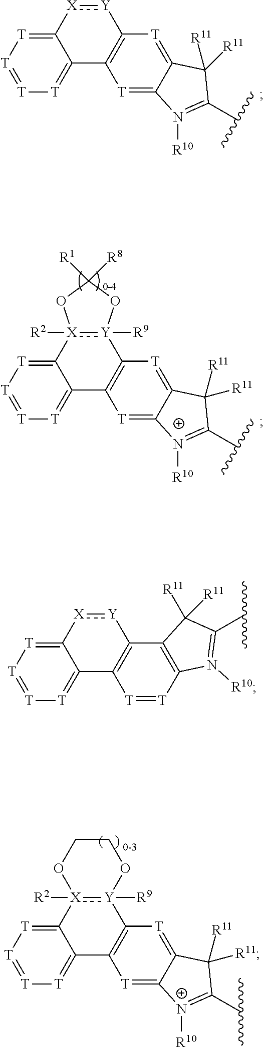

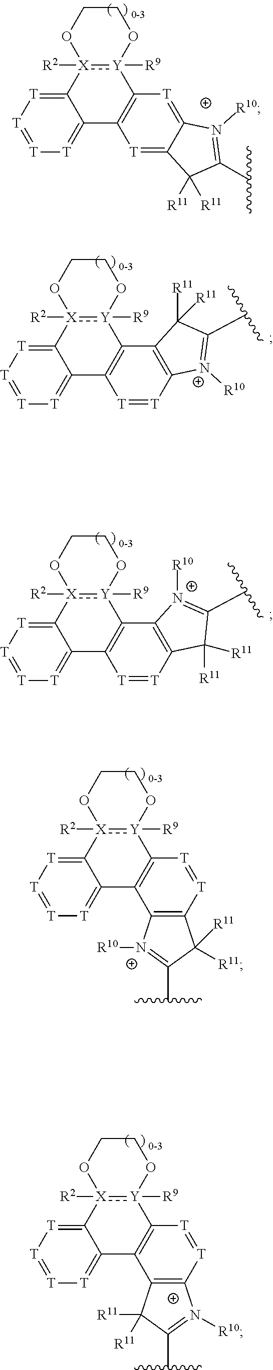

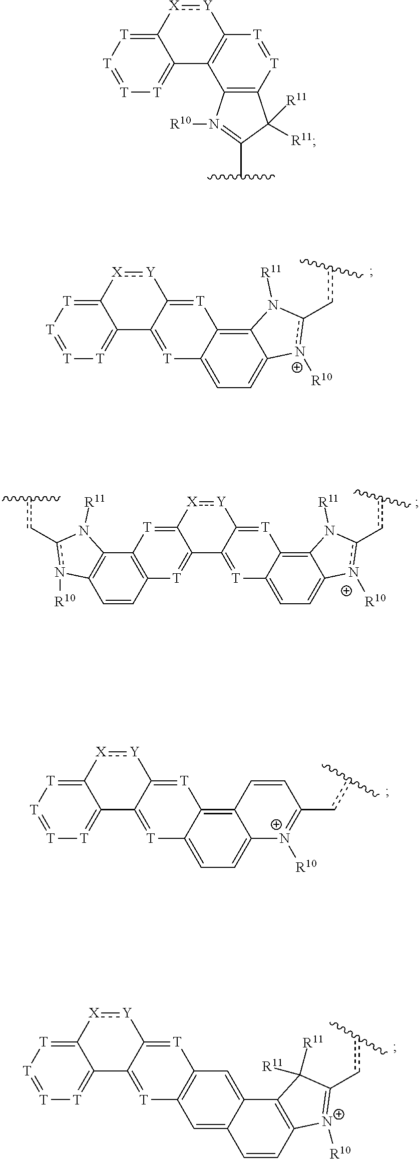

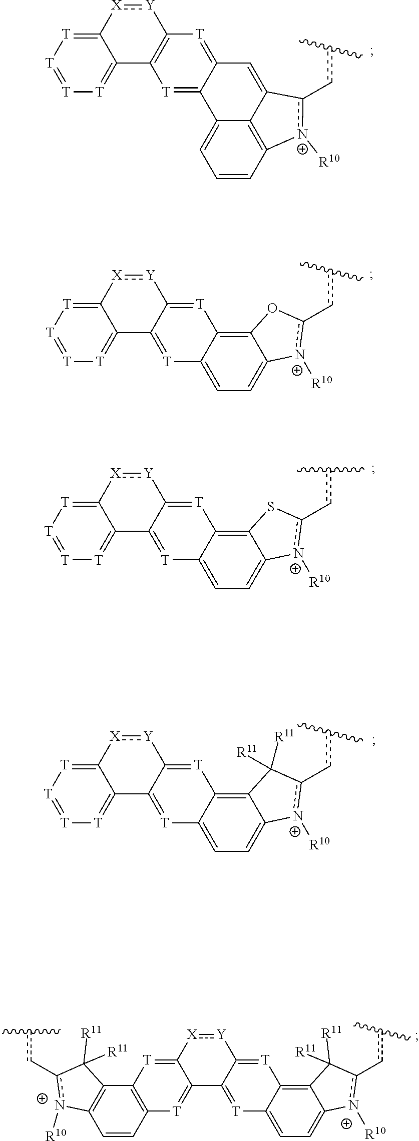

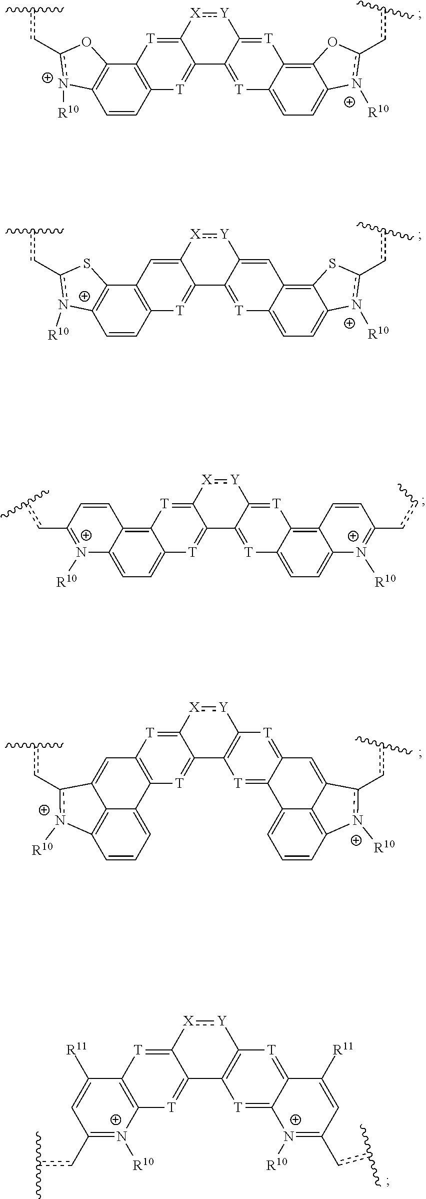

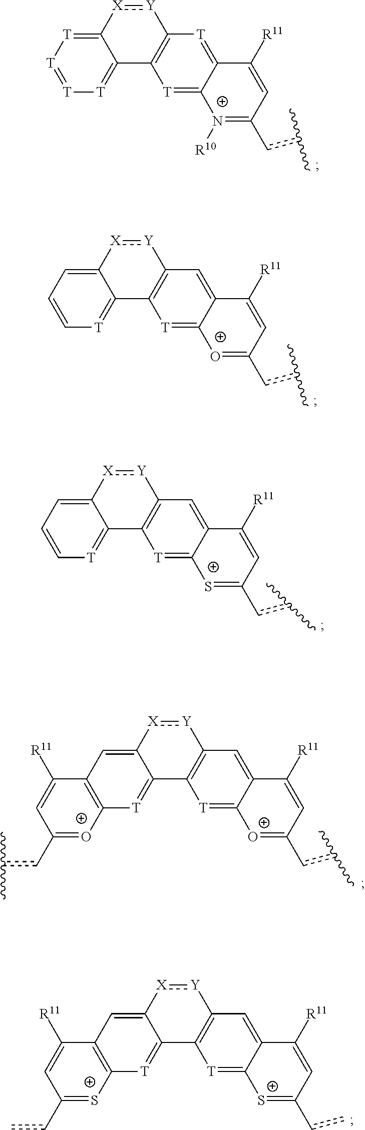

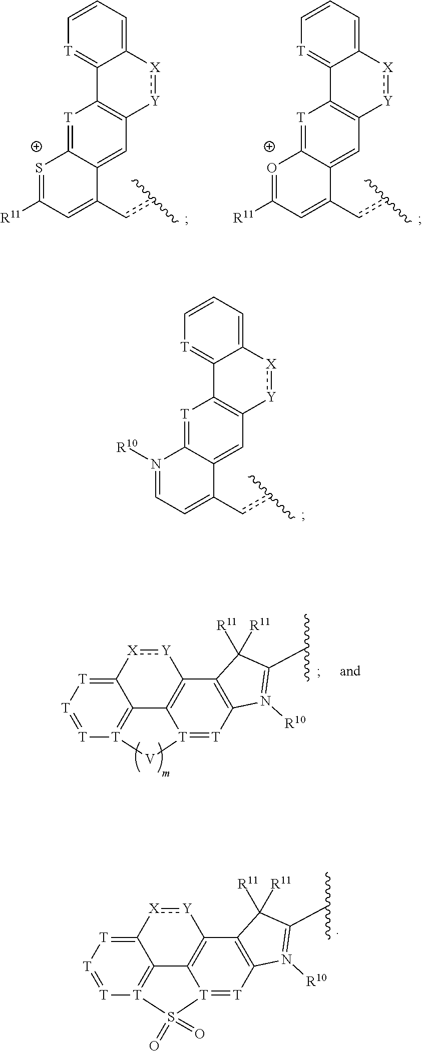

In some embodiments, J is selected from the group consisting of

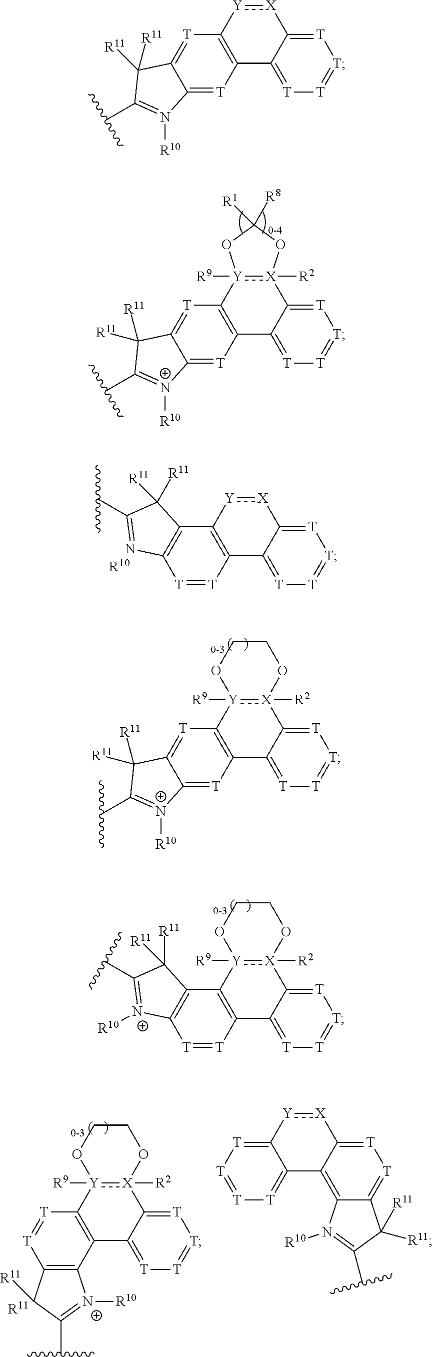

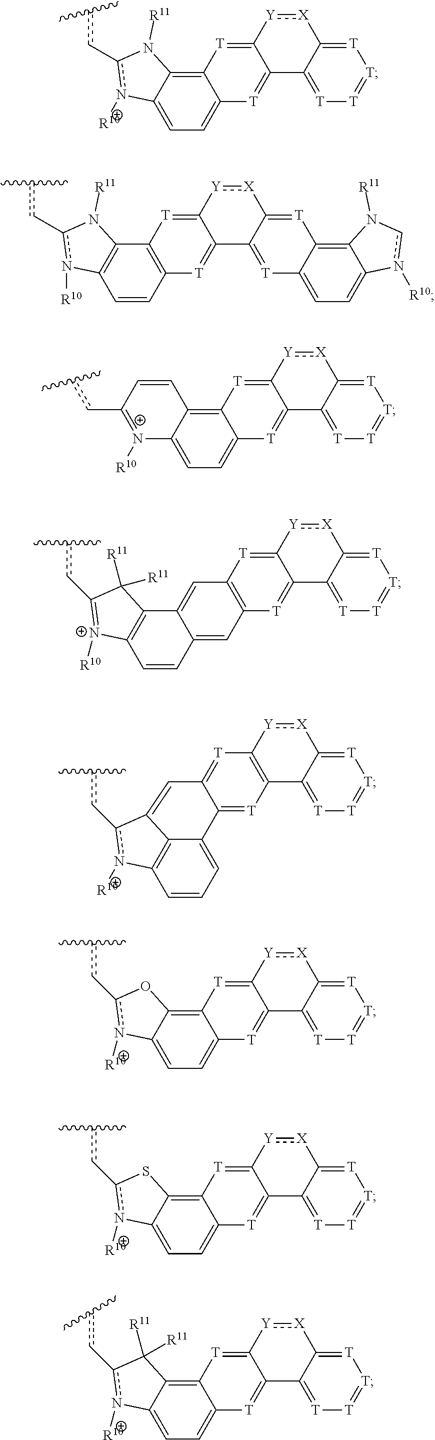

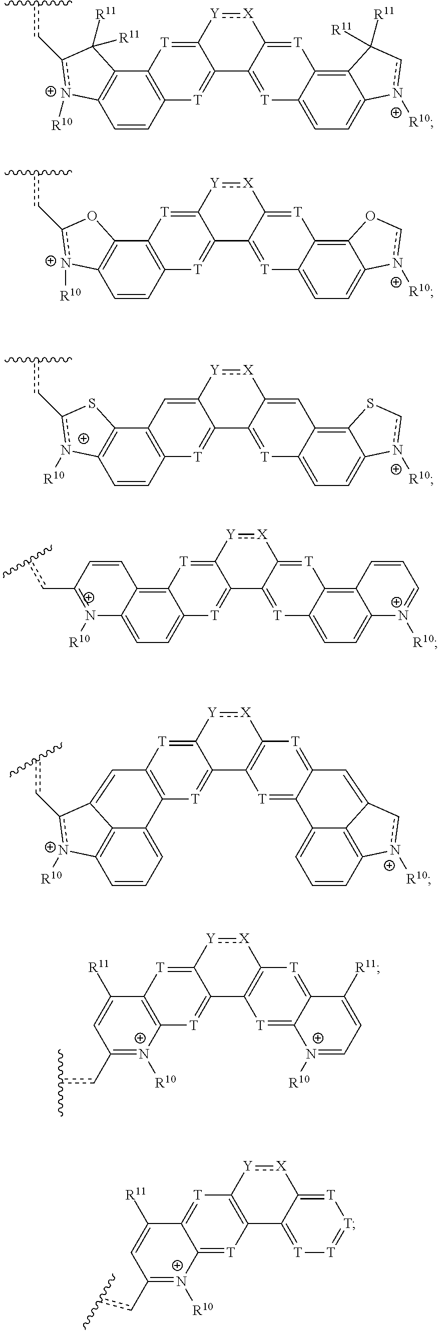

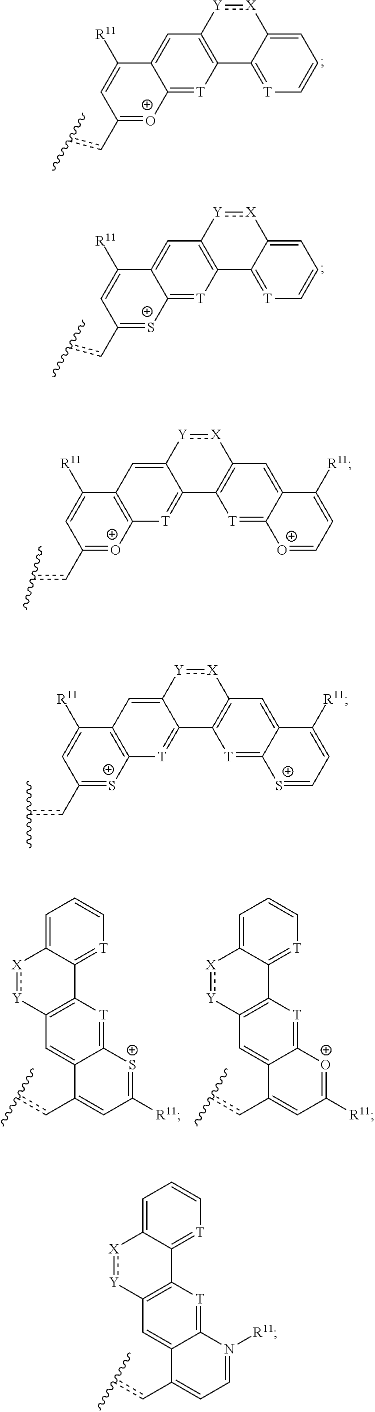

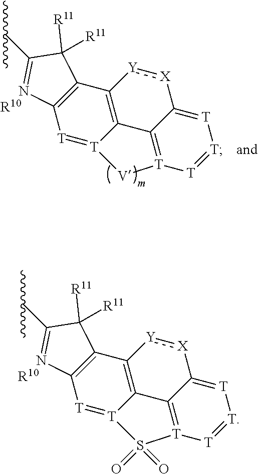

In some embodiments, R¹⁰ is selected from the group consisting of a water-solubilizing moiety, a linker moiety, a linked water-solubilizing moiety, hydrogen, alkyl, alkene, alkyne, cycloalkyl, haloalkyl, (hetero)aryloxy, aryl, heteroaryl, (hetero)arylamino, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide, alkoxy sulfonamide PEG, alkoxy sulfonate, alkyl sulfonate, alkyl sulfonate salt,

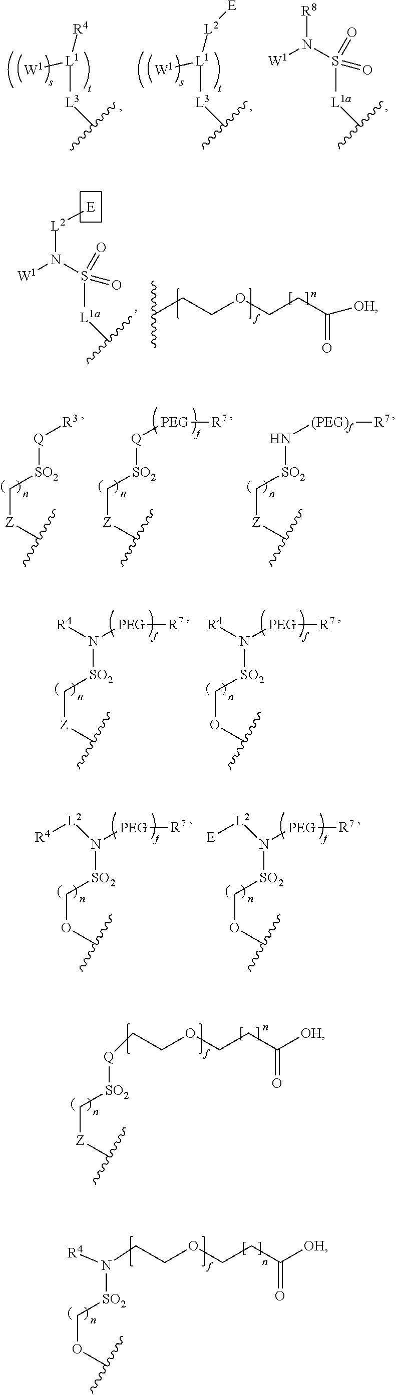

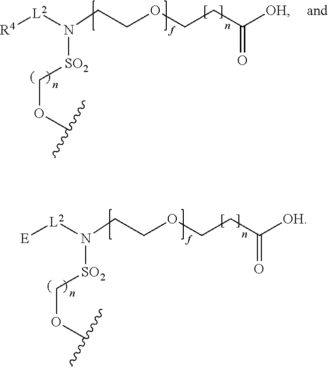

In some cases, a fluorescent dye is provided comprising a structure according to any one of

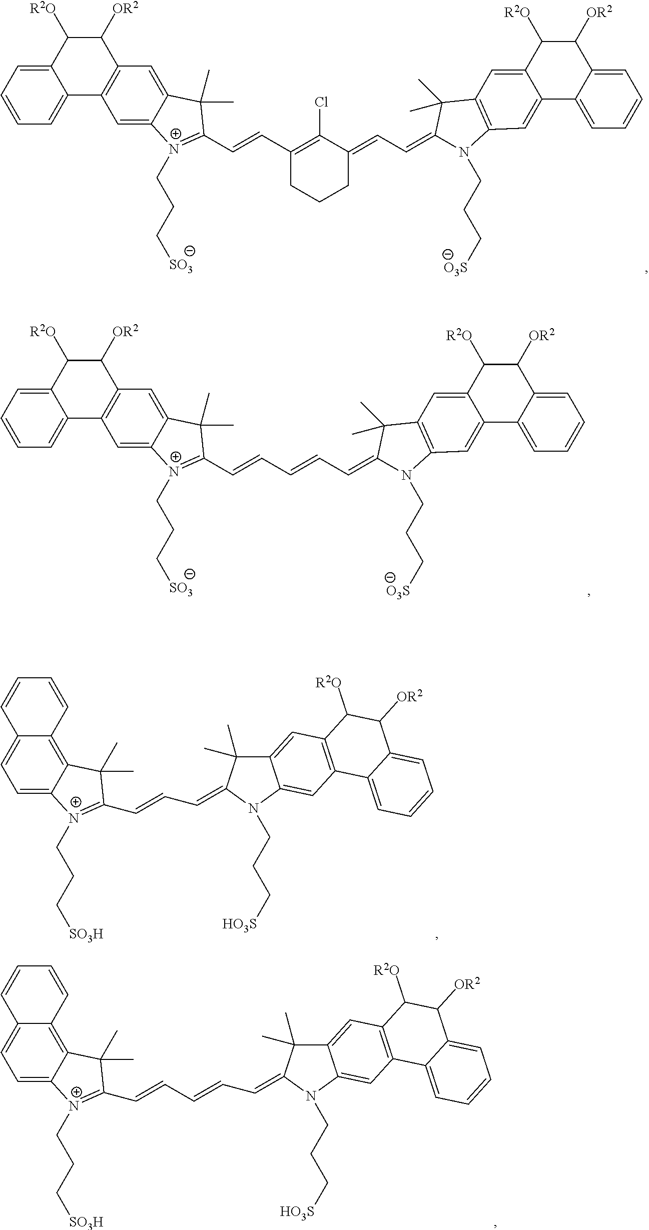

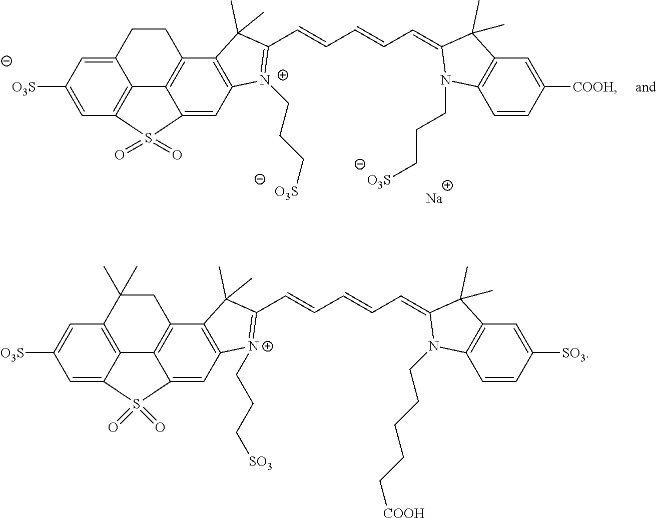

In some cases, an acceptor dye is provided comprising a DHP-cyanine compound having a structure according to formula (II).

The disclosure provides a polymer dye comprising a monomer having a structure according to Formula (IV) or (V):

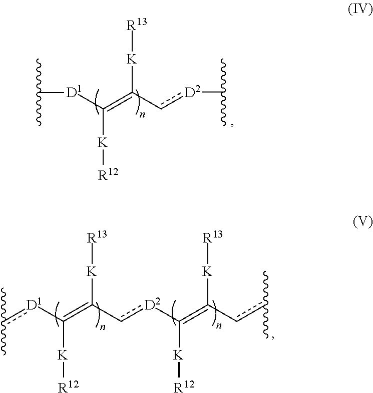

wherein each [symbol] in (IV) or (V) is a point of attachment to the polymer dye backbone; each D¹ and D² is independently selected from the group consisting of an aryl group, heteroaryl group, and

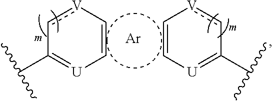

wherein each

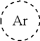

is independently selected from the group consisting of a substituted or unsubstituted benzene, benzene derivative, monocyclic aryl group, polycyclic aryl group, monocyclic heteroaryl group, and polycyclic heteroaryl group, with at least one of D¹ or D² being

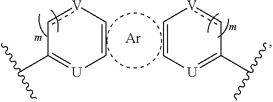

wherein

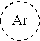

is

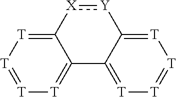

or a derivative thereof comprising an additional aryl or heteroaryl group fused at any available

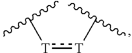

and wherein the

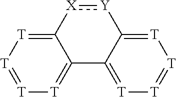

group or derivative thereof is fused to

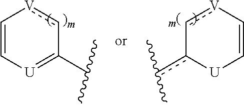

at any available

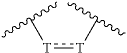

position
on the

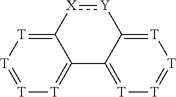

group or derivative thereof; and each of T, U, V, X, Y, R¹, R², R³, R⁴, R⁷, R⁸, R⁹, R¹⁰, R¹¹, R¹², R¹³, Q, Z, W¹, L¹, L², L³, E, k, f, n, m, n, s, t is as described herein. The derivative of

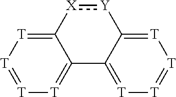

can comprise an additional aryl or heteroaryl group fused at any available

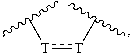

optionally

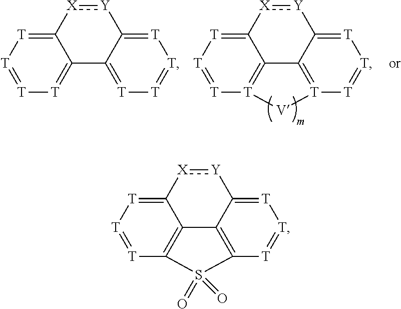

wherein V′ is SO₂, SO, S, NR¹¹, CR¹¹, C(R¹¹)₂, O, Si(R¹¹)₂, >C═O, >Se═O, —CH═CH—, or —N═CH—, and T, V, X, Y, and m are as defined herein above.

In some embodiments, the monomer of Formula (IV) or (V) comprises a structure selected from the group consisting of Formula (A1), (A2), (A3), (A4), (A5), (A6), (A7), (A8), (A9), (A10), (A11), (A12), (A13), (A14), (A15), (A16), (A17), (A18), and (A19):

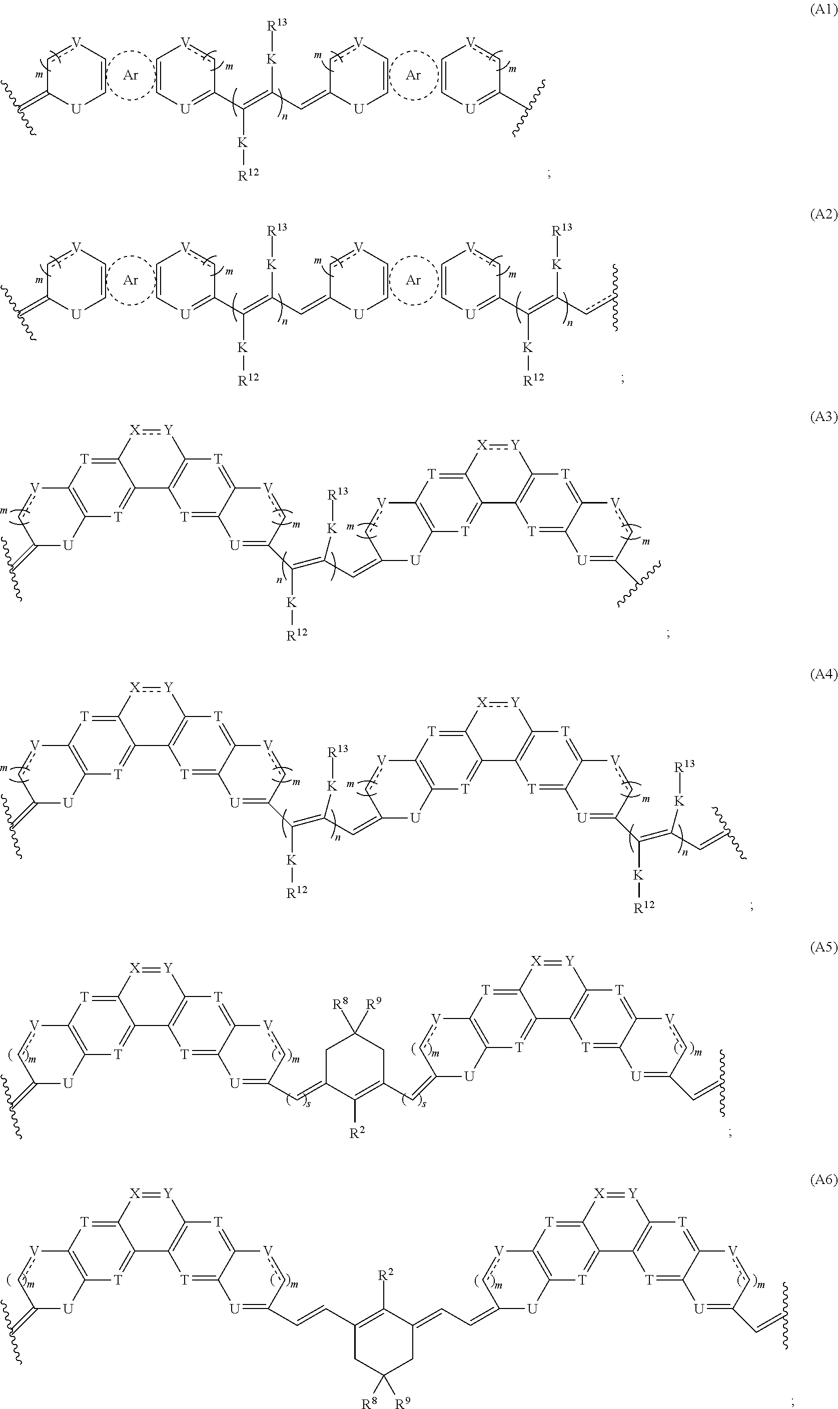

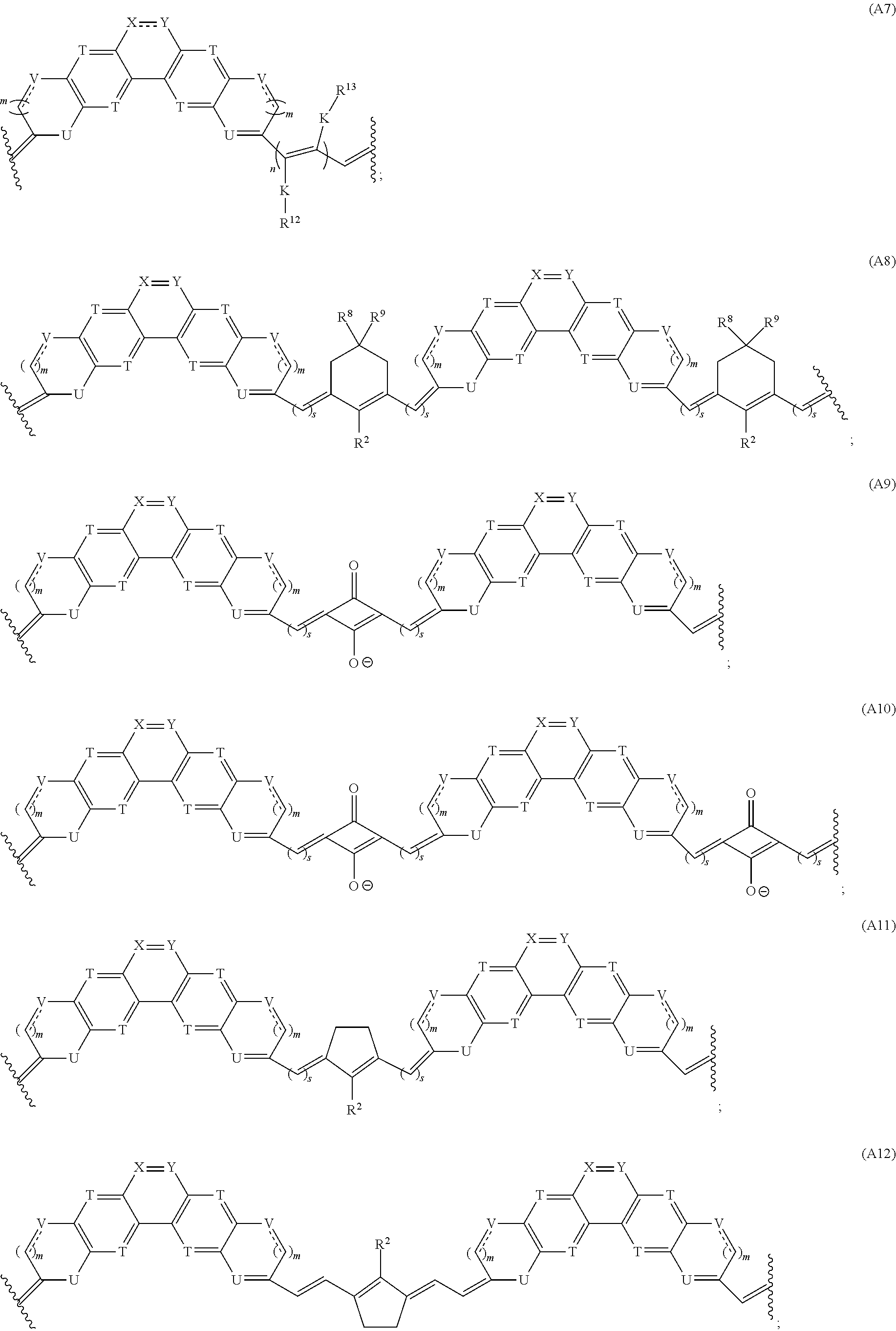

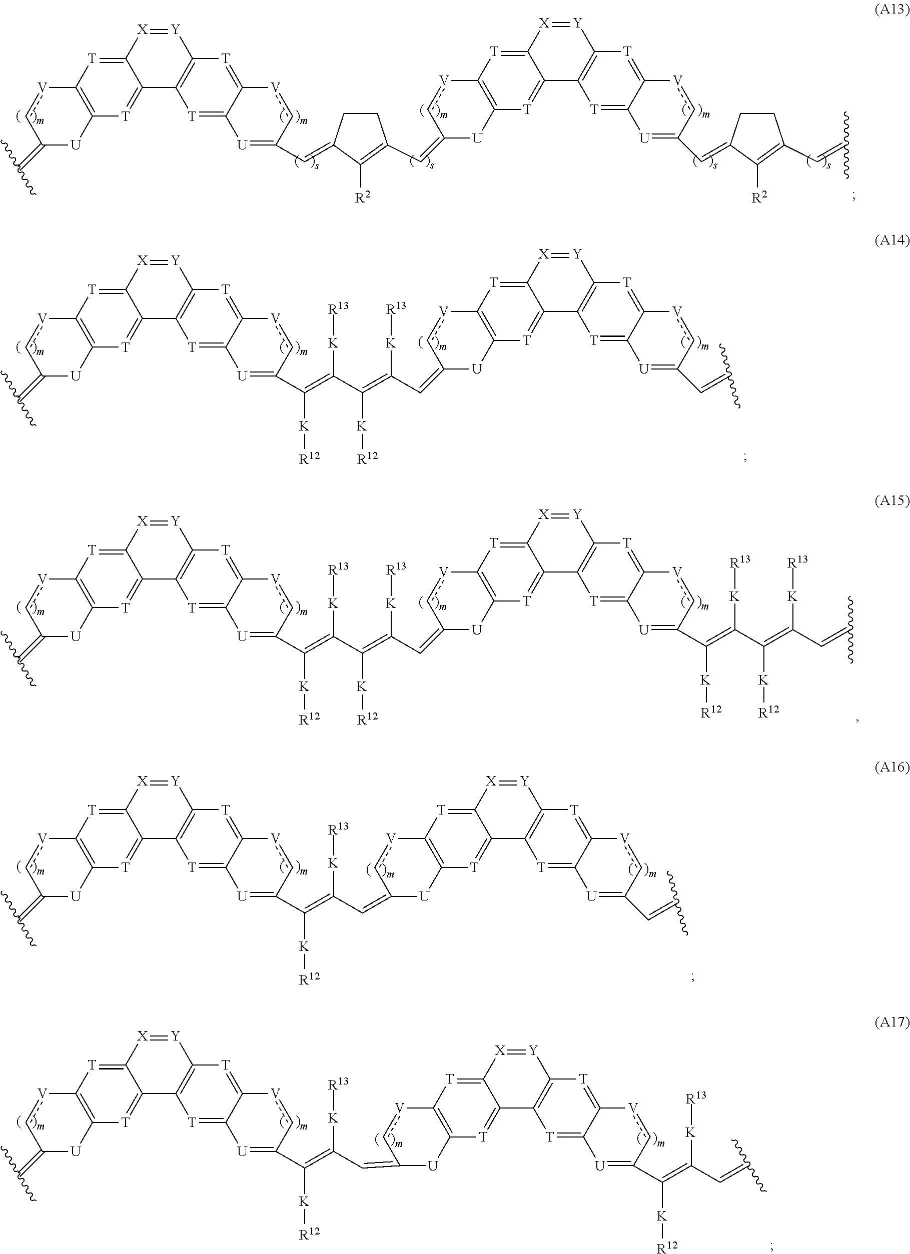

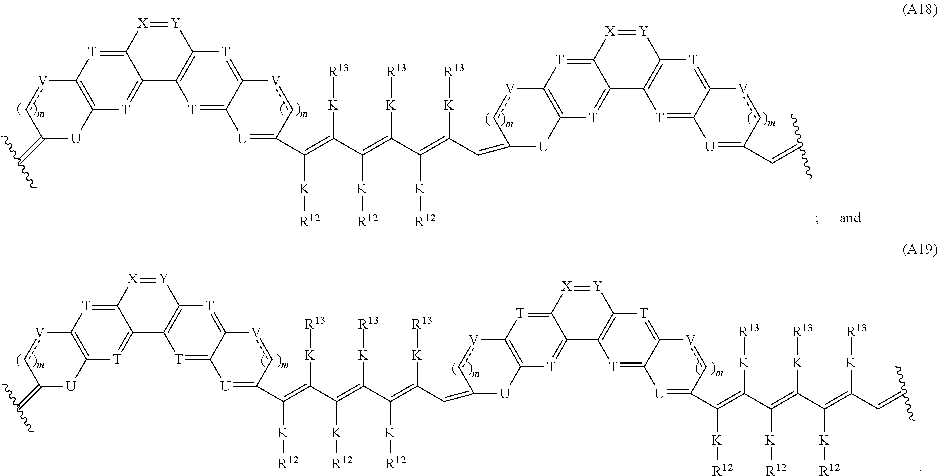

The disclosure provides a polymer dye comprising a structure according to Formula (VI) or (VII):

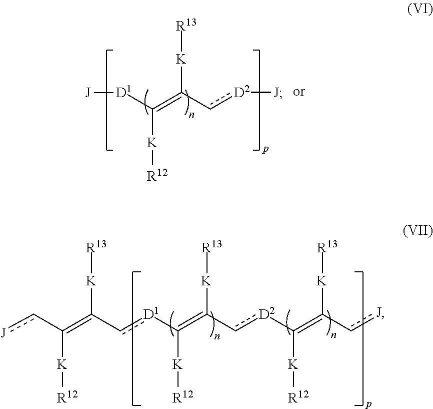

wherein
each D¹ and D² is independently selected from the group consisting of an aryl group, heteroaryl group, and

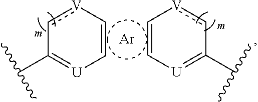

wherein each

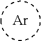

is independently selected from the group consisting of a substituted or unsubstituted benzene, benzene derivative, monocyclic aryl group, polycyclic aryl group, monocyclic heteroaryl group, and polycyclic heteroaryl group, with at least one of D¹ or D² being

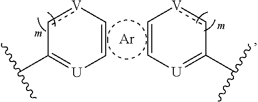

wherein

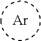

is

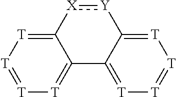

or a derivative thereof comprising an additional aryl or heteroaryl group fused at any available

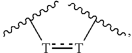

and wherein the

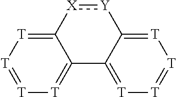

group or derivative thereof is fused to

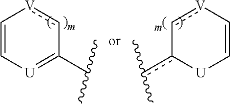

at any available

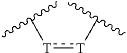

position on the

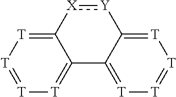

group or derivative thereof;
each J is independently aryl, heteroaryl,

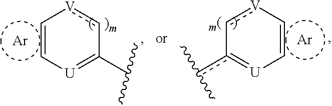

wherein

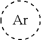

is a substituted or unsubstituted benzene, benzene derivative, monocyclic aryl group, polycyclic aryl group, monocyclic heteroaryl group, and polycyclic heteroaryl group; and p is an integer from 2 to 1,000; 5 to 500; or 10 to 100. Optionally at least one J group is

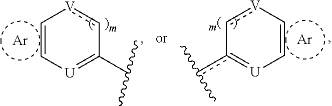

wherein the

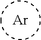

group is a

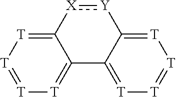

group or derivative thereof comprising an additional aryl or heteroaryl group fused at any available

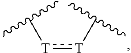

and wherein the

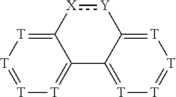

group or derivative thereof is fused to

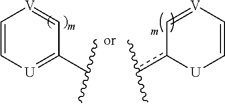

at any available

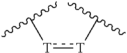

position on the

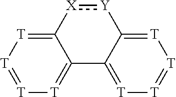

group or derivative thereof.

The disclosure provides a polymer dye having the structure of Formula (VI′):

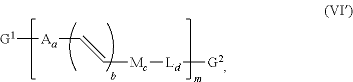

wherein
A is a monomer comprising a structure a [symbol] Formula (IV) or (V)

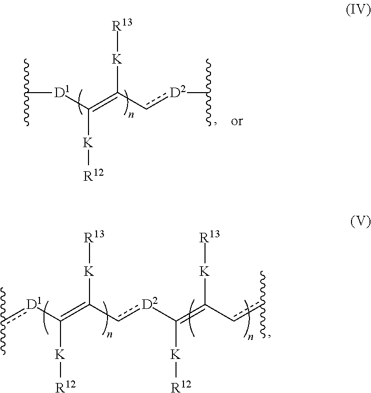

wherein
each [symbol] in formula (IV) or (V) is a point of attachment to the polymer dye backbone; each D¹ and D² is independently selected from the group consisting of an aryl group, heteroaryl group, and

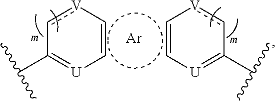

wherein each

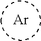

is independently selected from the group consisting of a substituted or unsubstituted benzene, benzene derivative, monocyclic aryl group, polycyclic aryl group, monocyclic heteroaryl group, and polycyclic heteroaryl group, with at least one of D¹ or D² being

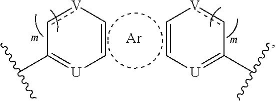

wherein

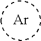

is

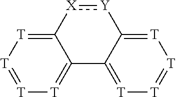

or a derivative thereof comprising an additional aryl or heteroaryl group fused at any available

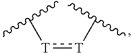

and wherein the

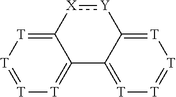

group or derivative thereof is fused to

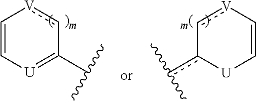

at any available

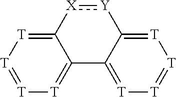

position on the

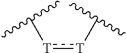

group or derivative thereof;
each optional M is a polymer modifying unit evenly or randomly distributed along the polymer main chain and is optionally substituted with one or more optionally substituted R¹, R², R³, or R⁴ groups;
each optional L is a linker;
G¹ and G² are each independently selected from an unmodified polymer terminus and a modified polymer terminus, optionally conjugated to E;
a, c, and d define the mol % of each unit within the structure which each can be evenly or randomly repeated and where each a is a mol % from 10 to 100%, each c is a mol % from 0 to 90%, and each d is a mol % from 0 to 25%;
each b is independently 0 or 1;
   p is an integer from 1 to about 10,000; and each of T, U, V, X, Y, R¹, R², R³, R⁴, R⁷, R⁸, R⁹, R¹⁰, R¹, R¹², R¹³, Q, Z, W¹, L¹, L², L³, E, k, f, n, m, n, s, t is as described herein.

In some embodiments, G¹ and G² is independently selected from the group consisting of hydrogen, halogen, alkyne, optionally substituted aryl, optionally substituted heteroaryl, halogen substituted aryl, silyl, diazonium salt, triflate, acetyloxy, azide, sulfonate, phosphate, boronic acid substituted aryl, boronic ester substituted aryl, boronic ester, boronic acid, optionally substituted tetrahydropyrene (THP), optionally substituted fluorene, optionally substituted dihydrophenanthrene (DHP), aryl or heteroaryl substituted with one or more pendant chains terminated with a functional moiety selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof, optionally conjugated to a substrate, or a binding partner.

In some embodiments, D¹ and D² are the same. In some embodiments, D¹ and D² are different.

In some embodiments, at least one of or at least two of R¹, R², R³, R⁴, R⁸, R⁹, R¹⁰, and R¹¹ comprises a water-solubilizing moiety or a linked water-solubilizing moiety.

The disclosure provides a labeled specific binding partner, comprising a fluorescent compound or polymer according to the present disclosure; and a specific binding partner covalently linked to the fluorescent compound or polymer. The specific binding partner may be selected from the group consisting of a protein, peptide, affinity ligand, antibody, antibody fragment, carbohydrate, lipid, nucleic acid, and an aptamer. The specific binding partner may be an antibody. The specific binding partner may be an antibody specific for a target analyte.

The disclosure provides a tandem dye, comprising: the fluorescent compound, polymer, or labeled specific binding partner according to the present disclosure; and an acceptor chromophore covalently linked to the fluorescent compound, polymer, or labeled specific binding partner. In some cases, the fluorescent compound of the invention is an acceptor dye.

The fluorescent compound, polymer, labeled specific binding partner, or tandem dye may be water-soluble.

A method is provided for detecting a target analyte in a sample comprising: providing a sample that is suspected of containing the analyte; and contacting the sample with a specific binding partner conjugated to a water-soluble fluorescent compound, polymer, or tandem dye according to the present disclosure, wherein the binding partner is capable of interacting with the target analyte.

The method may include one or more of wherein the method is configured for flow cytometry; the water-soluble fluorescent polymer is bound to a substrate; the analyte is a protein expressed on a cell surface; the method is configured as an immunoassay; and/or the method further comprises providing additional binding partners for detecting additional analytes simultaneously.

The disclosure provides a kit comprising at least one fluorescent compound, polymer, labeled specific binding partner, or tandem dye according to the present disclosure. The compound, polymer or tandem dye according to the disclosure may include a conjugation tag.

### BRIEF DESCRIPTION OF THE DRAWINGS

FIG. 1 shows synthetic scheme 1 for production of DHP-indole, DHP-thiazole, DHP-oxazole, and DHP-quinoline intermediates which can be prepared from a bromo-DHP core molecule and converted to DHP-cyanine or DHP-squaraine compounds. The dashed-line circle represents the heterocyclic ring portion of the intermediate precursor.

FIG. 2 shows synthetic scheme 2 for modifying dihydrophenanthrene followed by synthesis of a DHP-Cy5 type monomeric dye.

FIG. 3 shows synthetic scheme 3 for preparing DHP-cyanine polymeric dyes from dihydrophenanthrene core. DHP-cyanine and DHP-squaraine polymeric dyes can exhibit excitation λmax in a range of from 420 to 900 nm.

FIG. 4 shows synthetic scheme 4 to modify dihydrophenanthrene followed by synthesis of a polymeric DHP-Cy5 dye.

FIG. 5 shows synthetic scheme 5 for preparing an asymmetric DHP-squaraine monomeric dye from a dihydrophenanthrene type core and a semisquaraine; and synthetic scheme 6 for preparing a symmetric DHP-squaraine monomeric dye from a dihydrophenanthrene type core and squaric acid.

FIG. 6 shows synthetic scheme 7 for preparing N-propyl sulfonate DHP-indole intermediate compound 21 from dihydrophenanthrene.

FIG. 7 shows synthetic schemes 8 and 9 for preparing sulfoxide bridged DHP indole cyanine dye 25 from -propyl sulfonate DHP-indole intermediate compound 21.

FIG. 8 shows synthetic scheme 10 for preparing sulfoxide bridged DHP indole cyanine dye antibody conjugate 27 from sulfoxide bridged DHP indole cyanine dye 25.

FIG. 9 shows exemplary synthetic schemes 12 and 13 for preparing and purifying a DHP-cyanine-antibody conjugate or a polymer-antibody conjugate, respectively.

FIG. 10 shows exemplary synthetic scheme 14 for preparing sulfoxide bridged DHP indole cyanine dye 36 from N-propyl sulfonate DHP-indole intermediate compound 33 which is prepared from chloroaminobenzoic acid 28.

### DETAILED DESCRIPTION OF THE INVENTION

### I. General

The present disclosure provides novel DHP-cyanine and DHP-squaraine fluorescent compounds and polymers thereof. In some embodiments, DHP-cyanine and DHP-squaraine fluorescent compounds and polymer dyes have been designed to be water soluble. The present disclosure also provides labeled specific binding partners comprising DHP-cyanine and DHP-squaraine fluorescent compounds and polymers thereof. The disclosure also provides tandem dyes comprising the DHP-cyanine and DHP-squaraine fluorescent compounds and polymers thereof. The DHP-cyanine and DHP-squaraine fluorescent compounds can be an acceptor dye.

Methods are provided for detecting target analytes in a sample using fluorescent DHP-cyanine and DHP-squaraine compounds or polymers conjugated to binding partners. The various DHP-cyanine and DHP-squaraine fluorescent compounds and polymers of the present disclosure demonstrate water solubility and can be excited using UV, violet, blue, yellow, green, red, or NIR wavelengths. There is an increasing demand for a variety of fluorescent dyes for use in current flow cytometers as well as spectral flow instruments.

In the present disclosure, dihydrophenanthrene monomers were modified to include a fused heterocyclic ring. For example, DHP monomers were modified into electron rich indole containing DHP-indole moieties using Fischer Indole synthesis that produces the aromatic heterocyclic indole from a substituted phenylhydrazine and an aldehyde or ketone under acidic conditions. This can be carried out in two steps using bromo dihydrophenanthrene as the starting material, for example, as illustrated in Scheme 2. Later classic cyanine dye synthesis was performed on the DHP-indole ring to allow formation of DHP-cyanine dyes that can be excited using UV, violet, blue, green, red, or NIR light, depending on the number of carbon atoms in the linker molecule used for the cyanine synthesis.

Various chemical modifications of the core DHP-cyanine molecule allowed preparation of dyes which can be excited using different lasers. Both small organic dyes as well as polymer dyes were prepared using this approach. Other family of dyes such as squaraines were also prepared from the electron rich DHP ring core. Symmetric as well as unsymmetric dyes were made using this approach which allows fine tuning of the absorption and emission of the dyes. DHP-cyanine and DHP-squaraine dyes according to the present disclosure exhibit excitation maxima in a range of from about 400-900 nm for monomeric dyes.

Kits comprising at least one fluorescent DHP-cyanine compound, DHP-squaraine fluorescent compound or polymer thereof, labeled specific binding partner, or tandem dye according to the present disclosure are also provided. The fluorescent DHP-cyanine, DHP-squaraine compound, or polymer thereof, or tandem dye may include a conjugation tag.

### II. Definitions

The abbreviations used herein have their conventional meaning within the chemical and biological arts.

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls. In the methods described herein, the acts can be carried out in any order without departing from the principles of the present disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process. The term “about” as used herein can allow for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “reactive group” refers to a functional group that can selectively react with another compatible functional group to form a covalent bond, in some cases, after optional activation of one of the functional groups. Chemoselective functional groups of interest include, but are not limited to, thiols, maleimides, halogenated maleimides, iodoacetamides, amines, alkyl carboxylates, alkyl sulfonates, carboxylic amines, carbamate, carboxylate esters, N-hydroxysuccinimidyl (NHS), imido ester, halogen, boronic esters, boronic acids, hydrazonyl, carboxylic acids or active esters thereof, as well as groups that can react with one another via Click chemistry, e.g., azide and alkyne groups (e.g., cyclooctyne groups), tetrazine and alkene groups (e.g., cyclooctene groups), dienes and dienophiles, sulfur (VI) fluoride exchange chemistry (SuFEX), sulfonyl fluoride, as well as hydroxyl, hydrazido, hydrazino, aldehyde, ketone, azido, alkyne, phosphine, epoxide, and the like, or protected groups thereof. The reactive group may be a conjugation tag. The chemoselective functional group may be protected or unprotected.

The term “amine-reactive group” refers to any group that forms a chemical bond with a primary amine. Amine-reactive groups of interest include, but are not limited to, isothiocyanates, isocyanates, acyl azides, NHS esters, imidoesters, sulfonyl chlorides, aldehydes, glyoxals, epoxides, oxiranes, carbonates, aryl halides, imidoesters, carbodiimides, anhydrides, and fluorophenyl esters. The amine-reactive group can be a NHS ester or imidoesters.

In some cases, non-covalent linking may involve specific binding between two moieties of interest (e.g., two affinity moieties such as a hapten and an antibody or a biotin moiety and a streptavidin, etc.). In certain cases, non-covalent linking may involve absorption to a substrate.

The term “symmetric” in reference to a compound of the present disclosure refers to wherein each terminal heterocyclic ring system is the same, and substituents may be the same or different. In some symmetric compounds, n=2.

The term “asymmetric” in reference to a compound of the present disclosure refers to wherein each terminal heterocyclic ring system is different. In some asymmetric compounds, n=3.

The term “counterion” refers to an ion that is charge balancing to the fluorescent compound according to the disclosure. The counter ion may be a cation. The counterion may be an anion. In some cases the counterion may be a halogen ion, perchlorate ion, PF⁶⁻, phosphate ion, sulfate ion, and the like. The counterion may be, F⁻, Cl⁻, Br⁻, I⁻, ClO₄⁻, CF₃CO₂⁻, CH₃CO₂⁻, PO₄³⁻, SO₄²⁻, BF₄⁻, and the like. In some cases the counterion may be Na⁺, K⁺, Mg⁺⁺, Ca⁺⁺, and the like.

For example, DHP-cyanine dye or polymer-labeled antibodies according to the present disclosure find use in flow cytometry as reagents exhibiting a fluorescent signal. Additionally, orthogonal “functional group(s)” can be included that can be used for either bioconjugation of a binding partner to or the attachment of acceptor signaling chromophores in donor acceptor tandem dyes.

The term “organic group” as used herein refers to any carbon-containing functional moiety. Examples can include an oxygen-containing group such as an alkoxy group; aryloxy group; aralkyloxy group; oxo (carbonyl) group; an amine group, including alkyl amine amine esters, and sulfonamide groups; a carboxyl group including a carboxylic acid, carboxylate, and a carboxylate ester; a sulfur-containing group such as an alkyl and aryl sulfide group, thiol, thiol reactive group, and sulfone group; maleimide; iodoacetamide; azide group; alkyne group; and other heteroatom-containing groups. Non-limiting examples of organic groups include OR, OOR, OC(O)N(R)₂, CN, CF₃, OCF₃, R, C(O)R, methylenedioxy, ethylenedioxy, N(R)₂, N₃, S(H)R, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “heteroatom” as used herein refers to any appropriate atom that is not carbon, such as, for example, N, O, S, Se, P, B, Al, Si, and Ge, inserted between adjacent carbon atoms in an organic group. The organic group may be a cyclic, aryl, or straight or branched chain group (e.g., alkyl or alkene). More than one heteroatom (e.g., 1, 2, 3, 4 or 5 heteroatoms) may be inserted between adjacent carbon atoms. The heteroatoms can be oxidized to form moieties such as, but not limited to, —S(O)— and S(O)₂—, sulfinate, sulfonamide.

The term “substituted” as used herein in conjunction with a molecule or an organic group as defined herein refers to the state in which one or more hydrogen atoms contained therein are replaced by one or more non-hydrogen atoms, such as, for example an alkyl, aryl, or a functional group. The “substituted” group may include one or more groups selected from halogen, hydroxy, amino, alkylamino, amido, acyl, nitro, cyano, and alkoxy.

The term “functional group,” “functional moiety,” or “substituent” as used herein refers to a group that can be or is substituted onto a molecule or onto an organic group. Examples of substituents or functional groups include, but are not limited to, a halogen (e.g., F, Cl, Br, and I); alkene; cycloalkene; alkyne; cycloalkyne; an oxygen atom in groups such as hydroxy groups, alkoxy groups, aryloxy groups, aralkyloxy groups, oxo (carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, imides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl. The subject fluorescent compounds can include one or more “functional group(s)” (also referred to as a “conjugation tag”) that provide for bioconjugation a dye. In some cases, such functionality may be used to covalently attach a biomolecule or binding partner such as a protein, peptide, affinity ligand, antibody, antibody fragment, polynucleotide, or aptamer. In some cases the functional group or conjugation tag may be selected from the group consisting of amine, carbamate, carboxylic acid, carboxylate, maleimide, activated ester, N-hydroxysuccinimidyl, hydrazine, hydrazide, hydrazone, isothiocyanato, azide, alkyne, cycloalkyne (e.g., alkene, cycloalkene (e.g., cyclooctene), tetrazine, aldehyde, thiol, and protected groups thereof for conjugation to a substrate, acceptor dye, functional moiety, or binding partner. The functional group may be protected or unprotected. The functional group may be a reactive or chemoselective functional group that can react with another group via copper-free click chemistry, including strain-promoted azide-alkyne cycloaddition (SPAAC) and inverse-electron-demand Diels-Alder (iEDDA) reactions that enable fast and specific chemical conjugation. See Kim et al., Chem. Sci., 2019, 10, 7835; and Davis et al., J. Org. Chem. 2016, 81, 6816-6819, both incorporated herein by reference in their entireties. The functional group or conjugation tag can be, for example, cycloalkene (e.g., cyclooctene); alkyne; cycloalkyne (e.g., cyclooctyne group, such as, for example, bicyclo[6.1.0] nonyne (BCN)), Dibenzocyclooctyne (DBCO)); cycloalkene (e.g., cyclooctene group, such as, for example, trans-cyclooctene (TCO)); an azide group; or a tetrazine group.

As used herein, the term “activated ester” or “active esters” by itself or as part of another substituent refers to carboxyl-activating groups employed in peptide chemistry to promote facile condensation of a carboxyl group with a free amino group of an amino acid derivative. Descriptions of these carboxyl-activating groups are found in general textbooks of peptide chemistry, for example K. D. Kopple, “Peptides and Amino Acids”, W. A. Benjamin, Inc., New York, 1966, pp. 50-51 and E. Schroder and K. Lubke, “The Peptides”; Vol. 1, Academic Press, New York, 1965, pp. 77-128.

As used herein, the term “ammonium” by itself or as part of another substituent refers to a cation having the formula NHR₃⁺ where each R group, independently, is hydrogen or a substituted or unsubstituted alkyl, aryl, aralkyl, or alkoxy group. Preferably, each of the R groups is hydrogen.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein some or all the hydrogen atoms are substituted with other functional groups. The term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group. A hydrocarbylene group is a diradical hydrocarbon, e.g., a hydrocarbon that is bonded at two locations.

As used herein, the term “alkyl” by itself or as part of another substituent refers to a straight or branched, saturated, aliphatic radical having the number of carbon atoms indicated. Alkyl groups can be optionally substituted alkyl groups. For example, C₁-C₆ alkyl includes, but is not limited to, methyl, ethyl, propyl, isopropyl, butyl, isobutyl, sec-butyl, tert-butyl, pentyl, isopentyl, hexyl, etc. Other alkyl groups include, but are not limited to heptyl, octyl, nonyl, decyl, etc. Alkyl can include any number of carbons, such as 1-2, 1-3, 1-4, 1-5, 1-6, 1-7, 1-8, 1-9, 1-10, 2-3, 2-4, 2-5, 2-6, 3-4, 3-5, 3-6, 4-5, 4-6 and 5-6. Alkyl can also refer to alkyl groups having up to 20 carbons atoms, such as, but not limited to heptyl, octyl, nonyl, decyl, etc. Alkyl groups can be substituted or unsubstituted. Unless otherwise specified, “substituted alkyl” groups can be substituted with one or more groups selected from halo, hydroxy, amino, alkylamino, amido, acyl, nitro, cyano, and alkoxy. The alkyl group is typically monovalent, but can be divalent, such as when the alkyl group links two moieties together.

As used herein, the term “alkylene” refers to an alkyl group, as defined above, linking at least two other groups (i.e., a divalent alkyl radical). The two moieties linked to the alkylene group can be linked to the same carbon atom or different carbon atoms of the alkylene group.

As used herein, the term “alkoxy” by itself or as part of another substituent refers to an alkyl group, as defined above, having an oxygen atom that connects the alkyl group to the point of attachment. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. For example, the alkoxy groups can be substituted with halogens to form a “halo-alkoxy” group.

As used herein, the term “alkene” or “alkenyl” by itself or as part of another substituent refers to either a straight chain, branched chain, or cyclic hydrocarbon, having at least one double bond between two carbon atoms. Examples of alkene groups include, but are not limited to, vinyl, propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. The alkene group is typically monovalent, but can be divalent, such as when the alkenyl group links two moieties together.

As used herein, the term “alkyne” or “alkynyl” by itself or as part of another substituent refers to either a straight chain or branched hydrocarbon, having at least one triple bond between two carbon atoms. Examples of alkynyl groups include, but are not limited to, acetylenyl, propynyl, 1-butynyl, 2-butynyl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. The alkynyl group is typically monovalent, but can be divalent, such as when the alkynyl group links two moieties together.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acyl group can optionally also include heteroatoms within the meaning herein. Examples of acyl groups include, but are not limited to, a nicotinoyl group (pyridyl-3-carbonyl) acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

As used herein, the term “aldehyde” by itself or as part of another substituent refers to a chemical compound that has a —CHO group.

As used herein, the term “aryl” by itself or as part of another substituent refers to cyclic aromatic hydrocarbon groups that do not contain heteroatoms in the aromatic ring assembly. “Aryl” groups can be a monocyclic or fused bicyclic, tricyclic, tetracyclic, pentacyclic, or greater, aromatic ring assembly containing 6 to 22, 14 to 22, 17 to 22, or 6 to 16 ring carbon atoms. For example, aryl may be, but is not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, benzyl, or naphthyl, and the like. Aryl groups can include substituted aryl groups. Substituted aryl groups include, e.g., but are not limited to, naphthyl or phenyl, optionally mono- or disubstituted by alkoxy, phenyl, halogen, alkyl or trifluoromethyl, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, carboxylate, carboxylic acid, C₂-C₁₂ alkyl carboxylic acid, C₂-C₁₂ alkyl carboxylate, C₂-C₁₂ alkyl carboxylate ester, C₁-C₁₂ alkoxy, a water-solubilizing group (WSG), a functional group, sulfonic acid, sulfonate, C₁-C₁₂ alkyl sulfonate. In some cases, the substituted aryl group, such as for example naphthyl or phenyl, may be mono- or disubstituted by a functional group, a WSG, optionally a WSG comprising a functional group, alkoxy, halogen or trifluoromethyl. The WSG can be a branched WSG, optionally comprising a functional group, such as, for example, a WSG comprising PEG and a functional group.

Preferred as aryl is naphthyl, phenyl or phenyl mono- or disubstituted by alkoxy, phenyl, halogen, alkyl or trifluoromethyl, especially phenyl or phenyl-mono- or disubstituted by alkoxy, halogen or trifluoromethyl, and in particular, phenyl.

The term “monocyclic heteroaryl” refers to an unsubstituted or substituted heteroaryl ring system comprising 1 aryl ring, and wherein the monocyclic ring system comprises one or more, two or more, three or more, or four or more heteroatoms. Nonlimiting examples of a monocyclic heteroaryl group are substituted or unsubstituted pyridinyl, pyranyl, thiophenyl, furanyl, pyrrolyl, pyrazolyl, imidazolyl, thiazolyl, oxazolyl.

The term “polycyclic aryl” refers to an unsubstituted or substituted polycyclic ring system comprising 2 to 9, 2 to 8, or 2 to 6 aryl rings with or without fused cycloalkyl or cycloalkenyl rings. Examples of polycyclic aryl groups may include fluorene, 9H-fluorene, phenanthrene, dihydrophenanthrene, 9,10-dihydrophenanthrene, naphthalene, anthracene, tetracene, pentacene, and the like.

The term “polycyclic heteroaryl” refers to an unsubstituted or substituted polycyclic ring system comprising 2 to 9, 2 to 8, or 2 to 6 aryl rings with or without fused cycloalkyl or cycloalkenyl rings, and wherein the polycyclic ring system comprises one or more, two or more, three or more, or four or more heteroatoms. Non-limiting examples of polycyclic heteroaryl systems may include quinoline, benzoxazole, benzothiazole, benzimidazole, indole, benzindole, pyridinium, benzopyrylium, thiopyrylium, 6,8-dihydro-5H-naphtho[2,1-f]indole, 4,5-dihydro-3H-naphtho[2,1-e]indole, 6,7-dihydro-3H-naphtho[2,1-g]indole, 5,6-dihydrophenanthro[3,2-d]thiazole, 4,5-dihydrophenanthro[2,1-d]thiazole, 6,7-dihydrophenanthro[4,3-d]thiazole, 5,6-dihydrophenanthro[3,2-d]oxazole, 4,5-dihydrophenanthro[2,1-d]oxazole, 6,7-dihydrophenanthro[4,3-d]oxazole, 5,6-dihydronaphtho[2,1-g]quinoline, 7,8-dihydronaphtho[2,1-h]quinoline, 5,6-dihydronaphtho[2,1-f]quinoline, 5,6-dihydro-8λ2-phenanthro[3,2-d]imidazole, 4,5-dihydro-3λ2-phenanthro[2,1-d]imidazole, 6,7-dihydro-3λ2-phenanthro[4,3-d]imidazole, 5,6-dihydronaphtho[1,2-g]quinoxaline, 5,6-dihydronaphtho[2,1-f]quinoxaline, 7,8-dihydronaphtho[1,2-f]quinoxaline, 5,6-dihydropentapheno[3,2,1-cd:10,11,12-c′d′]diindole, 3,8-dihydrophenanthro[2,3-e:7,6-e′]diindole, 3,5,6,8-tetrahydrophenanthro[2,3-e:7,6-e′]diindole, 1,2,3,5,6,8-hexahydrophenanthro[2,3-e:7,6-e′]diindole-1,10-diium salt, 3,5,6,8-tetrahydrophenanthro[2,3-e:7,6-e′]diindole-ethane (1/1), 5,6-dihydropentapheno[3,4-d:10,9-d′]bis(oxazole), 1,2,5,6,9,10-hexahydropentapheno[3,4-d:10,9-d′]bis(thiazole)-1,10-diium, pentapheno[3,4-d:10,9-d′]bis(thiazole), 1,2,9,10-tetrahydropentapheno[3,4-d:10,9-d′]bis(thiazole)-1,10-diium, 6,7-dihydrophenanthro[2,3-f:7,6-f]diquinoline-1,12-diium, 6,7-dihydrophenanthro[2,3-f:7,6-f]diquinoline, 2,6,7,11-tetrahydrobenzo[1,2-g:4,3-g′]dichromene, 2,11-dihydrobenzo[1,2-g:4,3-g′]dichromene, 5,10-dihydro-6H-naphtho[2,1-g]chromene, 10H-naphtho[2,1-g]chromene, 2,6,7,11-tetrahydrobenzo[1,2-g:4,3-g′]bis(thiochromene), 2,11-dihydrobenzo[1,2-g:4,3-g′]bis(thiochromene), and the like.

The term “Arylene” refers to a divalent radical derived from an aryl group. Aryl groups can be mono-, di- or tri-substituted by one, two or three radicals selected from alkyl, alkoxy, aryl, hydroxy, halogen, cyano, amino, amino-alkyl, trifluoromethyl, alkylenedioxy and oxy-C₂-C₃-alkylene; all of which are optionally further substituted, for instance as hereinbefore defined; or 1- or 2-naphthyl; or 1- or 2-phenanthrenyl. Alkylenedioxy is a divalent substitute attached to two adjacent carbon atoms of phenyl, e.g., methylenedioxy or ethylenedioxy. Oxy-C₂-C₃-alkylene is also a divalent substituent attached to two adjacent carbon atoms of phenyl, e.g., oxyethylene or oxypropylene. An example for oxy-C₂-C₃-alkylene-phenyl is 2,3-dihydrobenzofuran-5-yl. The polycyclic heteroaryl may be substituted or unsubstituted.

As used herein, the term “aryloxy” by itself or as part of another substituent refers to a O-aryl group, wherein aryl is as defined above. An aryloxy group can be unsubstituted or substituted with one or two suitable substituents. The term “phenoxy” refers to an aryloxy group wherein the aryl moiety is a phenyl ring. The term “(hetero)aryloxy” as used herein means an —O-heteroaryl group, wherein heteroaryl is as defined below. The term “(hetero)aryloxy” is used to indicate the moiety is either an aryloxy or (hetero)aryloxy group.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

As used herein, the term “amine” by itself or as part of another substituent as used herein refers to an alkyl groups as defined within, having one or more amino groups. The amino groups can be primary, secondary or tertiary. The alkyl amine can be further substituted with a hydroxy group. Amines useful in the present disclosure include, but are not limited to, ethyl amine, propyl amine, isopropyl amine, ethylene diamine and ethanolamine. The amino group can link the alkyl amine to the point of attachment with the rest of the compound, be at the omega position of the alkyl group, or link together at least two carbon atoms of the alkyl group. One of skill in the art will appreciate that other alkyl amines are useful in the present disclosure.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group may include a monoalkylamino, dialkylamino, or trialkylamino group.

The term “amide” refers to a functional group having a carbonyl group attached to an amine group, having the general formula RC(═O)NR′R″, where R, R′, and R″ represent organic groups or hydrogen atoms. The term “amido” refers to a substituent containing an amide group.

As used herein, the term “carbamate” by itself or as part of another substituent refers to the functional group having the structure —NR″CO₂R′, where R′ and R″ are independently selected from hydrogen, (C₁-C₅)alkyl and heteroalkyl, unsubstituted aryl and heteroaryl, (unsubstituted aryl)-(C₁-C₄)alkyl, and (unsubstituted aryl)oxy-(C₁-C₄)alkyl. Examples of carbamates include t-Boc, Fmoc, benzyloxy-carbonyl, alloc, methyl carbamate, ethyl carbamate, 9-(2-sulfo)fluorenylmethyl carbamate, 9-(2,7-dibromo)fluorenylmethyl carbamate, Tbfmoc, Climoc, Bimoc, DBD-Tmoc, Bsmoc, Troc, Teoc, 2-phenylethyl carbamate, Adpoc, 2-chloroethyl carbamate, 1,1-dimethyl-2-haloethyl carbamate, DB-t-BOC, TCBOC, Bpoc, t-Bumeoc, Pyoc, Bnpeoc, V-(2-pivaloylamino)-1,1-dimethylethyl carbamate, NpSSPeoc.

As used herein, the term “carboxylic acid” by itself or as part of another substituent refers to a structure R—COOH where R is a carbon-containing group of atoms.

As used herein, the term “carboxylate” by itself or as part of another substituent refers to the conjugate base of a carboxylic acid, which generally can be represented by the formula RCOO⁻. For example, the term “magnesium carboxylate” refers to the magnesium salt of the carboxylic acid The term “carboxylate ester” as used herein by itself or as part of another substituent refers to a compound derived from a carboxylic acid, which generally can be represented by the formula RCOOR′ where R′ can be an alkyl, alkene, alkyne, haloalkyl, heteroalkyl, cycloalkyl, aryl, heteroaryl, (unsubstituted aryl)alkyl, and (unsubstituted aryl)oxy-alkyl or other carbon-containing group of atoms. R′ can optionally contain functional groups.

As used herein, the term “cycloalkyl” by itself or as part of another substituent refers to a saturated or partially unsaturated, monocyclic, fused bicyclic or bridged polycyclic ring assembly containing from 3 to 12 ring atoms or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted. Unless otherwise specified, “substituted cycloalkyl” groups can be substituted with one or more groups selected from halo, hydroxy, amino, alkylamino, amido, acyl, nitro, cyano, and alkoxy. The term “lower cycloalkyl” refers to a cycloalkyl radical having from three to seven carbons including, for example, cyclopropyl, cyclobutyl, cyclopentyl, cyclohexyl, and cycloheptyl. Monocyclic rings include, for example, cyclopropyl, cyclobutyl, cyclopentyl, cyclohexyl, and cyclooctyl. Bicyclic and polycyclic rings include, for example, norbornane, decahydronaphthalene and adamantane. For example, C₃₋₈cycloalkyl includes cyclopropyl, cyclobutyl, cyclopentyl, cyclohexyl, cyclooctyl, and norbornane. Polycyclic ring systems may be substituted or unsubstituted.

As used herein, the term “cycloalkylene” refers to a cycloalkyl group, as defined above, linking at least two other groups (i.e., a divalent cycloalkyl radical). The two moieties linked to the cycloalkylene group can be linked to the same atom or different atoms of the cycloalkylene group.

As used herein, the term “haloalkyl” by itself or as part of another substituent refers to alkyl as defined above where some or all of the hydrogen atoms are substituted with halogen atoms. Halogen (halo) preferably represents chloro or fluoro, but may also be bromo or iodo. For example, haloalkyl includes trifluoromethyl, flouromethyl, 1,2,3,4,5-pentafluoro-phenyl, etc. The term “perfluoro” defines a compound or radical which has at least two available hydrogens substituted with fluorine. For example, perfluorophenyl refers to 1,2,3,4,5-pentafluorophenyl, perfluoromethane refers to 1,1,1-trifluoromethyl, and perfluoromethoxy refers to 1,1,1-trifluoromethoxy.

As used herein, the term “halogen” by itself or as part of another substituent refers to fluorine, chlorine, bromine, and iodine.

As used herein, the term “heteroaryl” by itself or as part of another substituent refers to a monocyclic or fused polycyclic, such as bicyclic, tricyclic, tetracyclic, or pentacylic aromatic ring assembly, for example, containing 5 to 22, 14 to 22, 17 to 22, 6 to 16, or 5 to 16 ring atoms, where from 1 to 4 of the ring atoms may be a heteroatom, such as N, O, or S. Additional heteroatoms can also be useful, including, but not limited to, B, Al, Si, or P. The heteroaryl may be substituted or unsubstituted. Substituted heteroaryl may include one or more K—R¹³, halogen, O—C₁₋₆ alkyl, S—C₁₋₆ alkyl, O-aryl, S-aryl, NHC₁₋₆alkyl, Ph-NCS, Ph-CO₂H, Ph-(CH₂)₁₋₄CO₂H substituents. The heteroatoms can be oxidized to form moieties such as, but not limited to, —S(O)— and —S(O)₂—. For example, heteroaryl may include pyridyl, indolyl, indazolyl, quinoxalinyl, quinolinyl, isoquinolinyl, benzothienyl, benzofuranyl, furanyl, pyrrolyl, thiazolyl, benzothiazolyl, oxazolyl, isoxazolyl, triazolyl, tetrazolyl, pyrazolyl, imidazolyl, thienyl, or any other radicals substituted, especially mono- or di-substituted, by, e.g., alkyl, nitro or halogen. Pyridyl may represent 2-, 3- or 4-pyridyl, advantageously 2- or 3-pyridyl. Thienyl may represent 2- or 3-thienyl. Quinolinyl may represent preferably 2-, 3- or 4-quinolinyl. Isoquinolinyl may represent preferably 1-, 3- or 4-isoquinolinyl. Benzopyranyl, benzothiopyranyl represents preferably 3-benzopyranyl or 3-benzothiopyranyl, respectively. Thiazolyl may represent preferably 2- or 4-thiazolyl, and most preferred, 4-thiazolyl. Triazolyl may represent preferably 1-, 2- or 5-(1,2,4-triazolyl). Tetrazolyl is preferably 5-tetrazolyl. The heteroaryl may include aryloxy or arylamino groups. In some embodiments, heteroaryl is pyridyl, indolyl, quinolinyl, pyrrolyl, thiazolyl, isoxazolyl, triazolyl, tetrazolyl, pyrazolyl, imidazolyl, thienyl, furanyl, benzothiazolyl, benzofuranyl, isoquinolinyl, benzothienyl, oxazolyl, indazolyl, or any of the radicals substituted, especially mono- or di-substituted.

As used herein, the terms “heteroalkyl” or “heteroalkoxy” by itself or as part of another substituent refers to an alkyl or alkoxy group, preferably a C₁-C₁₂ alkyl group or C₁-C₁₂ alkoxy group where a C is substituted by a heteroatom such as N, O or S. For example, heteroalkyl or heteroalkoxy can include ethers, thioethers and alkyl-amines. Additional heteroatoms can also be useful, including, but not limited to, B, Al, Si, or P. The heteroatoms can be oxidized to form moieties such as, but not limited to, —S(O)—, —S(O)₂—, sulfinate, sulfonamide. The heteroatom portion of the heteroalkyl can replace a hydrogen atom of the alkyl group to form a hydroxy, thio or amino group. Alternatively, the heteroatom portion can be the connecting atom, or be inserted between two carbon atoms.

As used herein, the term “heteroalkylene” refers to a heteroalkyl group, as defined above, linking at least two other groups (i.e., a divalent heteroalkyl radical). The two moieties linked to the heteroalkylene group can be linked to the same atom or different atoms of the heteroalkylene group.

As used herein, the term “(hetero)arylamino” by itself or as part of another substituent refers an amine radical substituted with an aryl group (e.g., —NH-aryl). An arylamino may also be an aryl radical substituted with an amine group (e.g., -aryl-NH₂). Arylaminos may be substituted or unsubstituted.

In some embodiments, substituents for the aryl, heteroaryl, and heteroalkylene groups are varied and are selected from: -halogen, —OR′, —OC(O)R′, —C(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′ C(O)NR″R″, —NH—C(NH₂)—NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R″ are independently selected from hydrogen, (C₁-C₅)alkyl and heteroalkyl, unsubstituted aryl and heteroaryl, (unsubstituted aryl)-(C₁-C₄)alkyl, and (unsubstituted aryl)oxy-(C₁-C₄)alkyl.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ₜ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X′—(CH₂)ₜ—, where s and t are independently integers of from 0 to 3, and X′ is —O—, —NR′—, —S—, —S(O)—, —S(O)₂—, or —S(O)₂NR′—. The substituent R′ in —NR′— and —S(O)₂NR′— is selected from hydrogen or unsubstituted (C₁-C₆)alkyl.

As used herein, the term “oligoether” is understood to mean an oligomer containing structural repeat units having an ether functionality. As used herein, an “oligomer” is understood to mean a molecule that contains one or more identifiable structural repeat units of the same or different formula.

As used herein, the terms “polyethylene glycol”, “PEG”, “polyethylene oxide” or “PEO” refer to the family of biocompatible water-solubilizing linear polymers based on the ethylene glycol monomer unit described by the formula —(CH₂—CH₂—O—)ₙ— or a derivative thereof. In some embodiments, “n” is 5000 or less, such as 1000 or less, 500 or less, 200 or less, 100 or less, 50 or less, 40 or less, 30 or less, 20 or less, 15 or less, such as 3 to 15, or 10 to 15. It is understood that the PEG polymeric group may be of any convenient length and may include a variety of terminal groups and/or further substituent groups, including but not limited to, alkyl, alkoxy, aryl, hydroxyl, amino, acyl, carboxylic acid, carboxylate ester, acyloxy, and amido terminal and/or substituent groups. As used herein, PEG groups include, but are not limited to, PEG, modified PEG, linked PEG, amide-PEG, sulfonamide-PEG, phosphoramide-PEG, alkyl sulfonamide-PEG and alkoxy sulfonamide-PEG. It is understood that the PEG polymeric moiety may be of any convenient length and may include a variety of terminal groups and/or further substituent groups, including but not limited to, alkyl, aryl, hydroxyl, amino, acyl, acyloxy, and amido terminal and/or substituent groups. PEG groups that may be adapted for use with the subject compounds include those PEGs described by S. Zalipsky in “Functionalized poly(ethylene glycol) for preparation of biologically relevant conjugates”, Bioconjugate Chemistry 1995, 6 (2), 150-165; by Zhu et al in “Water-Soluble Conjugated Polymers for Imaging, Diagnosis, and Therapy”, Chem. Rev., 2012, 112 (8), pp 4687-4735; by J. M. Harris in “Poly(ethylene glycol) Chemistry: Biotechnical and Biomedical Applications”, Plenum Press, New York, N.Y. (1992); and by J. M. Harris and S Zalipsky in “Poly(ethylene glycol) Chemistry and Biological Applications”, ACS (1997). In some instances, PEG and modified PEG moieties can be, for example, those taught in International Patent Applications: WO 90/13540, WO 92/00748, WO 92/16555, WO 94/04193, WO 94/14758, WO 94/17039, WO 94/18247, WO 94/28937, WO 95/11924, WO 96/00080, WO 96/23794, WO 98/07713, WO 98/41562, WO 98/48837, WO 99/30727, WO 99/32134, WO 99/33483, WO 99/53951, WO 01/26692, WO 95/13312, WO 96/21469, WO 97/03106, WO 99/45964 U.S. Pat. Nos. 4,179,337; 5,075,046; 5,089,261; 5,100,992; 5,134,192; 5,166,309; 5,171,264; 5,213,891; 5,219,564; 5,275,838; 5,281,698; 5,298,643; 5,312,808; 5,321,095; 5,324,844; 5,349,001; 5,352,756; 5,405,877; 5,455,027; 5,446,090; 5,470,829; 5,478,805; 5,567,422; 5,605,976; 5,612,460; 5,614,549; 5,618,528; 5,672,662; 5,637,749; 5,643,575; 5,650,388; 5,681,567; 5,686, 110; 5,730,990; 5,739,208; 5,756,593; 5,808,096; 5,824,778; 5,824,784; 5,840,900; 5,874,500; 5,880,131; 5,900,461; 5,902,588; 5,919,442; 5,919,455; 5,932,462; 5,965,119; 5,965,566; 5,985,263; 5,990,237; 6,011,042; 6,013,283; 6,077,939; 6,113,906; 6,127,355; 6,177,087; 6,180,095; 6,194,580; 6,214,966 each of which are incorporated herein by reference).

As used herein, the term “sulfonate functional group” or “sulfonate” either by itself or as part of another substituent refers to both the free sulfonate anion (—S(═O)₂O—) and salts thereof. Therefore, the term sulfonate encompasses sulfonate salts such as sodium, lithium, potassium and ammonium sulfonate.

As used herein, the term “sulfonamide” by itself or as part of another substituent refers to a group of formula —SO₂NR₂ where each R can independently be, for example, a water solubilizing moiety, hydrogen, alkyl, alkene, alkyne, cycloalkyl, haloalkyl, aryl, or functional group and can contain carboxylic groups. R can be a water-solubilizing polymer including, but not limited to, a polymer comprising 6 or more monomeric units, a non-ionic water-soluble polymer, such as PEG, modified PEG terminated with, for example, a carboxylic acid, a carboxylic ester, or an alkoxy group (e.g., OMethyl or OEthyl). The “sulfonamide” attached to another molecule by a linker or bond. The “sulfonamide” can be, for example, sulfonamide-PEG, alkyl sulfonamide, alkoxy sulfonamide, alkyl sulfonamide PEG, alkoxy sulfonamide PEG, alkyl sulfonamide PEG carboxylate, alkoxy sulfonamide PEG carboxylate.

As used herein, the term “sulfonamido” by itself or as part of another substituent refers to a group of formula —SO₂NR— where R can be, for example, a water solubilizing moiety, hydrogen, alkyl, alkene, alkyne, cycloalkyl, haloalkyl, aryl, or functional group and can contain carboxylic groups. R can be a water-solubilizing polymer including, but not limited to, a polymer comprising 6 or more monomeric units, a non-ionic water-soluble polymer, such as PEG, modified PEG terminated with a carboxylic acid or a carboxylic ester. The “sulfonamido” can be attached to another molecule by a linker or bond. The “sulfonamido” can be, for example, sulfonamido-PEG, alkyl sulfonamido, alkoxy sulfonamido, alkyl sulfonamido PEG, alkoxy sulfonamido PEG, alkyl sulfonamido PEG carboxylate, alkoxy sulfonamido PEG carboxylate.

As used herein, the term “sulfinamide” by itself or as part of another substituent refers to a group of formula —SONR₂ where each R can independently be, for example, a water solubilizing moiety, hydrogen, alkyl, alkene, alkyne, cycloalkyl, haloalkyl, aryl, or functional group and can contain carboxylic groups. R can be a water-solubilizing polymer including, but not limited to, a polymer comprising 6 or more monomeric units, a non-ionic water-soluble polymer, such as PEG, modified PEG terminated with a carboxylic acid or a carboxylic ester. The “sulfinamide” can be attached to another molecule by a linker or bond. The “sulfinamide” can be, for example, sulfinamide-PEG, alkyl sulfinamide, alkoxy sulfinamide, alkyl sulfinamide PEG, alkoxy sulfinamide PEG, alkyl sulfinamide PEG carboxylate, alkoxy sulfinamide PEG carboxylate.

As used herein, the terms “hydrazine” and “hydrazide” by themselves or as part of another substituent refer to compounds that contain singly bonded nitrogens, one of which is a primary amine functional group. For example, the term “hydrazine” refers to a moiety having the structure —NHNH₂.

As used herein, the term “thiol” by itself or as part of another substituent refers to a compound that contains the functional group composed of a sulfur-hydrogen bond. The general chemical structure of the thiol functional group is R—SH, where R represents an alkyl, alkene, aryl, or other carbon-containing group of atoms.

As used herein, the term “silyl” by itself or as part of another substituent refers to Si(R^{z})₃ wherein each R^{z} independently is alkyl, aryl or other carbon-containing group of atoms.

As used herein, the term “diazonium salt” by itself or as part of another substituent refers to a group of organic compounds with a structure of R—N₂⁺X′⁻, wherein R can be any organic group (e.g., alkyl or aryl) and X′ is an inorganic or organic anion (e.g., halogen).

As used herein, the term “triflate” by itself or as part of another substituent also referred to as trifluoromethanesulfonate, is a group with the formula CF₃SO₃.

As used herein, the term “boronic acid” by itself or as part of another substituent refers to a structure —B(OH)₂. It is recognized by those skilled in the art that a boronic acid may be present as a boronate ester at various stages in the synthesis of the quenchers. Boronic acid is meant to include such esters. The term “boronic ester” or “boronate ester” as used herein refers to a chemical compound containing a —B(Z¹)(Z²) moiety, wherein Z¹ and Z² together form a moiety where the atom attached to boron in each case is an oxygen atom. In some embodiments, the boronic ester moiety is a 5-membered ring. In some other embodiments, the boronic ester moiety is a 6-membered ring. In some other embodiments, the boronic ester moiety is a mixture of a 5-membered ring and a 6-membered ring.

As used herein, the term “maleimide” by itself or as part of another substituent refers a structure

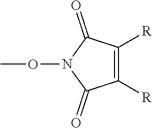

where R can be, for example, a water solubilizing moiety, hydrogen, alkyl, alkene, alkyne, cycloalkyl, haloalkyl, aryl, or other group and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

As used herein, the term “hydrazone” by itself or as part of another substituent refers to a structure

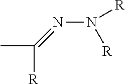

where R can be, for example, a water solubilizing moiety, hydrogen, alkyl, alkene, alkyne, cycloalkyl, haloalkyl, aryl, or other group and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

As used herein, the term “azide” by itself or as part of another substituent refers to a structure —N₃.

As used herein, the term “tetrazine” by itself or as part of another substituent refers to a compound having the molecular formula C₂H₂N₄ that consists of a six-membered heteroaromatic ring structure having 4 nitrogen atoms. The term “tetrazine” includes all of its isomers, namely 1,2,3,4-tetrazines, 1,2,3,5-tetrazines, and 1,2,4,5-tetrazines.

As used herein, the term “N-hydroxysuccinimidyl” by itself or as part of another substituent refers to a structure

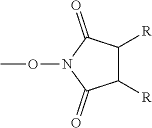

where R can be, for example, a water solubilizing moiety, hydrogen, alkyl, alkene, alkyne, cycloalkyl, haloalkyl, aryl, or other group and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

As used herein, the term “phosphoramide” by itself or as part of another substituent refers to a structure

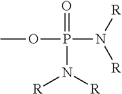

where R can be, for example, a water solubilizing moiety, hydrogen, alkyl, alkene, alkyne, cycloalkyl, haloalkyl, aryl, or other group and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester.

As used herein, the term “phosphonamidate” by itself or as part of another substituent refers to a structure

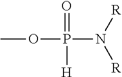

where R can be, for example, a water solubilizing moiety, hydrogen, alkyl, alkene, alkyne, cycloalkyl, haloalkyl, aryl, or other group and can contain carboxylic groups. R can be a water-solubilizing polymer including, but not limited to, a polymer comprising 6 or more monomeric units, a non-ionic water-soluble polymer, PEG, modified PEG terminated with a ca [symbol] or a carboxylic ester. The “phosphonamidite” can be attached to another molecule by a linker or bond. The “phosphonamidite” can be, for example, phosphonamidite-PEG, alkyl phosphonamidite, alkoxy phosphonamidite, alkyl phosphonamidite PEG, alkoxy phosphonamidite PEG, alkyl phosphonamidite PEG carboxylate, alkoxy phosphonamidite PEG carboxylate.

As used herein, the term “phosphinamide” by itself or as part of another substituent refers to a structure

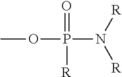

where R can be, for example, a water solubilizing moiety, hydrogen, alkyl, alkene, alkyne, cycloalkyl, haloalkyl, aryl, or other group and can contain carboxylic groups. R can be a water-solubilizing polymer including, but not limited to, a polymer comprising 6 or more monomeric units, a non-ionic water-soluble polymer, PEG, modified PEG terminated with a carboxylic acid or a carboxylic ester. The “phosphinamide” can be attached to another molecule by a linker or bond. The “phosphinamide” can be, for example, phosphinamide-PEG, alkyl phosphinamide, alkoxy phosphinamide, alkyl phosphinamide PEG, alkoxy phosphinamide PEG, alkyl phosphinamide PEG carboxylate, alkoxy phosphinamide PEG carboxylate.

The term “fluorescent” as used herein refers to a compound which, when irradiated by light of a wavelength that the compound absorbs, emits light of a (typically) different wavelength. Fluorescence is the emission of light by a substance that has absorbed light or other electromagnetic radiation. In most cases, the emitted light has a longer wavelength than the absorbed light.

The term “absorbance maxima” or “Abs λ max” or “max λ abs” refer to wavelength of maximum absorbance measured by UV Vis spectroscopy.

The term “excitation wavelength” or “λ ex” refers to the wavelength where the compound can be excited to induce fluorescence emission, it does not necessarily have to be at the Abs λ max.

The term “chromophore” refers to a molecular entity or a portion thereof consisting of an atom or a group of atoms in which the electronic transition responsible for a given spectral band is approximately localized. In some instances, the “chromophore” may itself be fluorescent. As used herein, the terms “fluorescent chromophore” and “fluorescent dye” are used interchangeably and refer t [symbol] d which has a structure capable of harvesting light with a particular absorption maximum wavelength and converting it to emitted light at a longer emission maximum wavelength. A chromophore may have a reactive group (e.g., a carboxylate moiety, an amino moiety, a haloalkyl moiety, or the like) that can be covalently bonded. Examples of suitable chromophores include, but are not limited to, those described in U.S. Pat. Nos. 7,687,282; 7,671,214; 7,446,202; 6,972,326; 6,716,979; 6,579,718; 6,562,632; 6,399,392; 6,316,267; 6,162,931; 6,130,101; 6,005,113; 6,004,536; 5,863,753; 5,846,737; 5,798,276; 5,723,218; 5,696,157; 5,658,751; 5,656,449; 5,582,977; 5,576,424; 5,573,909; and 5,187,288, which patents are incorporated herein by reference in their entirety.

The term “moiety” refers to a group as a portion of a molecule, which may be a functional group, or a portion of a molecule with multiple groups which share common structural and/or functional aspects. Examples of group or moiety include but are not limited to a linker moiety, a functional group, a water-solubilizing moiety, a PEG moiety, according to the present disclosure.

The term “linker,” “linked” or “linkage” refers to a linking moiety that connects two groups and has a backbone of 100 atoms or less in length. A linker or linkage may be a covalent bond that connects two groups or a chain of between 1 and 100 atoms in length, for example a chain of 1, 2, 3, 4, 5, 6, 8, 10, 12, 14, 16, 18, 20 or more carbon atoms in length, where the linker may be linear, branched, cyclic or a single atom. In some embodiments, the linker is a branching linker that refers to a linking moiety that connects three or more groups In some embodiments, the linker backbone includes a linking functional group, such as an ether, thioether, amino, amide, carbonyl, acyl, sulfonamide, a sulfinamide, a disulfonamide, a disulfinamide, a sultam, an amide, a secondary amine, a phosphonamide, a phosphinamide, a phosphonamidate, a selenonamide, a seleninamide, carbamate, thiocarbamate, urea, thiourea, ester, thioester or imine. In some embodiments, the linker backbone includes a linking functional group, such as an amino, amide, carbonyl, sulfonamide, sulfinamide, a disulfonamide, a disulfinamide, a sultam, an amide, a secondary amine, a phosphonamide, a phosphinamide, a phosphonamidate, a selenonamide, or a seleninamide. In certain cases, one, two, three, four or five or more carbon atoms of a linker backbone may be optionally substituted with a sulfur, nitrogen or oxygen heteroatom. In some embodiments, the linker backbone includes a linking functional group, such as an ether, thioether, amino, amide, carbonyl, acyl, sulfonamide, carbamate, thiocarbamate, urea, thiourea, ester, thioester or imine. The bonds between backbone atoms may be saturated or unsaturated, and in some cases not more than one, two, or three unsaturated bonds are present in a linker backbone. The linker may include one or more substituent groups, for example with an alkyl, aryl or alkenyl group. A linker may include, without limitations, polyethylene glycol, ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable.

A linker moiety can be attached to “L” or to “A”, as taught in U.S. Pat. No. 11,584,825 B2, which is incorporated herein by reference in its entirety. A linker moiety can comprise covalent bond, an alkoxy, sulfonamide, disulfonamide, a selenomide, a sulfinamide, a sultam, a disulfinamide, an amide, carbonyl, a seleninamide, a phosphonamide, a phosphinamide, a phosphonamidate, or a secondary amine.

As described therein, and as each pertains to a linker moiety, the term “sulfonamide,” refers to a moiety —S(O)₂NR—; the term “disulfonamide,” refers to a moiety —S(O)₂NRS(O)₂—; the term “selenonamide,” refers to a moiety —Se(O)₂NR—; the term “sulfinamide,” refers to a moiety —S(O)NR₂; the term “disulfinamide,” refers to a moiety —S(O)NRS(O)—; the term “seleninamide,” refers to a moiety —Se(O)NR—; the term “phosphonamide,” refers to a moiety —NR—PR(O)NR—; the term “phosphinamide,” refers to a moiety —PR(O)NR—; and the term “phosphonamidate,” refers to a moiety —O—PR(O)NR—; and the term “sultam” refers to a cyclic sulfonamide (e.g., wherein the R group is bonded to the sulfur atom via an alkylene moiety); wherein for each term the R group is independently H, alkyl, haloalkyl, or aryl.

The subject water-soluble fluorescent polymers feature termini on the conjugated polymer chains that can include a functional group that provides for bioconjugation. In some cases, such functionality is referred to as an end linker or end group. With these end linkers, a covalent bond can be formed to attach a biomolecule such as a protein, peptide, affinity ligand, antibody, antibody fragment, polynucleotide, or aptamer. For example, polymeric dye-labeled antibodies find use in flow cytometry as reagents exhibiting high brightness. Additionally, orthogonal functional groups can be installed along the conjugated polymer chain that can be used for either bioconjugation or the attachment of acceptor signaling chromophores in donor acceptor polymeric tandem dyes.

The phrase “conjugated water-soluble fluorescent polymer” refers to a water-soluble fluorescent polymer having a binding partner conjugated thereto.

In chemical structures, “ [symbol] ” represents either a single or double bond.

In chemical structures,

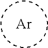

represents an optional aryl group.

The phrase “binding partner” or “binding member” refers to any molecule or complex of molecules capable of specifically binding to a target analyte. A binding partner of the present disclosure includes for example, a protein (e.g., an antibody or an antibody fragment), a carbohydrate (e.g., a polysaccharide), an oligonucleotide, a polynucleotide, a lipid, an affinity ligand, an aptamer, or the like. In some embodiments, the binding partner is an antibody or fragment thereof. Specific binding in the context of the present disclosure refers to a binding reaction which is determinative of the presence of a target analyte in the presence of a heterogeneous population. Thus, under certain assay conditions, the specified binding partners bind preferentially to a particular protein or isoform of the particular protein and do not bind in a significant amount to other proteins or other isoforms present in the sample.

In some cases, the antibody includes intravenous immunoglobulin (IVIG) and/or antibodies from (e.g., enriched from, purified from, e.g., affinity purified from) IVIG. IVIG is a blood product that contains IgG (immunoglobulin G) pooled from the plasma (e.g., in some cases without any other proteins) from many (e.g., sometimes over 1,000 to 60,000) normal and healthy blood donors. IVIG is commercially available. Aspects of IVIG are described, for example, in US. Pat. Appl. Pub. Nos. 2010/0150942; 2004/0101909; 2013/0177574; 2013/0108619; and 2013/0011388.

In some cases, the antibody is a monoclonal antibody of a defined sub-class (e.g., IgG1, IgG2, IgG3, or IgG4). If combinations of antibodies are used, the antibodies can be from the same subclass or from different subclasses. For example, the antibodies can be IgG1 antibodies. In some embodiments, the monoclonal antibody is humanized.

The phrase “water-soluble fluorescent polymer complex” refers to a water-soluble fluorescent polymer of the present disclosure conjugated with a binding partner.

The phrase “protected group” (also referred to as “protecting group”) refers to a reversibly formed derivative of an existing functional group in a molecule attached to decrease reactivity so that the protected functional group does not react under synthetic conditions to which the molecule is subjected. Examples of amine protecting groups include, but are not limited to, benzyloxycarbonyl; 9-fluorenylmethyloxycarbonyl (Fmoc); tert-butyloxycarbonyl (Boc); allyloxycarbonyl (Alloc); p-toluene sulfonyl (Tos); 2,2,5,7,8-pentamethylchroman-6-sulfonyl (Pmc); 2,2,4,6,7-pentamethyl-2,3-dihydrobenzofuran-5-sulfonyl (Pbf); mesityl-2-sulfonyl (Mts); 4-methoxy-2,3,6-trimethylphenylsulfonyl (Mtr); acetamido; phthalimido; and the like. These and other protecting groups for amines, carboxylic acids, alcohols, and further functional groups can be added to and removed from polymers of the present disclosure using known techniques as described, for example, by Green and Wuts (Protective Groups in Organic Synthesis, 4^{th} Ed. 2007, Wiley-Interscience, New York).

The term “sample” refers to a material or mixture of materials, in some cases in liquid form, containing one or more analytes of interest. In some embodiments, the term as used in its broadest sense, refers to any plant, animal or bacterial material containing cells or producing cellular metabolites, such as, for example, tissue or fluid isolated from an individual (including without limitation plasma, serum, cerebrospinal fluid, lymph, tears, saliva and tissue sections) or from in vitro cell culture constituents, as well as samples from the environment. The term “sample” may also refer to a “biological sample”. As used herein, the term “a biological sample” refers to a whole organism or a subset of its tissues, cells or component parts (e.g. body fluids, including, but not limited to, blood, mucus, lymphatic fluid, synovial fluid, cerebrospinal fluid, saliva, amniotic fluid, amniotic cord blood, urine, vaginal fluid and semen). A “biological sample” can also refer to a homogenate, lysate or extract prepared from a whole organism or a subset of its tissues, cells or component parts, or a fraction or portion thereof, including but not limited to, plasma, serum, spinal fluid, lymph fluid, the external sections of the skin, respiratory, intestinal, and genitourinary tracts, tears, saliva, milk, blood cells, tumors and organs. In certain embodiments, the sample has been removed from an animal or plant. Biological samples may include cells. The term “cells” is used in its conventional sense to refer to the basic structural unit of living organisms, both eukaryotic and prokaryotic, having at least a nucleus and a cell membrane. In certain embodiments, cells include prokaryotic cells, such as from bacteria. In other embodiments, cells include eukaryotic cells, such as cells obtained from biological samples from animals, plants or fungi.

The term “substrate” refers to a solid material having a variety of configurations. The substrate can be, for example, a sheet, bead, or other structure, such as a plate with wells, a polymer, particle, a semiconductor surface, nanotubes, a fibrous mesh, hydrogels, porous matrix, a pin, a microarray surface, a chromatography support, and the like. In some instances, the substrate is selected from the group consisting of a particle, a planar solid substrate, a fibrous mesh, a hydrogel, a porous matrix, a pin, a microarray surface and a chromatography support.

The term “water” as used herein refers to any aqueous solution that is primarily water and is compatible with physiological conditions. In some instances, the aqueous solution contains more than 50% water, such as more than 60% water, more than 70% water, more than 80% water, more than 90% water, or more than 95% water. The term “water” includes, for example, biological buffers and other aqueous solutions that may contain additives such as salts, detergents, stabilizers, and other water-soluble components, for example, sugars, proteins, amino acids, and nucleotides. In some instances, “water” may be an aqueous solution containing up to 10% miscible organic solvent (e.g., up to 10% DMSO in water). The term “water” does not include pure solvents or solvent combinations different from water, such as pure alcohols, for example pure methanol or ethanol, pure ethers, for example pure diethyl ether or tetrahydrofuran, or any other pure solvent either miscible or not miscible with water.

The term “water solubilizing moiety” as used herein by itself or part of another group refers to any hydrophilic group that is well solvated in aqueous environments, for example such as under physiological conditions, and is capable of increasing the water solubility of the molecule to which it is attached. The increase in water solubility of the molecule can vary depending upon the moiety attached. In some instances, the increase in water solubility (as compared to the solubility of the molecule without the moiety attached) is 2 fold or more, 5 fold or more, 10 fold or more, 25 fold or more, 50 fold or more, or 100 fold or more. “Water-solubilizing moiety” includes moieties, such as, but not limited to, PEG groups, carboxy groups including but not limited to carboxylic acids and carboxylates, polyvinyl alcohol, glycols, peptides, polyphosphates, polyalcohols, sulfonates, phosphonates, boronates, amines, ammoniums, sulfoniums, phosphonium, alcohols, polyols, oxazolines, zwitterionic derivatives, carbohydrates, nucleotides, polynucleotides, substituted PEG groups, substituted carboxy groups including but not limited to substituted carboxylic acids and substituted carboxylates, substituted glycols, substituted peptides, substituted polyphosphates, substituted polyalcohols, substituted sulfonates, substituted phosphonates, substituted boronates, substituted amines, substituted ammoniums, substituted sulfoniums, substituted phosphonium, alcohols, substituted zwitterionic derivatives, substituted carbohydrates, substituted nucleotides, substituted polynucleotides, and combinations thereof.

The term “water-solubilizing moiety” or “water-solubilizing group” (WSG or W) as used herein by itself or part of another group refers to any hydrophilic group that is well solvated in aqueous environments, for example such as under physiological conditions, and is capable of increasing the water solubility of the molecule to which it is attached. Any convenient WSG may be included in the dyes described herein to provide for increased water-solubility. A water-solubilizing moiety can increase the solubility of a compound in a predominantly aqueous solution, as compared to a control compound which lacks the water-solubilizing moiety. The water-solubilizing moiety may be any convenient hydrophilic moiety that is well solvated in aqueous environments. In some instances, the water-solubilizing moiety can be capable of imparting solubility in water (e.g., aqueous buffer)>1 mg/mL, >2 mg/mL, >3 mg/mL, >4 mg/mL, >5 mg/mL, >6 mg/mL, >7 mg/mL, >8 mg/mL, >9 mg/mL, or >10 mg/mL. In some instances, the water-solubilizing moiety can be capable of imparting solubility in water of >10 mg/mL, >20 mg/mL, >30 mg/mL, >40 mg/mL, >50 mg/mL, >60 mg/mL, >70 mg/mL, >80 mg/mL, >90 mg/mL or >100 mg/mL.

The increase in water solubility of the molecule can vary depending upon the moiety attached. In some instances, the increase in water solubility (as compared to the solubility of the molecule without the moiety attached) is 2 fold or more, 5 fold or more, 10 fold or more, 25 fold or more, 50 fold or more, or 100 fold or more. In some cases, the water-solubilizing moiety is charged, e.g., a positively or negatively charged hydrophilic moiety. In some instances, the water-solubilizing moiety is a neutral hydrophilic moiety. In some instances, the water-solubilizing moiety is branched (e.g., as described herein). In some instances, the water-solubilizing moiety is linear. Water-solubilizing moieties include, but are not limited to, those taught in US Patent Publication No. 2022/0348770 which is incorporated herein by referenced in its entirety.

A “water-soluble compound” may exhibit solubility in water (e.g., aqueous buffer) of >1 mg/mL, >2 mg/mL, >3 mg/mL, >4 mg/mL, >5 mg/mL, >6 mg/mL, >7 mg/mL, >8 mg/mL, >9 mg/mL, or >10 mg/mL at ambient room temperature. In some instances, the water-soluble compound can exhibit solubility in water of >10 mg/mL, >20 mg/mL, >30 mg/mL, >40 mg/mL, >50 mg/mL, >60 mg/mL, >70 mg/mL, >80 mg/mL, >90 mg/mL and/or >100 mg/mL at ambient room temperature.

Any convenient WSG may be included in the dyes described herein to provide for increased water-solubility. WSGs may be, but are not limited to, carboxylate, phosphonate, phosphate, sulfonate, sulfate, sulfinate, sulfonium, ester, polyethylene glycols (PEG) and modified PEGs, linear PEG groups, branched PEG groups, hydroxyl, amine, amino acid, ammonium, guanidinium, pyridinium, polyamine and sulfonium, polyalcohols, straight chain or cyclic saccharides, primary, secondary, tertiary, or quaternary amines and polyamines, phosphonate groups, phosphinate groups, ascorbate groups, glycols, including, polyethers, a zwitterionic derivative, a peptide sequence, nucleotides (DNA and RNA), a peptoid, a carbohydrate, an oxazoline, a polyol, a dendron, a dendritic polyglycerol, a cellulose, a chitosan, —COOM′, —SO₃M′, —PO₃M′, —NR³₊, Y′, (CH₂CH₂O)ₚR and mixtures thereof, where Y′ can be any halogen, sulfate, sulfonate, or oxygen containing anion, p can be 1 to 500, each R can be independently H or an alkyl (such as methyl) and M′ can be a cationic counterion or hydrogen, —(CH₂CH₂O)_{yy}CH₂CH₂XR^{yy}, —(CH₂CH₂O)_{yy}CH₂CH₂X—, —X(CH₂CH₂O)_{yy}CH₂CH₂—, glycol, and polyethylene glycol, wherein yy is selected from 1 to 1000, X is selected from O, S, and NR^{ZZ}, and R^{ZZ} and R^{YY} are independently selected from H and C₁₋₃ alkyl, and combinations or derivatives thereof. In some instances, WSGs include, but are not limited to, PEG, a modified PEG, a peptide sequence, a peptoid, a carbohydrate, an oxazoline, a polyol, a dendron, a dendritic polyglycerol, a cellulose, a chitosan, or a derivative thereof. WSGs may be unsubstituted or substituted.

In some instances, the WSGs may be a hydrophilic polymer. For example, hydrophilic polymers that can be utilized in the WSG include, but are not limited to, polyalkylene oxide based polymers comprising an ethylene oxide repeat unit of the formula —(CH₂—CH₂—O)ₙ— or —(O—CH₂—CH₂)ₙ—, such as, for example, PEG, polyamide alkylene oxide, or derivatives thereof. Further examples of polymers of interest include a polyamide having a molecular weight greater than 1,000 Daltons of the formula —[C(O)—X—C(O)—NH—Y—NH]ₙ— or —[NH—Y—NH—C(O)—X—C(O)]ₙ—, where X and Y are divalent radicals that may be the same or different and may be branched or linear, and n is a discrete integer from 2-100, such as from 2 to 50, and where either or both of X and Y comprises a biocompatible, substantially non-antigenic water-soluble repeat unit that may be linear or branched. The number of such water-soluble repeat units can vary significantly, with the number of such units being from 2 to 500, 2 to 400, 2 to 300, 2 to 200, 2 to 100, 6-100, for example from 2 to 50 or 6 to 50. An example of an embodiment is one in which one or both of X and Y is selected from: —((CH₂)ₙ₁—(CH₂—CH₂—O)ₙ₂—(CH₂)— or —((CH₂)ₙ₁—(O—CH₂—CH₂)ₙ₂—(CH₂)ₙ₁—), where n1 is 1 to 6, 1 to 5, 1 to 4, or 1 to 3, and where n2 is 2 to 50, 2 to 25, 2 to 15, 2 to 10, 2 to 8, or 2 to 5. In some instances, the water-soluble polymer is a group of 1-50 monomeric units, such as 1-40, 1-30, 1-20, 2-24, 2-20, 2-10 or 2-6 monomeric units. A further example of an embodiment is one in which X is —(CH₂—CH₂)—, and where Y is —(CH₂—(CH₂—CH₂—O)₃—CH₂—CH₂—CH₂)— or —(CH₂—CH₂—CH₂—(O—CH₂—CH₂)₃—CH₂)—. In certain instances, any one of the formulae described herein may be substituted with a water-soluble moiety that is a dendron, as known in art.

In some instances, hydrophilic polymers can be, for example, PEG, a peptide sequence, a peptoid, a carbohydrate, an oxazoline, a polyol, a dendron, a dendritic polyglycerol, a cellulose, a chitosan, or a derivative thereof.

In some cases, a WSG is (CH₂)ₓ(OCH₂CH₂)_{y}OCH₃ where each x is independently an integer from 0-20, each y is independently an integer from 0 to 50. In some instances, the water-soluble polymer is a PEG group or modified PEG polymer of 6-24 monomeric units, such as 10-30, 10-24, 10-20, 12-24, 12-20, 12-16 or 16-20 monomeric units.

In some cases, the WSG includes a non-ionic polymer (e.g., a PEG polymer) substituted at the terminal with an ionic group (e.g., a sulfonate). In some embodiments of the formulae, the WSG includes a substituent selected from (CH₂)ₓ(OCH₂CH₂)_{y}OCH₃ where each x is independently an integer from 0-20, each y is independently an integer from 0 to 50; and a benzyl optionally substituted with one or more halogen, hydroxyl, C₁-C₁₂ alkoxy, or (OCH₂CH₂)_{z}OCH₃ where each z is independently an integer from 0 to 50. In some instances, the WSG is (CH₂)₃(OCH₂CH₂)₁₁OCH₃. In some embodiments, one or more of the substituents is a benzyl substituted with at least one WSG groups (e.g., one or two WSG groups) selected from (CH₂)ₓ(OCH₂CH₂)_{y}OCH₃ where each x is independently an integer from 0-20 and each y is independently an integer from 0 to 50. It is understood that hydroxy-terminated polymer chains (e.g., PEG chains) instead of methoxy-terminated polymer chains (e.g., PEG chains) may be utilized in any of the water-solubilizing moieties.

The term modified polymer, such as a modified PEG, refers to water soluble polymers that have been modified or derivatized at either or both terminals, e.g., to include a terminal substituent (e.g., a terminal alkyl, substituted alkyl, alkoxy or substituted alkoxy, etc.) and/or a terminal linking functional group (e.g., an amino or carboxylic acid group suitable for attachment via amide bond formation) suitable for attached of the polymer to a molecule of interest (e.g., to a light harvesting chromophore via a branching group). The subject water-soluble polymers can be adapted to include any convenient linking groups. It is understood that in some cases, the water-soluble polymer can include some dispersity with respect to polymer length, depending on the method of preparation and/or purification of the polymeric starting materials. In some instances, the water-soluble polymers are monodisperse.

The water-soluble polymer can include one or more spacers or linkers. Examples of spacers or linkers include linear or branched moieties comprising one or more repeat units employed in a water-soluble polymer, diamino and or diacid units, natural or unnatural amino acids or derivatives thereof, as well as aliphatic moieties, including alkyl, aryl, heteroalkyl, heteroaryl, alkoxy, and the like, which can contain, for example, up to 18 carbon atoms or even an additional polymer chain.

The water-soluble polymer moiety, or one or more of the spacers or linkers of the polymer moiety when present, may include polymer chains or units that are biostable or biodegradable. For example, polymers with repeat linkages have varying degrees of stability under physiological conditions depending on bond lability. Polymers with such bonds can be categorized by their relative rates of hydrolysis under physiological conditions based on known hydrolysis rates of low molecular weight analogs, e.g., from less stable to more stable, e.g., polyurethanes (—NH—C(O)—O—)>polyorthoesters (—O—C((OR)(R′))—O—)>polyamides (—C(O)—NH—). Similarly, the linkage systems attaching a water-soluble polymer to a target molecule may be biostable or biodegradable, e.g., from less stable to more stable: carbonate (—O—C(O)—O—)>ester (—C(O)—O—)>urethane (—NH—C(O)—O—)>orthoester (—O—C((OR)(R′))—O—)>amide (—C(O)—NH—). In general, it may be desirable to avoid use of a sulfated polysaccharide, depending on the lability of the sulfate group. In addition, it may be less desirable to use polycarbonates and polyesters. These bonds are provided by way of example, and are not intended to limit the types of bonds employable in the polymer chains or linkage systems of the water-soluble polymers useful in the WSGs disclosed herein.

In some instances, the water-solubilizing moieties include, but are not limited to, hydroxy, alkoxy, (hetero)aryloxy, (hetero)arylamino, PEG, linked PEG, amide-PEG, sulfonamide-PEG, phosphoramide-PEG, ammonium alkyl salt, ammonium alkyloxy salt, ammonium oligoether salt, carbonyl, acyl, sulfonate, alkyl sulfonate, alkyl carboxylate, sulfonate alkyl salt, sulfonate alkoxy salt, sulfonate oligoether salt, sulfonamido oligoether, sulfonamide, sulfinamide, phosphonamidate, phosphinamide, alkoxy sulfonamide PEG, alkylcarboxylate, alkylamide, alkoxy sulfonate, alkyl sulfonate, alkyl sulfonate salt,

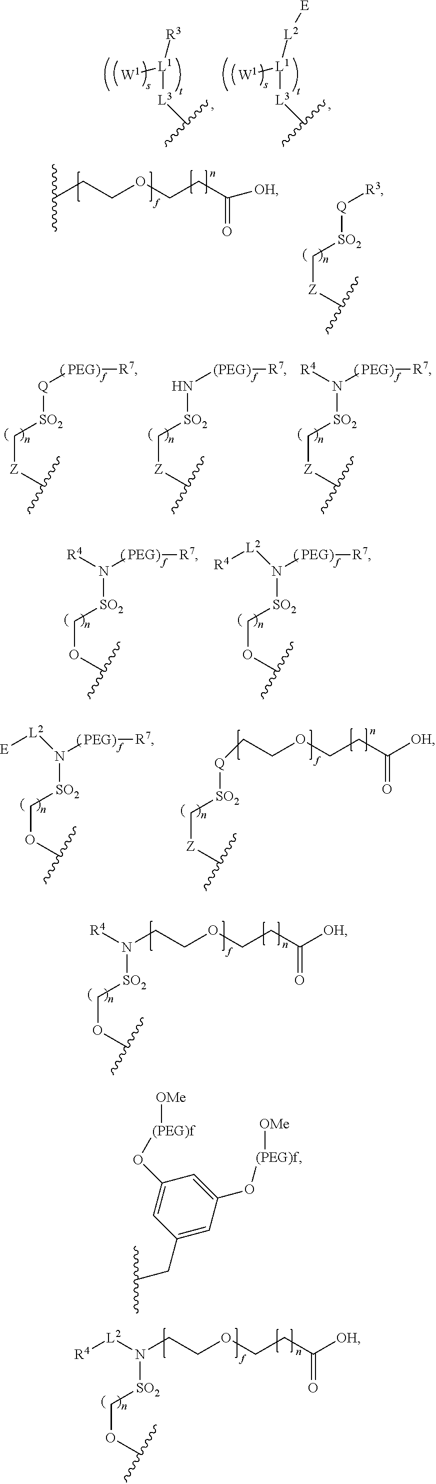

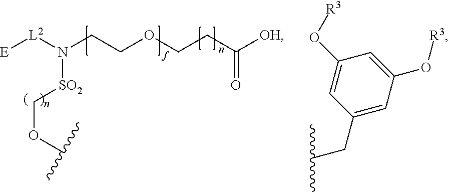

and

In some instances, the subject compounds may comprise multiple water-solubilizing moieties attached at a single location in the subject compounds, for example, via a branching linker, such as, for example, an aralkyl substituent further di-substituted with water solubilizing groups. As such, in some cases, the branching linker group is a substituent of the dye that connects the dye to two or more water solubilizing groups. In some instances, multiple water-solubilizing moieties may be attached to the subject compounds via groups having, for example, the following formulas:

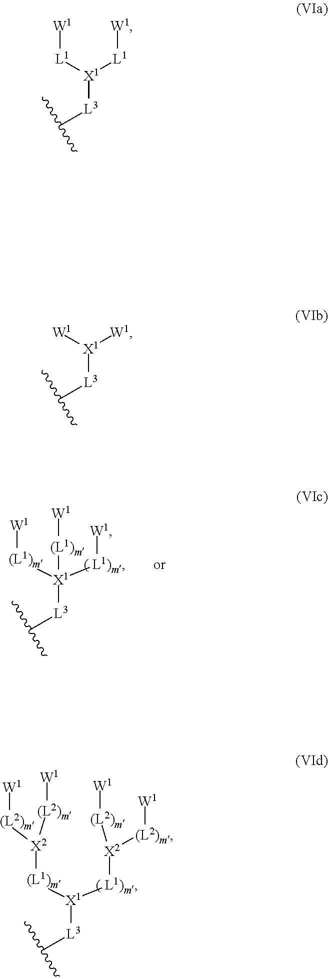

wherein X¹, X² are branching points, L¹, L², L³ are linkers, m′ is an integer from 1, 2, or 3; W¹ is a water-solubilizing moiety.

In some instances, one or more water-solubilizing moieties may be attached to the subject compounds via a group comprising linkers according to the disclosure, for example, as taught in US Published Application No. 2020/0190253A1, which is incorporated herein by reference in its entirety. A linker moiety can be attached to the cyanine bridge or the heterocycloaryl groups of the fluorescent compounds of the instant disclosure. A linker may be cleavable or non-cleavable.

One or more water-solubilizing moieties can also be attached to the subject compounds via a group comprising linkers, such as, for example, but not limited to, the following linker formula (VIe):

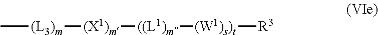

wherein:
each optional L¹ and L³ is an independently selected linker moiety;
X¹, optionally present, is a branching point;
W¹ is a water-soluble moiety, including, but not limited to, a water-soluble polymer comprising 2-50, 4-30, or 6-24 monomeric units;
each m is independently 0 or 1; each m′ is independently 0 or 1; each m″ is independently 0 or 1;
each s is independently 1 or 2;
each tis independently 0, 1, 2, or 3; and
R³ is as defined herein.

In some instances, L¹, L³, and X are absent and W¹ is a water-solubilizing moiety, for example, a water-soluble polymer comprising 2-50, 4-30, or 6-24 monomeric units, such as 10-30, 10-24, 10-20, 12-24, 12-20, 12-16 or 16-20 monomeric units. In some cases, the water-solubilizing moiety may be a linear water-solubilizing moiety. For example, L¹ and X may be absent, L³ is a linker (e.g., as disclosed herein), and W¹ is a water-solubilizing moiety.

In some cases, at least one of, at least two of, or all three of L¹, L² and/or L³ may be selected from an alkyl or substituted alkyl linker, an alkenyl or substituted alkenyl linker, an alkynyl or substituted alkynyl linker, an alkoxy or substituted alkoxy linker, a PEG linker, a sulfonamido-alkyl or substituted sulfonamido-alkyl linker, an amido-alkyl or substituted amido-alkyl linker and an alkyl-amido-alkyl or substituted alkyl-amido-alkyl linker. In certain cases, the linker comprises a carbonyl group. A linker moiety can be a covalent bond, an alkoxy, sulfonamide, disulfonamide, a selenomide, a sulfinamide, a sultam, a disulfinamide, an amide, carbonyl, a seleninamide, a phosphonamide, a phosphinamide, a phosphonamidate, or a secondary amine.

In some instances, L² and L³ may be linker moieties each independently selected from the group consisting of a covalent bond, C₁₋₈ alkylene, 2- to 8-membered heteroalkylene, and a chain of between 2 and 200 backbone atoms in length, wherein the chain comprises a linear chain, a branched chain, and/or a cyclic moiety.

In some instances, L¹ can be a sulfonamide, a sulfinamide, a disulfonamide, a disulfinamide, a sultam, an amide, a secondary amine, a phosphonamide, a phosphinamide, a phosphonamidate, a selenonamide, or a seleninamide.

In some instances, L³ can be a linker having a backbone of 20 atoms or less in length and W¹ is a water-solubilizing moiety (e.g., as described herein). In some instances, L³ can be selected from an alkyl or substituted alkyl linker, an alkenyl or substituted alkenyl linker, an alkynyl or substituted alkynyl linker, an acyl or substituted acyl, an alkoxy or substituted alkoxy linker, a PEG linker, a sulfonamido-alkyl or substituted sulfonamido-alkyl linker, an amido-alkyl or substituted amido-alkyl linker and an alkyl-amido-alkyl or substituted alkyl-amido-alkyl linker. In some instances, L³ can be a bond. In some instances, L³ can be an alkyl or substituted alkyl linker, an alkenyl or substituted alkenyl linker, an alkynyl or substituted alkynyl linker, an alkoxy or substituted alkoxy linker and X can be an aryl group.

In some instances, L¹ and L³ are each independently selected from a C₁-C₁₂ alkyl or substituted alkyl linker, a C₁-C₁₂ alkenyl or substituted alkenyl linker, a C₁-C₁₂ alkynyl or substituted alkynyl linker, a C₁-C₁₂ acyl or substituted acyl linker, a C₁-C₁₂ alkoxy or substituted alkoxy linker, a C₁-C₁₂ amido-alkyl or substituted amido-alkyl linker, a C₁-C₁₂ alkyl-amido-alkyl or substituted alkyl-amido-alkyl linker, a sulfonamide, a sulfinamide, a disulfonamide, a disulfinamide, a sultam, an amide, a secondary amine, a phosphonamide, a phosphinamide, a phosphonamidate, a selenonamide, and a seleninamide. In certain cases, L³ comprises a carbonyl group or alkoxy group, and L¹ is a C₁-C₁₂ alkyl or substituted alkyl, a sulfonamide, a sulfinamide, a disulfonamide, a disulfinamide, a sultam, an amide, a secondary amine, a phosphonamide, a phosphinamide, a phosphonamidate, a selenonamide, and a seleninamide. In some instances, L³ can be an alkoxy or substituted alkoxy linker, X can be absent, and L¹ can be a sulfonamide, a sulfinamide, a disulfonamide, a disulfinamide, a sultam, an amide, a secondary amine, a phosphonamide, a phosphinamide, a phosphonamidate, a selenonamide, or a seleninamide.

In some instances, the branching point X¹ is selected from N, CR′, C(═O)N, SO₂N, a tri-substituted aryl moiety (e.g., a 1,3,5-phenyl), a tetra-substituted aryl moiety (e.g., a 1, 3, 4, 5-phenyl), and a tri-substituted heteroaryl group. In certain instances, the branching point X¹ is a nitrogen atom. In other instances, the branching point X¹ is CR′, where R′ is selected from hydrogen, alkyl, substituted alkyl, or -L³-W¹ (e.g., as described herein).

The term “water soluble” when referring to a polymer as used herein refers to a polymer having solubility in “water” as used herein of 1 mg/mL or more, such as 3 mg/mL or more, 10 mg/mL or more, 20 mg/mL or more, 30 mg/mL or more, 40 mg/mL or more, 50 mg/mL or more, 60 mg/mL or more, 70 mg/mL or more, 80 mg/mL or more, 90 mg/mL or more, 100 mg/mL or more, or even more. It is understood that water soluble polymers may, under certain conditions, form discrete water-solvated nanoparticles in aqueous systems and can be resistant to aggregation. The fluorescent compounds of the disclosure can be water soluble. The fluorescent polymers of the disclosure can be water soluble.

The term “cyanine” as used herein refers to a substituted or unsubstituted bridge unit permitting delocalization across the molecules or monomers of the invention. In some embodiments, the “cyanine” is a substituted or unsubstituted methine or polymethine unit, such as a tri-, penta- or heptamethine unit. For example, in some embodiments, “cyanine” refers to substituted and unsubstituted groups, such as the following:

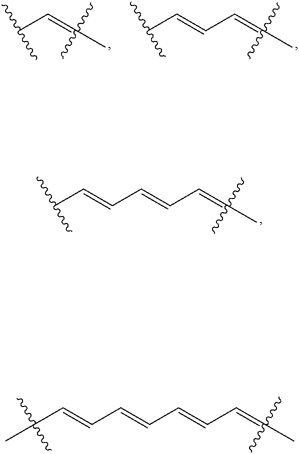

In some embodiments, cyanine is

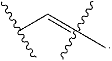 [SMILES: *C=*]

In other embodiments, cyanine is

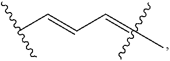 [SMILES: */C=C/C=*]

In still other embodiments, cyanine is

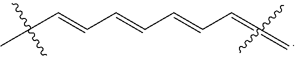 [SMILES: */C=C/C=C/C=C/C=*]

The term “squaraine” as used herein refers to a substituted or unsubstituted cyclic group, such as a 4-membered ring. In addition, the molecules and monomers of the invention include other “squaraine” like cyclic groups and heterocyclic groups, including substituted or unsubstituted five- or six-membered cyclic and heterocyclic groups. For example, squaraine and squarine like groups include, but are not limited to, substituted or unsubstituted cyclic and heterocyclic groups such as the following:

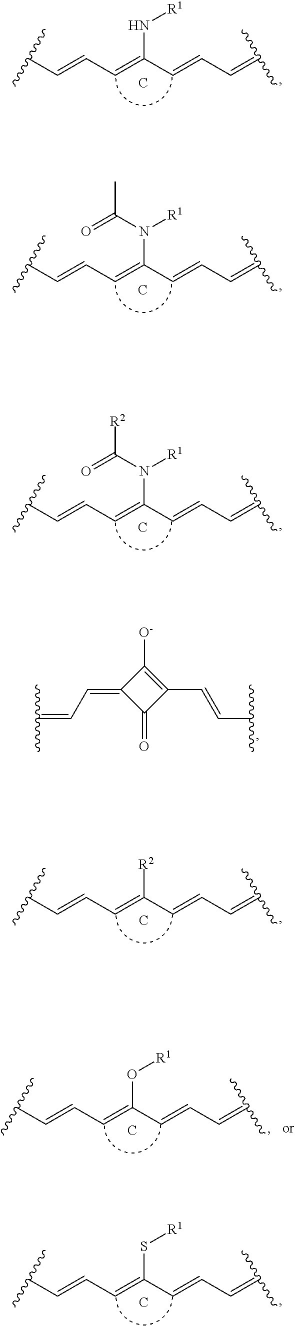

wherein C is a four-, five- or six-membered cyclic group. In some embodiments, the cyclic squaraine group is

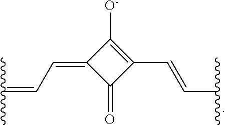 [SMILES: */C=C/C1=C([O-])C(=C/C=*)/C1=O]

In some embodiments, the cyclic squaraine like cyclic group is

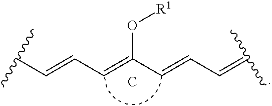

In some embodiments, the cyclic squaraine like cyclic groups is

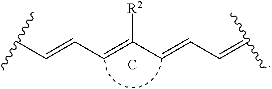

In other embodiments, the cyclic squaraine like cyclic group is

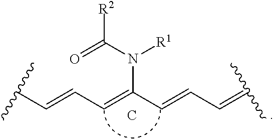

In still other embodiments, the cyclic squaraine like cyclic group is

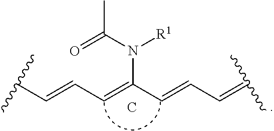

### III. DHP-Cyanine and DHP-Squaraine Monomers

The disclosure provides DHP-cyanine and DHP-squaraine organic dye monomeric compounds, polymers, and methods for making. Both small organic dyes as well as polymer dyes can be prepared according to methods provided in the present disclosure. Resulting DHP-cyanine and DHP-squaraine dyes exhibit excitation maxima in a range of from about 420-900 nm for monomeric dyes. In addition to the specific structures disclosed herein, structural isomers of the disclosed structures are also included.

The DHP-cyanine and DHP-squaraine compounds of the present disclosure each comprise at least one 9,10-dihydrophenanthrene (DHP) moiety or derivative thereof. The DHP-cyanine or DHP-squaraine compound according to the disclosure may comprise a structure according to Formula (I)

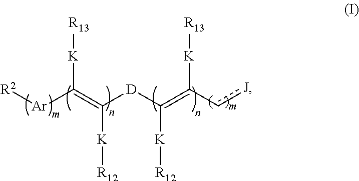

wherein
D is an aryl group, heteroaryl group,

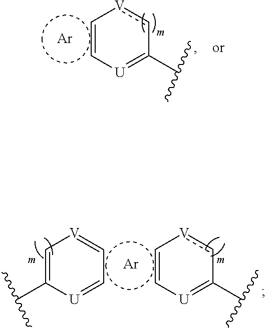

J is an aryl group, a heteroaryl group, or

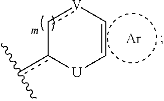

and
at least one of D or J is independently selected from the group consisting of

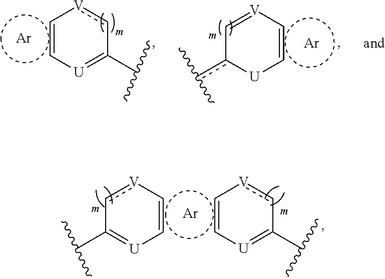

wherein the optional Ar and optional

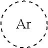

are each independently selected from the group consisting of a substituted or unsubstituted benzene, benzene derivative, monocyclic aryl group, polycyclic aryl group, monocyclic heteroaryl group, and polycyclic heteroaryl group, wherein at least one

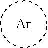

of D or J must be present and comprises a DHP structure

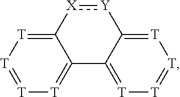

or a derivative thereof comprising an additional aryl or heteroaryl group fused at any available

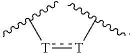

In some instances, D is

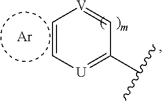

J is

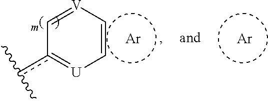

is

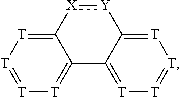

or a derivative thereof comprising an additional aryl or heteroaryl group fused at any available

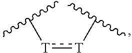

optionally wherein the derivative is

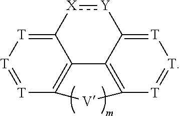

In some instances, D is

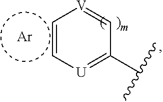

J is

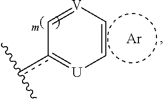

the

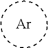

of D is

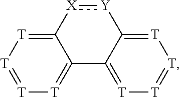

or a derivative thereof comprising an additional aryl or heteroaryl group fused at any available

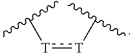

optionally wherein the derivative is

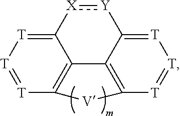

and the

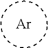

of J is substituted or unsubstituted benzene, or a derivative thereof. In some instances, D is

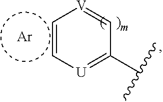

J is

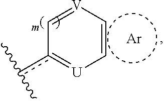

the

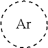

of D is substituted or unsubstituted napthalene, and the

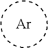

of J is

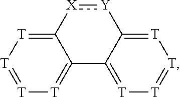

or a derivative thereof comprising an additional aryl or heteroaryl group fused at any available

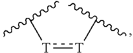

optionally wherein the derivative is

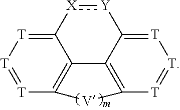

The

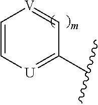

and/or

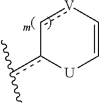

may be fused to the DHP structure

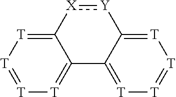

or derivative thereof at any available

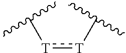

position, e.g., at the 1,2-, 2,3-, 3,4-, 5,6-, 6,7- and 7,8-positions of the DHP structure or derivative thereof.

Each T may independently be selected from the group consisting of C(R¹), N, P, O, S, and Si(R¹). Each T may independently be C(R¹) or N.

Each U may independently be selected from the group consisting of NR¹⁰, O, P, and S. Each U may independently be NR¹⁰.

Each V may independently be selected from the group consisting of NR¹¹, CR¹¹, C(R¹¹)₂, S, O, and Si(R¹¹)₂. Each V may independently be CR¹¹, C(R¹¹)₂, S, or O. Each V may independently be CR¹¹ or C(R¹¹)₂.

Each V′ may independently be selected from the group consisting of SO₂, SO, S, NR¹¹, CR¹¹, C(R¹¹)₂, O, Si(R¹¹)₂, >C═O, >Se═O, —CH═CH—, and —N═CH—. Each V′ may may independently be selected from the group consisting of SO₂, SO, and S.

Each X may independently be CR¹R² or SiR¹R². In some cases, each X may independently be CR¹R².

Each Y may independently be CR⁸R⁹ or SiR⁸R⁹. In some cases, each Y may independently be CR⁸R⁹.

Each R¹, R², R⁸, and R⁹ may independently be selected from the group consisting of a water-solubilizing moiety, a linker moiety, a linked E, hydrogen, hydroxy, halogen, alkoxy, alkyl, alkenyl, alkynyl, cycloalkyl, haloalkyl, aryl, heteroaryl, (hetero)aryloxy, (hetero)arylamino, sulfonamide-PEG, phosphoramide-PEG, ammonium alkyl salt, ammonium alkyloxy salt, ammonium oligoether salt, carbonyl, acyl, sulfonate alkyl salt, sulfonate alkoxy salt, sulfonate oligoether salt, sulfonamido oligoether, sulfonamide, sulfinamide, phosphonamidate, phosphinamide, alkoxy sulfonamide PEG, alkoxy sulfonate, alkyl sulfonate, alkyl sulfonate salt,

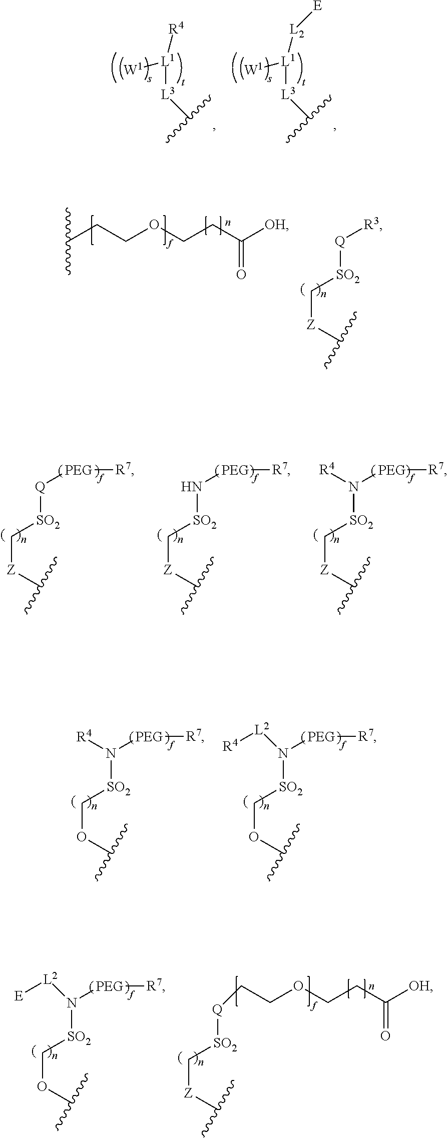

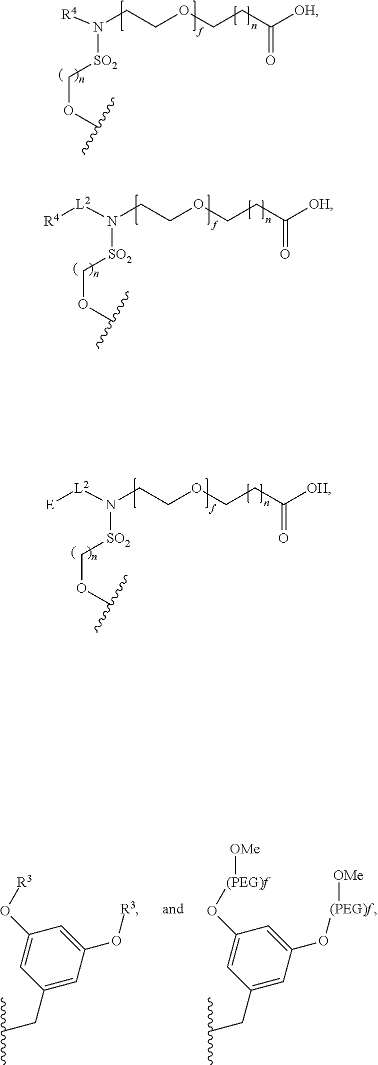

or R¹ and R⁸ together form an unsubstituted or substituted cycloalkyl, cycloalkenyl, heterocycloalkyl, heterocycloalkenyl, cycloalkoxy, aryl, or heteroaryl having 3 to 9 ring members.

Each R³ may independently be selected from the group consisting of H, alkyl, alkene, alkyne, cycloalkyl, haloalkyl, alkoxy, (hetero)aryloxy, aryl, (hetero)arylamino, a water-solubilizing moiety, a chromophore, functional moiety, binding partner, and a PEG group.

Each Q may independently be a bond, NH, NR⁴, C₁-C₁₂ alkylene, CHR⁴, and CH₂CH₂, CHR⁴, O, NR⁴, or NH; each Z is independently CH₂, CHR⁴, O, NR⁴, or NH.

Each W¹ may independently be a water-solubilizing moiety.

Each L¹, L², and L³ may each independently be selected linker moieties; each E is independently selected from the group consisting of a chromophore, a functional moiety, a substrate, and a binding partner.

Each R⁴ may independently be selected from the group consisting of H, alkyl, PEG, a water-solubilizing moiety, a linker moiety, a chromophore, carboxylic amine, amine, carbamate, carboxylic acid, carboxylate ester, maleimide, activated ester, N-hydroxysuccinimidyl, hydrazine, hydrazide, hydrazone, azide, alkyne, aldehyde, and thiol, or protected groups thereof.

Each R⁷ may independently be selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester, and C₁-C₁₂ alkoxy, a water-solubilizing moiety, a PEG moiety, a functional group, a chemoselective functional group, conjugation tag, linked conjugation tag, a linker, sulfonic acid, sulfonate, C₁-C₁₂ alkyl sulfonate, sulfonamide. In some cases, each R⁷ may independently be selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester, and C₁-C₁₂ alkoxy. In some cases, each R7 may independently be selected from the group consisting of a functional group, a chemoselective functional group, conjugation tag, linked conjugation tag, a linker, sulfonic acid, sulfonate, C₁-C₁₂ alkyl sulfonate, and sulfonamide.

Each R¹⁰ may independently be selected from the group consisting of hydrogen, a linker moiety, a linked reactive group, a linked ionic group, a linked chromophore, a linked water-solubilizing moiety, a water-solubilizing moiety, a chromophore, a binding partner, a linked binding partner.

Each R¹¹ may be independently selected from the group consisting of hydrogen, a linker moiety, a linked reactive group, a linked ionic group, a linked chromophore, a linked water-solubilizing moiety, a water-solubilizing moiety, a chromophore, a binding partner, and a linked binding partner.

Each R¹² and R¹³ may be independently selected from the group consisting of hydrogen, C₁-C₆ alkyl, C₁-C₆ alkyl, C₃-C₁₀ cycloalkyl, substituted or unsubstituted aryl, and substituted or unsubstituted heteroaryl; or R¹² and R¹³ together form an unsubstituted or substituted unsaturated cycloalkyl group having 3 to 8 ring members or substituted or unsubstituted heterocycloalkyl having 3 to 8 ring members optionally substituted with O. Each K may be independently a covalent bond, O, S, P or CH₂.

Each f may independently be an integer from 0 to 50, 1 to 40, 2 to 30, 3 to 25, or 4 to 20.

Each m may independently be 0 or 1.

Each n may independently be 0, 1, 2, 3, or 4.

Each s may independently be 1 or 2.

Each t may independently be 0, 1, 2, or 3.

In some examples, at least one of R¹, R², R³, R⁴, R⁸, R⁹, R¹⁰, and R¹¹ comprises a water-solubilizing moiety or a linked water-solubilizing moiety.

The derivative of

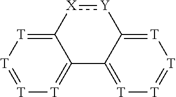

comprising an additional aryl or heteroaryl group fused at any available

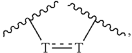

may optionally comprise

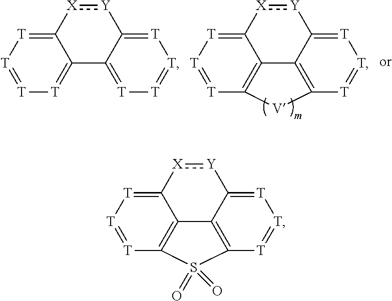

wherein each V′ is independently selected from SO₂, SO, S, NR¹¹, CR¹¹, C(R¹¹)₂, O, Si(R¹¹)₂, >C═O, >Se═O, —CH═CH—, or —N═CH—, and T, V, X, Y, and m are as defined herein above.

The DHP-cyanine or DHP-squaraine compound according to the disclosure may comprise a structure according to Formula (II):

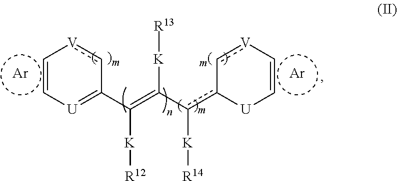

wherein
each

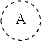

is independently selected from the group consisting of a substituted or unsubstituted benzene, benzene derivative, monocyclic aryl group, polycyclic aryl group, monocyclic heteroaryl group, and polycyclic heteroaryl group, wherein at least one

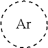

comprises

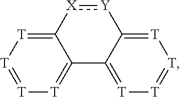

or a derivative thereof, wherein the derivative optionally comprises an additional aryl or heteroaryl group fused at any available

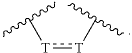

In some cases, the derivative of

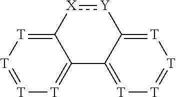

is

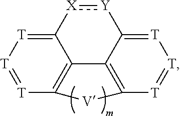

wherein
each V′ is independently SO₂, SO, S, NR¹¹, CR¹¹, C(R¹¹)₂, O, Si(R¹¹)₂, >C═O, >Se═O, —CH═CH—, or —N═CH—;
each T is independently selected from the group consisting of C, C(R¹), N, P, O, S, and Si(R¹);
each U is independently selected from the group consisting of NR¹⁰, O, P, Se, Te, and S;
each V is independently selected from the group consisting of NR¹¹, CR¹¹, C(R¹¹)₂, S, SO₂, O, Se, Te, and Si(R¹¹)₂;
each X is independently CR¹R² or SiR¹R²;
each Y is independently CR⁸R⁹ or SiR⁸R⁹; and
each m is independently 0 or 1.

In some cases, each

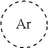

is independently

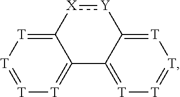

wherein each T is independently selected from the group consisting of C and C(R¹);
each U is independently selected from the group consisting of NR¹⁰, O, P, Se, Te, and S;
each V is independently selected from the group consisting of NR¹¹, CR¹¹, C(R¹¹)₂, S, SO₂, O, Se, Te, and Si(R¹¹)₂;
each X is independently CR¹R² or SiR¹R²,
each Y is independently CR⁸R⁹ or SiR⁸R⁹; and
each m is independently 0 or 1.

In some cases, each

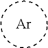

is independently

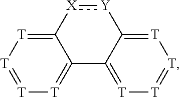

wherein each T is independently selected from the group consisting of C, and C(R¹);
each U is independently NR¹⁰;
each V is independently selected from the group consisting of CR¹¹, and C(R¹¹)₂;
each X is independently CR¹R²;
each Y is independently CR⁸R⁹; and
each m is independently 0 or 1.

In some cases, one

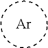

group is a substituted or unsubstituted benzene, benzene derivative, or substituted or unsubstituted naphthalene, or naphthalene derivative, and the other

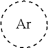

group comprises

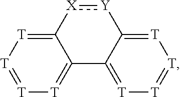

or a derivative thereof, wherein the derivative optionally comprises an additional aryl or heteroaryl group fused at any available

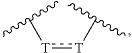

optionally

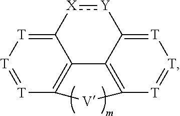

wherein each T is independently selected from the group consisting of C, and C(R¹);
each U is independently NR¹⁰;
each V is independently selected from the group consisting of CR¹¹, and C(R¹¹)₂;
each V′ is independently selected from the group consisting of SO₂, SO, and S;
each X is independently CR¹R²,
each Y is independently CR⁸R⁹; and
each m is independently 0 or 1.

In some cases, V is CR¹¹ or C(R¹¹)₂.

Each R¹, R², R⁸, and R⁹ can be independently selected from the group consisting of a water-solubilizing moiety, a linked water-solubilizing moiety, a linker moiety, a linked E, a reactive group, a linked reactive group, binding partner, linked binding partner, a functional group, hydrogen, hydroxy, halogen, alkoxy, alkyl, alkenyl, alkynyl, cycloalkyl, haloalkyl, aryl, heteroaryl, (hetero)aryloxy, (hetero)arylamino, sulfonamide-PEG, phosphoramide-PEG, ammonium alkyl salt, ammonium alkyloxy salt, ammonium oligoether salt, carbonyl, acyl, sulfonate alkyl salt, sulfonate alkoxy salt, sulfonate oligoether salt, sulfonamido oligoether, sulfonamide, sulfinamide, phosphonamidate, phosphinamide, alkoxy sulfonamide PEG, alkoxy sulfonate, alkyl sulfonate, alkyl sulfonate salt,

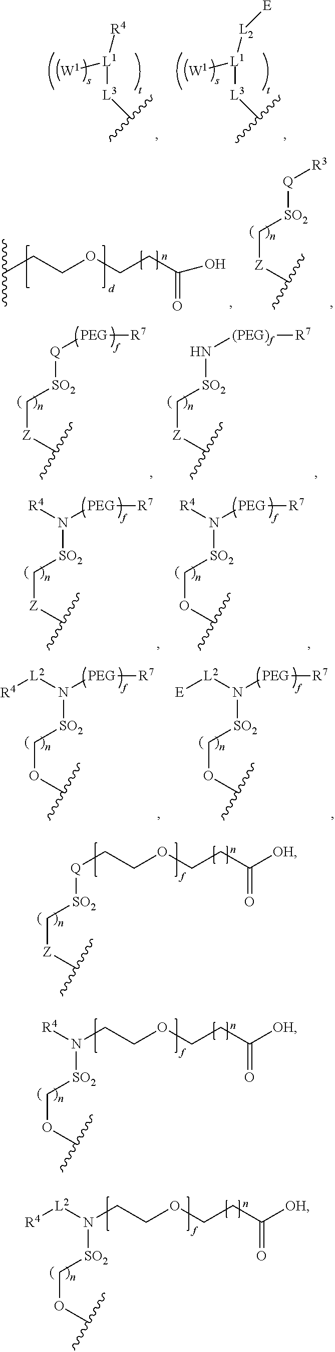

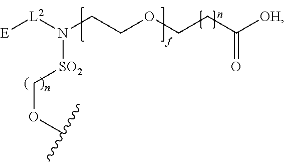

In some cases, each R¹, R², R⁸, and R⁹ can be independently selected from the group consisting of a water-solubilizing moiety, a linked water-solubilizing moiety, a linker moiety, a linked E, a reactive group, a linked reactive group, binding partner, linked binding partner, a functional group, hydrogen, hydroxy, halogen, alkoxy, alkyl, alkenyl, alkynyl, cycloalkyl, and haloalkyl. In some cases, each R¹, R², R⁸, and R⁹ can be independently selected from the group consisting of a water sulfonamide-PEG, phosphoramide-PEG, ammonium alkyl salt, ammonium alkyloxy salt, ammonium oligoether salt, carbonyl, acyl, sulfonate alkyl salt, sulfonate alkoxy salt, sulfonate oligoether salt, sulfonamido oligoether, sulfonamide, sulfinamide, phosphonamidate, phosphinamide, alkoxy sulfonamide PEG, alkoxy sulfonate, alkyl sulfonate, alkyl sulfonate salt,

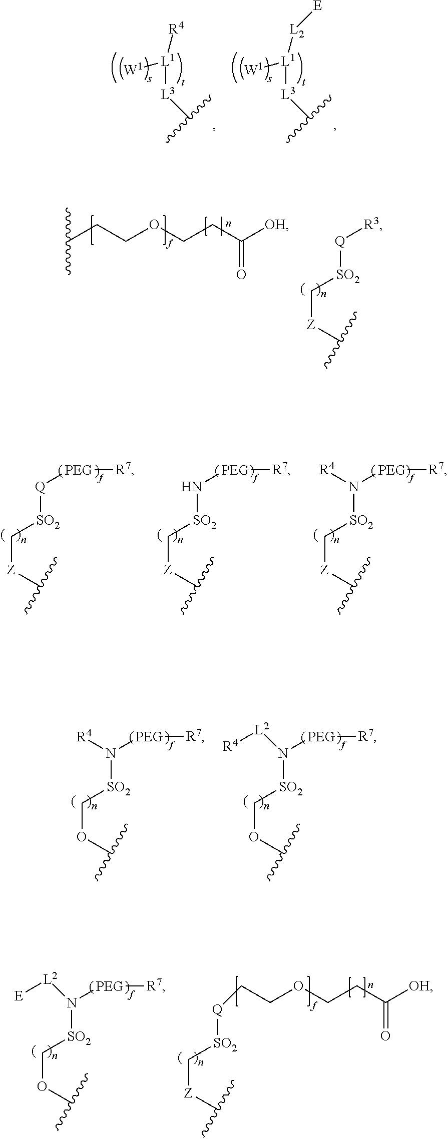

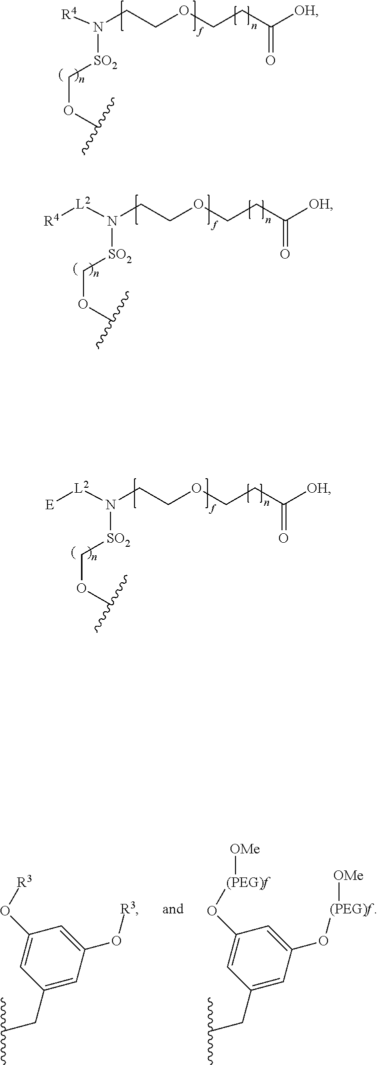

In some cases, each R¹, R², R⁸, and R⁹ can be independently selected from the group consisting of a water-solubilizing moiety, a linked water-solubilizing moiety, a linker moiety, a linked E, a reactive group, a linked reactive group, binding partner, linked binding partner, a functional group. In some cases, each R¹, R², R⁸, and R⁹ can be independently selected from the group consisting of hydrogen, hydroxy, halogen, alkoxy, alkyl, alkenyl, alkynyl, cycloalkyl, and haloalkyl.

In some cases, R¹ and R⁸ together form an unsubstituted or substituted cycloalkyl, cycloalkenyl, heterocycloalkyl, heterocycloalkenyl, cycloalkoxy, aryl, or heteroaryl having 3 to 9 ring members.

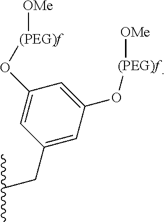

In some cases, each R³ is independently selected from the group consisting of H, alkyl, alkene, alkyne, cycloalkyl, haloalkyl, alkoxy, (hetero)aryloxy, aryl, (hetero)arylamino, a water-solubilizing moiety, a chromophore, functional moiety, binding partner, and a PEG group. In some cases, each R³ is independently selected from the group consisting of a water-solubilizing moiety, a chromophore, functional moiety, binding partner, and a PEG group (e.g., (PEG)_{f}-OMe and (PEG)_{f}-OEt).

In some cases, each Q is independently a bond, NH, NR⁴, C₁-C₁₂ alkylene, CHR⁴, or CH₂. In some cases, each Q is independently a bond, NH, or NR⁴.

In some cases, each Z is independently CH₂, CHR⁴, O, NR⁴, or NH. In some cases, Z is O.

In some cases, each W¹ is independently a water-solubilizing moiety.

In some cases, L¹, L², and L³ are each independently selected linker moieties.

In some cases, each E is independently selected from the group consisting of a chromophore, a functional moiety, a conjugation tag, a substrate, and a binding partner.

In some cases, each R⁴ is independently selected from the group consisting of H, alkyl, PEG, a water-solubilizing moiety, a linker moiety, a chromophore, a linked chromophore, a functional group, a conjugation tag, carboxylic amine, amine, carbamate, carboxylic acid, carboxylate ester, maleimide, activated ester, N-hydroxysuccinimidyl, hydrazine, hydrazide, hydrazone, azide, alkyne, cycloalkyne, alkene, cycloalkene, tetrazine, aldehyde, and thiol, or protected groups thereof.

In some cases, each R⁷ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester, and C₁-C₁₂ alkoxy.

In some cases, each R¹⁰ is independently selected from the group consisting of hydrogen, a linker moiety, a linked reactive group, a linked ionic group, a linked chromophore, a linked water-solubilizing moiety, a water-solubilizing moiety, a chromophore, a binding partner, a linked binding partner.

In some cases, each R¹¹ is independently selected from the group consisting of hydrogen, a linker moiety, a linked reactive group, a linked ionic group, a linked chromophore, a linked water-solubilizing moiety, a chromophore, a binding partner, and a linked binding partner.

In some cases, each R¹², R¹³, and R¹⁴ is independently selected from the group consisting of hydrogen, halogen, substituted or unsubstituted C₁-C₆ alkyl, substituted or unsubstituted C₁-C₆ heteroalkyl, substituted or unsubstituted C₁-C₆ alkene, substituted or unsubstituted C₃-C₁₀ cycloalkyl, substituted or unsubstituted heterocycloalkyl, substituted or unsubstituted aryl, and substituted or unsubstituted heteroaryl, CO₂R¹, CONR₁R², —O—CH₂CH₂-PEG-R⁷, —S—CH₂CH₂-PEG-R⁷, —N—CH₂CH₂-PEG-R⁷, O-aryl, S-aryl, N-aryl, —O-alkyl, S-alkyl, N-alkyl, wherein each alkyl or aryl can optionally be substituted with one or more R⁷, PEG, PEG-R⁷, or linking groups, optionally wherein each R¹², R¹³ and R¹⁴ is independently substituted with a R⁷ group; or at least two of R¹⁰, R¹¹, R¹², R¹³, and R¹⁴ together, including but not limited to two, three, or four of R¹⁰, R¹¹, R¹², R¹³, and R¹⁴ together, form an unsubstituted or substituted unsaturated or partially unsaturated C₃-C₁₀ cycloalkyl group, unsubstituted or substituted unsaturated or partially unsaturated C₃-C₁₀ heterocycloalkyl optionally substituted with O, an unsubstituted or substituted unsaturated or partially unsaturated cycloalkyl group having 3 to 8 ring members, or substituted or unsubstituted heterocycloalkyl having 3 to 8 ring members optionally substituted with O;

In some cases, each K is independently a covalent bond, O, S, P, NR¹, Se, Te, CR¹R², or CH₂.

In some cases, each f is independently an integer from 0 to 50.

In some cases, each m is independently 0 or 1.

In some cases, each n is independently 0, 1, 2, 3, or 4.

In some cases, each s is independently 1 or 2.

In some cases, each t is independently 0, 1, 2, or 3.

In some examples, the DHP-cyanine and DHP-squaraine compounds of the present disclosure may be selected from Formula (IIa), (IIb), (IIc), (IId), (IIe), (IIf), (IIg), (IIh), (IIi), (IIj), (Ik), (IIl), (IIm), (IIn), (IIo), (IIp), (IIq), (IIr), (IIs), (IIt), (IIu), (IIv), (IIw), (IIx), (IIy), or (IIz):

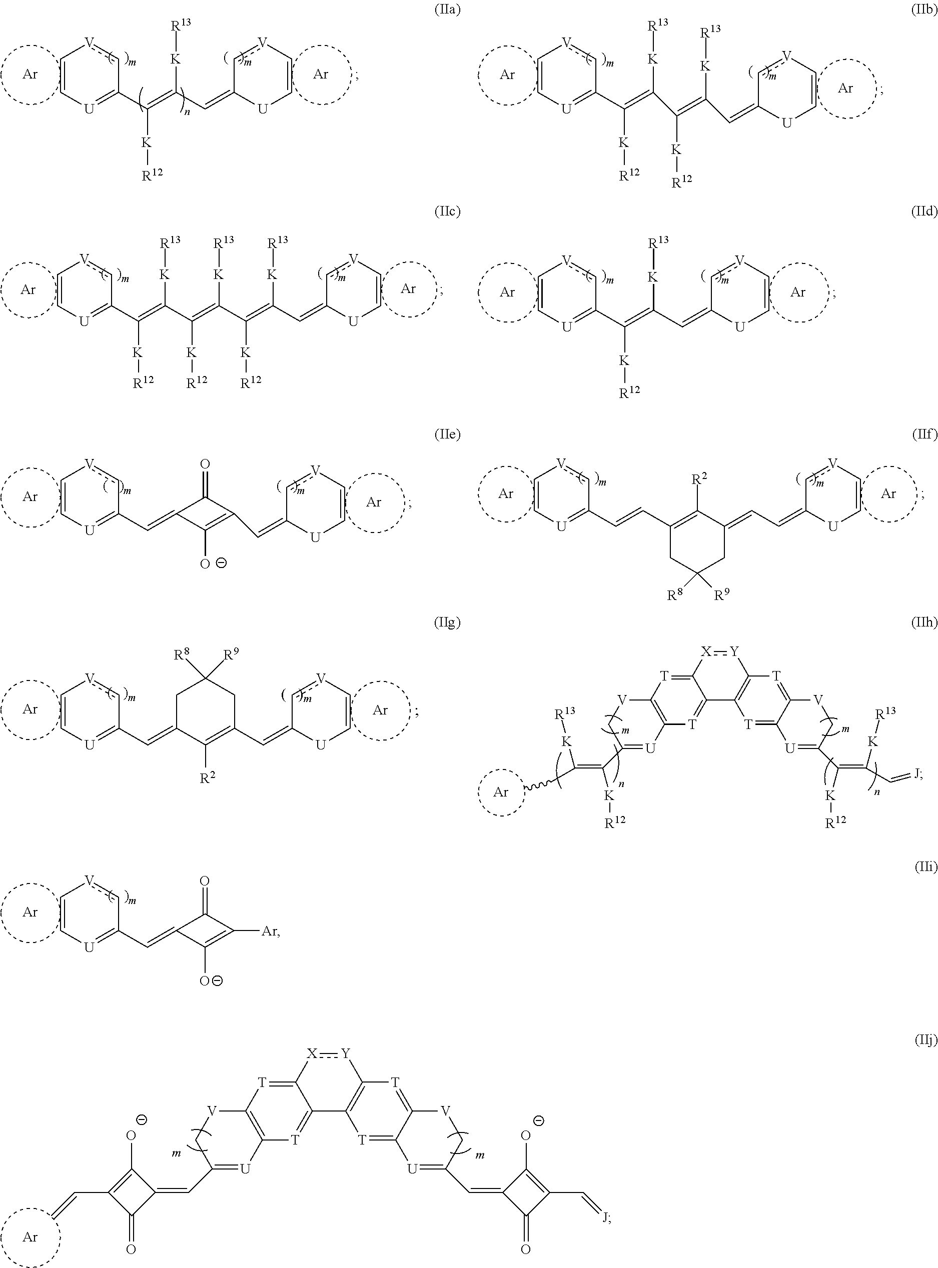

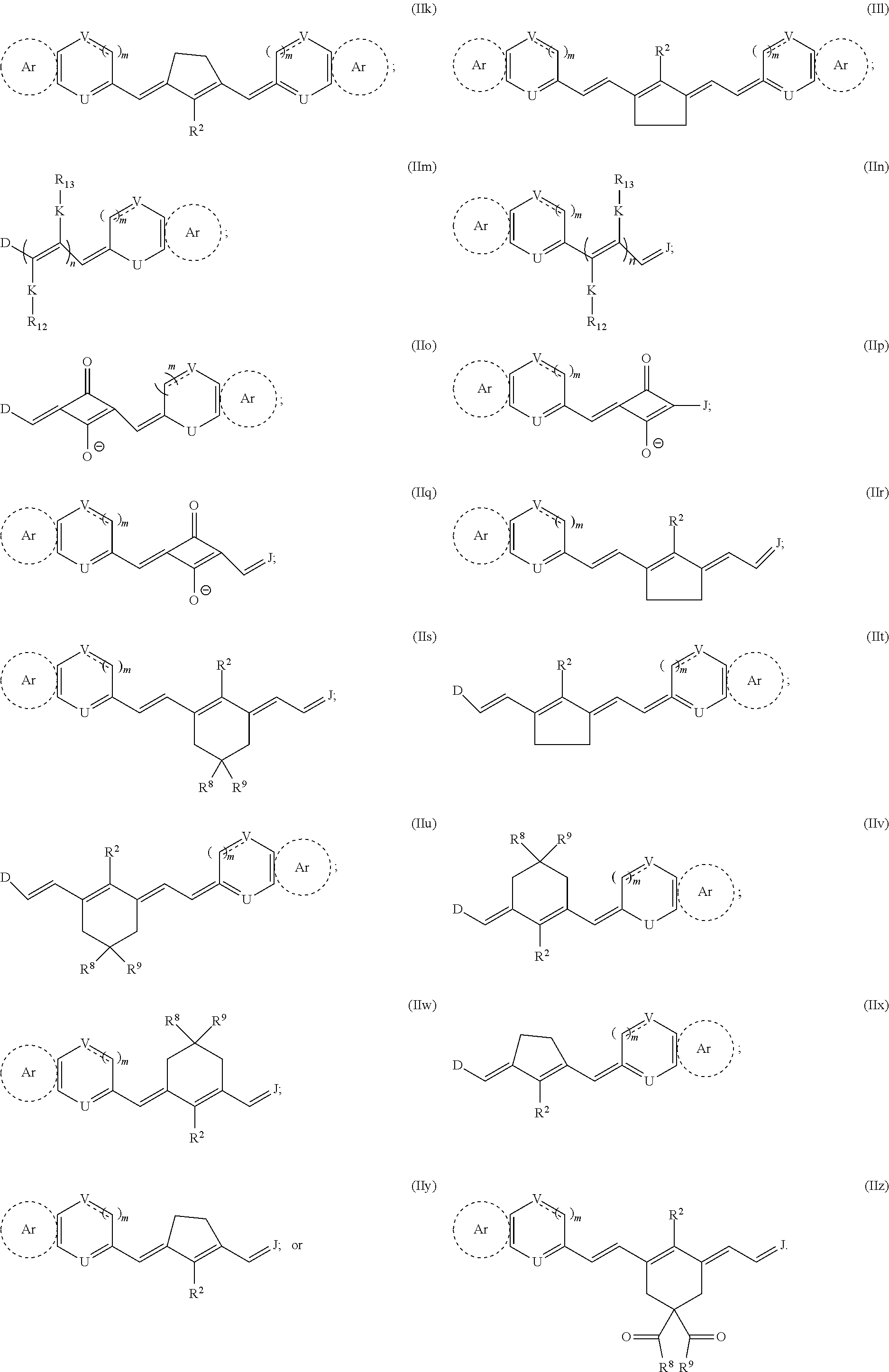

In some examples, the DHP-cyanine and DHP-squaraine compounds of the present disclosure may be selected from Formula (IIIa), (IIIb), (IIIc), (IIId), (IIIe), (IIIf), (IIIg), (IIIh), (IIIi), (IIIj), (IIIk), (IIIl), (IIIm), (IIIn), (IIIo), (IIIp), (IIIq), (IIIr), (IIIs), (IIIt), (IIIu), (IIIv), (IIIw), (IIIx), (Illy), (IIIz), (IIIaa), (IIIbb), (IIIcc), (IIIdd), or (IIIee):

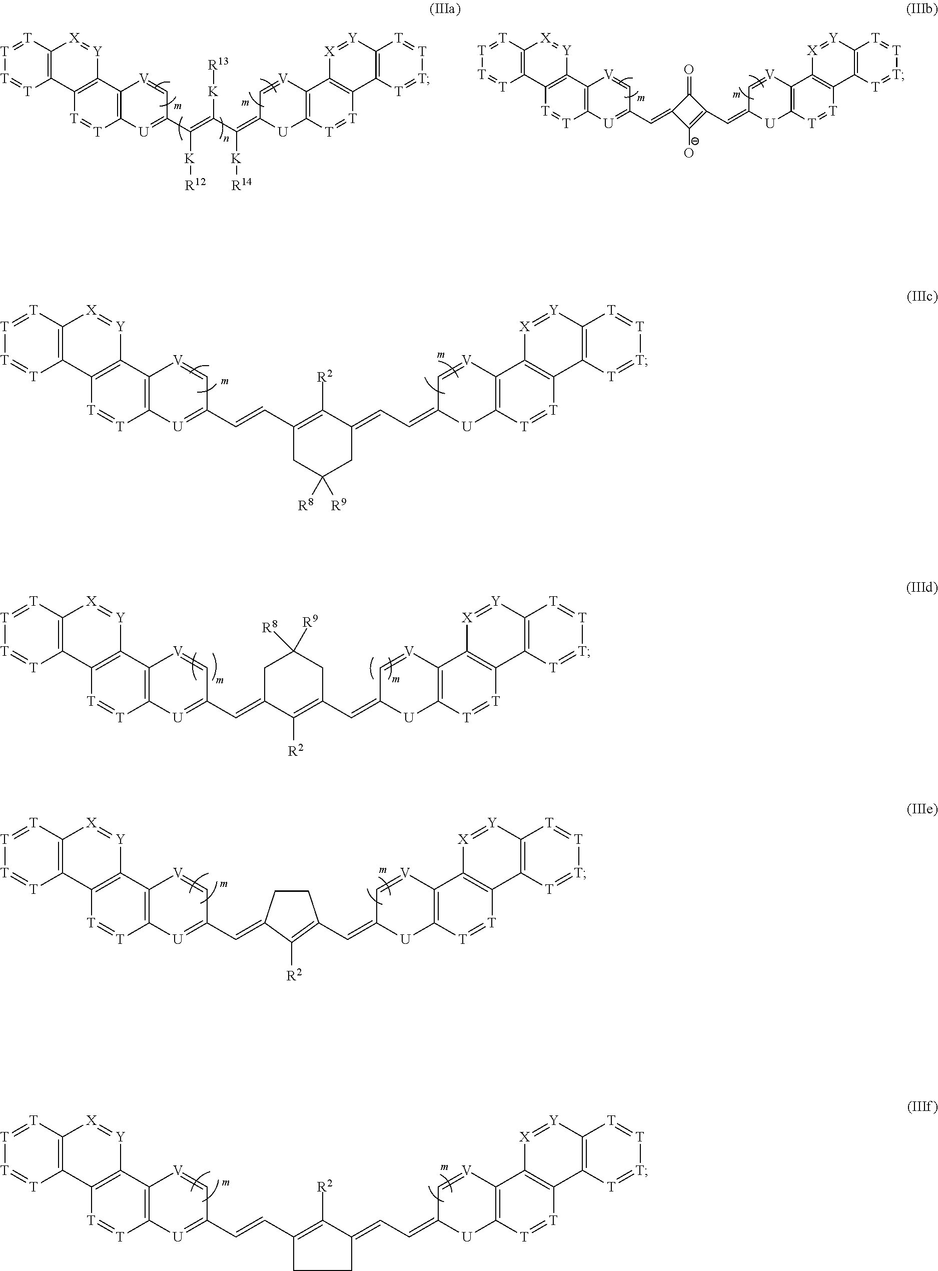

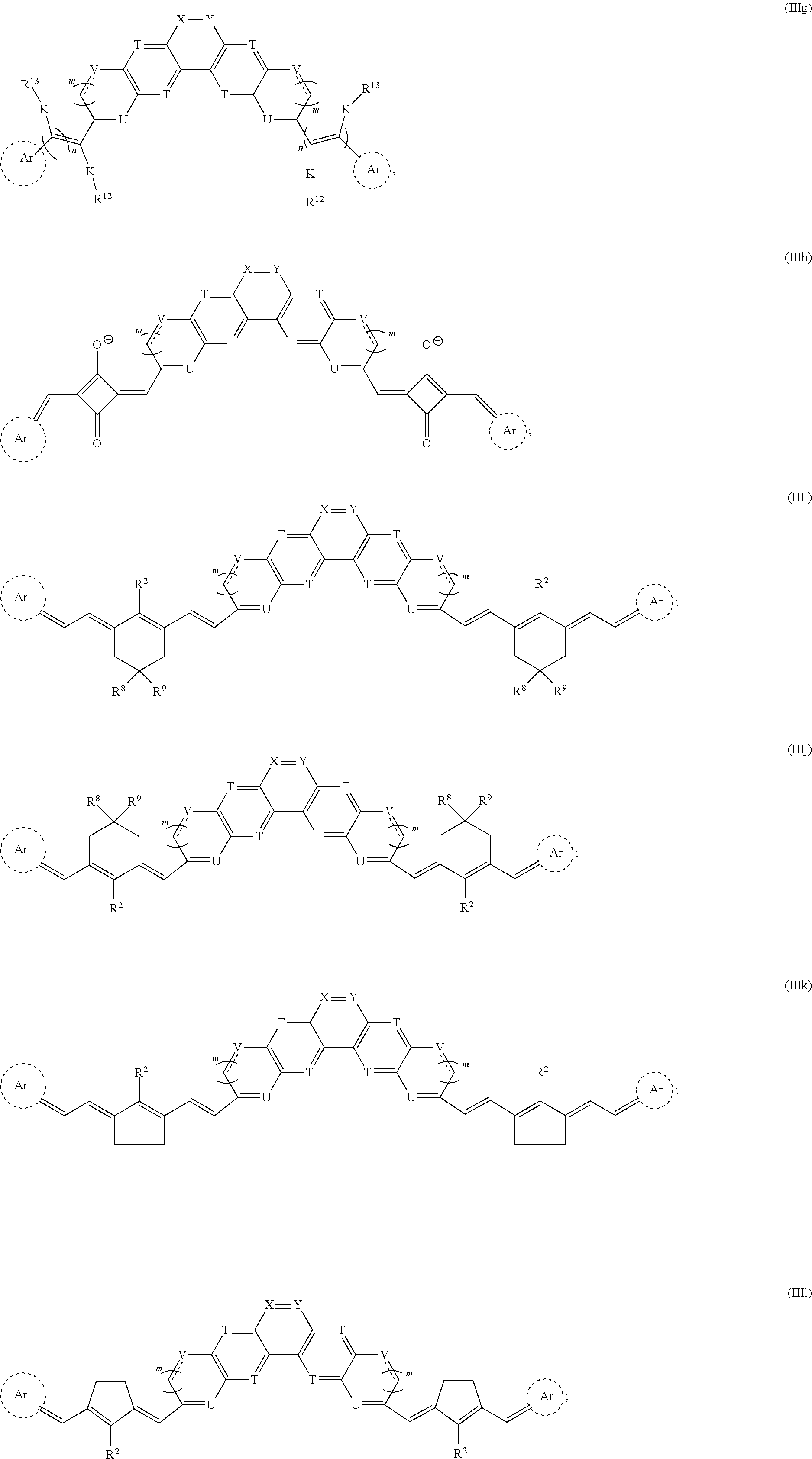

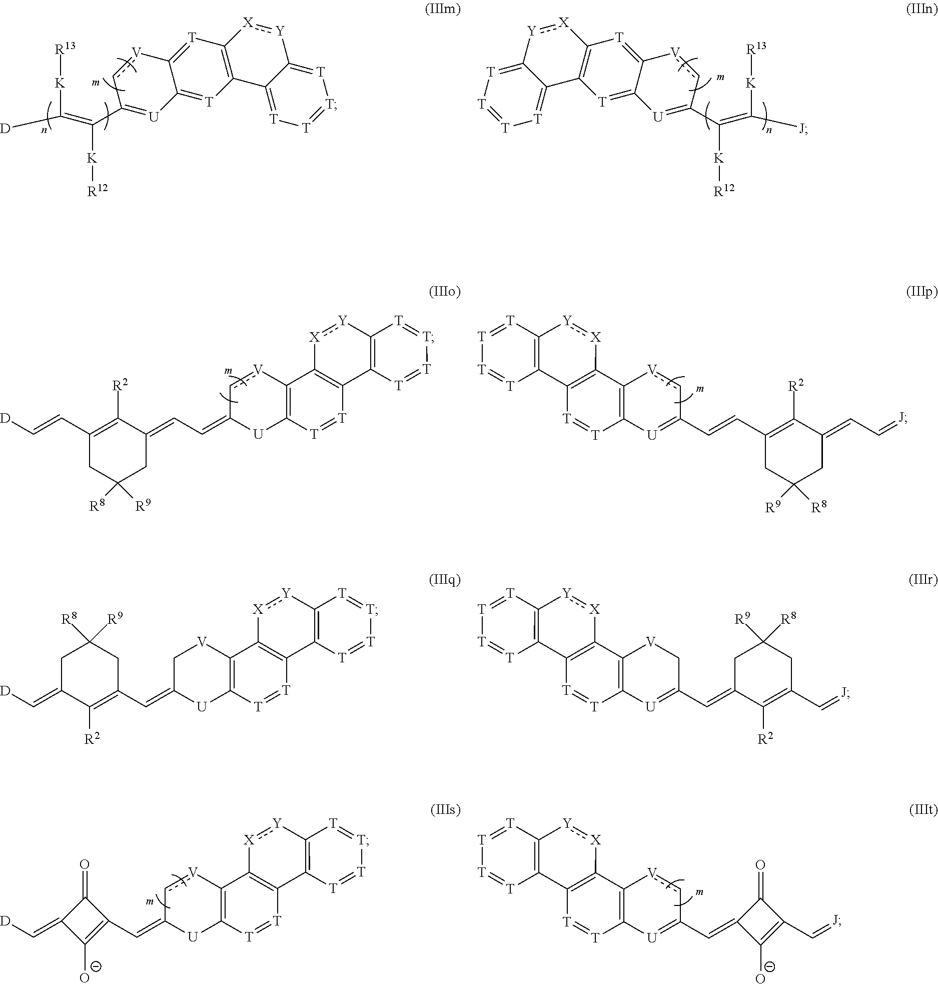

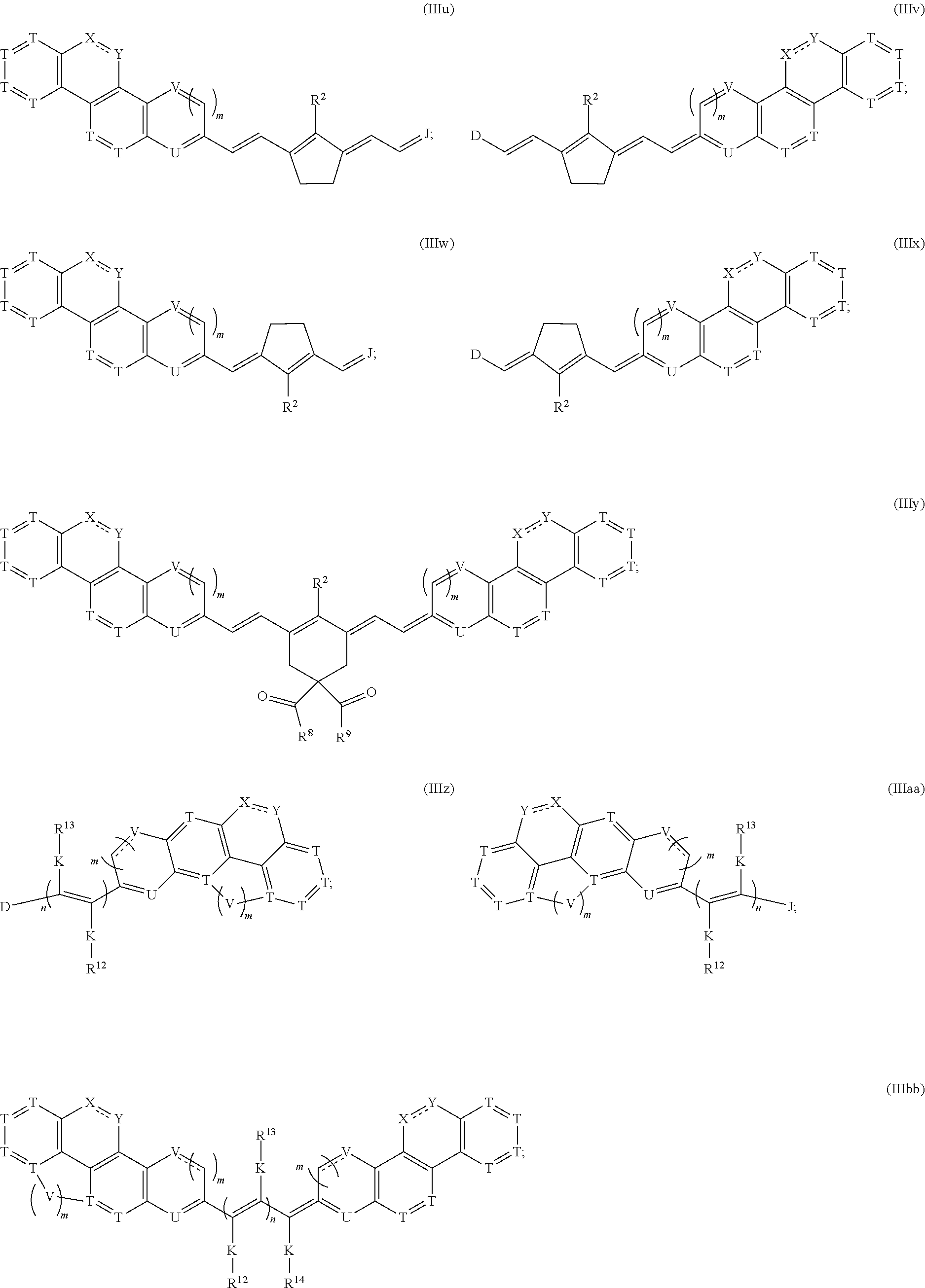

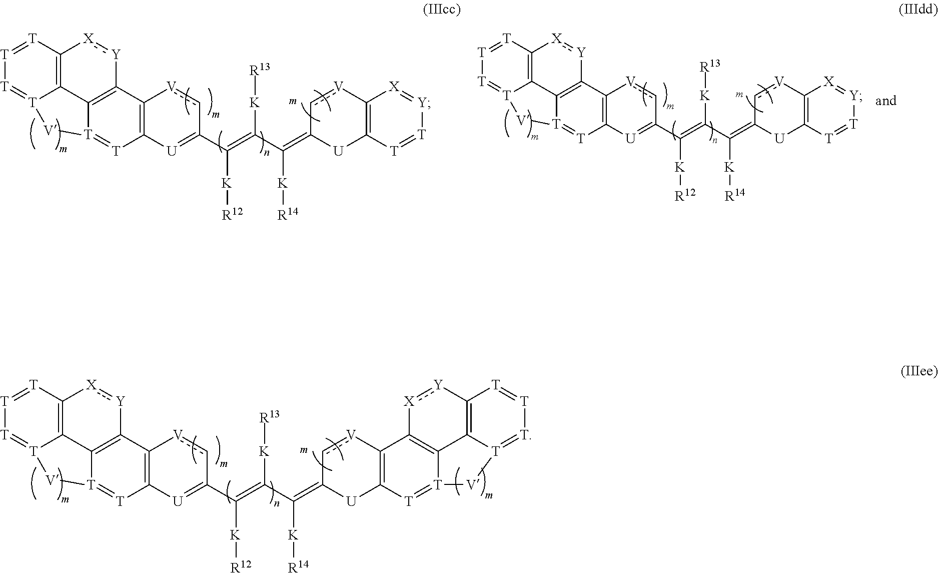

In some examples, the

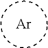

groups in both D and J may comprise the same or different

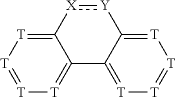

group or derivative thereof. In some examples, the

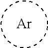

groups in both D and J comprise the same

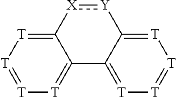

group or derivative thereof. In some examples, the

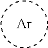

groups in both D and J comprise different

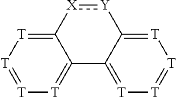

groups or derivatives thereof.

In some examples, D is

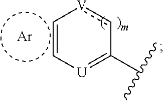

wherein

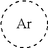

### of D is

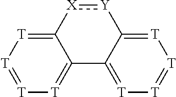

or a derivative thereof, and J is an aryl, heteroaryl or

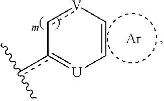

wherein

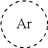

of J is an unsubstituted or substituted benzene, benzene derivative comprising fused monocyclic aryl, polycyclic aryl, monocyclic heteroaryl, or polycyclic heteroaryl group.

In some examples, J is

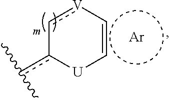

wherein

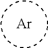

of J is an unsubstituted or substituted benzene or unsubstituted or substituted naphthalene; U is N; and V is N, O, or S.

In some examples, J is an unsubstituted or substituted quinoline, benzoxazole, benzothiazole, benzimidazole, indole, benzindole, pyridinium, benzopyrylium, thiopyrylium, fluorene, 9H-fluorene, phenanthrene, dihydrophenanthrene, 9,10-dihydrophenanthrene, 6,8-dihydro-5H-naphtho[2,1-f]indole, 4,5-dihydro-3H-naphtho[2,1-e]indole, 6,7-dihydro-3H-naphtho[2,1-g]indole, 5,6-dihydrophenanthro[3,2-d]thiazole, 4,5-dihydrophenanthro[2,1-d]thiazole, 6,7-dihydrophenanthro[4,3-d]thiazole, 5,6-dihydrophenanthro[3,2-d]oxazole, 4,5-dihydrophenanthro[2,1-d]oxazole, 6,7-dihydrophenanthro[4,3-d]oxazole, 5,6-dihydronaphtho[2,1-g]quinoline, 7,8-dihydronaphtho[2,1-h]quinoline, 5,6-dihydronaphtho[2,1-f]quinoline, 5,6-dihydro-8λ2-phenanthro[3,2-d]imidazole, 4,5-dihydro-3λ2-phenanthro[2,1-d]imidazole, 6,7-dihydro-3λ2-phenanthro[4,3-d]imidazole, 5,6-dihydronaphtho[1,2-g]quinoxaline, 5,6-dihydronaphtho[2,1-f]quinoxaline, 7,8-dihydronaphtho[1,2-f]quinoxaline, phenyl, 1H-benzo[e]indol-3-ium, and 1H-benzo[e]indole.

In some examples, D is aryl, heteroaryl or

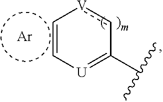

wherein

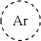

of D is a substituted or unsubstituted benzene, benzene derivative, monocyclic aryl group, polycyclic aryl group, monocyclic heteroaryl group, and polycyclic heteroaryl group; and J is

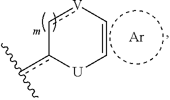

wherein

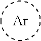

### of J is

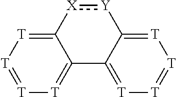

or a derivative thereof.

In some examples, D is

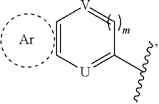

wherein

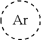

of D is an unsubstituted or substituted benzene or unsubstituted or substituted naphthalene; U is N; and V is N, O, or S.

In some examples, D is selected from unsubstituted or substituted quinoline, benzoxazole, benzthiazole, benzimidazole, indole, benzindole, pyridinium, benzopyrylium, thiopyrylium, fluorene, 9H-fluorene, phenanthrene, dihydrophenanthrene, 9,10-dihydrophenanthrene, 6,8-dihydro-5H-naphtho[2,1-f]indole, 4,5-dihydro-3H-naphtho[2,1-e]indole, 6,7-dihydro-3H-naphtho[2,1-g]indole, 5,6-dihydrophenanthro[3,2-d]thiazole, 4,5-dihydrophenanthro[2,1-d]thiazole, 6,7-dihydrophenanthro[4,3-d]thiazole, 5,6-dihydrophenanthro[3,2-d]oxazole, 4,5-dihydrophenanthro[2,1-d]oxazole, 6,7-dihydrophenanthro[4,3-d]oxazole, 5,6-dihydronaphtho[2,1-g]quinoline, 7,8-dihydronaphtho[2,1-h]quinoline, 5,6-dihydronaphtho[2,1-f]quinoline, 5,6-dihydro-8λ2-phenanthro[3,2-d]imidazole, 4,5-dihydro-3λ2-phenanthro[2,1-d]imidazole, 6,7-dihydro-3λ2-phenanthro[4,3-d]imidazole, 5,6-dihydronaphtho[1,2-g]quinoxaline, 5,6-dihydronaphtho[2,1-f]quinoxaline, 7,8-dihydronaphtho[1,2-f]quinoxaline, phenyl, 3,5,6,8-tetrahydrophenanthro[2,3-e:7,6-e′]diindole, 5,6-dihydropentapheno[3,4-d:10,9-d′]bis(oxazole), 5,6-dihydropentapheno[3,4-d:10,9-d′]bis(thiazole), 6,7-dihydrophenanthro[2,3-f:7,6-f]diquinoline, 5,6-dihydropentapheno[3,2,1-cd:10,11,12-c′d′]diindole, 2,6,7,11-tetrahydrobenzo[1,2-g:4,3-g′]dichromene, or 2,6,7,11-tetrahydrobenzo[1,2-g:4,3-g′]bis(thiochromene).

In some examples, D is

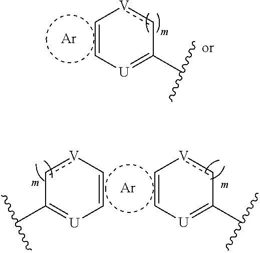

### or J is

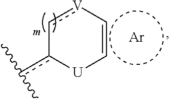

wherein
at least one

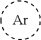

is a

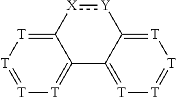

group or derivative thereof,
which is fused to

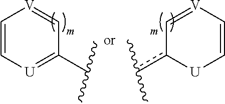

at any available

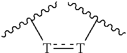

position on the

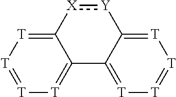

group or derivative thereof.

In some examples, D is

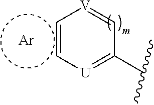

and the

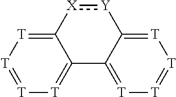

group or derivative thereof is fused to

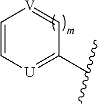

at any available

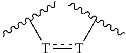

position on the

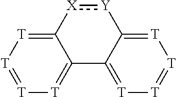

group or derivative thereof.

In some examples, D may be selected from the group consisting of:

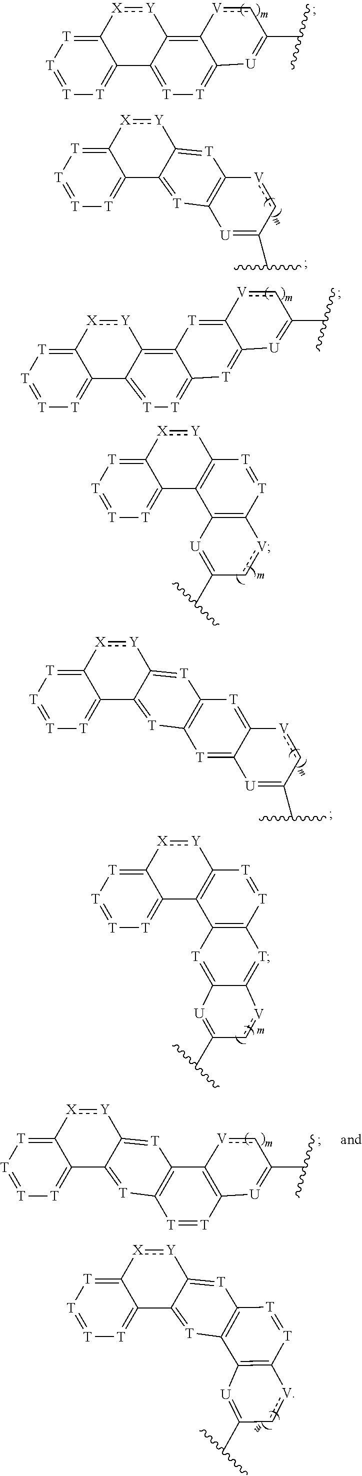

In some examples, D is

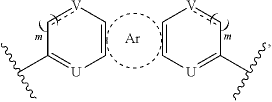

wherein the

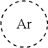

### of D is a

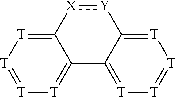

group or derivative thereof, which is fused to

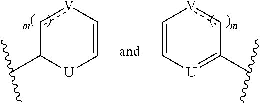

at any available

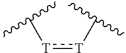

positions on the

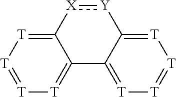

group or derivative thereof.

In some examples, J is

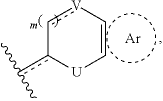

wherein the

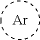

group is a

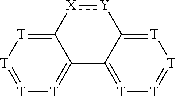

group or derivative thereof that is fused to

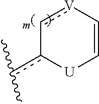

at any available

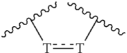

position on the

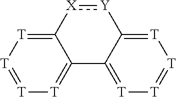

group or derivative thereof.

In some examples, J is selected from the group consisting of J is selected from the group consisting of

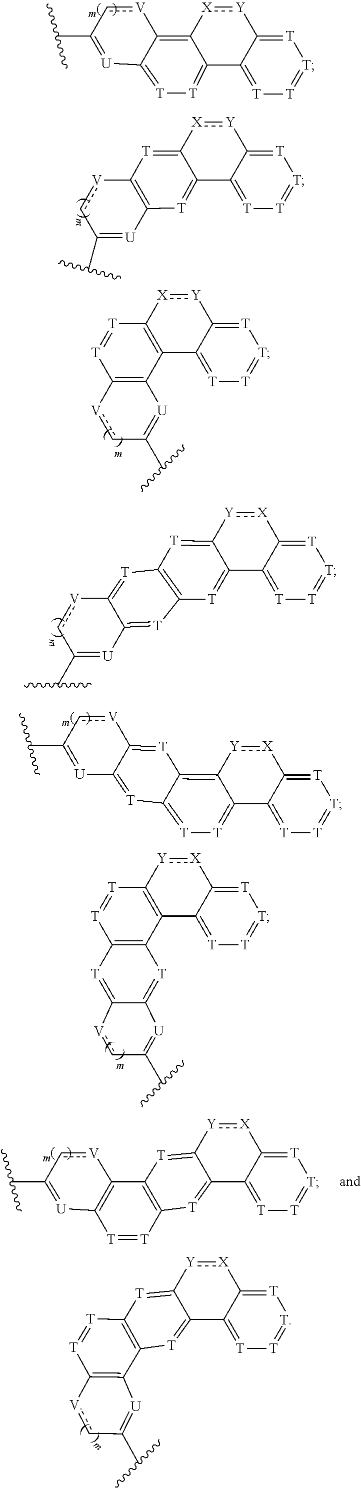

In some examples, the at least one

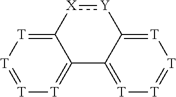

group or derivative thereof is selected from the group consisting of

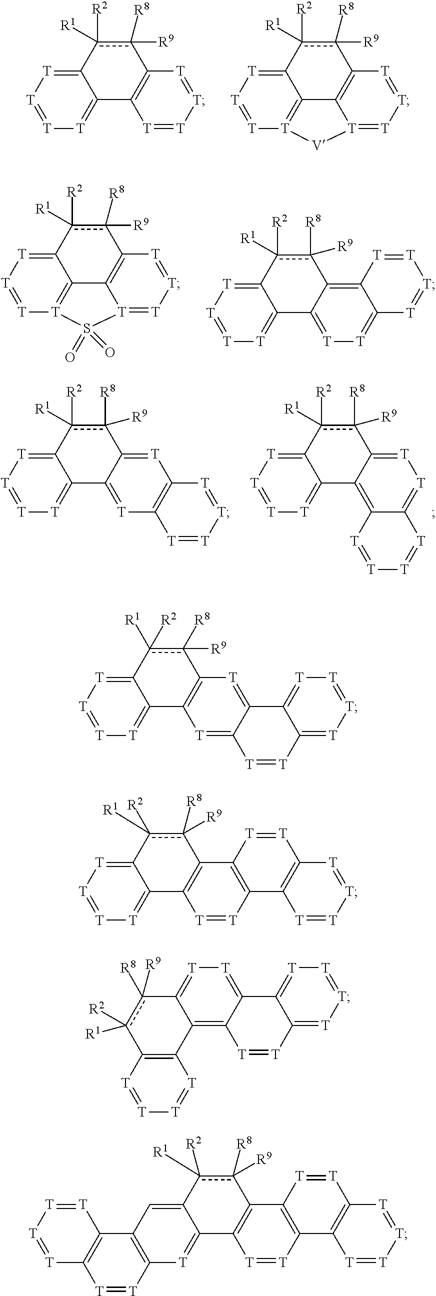

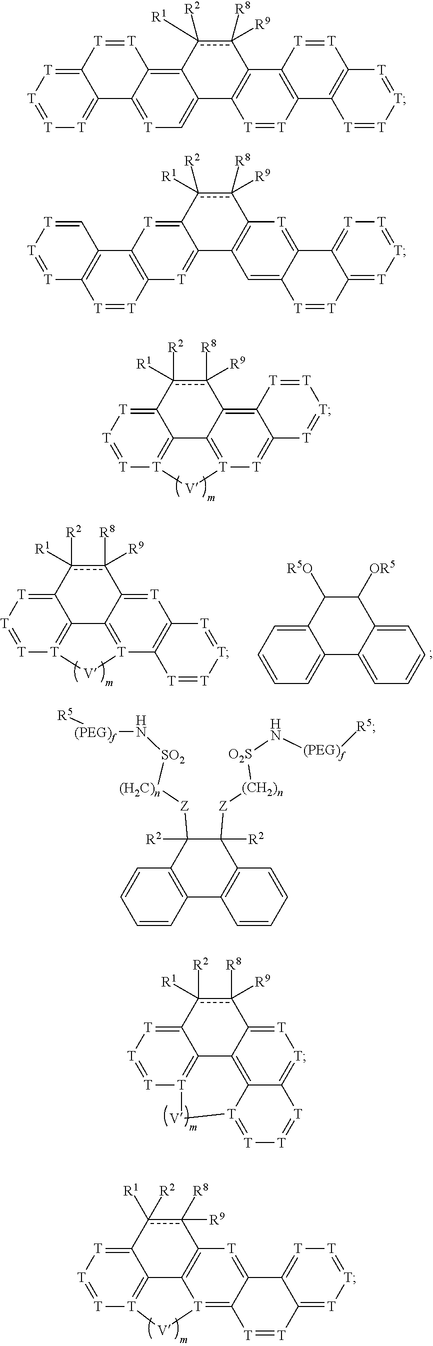

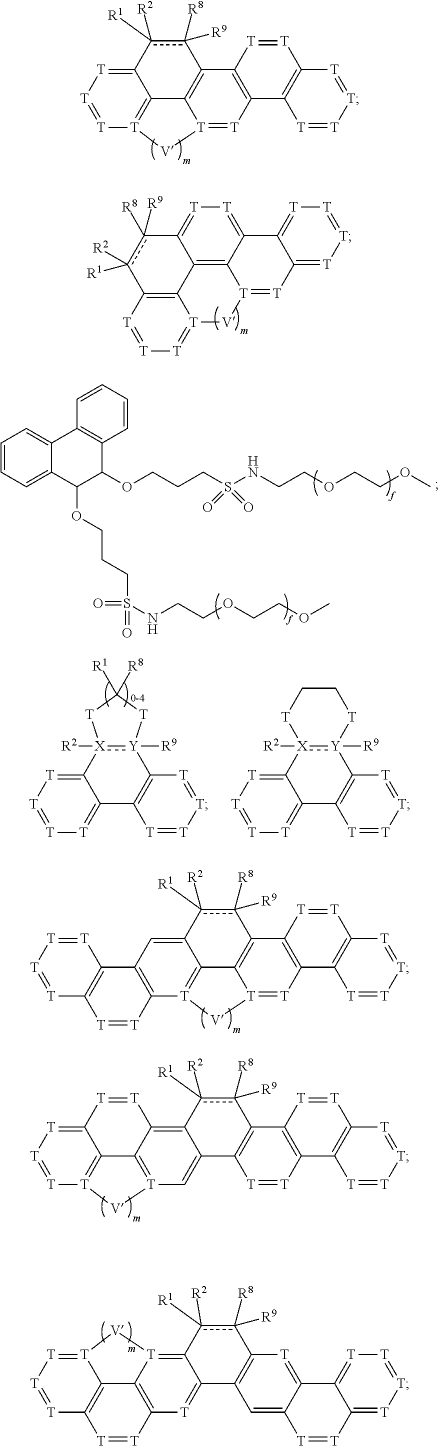

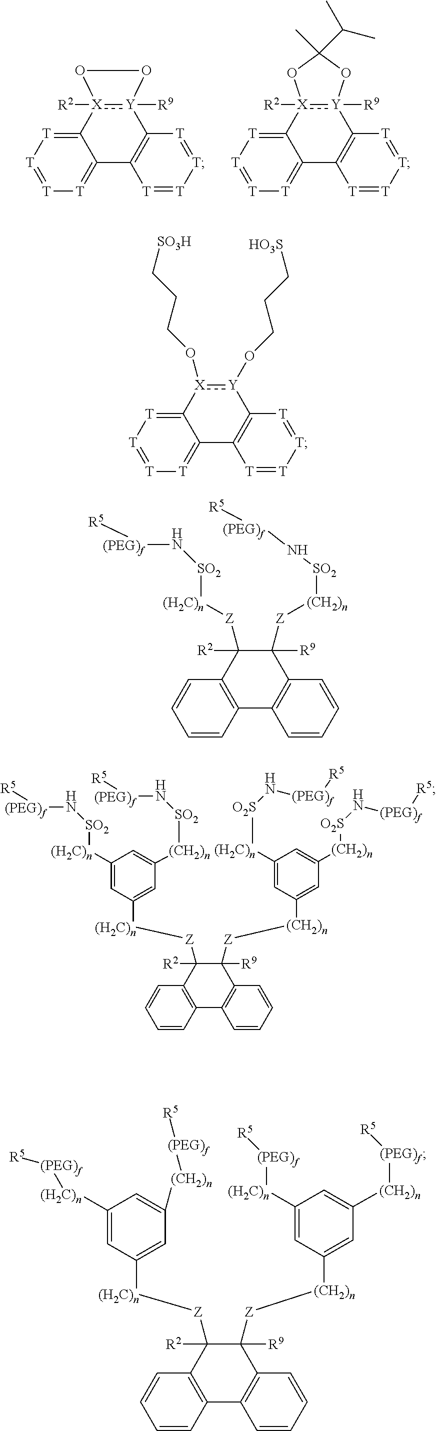

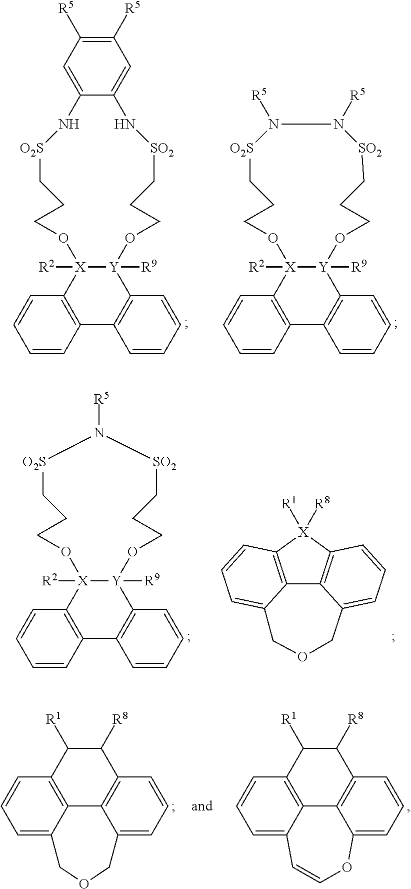

wherein each R⁵ is independently selected from the group consisting of halogen, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, a C₂-C₁₈ (hetero)aryl group, C₂-C₁₈ (hetero)aryloxy, C₂-C₁₈ (hetero)arylamino, carboxylic acid, carboxylate ester, (CH₂)_{x′}(OCH₂—CH₂)_{y′}OCH₃, and (CH₂)_{x′}(OCH₂—CH₂)_{y′}OCF₃, where each x′ is independently an integer from 0-20, and each y′ is independently an integer from 0-50.

In some examples, D is selected from the group consisting of

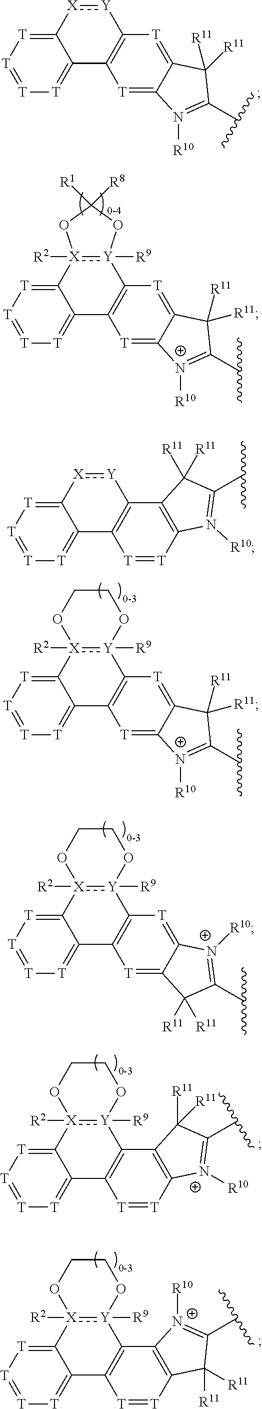

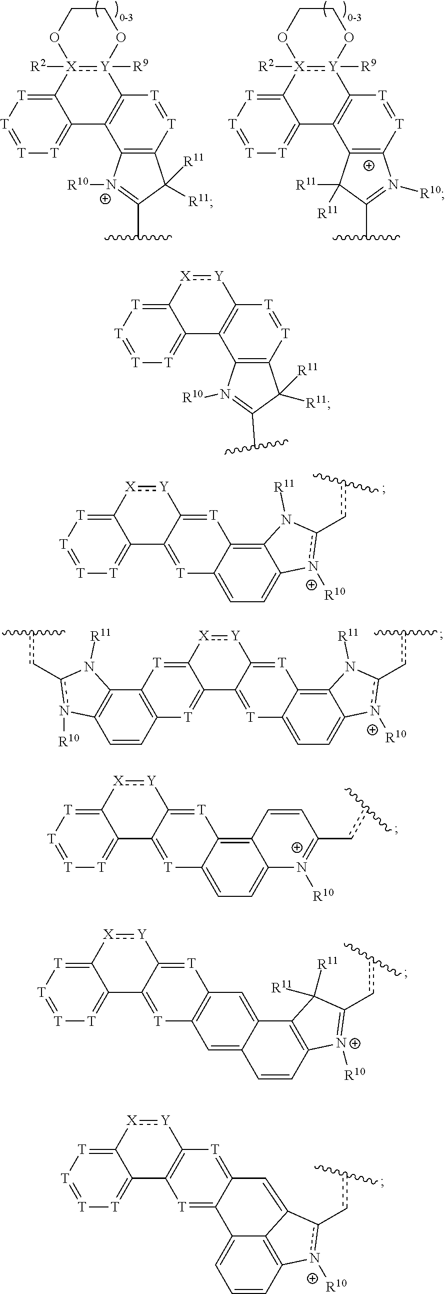

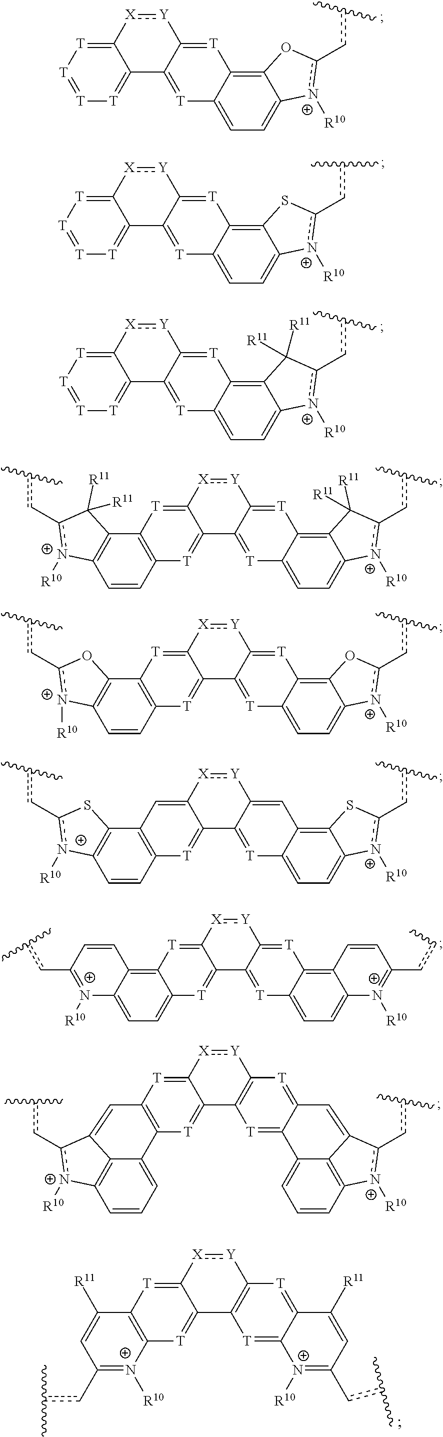

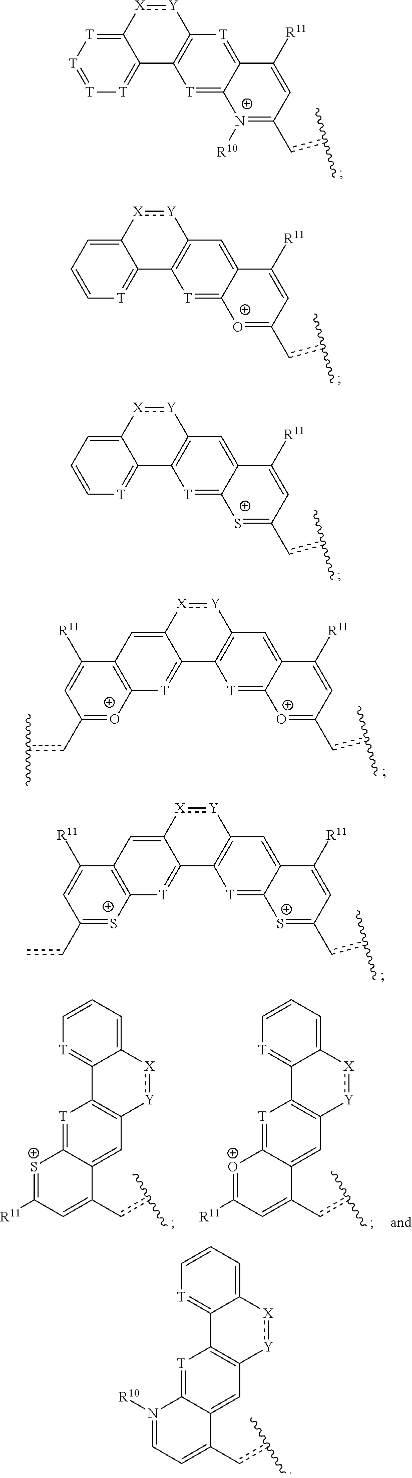

In some examples, J is selected from the group consisting of

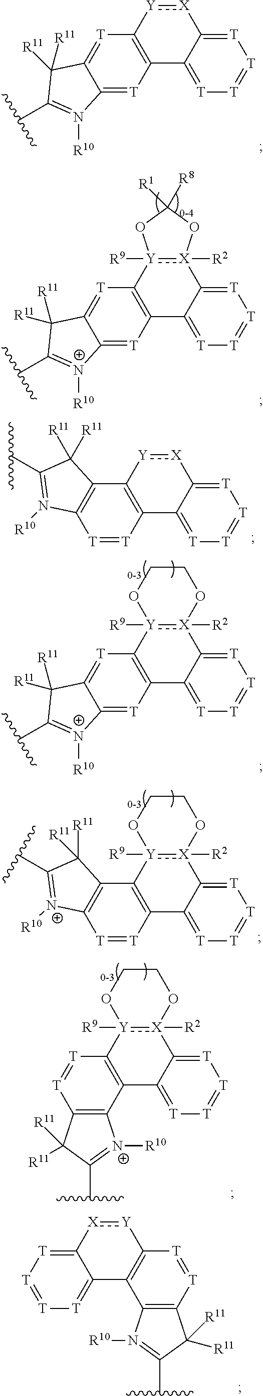

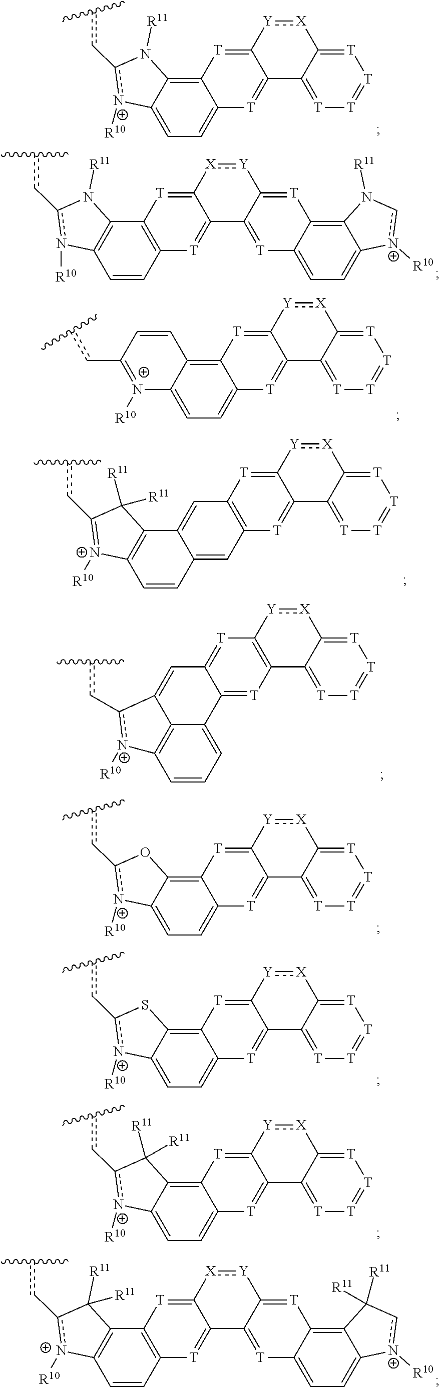

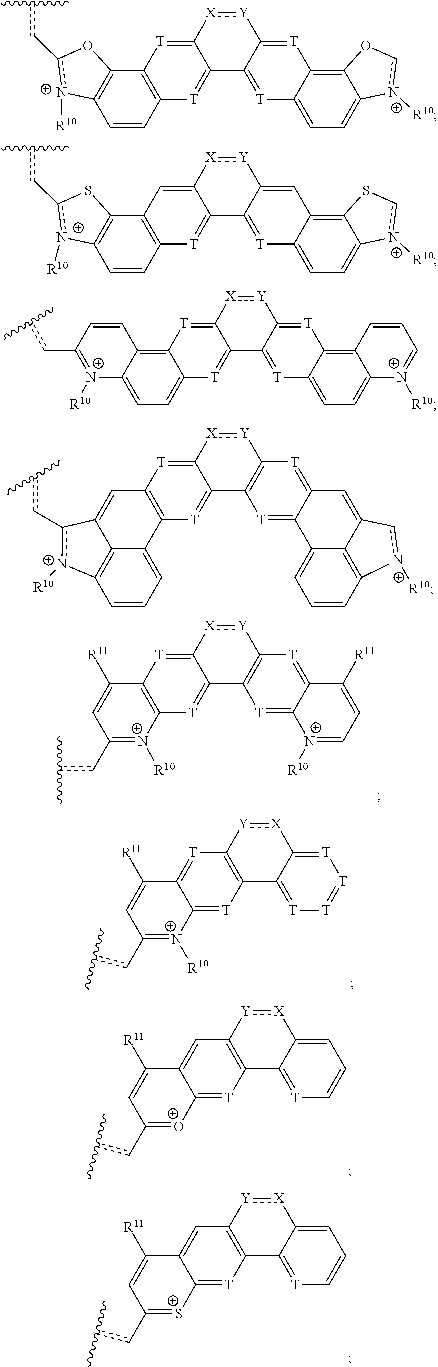

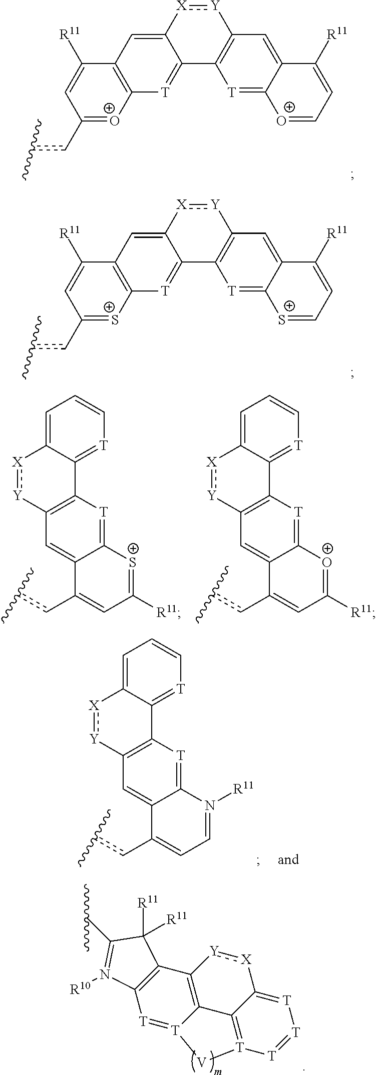

In some examples, each R¹⁰ and each R¹¹ is independently selected from the group consisting of a water-solubilizing moiety, a linker moiety, a linked water-solubilizing moiety, hydrogen, alkyl, alkene, alkyne, cycloalkyl, haloalkyl, (hetero)aryloxy, aryl, heteroaryl, (hetero)arylamino, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide, alkoxy sulfonamide PEG, alkoxy sulfonate, alkyl sulfonate, alkyl sulfonate salt,

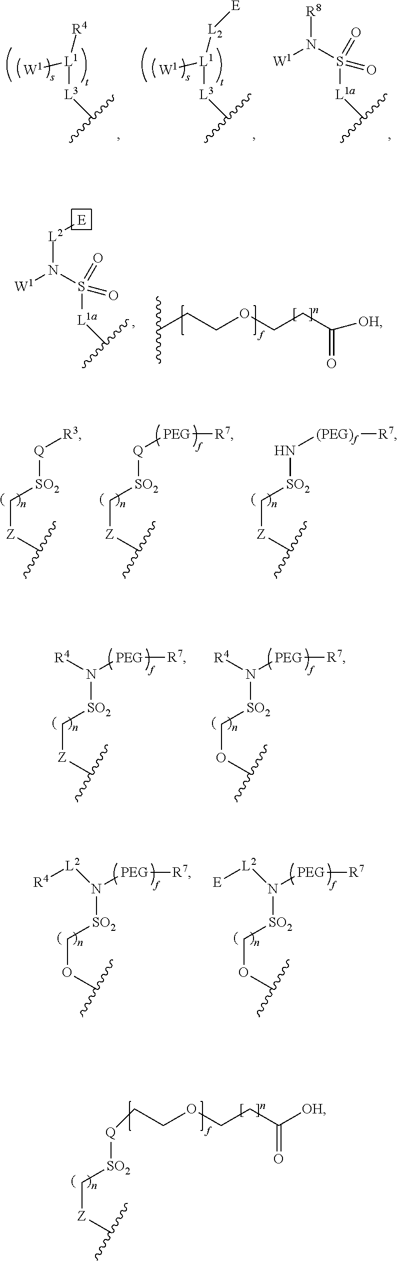

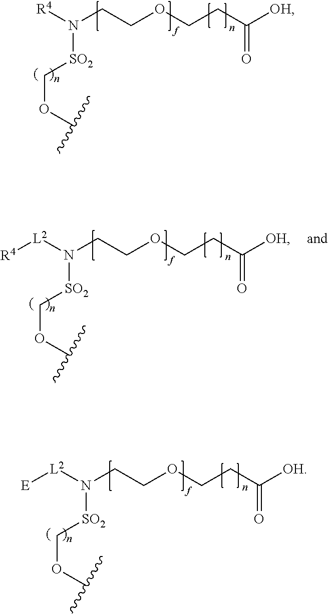

In some cases, each R¹⁰ and each R¹¹ independently comprises a moiety selected from the group consisting of sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide, alkoxy sulfonamide PEG, alkoxy sulfonate, alkyl sulfonate, alkyl sulfonate salt,

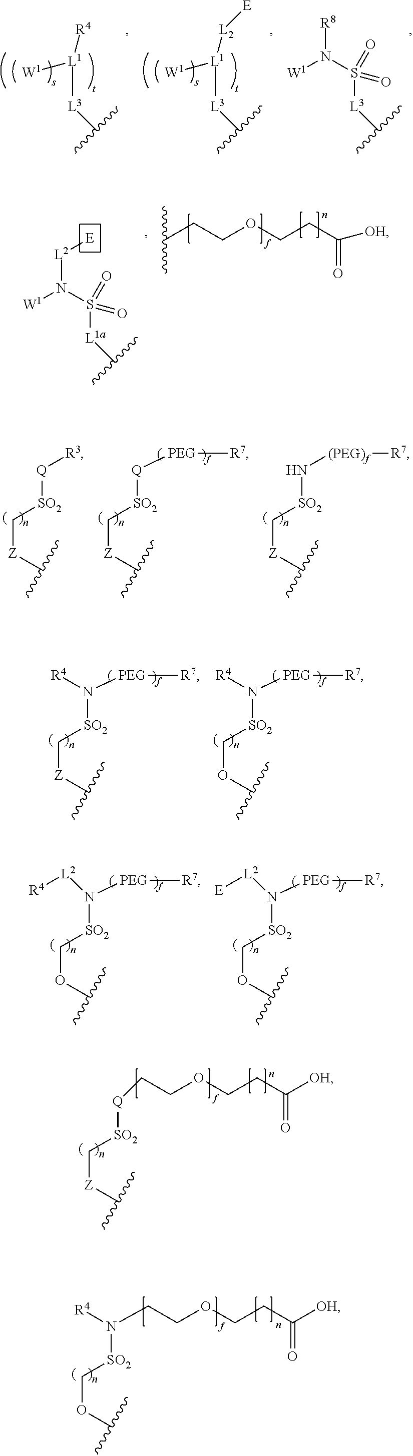

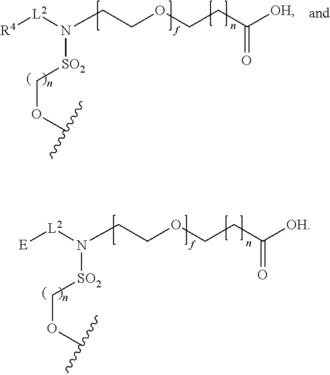

In some cases, each R¹⁰ and each R¹¹ independently comprises a moiety selected from the group consisting of sulfonamide-PEG, sulfonamido oligoether, sulfonamide, sulfinamide, alkoxy sulfonamide PEG,

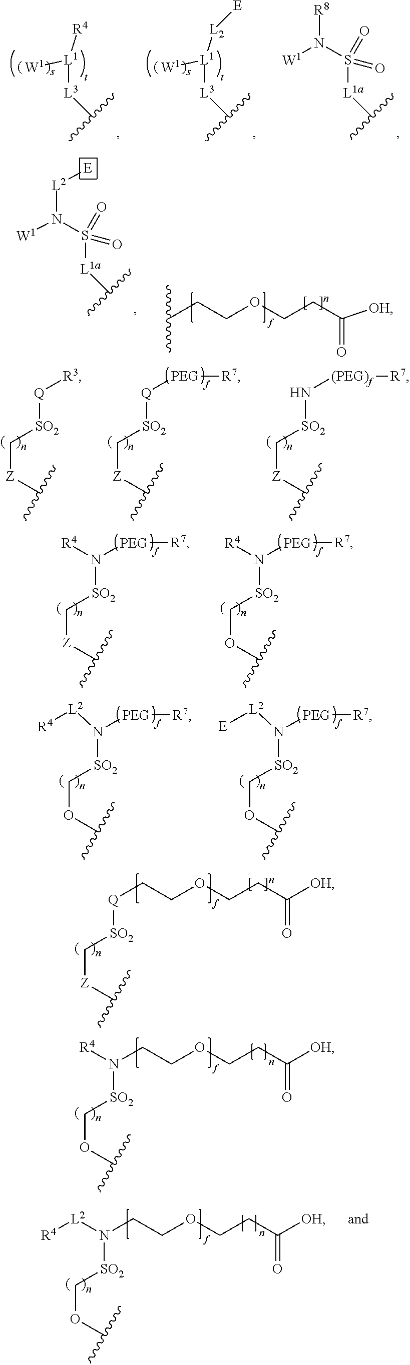

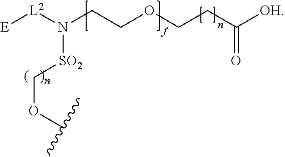

The DHP-cyanine and DHP-squaraine compounds of the present disclosure may include any of the following compounds:

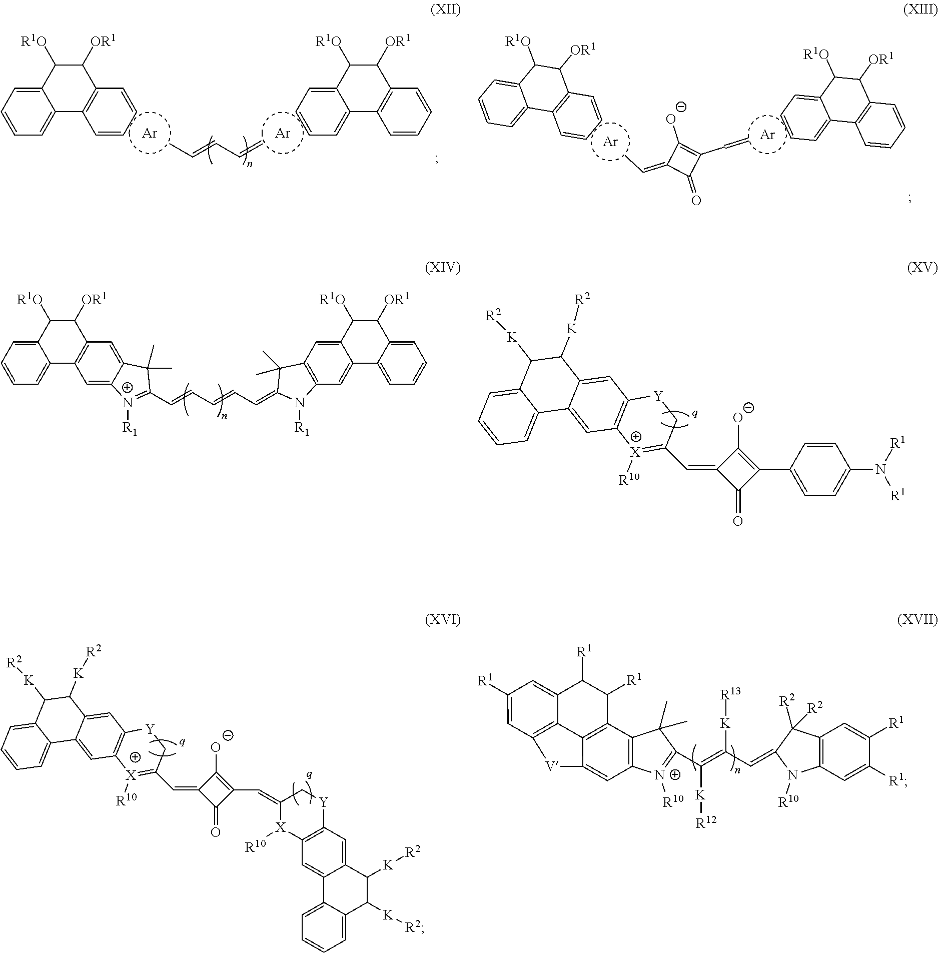

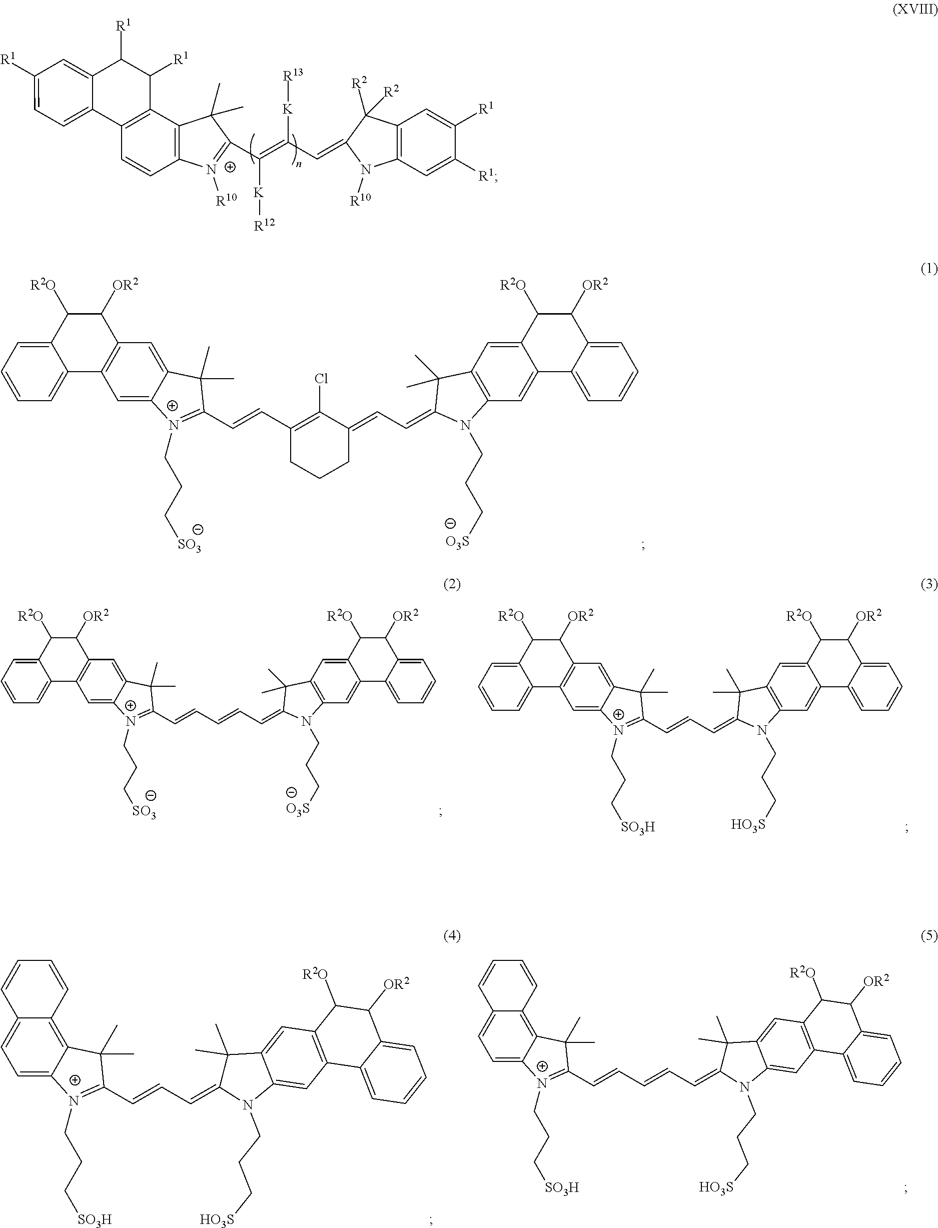

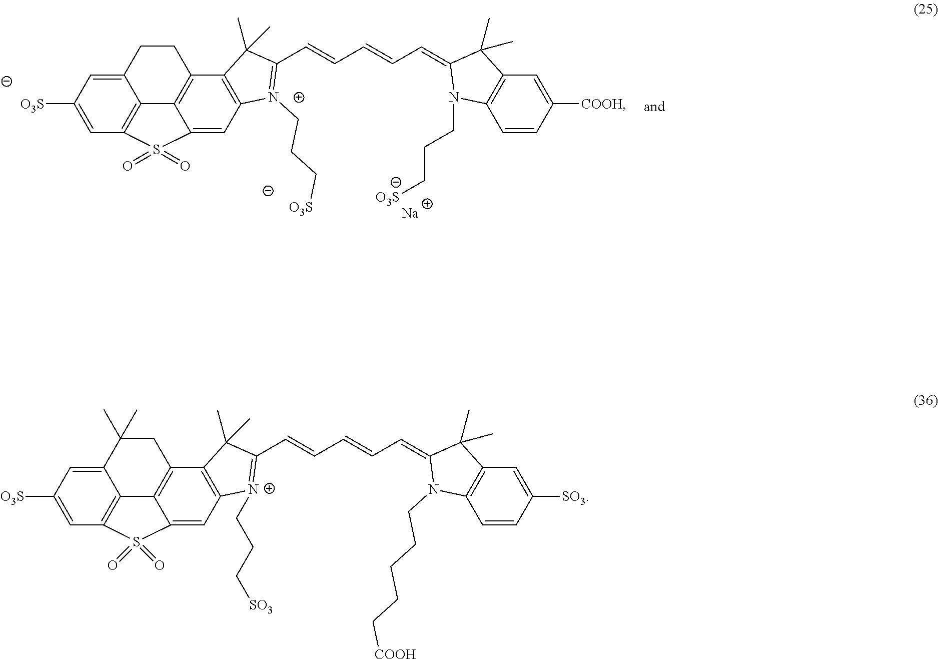

The DHP-cyanine and DHP-squaraine compounds of the present disclosure may be water-soluble.

Methods of making DHP-cyanine and DHP-squaraine compounds of the present disclosure are also provided.

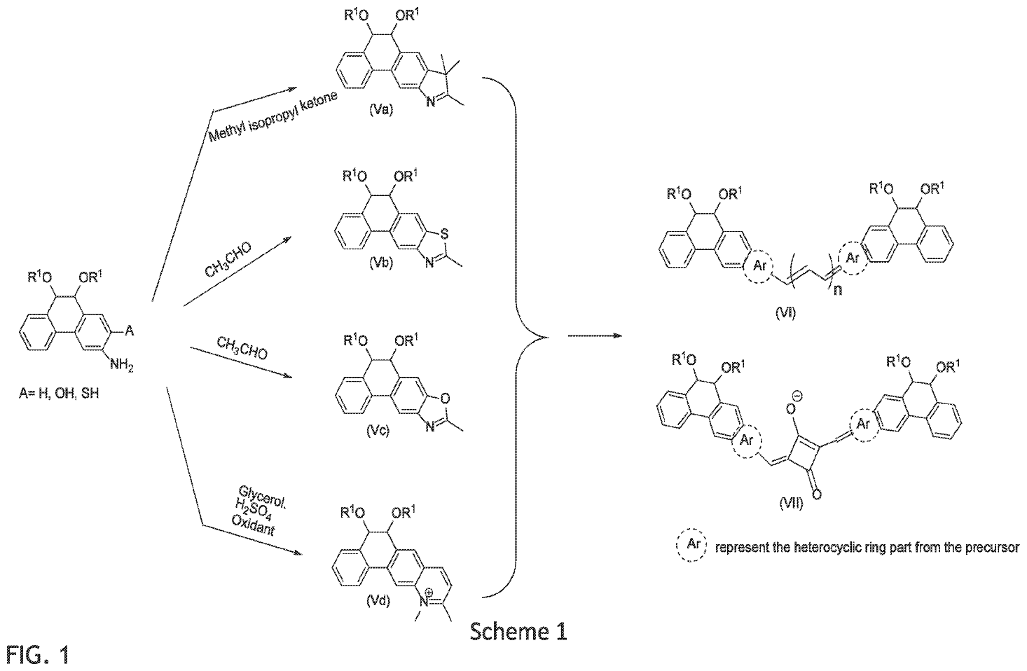

FIG. 1 shows exemplary synthetic scheme 1 with selected steps in DHP-cyanine and DHP-squaraine syntheses from a modified dihydrophenanthrene (DHP) core. DHP-indole (Va), DHP-thiazole (Vb), DHP-oxazole (Vc), and DHP-quinoline (Vd) intermediates can be prepared from a DHP core molecule. Treatment with either an appropriate aldehyde dianiline hydrochloride or a squaric acid yields the DHP-cyanine compounds of formula (VI), where n=0, 1, 2, or 3, or DHP-squaraine compounds of formula (VII), respectively.

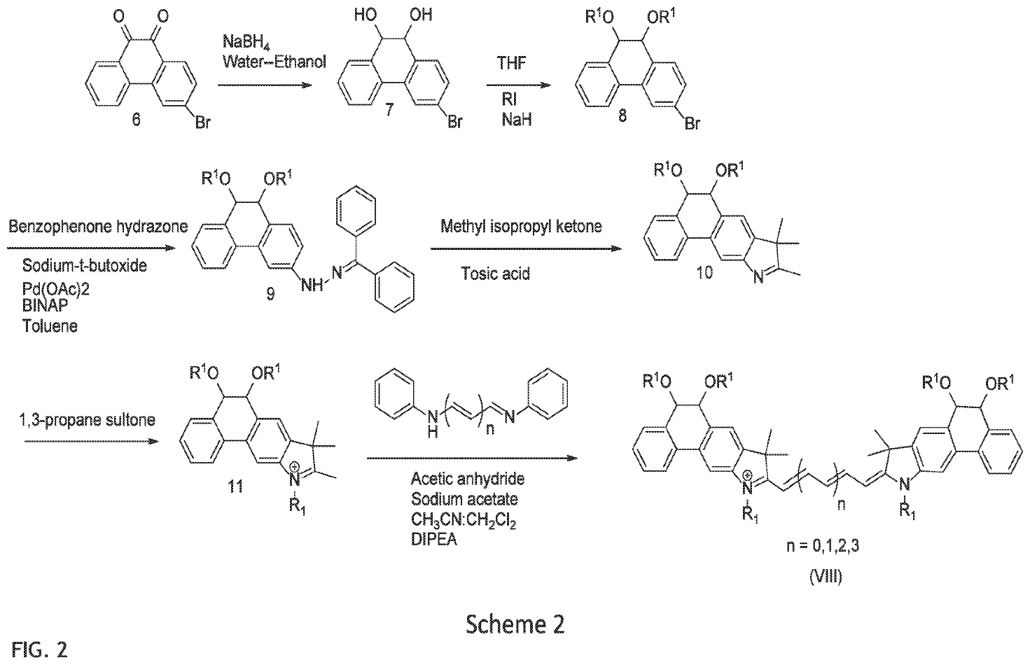

FIG. 2 shows a representative synthetic scheme 2 used to modify dihydrophenanthrene followed by synthesis of a DHP-Cy5 type monomeric dye. Commercially available 3-bromophenanthene-9,10-dione 6 may be reduced with sodium borohydride NaBH₄ in water-ethanol to provide 3-bromo-9,10-dihydrophenanthrene-9,10-diol intermediate 7. The DHP-diol Z is treated with, for example, an alkyl iodide in the presence of sodium hydride NaH to provide 3-bromo-DHP-OR¹ intermediate 8. Treatment of 8 with benzophenone hydrazone in the presence of sodium t-butoxide, Pd(OAc)₂, (±)-BINAP, in toluene provides DHP-hydrazine intermediate 9. Treatment of 2 with methyl isopropyl ketone in acidic condition provides DHP-indole intermediate 10. Treatment of 10 with 1,3-propane sulfone yields 3-sulfopropyl-naphthoindole intermediate 11. Treatment of 11 with an appropriate aldehyde dianiline hydrochloride (e.g., glutaconic aldehyde dianilide hydrochloride), in the presence of acetic anhydride, sodium acetate, and DIPEA in CH₃CN/CH₂Cl₂ yields a DHP-Cyanine dye of structure (VIII) according to the disclosure. This protocol and variations thereof may be used for preparation of compounds 1-5 as shown in Table 1.

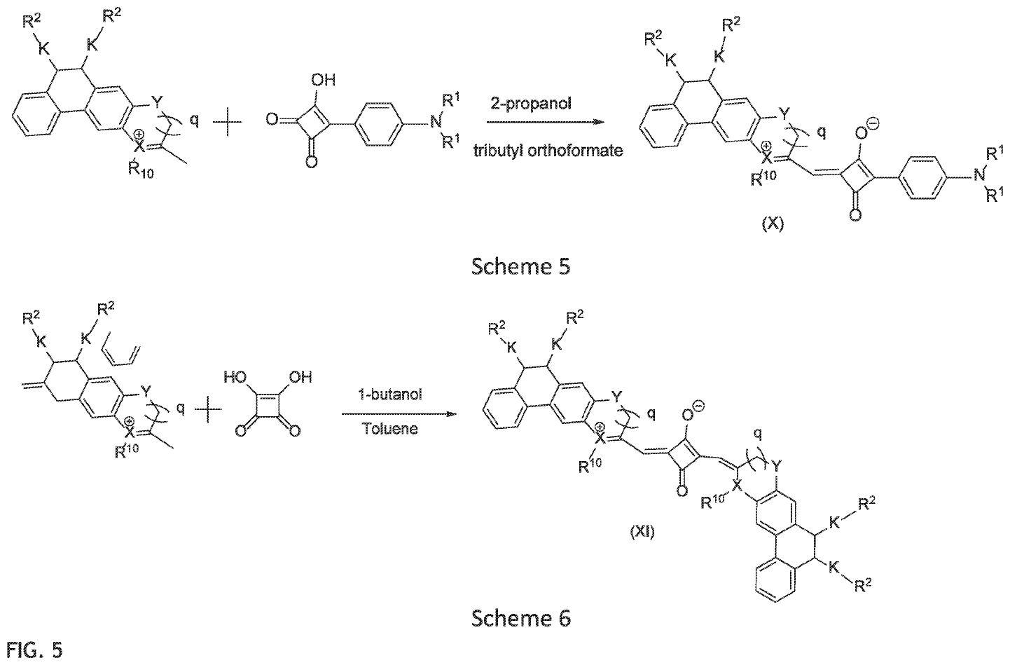

FIG. 5 shows synthetic scheme 5 for preparing an asymmetric DHP-squaraine monomeric dye of formula (X) from a dihydrophenanthrene type core and a semisquaraine. FIG. 5 also shows synthetic scheme 6 for preparing a symmetric DHP-squaraine monomeric dye of formula (XI) from a dihydrophenanthrene type core and squaric acid.

Table 1 shows exemplary symmetric and unsymmetric DHP-cyanine compounds 1-5 of the present disclosure along with their fluorescence properties including maximum excitation wavelength (λex) and emission wavelengths (hem).

TABLE 1
Exemplary DHP-Cyanine Dyes with Fluorescence Properties

| Cpd. | | λex | λem |
|---|---|---|---|
| No. | Structure | (nm) | (nm) |
| 1 | 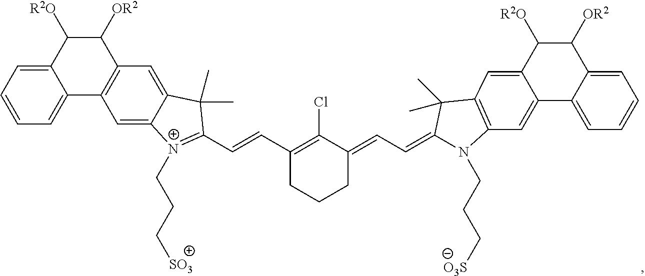 | 840 | To be measured |
| | R² = CH₃ | | |
| 2 | 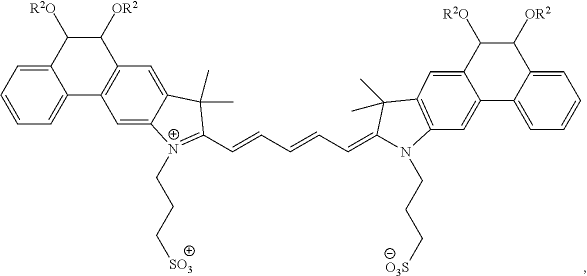 | 694 | 732 |
| | R² = CH₃ | | |
| 3 | 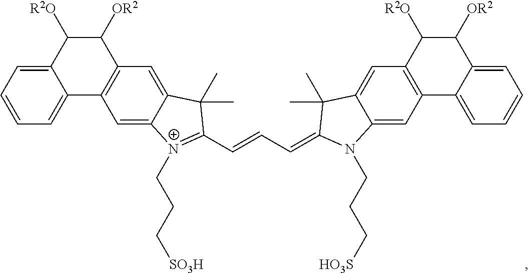 | 606 | 634 |
| | R² = CH₃ | | |
| 4 | 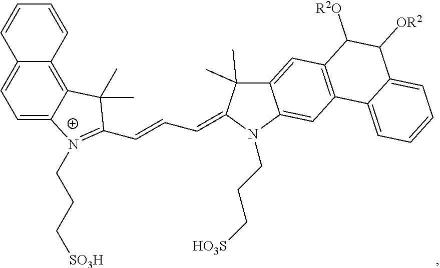 | 590 | 610 |
| | R² = CH₃ | | |
| 5 | 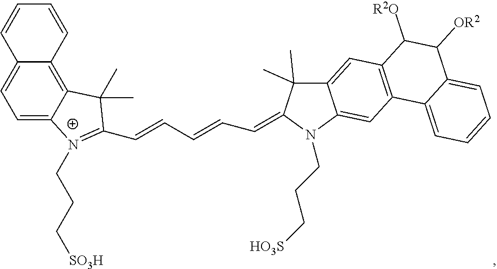 | 685 | 720 |
| | R² = CH₃ | | |
| 25 | 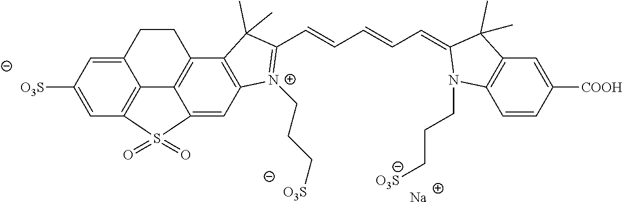 | 666 | 686 |
| 36 | 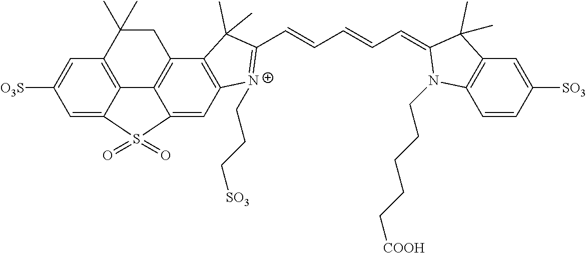 | 663 | 683 |

### IV. DHP-Cyanine and DHP-Squaraine Polymers

The disclosure provides DHP-cyanine polymers and DHP-squaraine polymers and methods of making. DHP-cyanine and DHP-squaraine polymeric dyes can exhibit excitation λmax in a range of from 420 to 900 nm. Dihydrophenanthrenes having substituted bromines in the 3 and 6 position will result in dyes which could be different from dyes made using previously synthesized monomer A where the bromine atoms are substituted in the 2 and 7 positions. Therefore, several options to make different dyes are available from this technology.

Polymer dyes are provided in the present disclosure comprising a monomer having a structure according to Formula (IV) or (V):

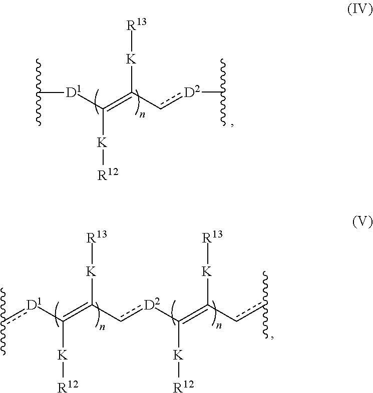

wherein each [symbol] in (IV) or (V) is a point of attachment to the polymer dye backbone.

Each D¹ and D² is independently selected from the group consisting of an aryl group, heteroaryl group, and

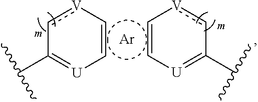

wherein each

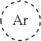

is independently selected from the group consisting of a substituted or unsubstituted benzene, benzene derivative, monocyclic aryl group, polycyclic aryl group, monocyclic heteroaryl group, and polycyclic heteroaryl group, with at least one of D¹ or D² being

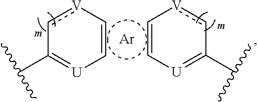

wherein

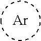

is

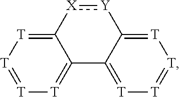

or a derivative thereof comprising an additional aryl or heteroaryl group fused at any available

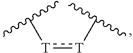

and wherein the

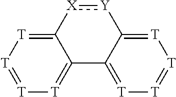

group or derivative thereof is fused to

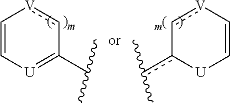

at any available

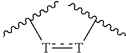

position on the

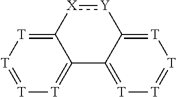

group or derivative thereof. Each T, U, V, X, Y, R¹, R², R³, R⁴, R⁷, R⁸, R⁹, R¹⁰, R¹¹, R¹², R¹³, Q, Z, W¹, L¹, L², L³, E, k, f, n, m, n, s, tis as described herein above.

The derivative of

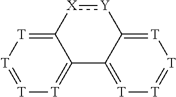

comprising an additional aryl or heteroaryl group fused at any available

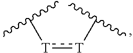

may optionally comprise

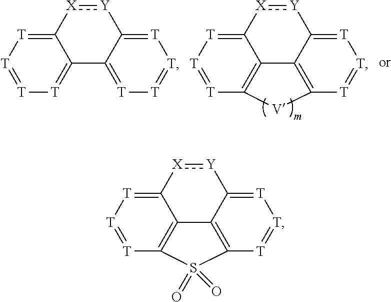

wherein V′ is SO₂, SO, S, NR¹¹, CR¹¹, C(R¹¹)₂, O, Si(R¹¹)₂, >C═O, >Se═O, —CH═CH—, or —N═CH—, and T, V, X, Y, and m are as defined herein above.

In examples, the monomer of Formula (IV) or (V) comprises a structure selected from the group consisting of Formula (A1), (A2), (A3), (A4), (A5), (A6), (A7), (A8), (A9), (A10), (A11), (A12), (A13), (A14), (A15), (A16), (A17), (A18), and (A19):

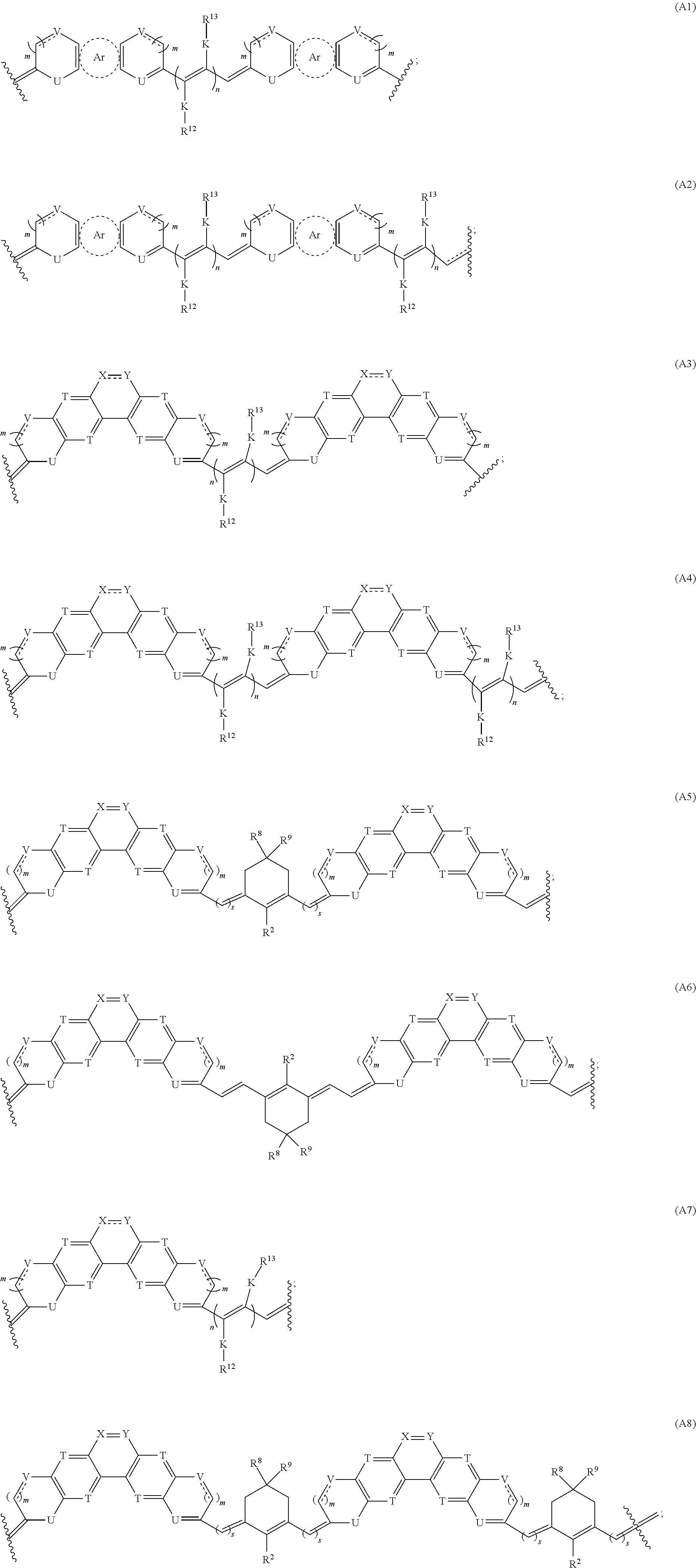

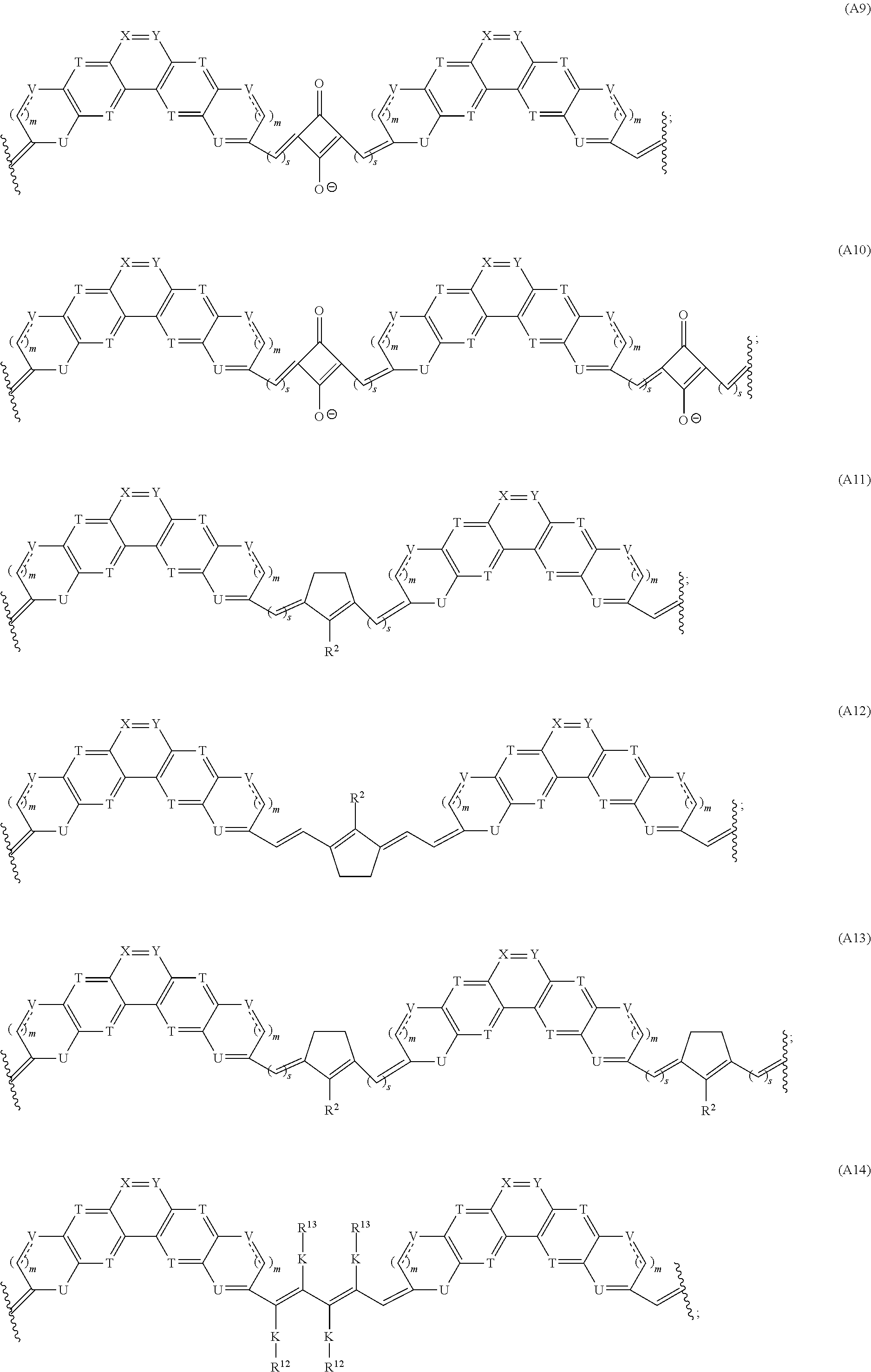

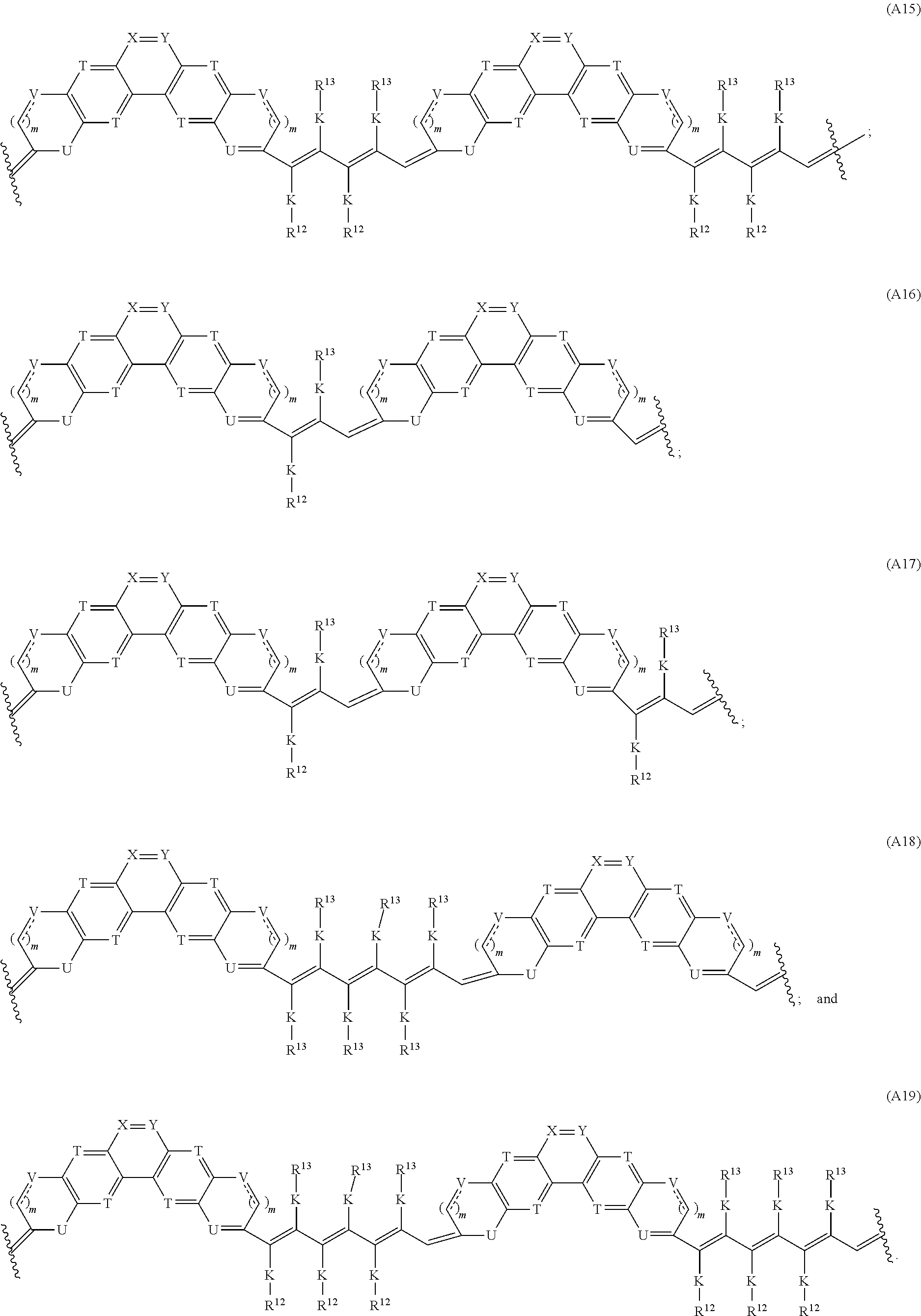

In some examples, the disclosure provides a DHP-cyanine or DHP-squaraine polymer dye according to Formula (VI) or (VII):

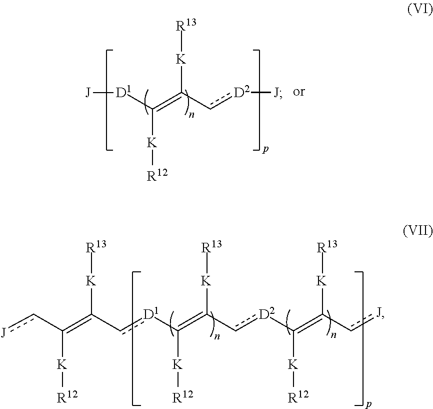

wherein
each D¹ and D² is independently selected from the group consisting of an aryl group, heteroaryl group, and

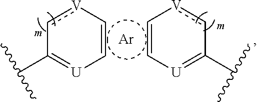

wherein
   each

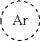

is independently selected from the group consisting of a substituted or unsubstituted benzene, benzene derivative, monocyclic aryl group, polycyclic aryl group, monocyclic heteroaryl group, and polycyclic heteroaryl group, with at least one of D¹ or D² being

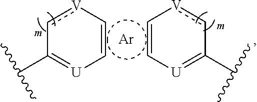

wherein

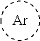

is

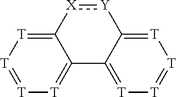

or a derivative thereof comprising an additional aryl or heteroaryl group fused at any available

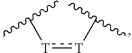

and wherein the

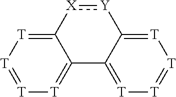

group or derivative thereof is fused to

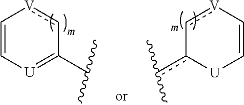

at any available

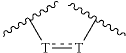

position on the

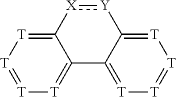

group or derivative thereof;
each J is independently aryl, heteroaryl,

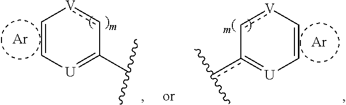

wherein

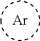

is a substituted or unsubstituted benzene, benzene derivative, monocyclic aryl group, polycyclic aryl group, monocyclic heteroaryl group, and polycyclic heteroaryl group, and p is an integer from 2 to 1,000; 5 to 500; or 10 to 100. In some embodiments, at least one J group is

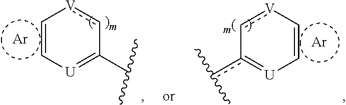

wherein the

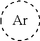

group is a

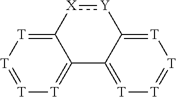

group or derivative thereof comprising an additional aryl or heteroaryl group fused at any available

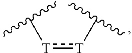

and wherein the

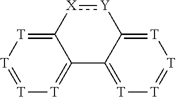

group or derivative thereof is fused to

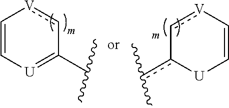

at any available

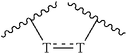

position on the

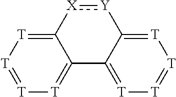

group or derivative thereof.

In some examples, the disclosure provides a DHP-cyanine or DHP-squaraine polymer dye according to Formula (VI′):

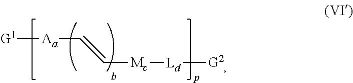

wherein A is a monomer comprising a structure according to formula (IV) or (V)

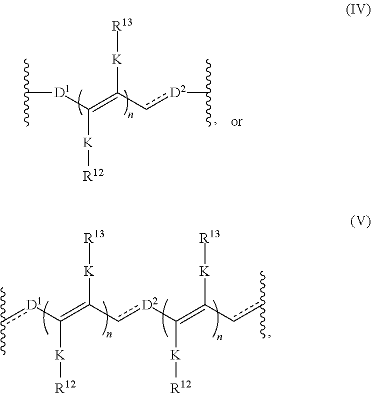

wherein
each [symbol] in formula (IV) or (V) is a point of attachment to the polymer dye backbone; each D¹ and D² is independently selected from the group consisting of an aryl group, heteroaryl group, and

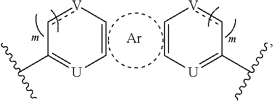

wherein
each

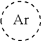

is independently selected from the group consisting of a substituted or unsubstituted benzene, benzene derivative, monocyclic aryl group, polycyclic aryl group, monocyclic heteroaryl group, and polycyclic heteroaryl group, with at least one of D¹ or D² being

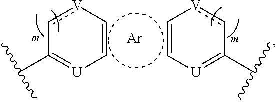

wherein

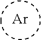

is

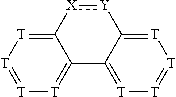

or a derivative thereof comprising an additional aryl or heteroaryl group fused at any available

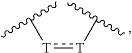

and wherein the

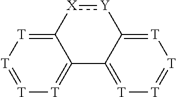

group or derivative thereof is fused to

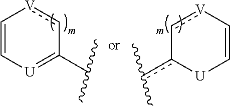

at any available

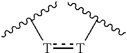

position on the

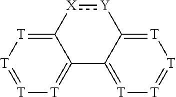

group or derivative thereof;
   each optional M is a polymer modifying unit evenly or randomly distributed along the polymer main chain and is optionally substituted with one or more optionally substituted R¹, R², R³, or R⁴ groups;
   each optional L is a linker;
   G¹ and G² are each independently selected from an unmodified polymer terminus and a modified polymer terminus, optionally conjugated to E;
   a, c, and d define the mol % of each unit within the structure which each can be evenly or randomly repeated and where each a is a mol % from 10 to 100%, each c is a mol % from 0 to 90%, and each d is a mol % from 0 to 25%;
   each b is independently 0 or 1; and
   p is an integer from 1 to about 10,000; wherein each T, U, V, X, Y, R¹, R², R³, R⁴, R⁷, R⁸, R⁹, R¹⁰, R¹¹, R¹², R¹³, Q, Z, W¹, L¹, L², L³, E, k, f, n, m, n, s, tis as described herein above.

The derivative of

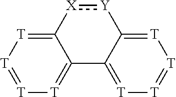

comprising an additional aryl or heteroaryl group fused at any available

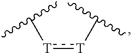

may optionally comprise

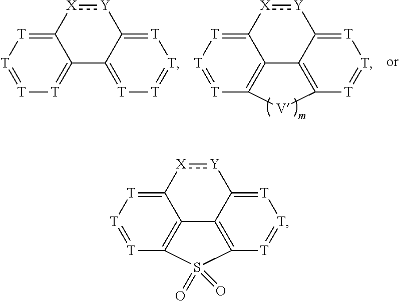

wherein V′ is SO₂, SO, S, NR¹¹, CR¹¹, C(R¹¹)₂, O, Si(R¹¹)₂, >C═O, >Se═O, —CH═CH—, or —N═CH—, and T, V, X, Y, and m are as defined herein above.

In some examples, the monomer of Formula (IV) or (V) comprises a structure selected from the group consisting of Formula (A1), (A2), (A3), (A4), (A5), (A6), (A7), (A8), (A9), (A10), (A11), (A12), (A13), (A14), (A15), (A16), (A17), (A18), and (A19) as shown herein.

In some examples, each G¹ and G² is independently selected from the group consisting of hydrogen, halogen, alkyne, optionally substituted aryl, optionally substituted heteroaryl, halogen substituted aryl, silyl, diazonium salt, triflate, acetyloxy, azide, sulfonate, phosphate, boronic acid substituted aryl, boronic ester substituted aryl, boronic ester, boronic acid, optionally substituted tetrahydropyrene (THP), optionally substituted fluorene, optionally substituted dihydrophenanthrene (DHP), aryl or heteroaryl substituted with one or more pendant chains terminated with a functional moiety selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof, optionally for conjugation to a substrate, or a binding partner.

In some examples, D¹ and D² are the same or different. In some examples D¹ and D² are the same. In some examples, D¹ and D² are different.

The polymer according to the disclosure may be water-soluble.

Methods of making DHP-cyanine polymers and DHP-squaraine polymers of the present disclosure are also provided.

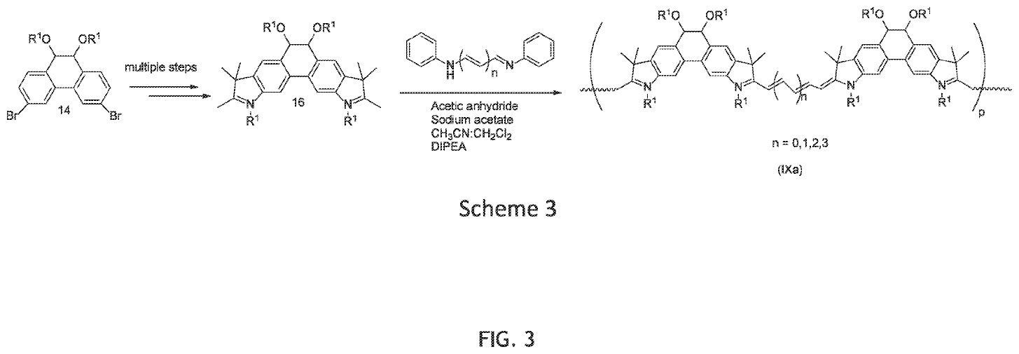

FIG. 3 shows an exemplary synthetic scheme for production of DHP-cyanine polymers according to the present disclosure. The starting 3,6-dibromo 9,10-substituted-9,10-dihydrophenanthrene 14 may be produced according to U.S. Pat. No. 11,208,527, which is incorporated by reference herein in its entirety. Production of the DHP-di-indole intermediate 16 may be performed by any appropriate protocol. For example, the DHP-di-indole 16 may be produced in an analogous fashion to scheme 2. Treatment with an appropriate aldehyde dianiline hydrochloride in the presence of DIEA and NaOAc results in formation the DHP-cyanine polymers according to formula (IXa) as shown in Scheme 3, wherein p may be an integer from 2 to 1,000; 5 to 500; or 10 to 100.

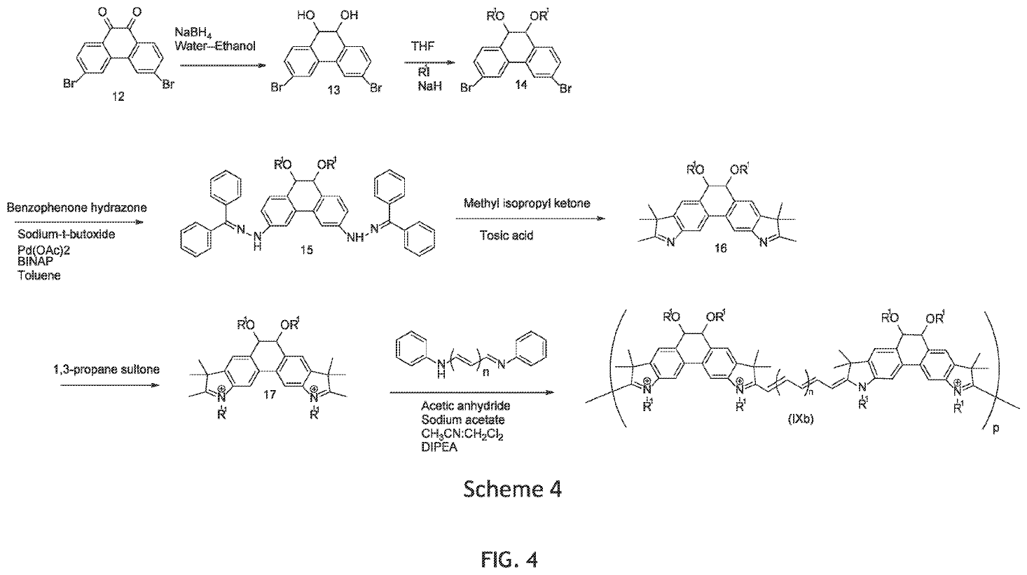

FIG. 4 shows synthetic scheme 4 for providing DHP-cyanine polymers according to formula (IXb). Commercially available 3,6-dibromophenanthene-9,10-dione 12 is reduced with sodium borohydride NaBH₄ in water-ethanol to provide 3,6-di-bromo-9,10-dihydrophenanthrene-9,10-diol intermediate 13. The dibromo-DHP-diol 13 is treated with, for example, an alkyl iodide in the presence of sodium hydride NaH to provide 3,6-dibromo-DHP-OR¹ intermediate 14. Treatment of 14 with benzophenone hydrazone in the presence of sodium t-butoxide, Pd(OAc)₂, (±)-BINAP, in toluene provides DHP-dihydrazine intermediate 15. Treatment of 15 with methyl isopropyl ketone in acidic condition provides DHP-diindole intermediate 16. Treatment of 16 with 1,3-propane sulfone yields di-sulfopropyl-naphthoindole intermediate 17. Treatment of 17 with an appropriate aldehyde dianiline hydrochloride (e.g., glutaconic aldehyde dianilide hydrochloride), in the presence of acetic anhydride, sodium acetate, and DIPEA in CH₃CN/CH₂Cl₂ yields a DHP-Cyanine dye of structure (IXb), wherein p may be an integer from 2 to 1,000; 5 to 500; or 10 to 100.

### V. Capping Units

Linkers and capping units can be conjugated to a fluorescent polymer backbone of this disclosure via similar mechanisms as described previously. For example, bromo- and boronic esters of capping units can be used to append one or both ends of a polymer. Utilizing both bromo- and boronic esters of capping units will append both ends of polymer. Utilizing only one form, either a bromo- or boronic ester of a capping unit, will append only those ends terminated with its respective complement and for symmetric polymerizations can be used to statistically modify only one end of a polymer. For asymmetric polymers this approach is used to chemically ensure the polymers are only modified at a single chain terminus. Capping units can also be appended asymmetrically by first reacting a bromo-capping unit with a polymer with Y ends and subsequently reacting the polymer with a boronic ester capping unit.

For example, capping agents of the present disclosure can be made as shown in scheme (VII):

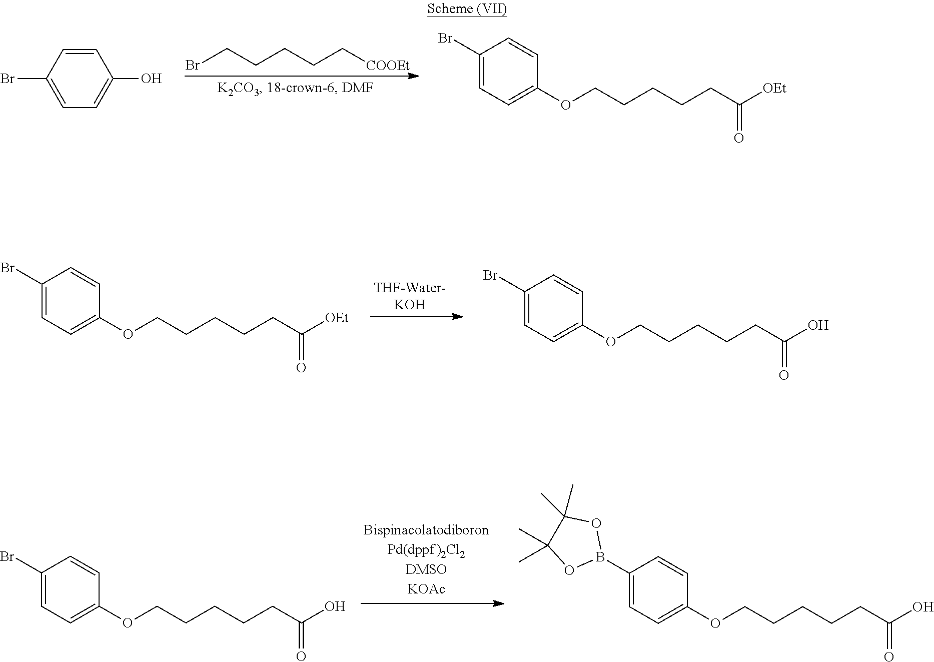

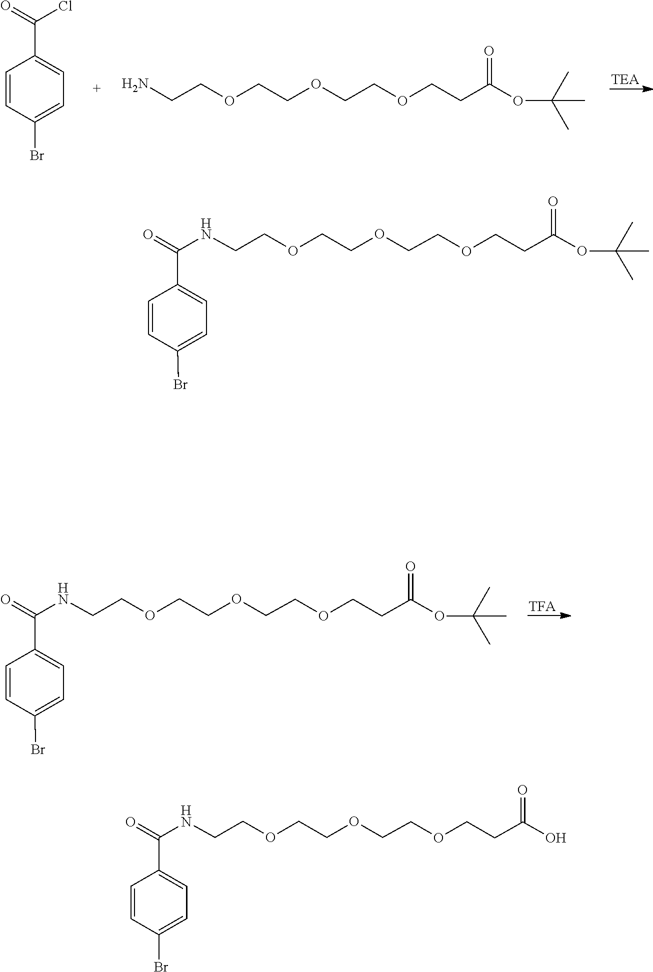 [SMILES: O=C(Cl)c1ccc(Br)cc1 ; CC(C)(C)OC(=O)CCOCCOCCOCCN ; CC(C)(C)OC(=O)CCOCCOCCOCCNC(=O)c1ccc(Br)cc1 ; CC(C)(C)OC(=O)CCOCCOCCOCCNC(=O)c1ccc(Br)cc1 ; O=C(O)CCOCCOCCOCCNC(=O)c1ccc(Br)cc1]

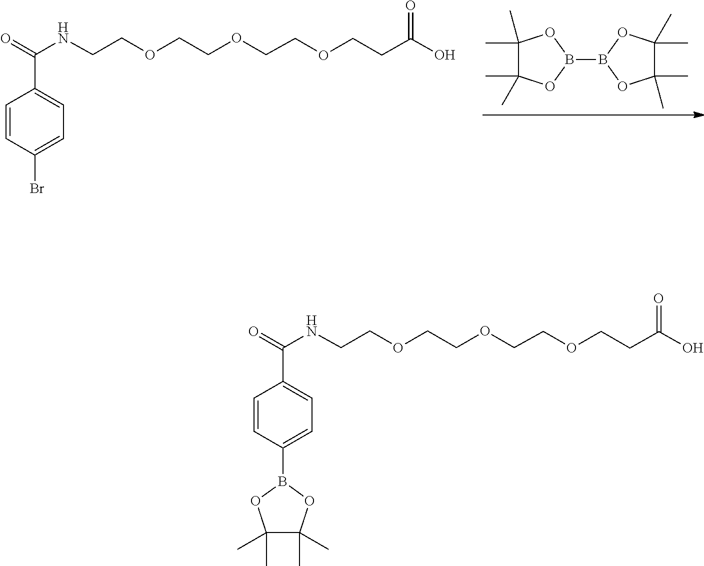

### VI. Binding Partners

A “binding partner” or “specific binding partner” of the present disclosure can be any molecule or complex of molecules capable of specifically binding to a target analyte. A binding partner of this disclosure includes, for example, proteins, small organic molecules, carbohydrates (including polysaccharides), oligonucleotides, polynucleotides, lipids, affinity ligand, antibody, antibody fragment, an aptamer and the like. In some embodiments, the binding partner is an antibody or fragment thereof. Specific binding in the context of the present disclosure refers to a binding reaction which is determinative of the presence of a target analyte in the presence of a heterogeneous population. Thus, under designated assay conditions, the specified binding partners bind preferentially to a particular protein or isoform of the particular protein and do not bind in a significant amount to other proteins or other isoforms present in the sample.

When the binding partners are antibodies, they may be monoclonal or polyclonal antibodies. The term antibody as used herein refers to immunoglobulin molecules and immunologically active portions of immunoglobulin (Ig) molecules. Such antibodies include, but are not limited to, polyclonal, monoclonal, mono-specific polyclonal antibodies, antibody mimics, chimeric, single chain, Fab, Fab′ and F(ab′)₂ fragments, Fv, and a Fab expression library.

In general, water-soluble fluorescent compounds and polymers of the present disclosure can be conjugated to binding partners to form a conjugated water-soluble fluorescent compound or polymer complex using techniques known to those of skill in the art or using methods known in the art in combination with methods described herein.

A labeled specific binding partner is provided comprising the fluorescent compound or polymer according to the present disclosure; and a specific binding partner covalently linked to the fluorescent compound or polymer. The specific binding partner may be an antibody. The specific binding partner may be specific for a target analyte.

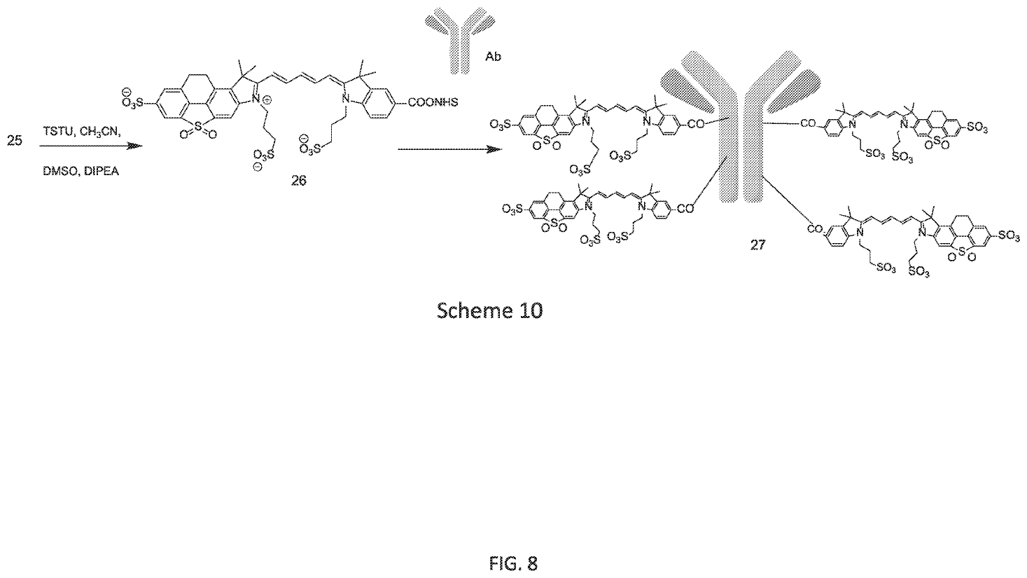

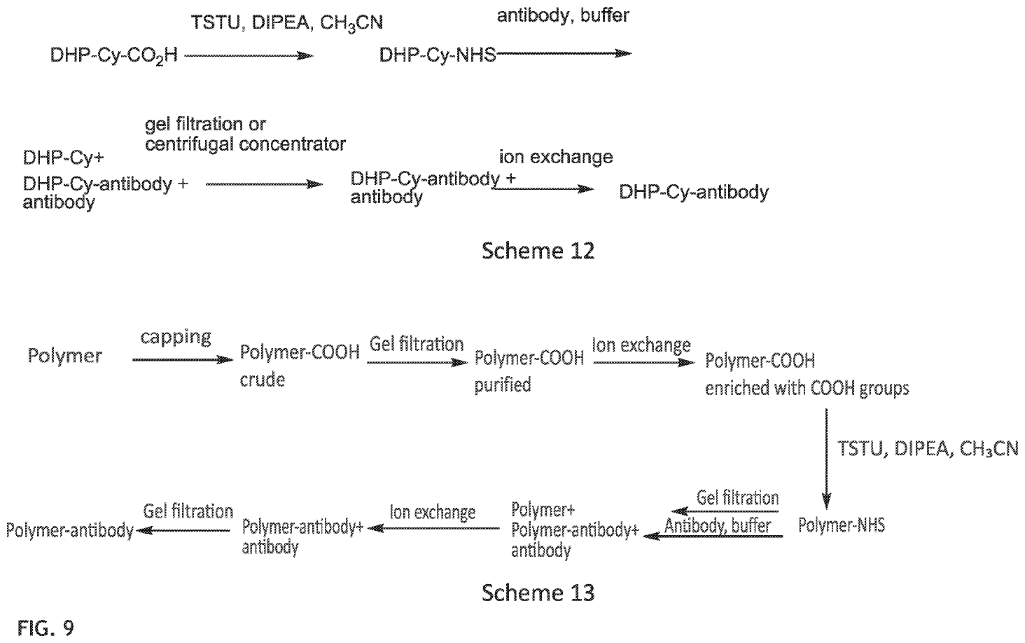

In some embodiments, fluorescent compounds or water-soluble fluorescent polymers of the present disclosure can be conjugated to binding partners using the method of direct modification of core polymers described in US2020/0190253, which is incorporated herein by reference in its entirety. For example, a DHP-cyanine dye-antibody conjugate can be prepared according to the general scheme as shown in scheme 12 (FIG. 9). As another example, a polymer-antibody conjugate can be prepared according to the general scheme as shown in scheme 13 (FIG. 9). As a further example, a sulfoxide bridged DHP-cyanine dye antibody conjugate can be prepared as shown in scheme 10 (FIG. 8).

For example, preparation of polymer NHS ester can proceed as follows. Using a clean vial, dissolve 5 mg of polymer in 1 mL dry CH₃CN. To this, add 15 mg N,N,N′,N′-tetramethyl-O—(N-succinimidyl) uranium tetrafluoroborate (TSTU) and stir for 2 more minutes. To this, add 100 uL N,N-diisopropylethylamine (DIPEA) and continue stirring overnight with the cap sealed with parafilm. Later evaporate off the organic solvents in the reaction mixture. Dissolve the crude NHS in about 750 uL of 1×BBS buffer (pH 8.8) by a quick vortex and transfer it to a Zeba column 40K MWCO. Spin down the sample at 2200 RPM for 2 min and use this polymer NHS immediately.

Conjugation of polymer NHS with anti-CD4 antibody can proceed as follows. Take the polymer NHS in 1×BBS (800 uL), spin down, add to 0.6 mg of CD4 and mix with 100 μL of 0.5M Borate buffer (pH 9.0). Vortex quickly for 30 seconds and allow to mix for 3-4 hours in the coulter mix.

Purification of polymer-antibody conjugate through Histrap HP column can proceed as follows. Approach 1: After the crude reaction purify the conjugate using a Histrap HP column. Load the sample using 1×PBS buffer and collect the unbound fraction. This can be done using 20 CV of buffer. Later change the buffer to wash the bound fraction which has both conjugate and free antibody. This can be done using 1×PBS with 0.25M imidazole running for 10 CV.

Approach 2: SP Sepharose FF column. Equilibrate the column and load the sample using 20 mM Citrate buffer pH 3.5 and collect the unbound fraction. This can be done using 20 CV of buffer. Later change the buffer to elute the bound fraction which has both conjugate and free antibody. This can be done using 20 mM Tris buffer pH 8.5 running for 20 CV.

Approach 3: Load the crude conjugate in a Tangential flow filtration system equipped with a 300K MWCO membrane. The conjugate is washed using 1×PBS until the filtrate show no absorption at 405 nm or 355 nm. Later the compound is concentrated.

Purification of polymer-antibody conjugate through SEC column can proceed as follows. Load the crude conjugate containing free antibody to the Size Exclusion Column, using 1×PBS. Pool the tubes after checking the absorption spectra and concentrate in an Amicon Ultra-15 having a 30 KDa MWCO centrifugal concentrator.

Purification of polymer-antibody conjugate through a Nuvia HR-S column (Bio-Rad Laboratories, Inc.) can proceed as follows. Load crude polymer-antibody conjugate mixture to the Nuvia HR-S column using a biological buffer having a pH between about 2 to about 14 and a conductivity less than 3 mS/cm. Due to charge-charge interactions between the matrix and biomolecules, the polymer antibody conjugate will bind to the resin while free polymer dye will not interact with the resin and will flow through. Disrupt the charge-charge interactions between the matrix and polymer antibody conjugate by using a salt (e.g., NaCl, KCl, phosphate etc.) at a concentration ranging from about 0.1 to 2 M. The salt concentration can be reduced by adjusting the pH of the elution buffer. For example, the conjugate can be eluted using a biological buffer and gradient of salt concentration (e.g., NaCl, KCl) between about 100 to 1000 mM at a pH of between about 6 to about 10.

Purification of conjugate through a Nuvia cPrime column (Bio-Rad Laboratories, Inc.) can proceed as follows. Load crude polymer-antibody conjugate mixture to the Nuvia cPrime column using a biological buffer having a pH between about 2 to about 14 and a salt concentration (e.g., NaCl, KCl) ranging from 0 to about 1 M. Unreacted polymers will flow through the column while the polymer-antibody conjugate will bind to the column. Elute the polymer-antibody conjugate by increasing the pH of the elution buffer. For example, the crude polymer antibody conjugate can be loaded into the Nuvia cPrime column using a biological buffer pH 5.0, 5 mM NaCl and eluted with a biological buffer pH 7.0 and gradient of salt concentration 5 to 500 mM.

Purification of conjugate through an Anti-mouse anti-H+L antibody-agarose bead can proceed as follows. Mix crude polymer-antibody conjugate mixture with anti-mouse anti-H+L antibody-agarose bead in a biological buffer having a pH between about 6 to about 8 for about 30 minutes at room temperature. The anti-mouse anti H+L antibody-agarose bead will bind to the polymer antibody conjugate. Remove unreacted polymers by washing with the above-mentioned biological buffer using a benchtop centrifuge with a speed of 300 g for 3 minutes. Repeat the washing process at least three times. To elute the polymer-antibody conjugate, apply an IgG elution buffer with a pH ranging from about 2 to about 4 to the washed antibody-agarose bead and incubate for about 10 to 15 min. Centrifuge to collect the flow through that contains the polymer antibody conjugate.

### VII. Methods of Detecting an Analyte

The present disclosure provides methods for detecting a target analyte in a sample, the method comprising:
providing a sample that is suspected of containing a target analyte; and
contacting the sample with a specific binding partner conjugated to a fluorescent compound or polymer of the present disclosure, wherein the binding partner is capable of interacting with the target analyte.

A light source is applied to the sample that can excite the fluorescent compound or polymer; and light emitted from the conjugated fluorescent compound or polymer complex is detected. In the typical assay, water-soluble fluorescent compounds or polymers of the present disclosure are excitable with a light having wavelength between about 420 nm and about 900 nm and the emitted light is typically between about 450 nm and about 1000 nm.

Alternatively, excitation light can have a wavelength between about 500 nm and about 850 nm and the emitted light can have a wavelength between about 550 nm and about 950 nm. The fluorescent compounds and polymers of the present disclosure may have an excitation spectrum tuned to the UV, violet, blue, yellow, green, red and NIR or another laser depending on design of the compound or polymers.

In the method of the present disclosure, the fluorescent compound or polymer can be any water-soluble fluorescent compound or polymer of the present disclosure as disclosed herein.

A method is provided for detecting a target analyte in a sample comprising: providing a sample that is suspected of containing the analyte; and contacting the sample with a specific binding partner conjugated to a fluorescent compound, polymer, or tandem dye according to the disclosure, wherein the binding partner is capable of interacting with the target analyte. The binding partner may be a protein, peptide, affinity ligand, antibody, antibody fragment, carbohydrate, lipid, nucleic acid or an aptamer. When the binding partner is an antibody, the method may be configured for flow cytometry; the water-soluble fluorescent polymer may be bound to a substrate; the analyte may be a protein expressed on a cell surface; the method may be configured as an immunoassay; or the method may further comprise providing additional specific binding partners for detecting additional analytes simultaneously.

### (VIII) Tandem Dyes

The compounds, polymers, and labeled specific binding partners of the present disclosure are capable of transferring energy to a linked acceptor chromophore or a linked donor chromophore. The compounds, polymers, and labeled specific binding partners of the disclosure can be covalently linked to an acceptor or donor chromophore in energy-receiving proximity such that excitation of the donor leads to energy transfer to, and emission from, the covalently attached acceptor signaling chromophore. Mechanisms for energy transfer between the compounds, polymers, and labeled specific binding partners of the present disclosure and a linked donor or acceptor chromophore include, for example, resonant energy transfer (e.g., Förster (or fluorescence) resonant energy transfer, FRET), quantum charge exchange (Dexter energy transfer) and the like. The terms “acceptor chromophore” and “acceptor fluorophore” are used interchangeably herein. The terms “donor chromophore” and “donor fluorophore” are used interchangeably herein.

As such, the present disclosure provides tandem dyes, comprising a fluorescent compound, polymer, or labeled specific binding partner according to the disclosure, and an acceptor chromophore or a donor chromophore covalently linked to the fluorescent compound, polymer, or labeled specific binding partner. In some embodiments, the fluorescent compounds, polymers, or water-soluble fluorescent compounds or polymers of the disclosure, and conjugates thereof, are donor dyes. In some cases, the fluorescent compounds, polymers, or water-soluble fluorescent compounds or polymers of the disclosure, and conjugates thereof, are acceptor dyes. In some cases, the fluorescent compounds, polymers, or water-soluble fluorescent compounds or polymers of the disclosure, and conjugates thereof, include additional acceptor dye(s) (e.g., fluorophores or chromophores) attached to a donor dye having a structure of the disclosure, or additional donor dye(s) (e.g., fluorophores or chromophores) attached to an acceptor dye having a structure of the disclosure.

When a light source excites the donor compound or polymer backbone, the fluorophores, acceptor dyes or chromophores can absorb energy of an appropriate wavelength and emit or transfer energy. The fluorophore (FP), chromophore, donor, or acceptor dye linked to the fluorescent dyes of the invention may have an absorption or emission profile with a degree of overlap with the absorption or emission profile of the DHP-cyanine compounds or polymers of the disclosure. The FP, chromophore, donor, or acceptor dye linked to the fluorescent dyes of the invention can be a fluorescent dye that has absorption maximum longer than 405 nm or 575 nm, and emission maximum longer than 428 nm, 450 nm, or 600 nm, and optionally may exhibit with fluorescence quantum yield larger than 2%, 3%, 4%, 5%, 6%, 7%, 8%, or 10%.

Any convenient fluorescent dyes may be utilized in the tandem dyes as an acceptor chromophore or donor chromophores. The chromophores and fluorophores may be selected from coumarins, fluoresceins, rhodamines, cyanines, bodipys, or other polycyclic aromatics. Many fluorophores are commercially available and may be selected from but are not limited to, for example, any dye available from Beckman Coulter, Inc., including, but not limited to, SuperNova polymer dyes; any dye available from Becton Dickinson Biosciences, including, but not limited to, BD Horizon Brilliant™ polymer dyes; any dye available from ThermoFisher Scientific, including, but not limited to, Super Bright polymer dyes, and Alexa Fluor dyes, including, but not limited to, Alexa Fluor 488, Alexa Fluor 532, Alexa Fluor 546, Alexa Fluor 568, Alexa Fluor 594, Alexa Fluor 633, Alexa Fluor 647, Alexa Fluor 660, Alexa Fluor 680; ATTO 390, ATTO 465, ATTO 488, ATTO 495, ATTO 514, ATTO 532, ATTO 550, ATTO 565, ATTO 590, ATTO 594, ATTO 610, ATTO 620, ATTO 633, ATTO 647, ATTO 647N, ATTO 655, ATTO 665, ATTO 680, ATTO 700, ATTO 725, ATTO 740, 5-carboxy-2,7-dichlorofluorescein, 5-Carboxyfluorescein (5-FAM), 5-Carboxynapthofluorescein, 5-Carboxytetramethylrhodamine (5-TAMRA), 5-FAM (5-Carboxyfluorescein), 5-ROX, 6-TAMRA, 6-Carboxyrhodamine 6G, 6-CR6G, 6-JOE, 6-FAM, 6-ROX, Bodipy 492/515, Bodipy 493/503, Bodipy 500/510, Bodipy 505/515, Bodipy 530/550, Bodipy 542/563, Bodipy 558/568, Bodipy 564/570, Bodipy 576/589, Bodipy 581/591, Bodipy 630/650-X, Bodipy 650/665-X, Bodipy 665/676, Bodipy Fl, Bodipy R6G, Bodipy TMR, Bodipy TR, CF 488A, CF 555, CF 568, CF 594ST, CF 633, CF 640R, CF 647, CF 660C, CF 680, CF680R, CF 750, CF 770, CF 790, CL-NERF, CMFDA, Cy2, Cy3, Cy3.5, Cy5, Cy5.5, Cy7, DDAO, DiA, DiD, DiI, DyLight 488, DyLight 550, DyLight 594, DyLight 633, DyLight 650, DyLight 680, DyLight 755, DyLight 800, DiO, DiR, DM-NERF, DsRed, DTAF, DY-490, DY-495, DY-505, DY-530, DY-547, DY-548, DY-549, DY-549P1, DY-550, DY-554, DY-555, DY-556, DY-560, DY-590, DY-591, DY-594, DY-605, DY-610, DY-615, DY-630, DY-631, DY-632, DY-633, DY-634, DY-635, DY-636, DY-647, DY-648, DY-649, DY-649P1, DY-650, DY-651, DY-652, DY-654, DY-675, DY-676, DY-677, DY-678, DY-679, DY-679P1, DY-680, DY-681, DY-682, DY-700, DY-701, DY-703, DY-704, DY-730, DY-731, DY-732, DY-734, DY-749, DY-750, DY-751, DY-752, DY-754, DY-776, DY-777, DY-778, DY-780, DY-781, DY-782, DY-800, DY-831, Eosin, Erythrosin, FITC, Fluo-3, Fluo-4, Fluor-Ruby, FluorX, FM 1-43, FM 1-46, iFluor 488, iFluor 555, iFluor 594, iFluor 647, iFluor 680, iFluor 700, iFluor 750, iFluor 780, Lyso Tracker Green, Lyso Tracker Yellow, Mitotracker Green, Mitotracker Orange, Mitotracker Red, NBD, Oregon Green 488, Oregon Green 514, PKH26, PKH67, Resorufin, RH 414, Rhod-2, Rhodamine, Rhodamine 110, Rhodamine 123, Rhodamine 6G, Rhodamine B, Rhodamine Green, Rhodamine Red, Rose Bengal, Spectrum Green, Spectrum Orange, Spectrum Red, SYTO 11, SYTO 12, SYTO 13, SYTO 14, SYTO 15, SYTO 16, SYTO 17, SYTO 18, SYTO 20, SYTO 21, SYTO 22, SYTO 23, SYTO 24, SYTO 25, SYTO 40, SYTO 41, SYTO 42, SYTO 43, SYTO 44, SYTO 45, SYTO 59, SYTO 60, SYTO 61, SYTO 62, SYTO 63, SYTO 64, SYTO 80, SYTO 81, SYTO 82, SYTO 83, SYTO 84, SYTO 85, SYTOX Blue, SYTOX Green, SYTOX Orange, Texas Red, Tide Fluor 2 (TF2), Tide Fluor 2WS (TF2WS), Tide Fluor 3 (TF3), Tide Fluor 3WS (TF3WS), Tide Fluor 4 (TF4), Tide Fluor 5WS (TF5WS), Tide Fluor 6WS (TF6WS), Tide Fluor 7WS (TF7WS), Tide Fluor 8WS (TF8WS), TRITC, and XTRITC. Acceptor dyes useful in the disclosure may include, for example, a cyanine dye, a xanthene dye, a coumarin dye, a thiazine dye, an acridine dye, FITC, CY3B, Cy55, Alexa 488, Texas red, Cy5, Cy7, Alexa 750, Cy55, Cy3B, Cy3.5, Alexa 750, 800 CW, Biotium CF 555, diethyl coumarin, DY705 (Dyomics), DY431, DY485XL, DY500XL, DY610, DY640, DY654, DY 682, DY 700, DY 701, DY 704, DY 730, DY 731, DY732, DY 734, DY 752, DY 778, DY 782, DY 800, DY 831 and 800CW. The acceptor dye may be a pendant acceptor dye. The tandem dye may comprise a DHP-cyanine or DHP-squaraine compound or polymer according to the present disclosure comprising one or more, two or more, three or more, 1-30, 2-20, or 2.5-10 acceptor dye moieties. The tandem dye may comprise a donor dye and one or more, two or more, three or more, 1-30, 2-20, or 2.5-10 acceptor DHP-cyanine or DHP-squaraine acceptor dye compounds according to the present disclosure.

Fluorescent tandem dyes can be prepared using techniques known to those of skill in the art or using methods known in the art in combination with methods described herein. The tandem dyes can be water-soluble.

In some embodiments, instead of being attached to the compound or polymer backbone, acceptor dyes, chromophores, fluorophores, functional moieties and binding partners can be attached to compounds or polymers of the present disclosure through a linker moiety using the method of direct modification of core polymers described in U.S. Pat. No. 11,584,825, which is incorporated herein by reference in its entirety.

In some embodiments, the present disclosure provides a tandem dye comprising: a fluorescent compound, polymer, or labeled specific binding partner according to the disclosure; and an acceptor chromophore covalently linked to the fluorescent compound, polymer, or labeled specific binding partner.

### Samples

The sample in the methods of the present disclosure can be, for example, blood, bone marrow, spleen cells, lymph cells, bone marrow aspirates (or any cells obtained from bone marrow), urine (lavage), serum, saliva, cerebral spinal fluid, urine, amniotic fluid, interstitial fluid, feces, mucus, or tissue (e.g., tumor samples, disaggregated tissue, disaggregated solid tumor). In certain embodiments, the sample is a blood sample. In some embodiments, the blood sample is whole blood. The whole blood can be obtained from the subject using standard clinical procedures. In some embodiments, the sample is a subset of one or more cells of whole blood (e.g., erythrocyte, leukocyte, lymphocyte (e.g., T cells, B cells or NK cells), phagocyte, monocyte, macrophage, granulocyte, basophil, neutrophil, eosinophil, platelet, or any cell with one or more detectable markers). In some embodiments, the sample can be from a cell culture.

The subject can be a human (e.g., a patient suffering from a disease), a commercially significant mammal, including, for example, a monkey, cow, or horse. Samples can also be obtained from household pets, including, for example, a dog or cat. In some embodiments, the subject is a laboratory animal used as an animal model of disease or for drug screening, for example, a mouse, a rat, a rabbit, or guinea pig.

### Analytes

An “analyte” or “target analyte” as used herein, refers to a substance, e.g., molecule, whose abundance/concentration is determined by some analytical procedure. For example, in the present disclosure, an analyte can be a protein, peptide, nucleic acid, lipid, carbohydrate small molecule, or a target-associated biomolecule.

The target analyte may be, for example, nucleic acids (DNA, RNA, mRNA, RNA, or rRNA), peptides, polypeptides, proteins, lipids, ions, monosaccharides, oligosaccharides, polysaccharides, lipoproteins, glycoproteins, glycolipids, or fragments thereof. In some embodiments, the target analyte is a protein and can be, for example, a structural microfilament, microtubule, and intermediate filament proteins, organelle-specific markers, proteasomes, transmembrane proteins, surface receptors, nuclear pore proteins, protein/peptide translocases, protein folding chaperones, signaling scaffolds, ion channels and the like. The protein can be an activatable protein or a protein differentially expressed or activated in diseased or aberrant cells, including but not limited to transcription factors, DNA and/or RNA-binding and modifying proteins, nuclear import and export receptors, regulators of apoptosis or survival and the like.

The compounds, polymers, labeled specific binding partner, or tandem dye according to the present disclosure may be a water-soluble fluorescent dye. The fluorescent compound, labeled specific binding partner, or tandem dye according to the present disclosure may exhibit solubility in water at ambient room temperature of >1 mg/mL, >2 mg/mL, >3 mg/mL, >4 mg/mL, >5 mg/mL, >6 mg/mL, >7 mg/mL, >8 mg/mL, >9 mg/mL, >10 mg/mL, >20 mg/mL, >30 mg/mL, >40 mg/mL, >50 mg/mL, >80 mg/mL, or >100 mg/mL.

The compounds, polymers, labeled specific binding partner, or tandem dye according to the present disclosure may exhibit a maximum excitation wavelength (λex) of >400 nm, >500 nm, >600 nm, >700 nm, >800 nm, >850 nm, or within a range of between about 400 nm to about 1,000 nm, or about 600 nm to about 950 nm. The DHP-cyanine compounds, polymer, labeled specific binding partner comprising a DHP-cyanine compound or DHP-cyanine tandem dye, or DHP-cyanine acceptor dye comprising a DHP-cyanine compound according to the present disclosure may exhibit an emission maxima (λem) of >550 nm, >650 nm, >750 nm, >850 nm, or >900 nm, or within a range of between about 600 to about 1200, or about 550 nm to about 1050 nm, or about 650 nm to about 1050 nm.

### Assays

The DHP-cyanine dyes, tandem dyes, labeled specific binding partners, compositions, methods and systems as described herein may find use in a variety of applications, including diagnostic and research applications, in which the labelling, detection and/or analysis of a target of interest is desirable. Such applications include methodologies such as, for example, cytometry, microscopy, immunoassays (e.g. competitive or non-competitive), fluorescence in situ hybridization (FISH), cell tracing, receptor labeling, fluorescence spectroscopy, assessment of a free analyte, assessment of receptor bound ligand, and so forth. The compositions, system and methods described herein may be useful in analysis of any of a number of samples, including but not limited to, biological fluids, cell culture samples, and tissue samples. In certain aspects, the compositions, system and methods described herein may find use in methods where analytes are detected in a sample, if present, using fluorescent labels, such as in fluorescent activated cell sorting or analysis, immunoassays, immunostaining, and the like. In certain instances, the compositions and methods find use in applications where the evaluation of a sample for the presence of a target analyte is of interest. In some cases, the methods and compositions find use in any assay format where the detection and/or analysis of a target from a sample is of interest, including but not limited to, flow cytometry, fluorescence microscopy, in-situ hybridization, enzyme-linked immunosorbent assays (ELISAs), western blot analysis, magnetic cell separation assays and fluorochrome purification chromatography. In certain instances, the methods and compositions find use in any application where the fluorescent labelling of a target molecule is of interest. The subject compositions may be adapted for use in any convenient applications where pairs of specific binding members find use, such as biotin-streptavidin and hapten-anti-hapten antibody.

Assay systems utilizing a binding partner and a fluorescent label to quantify bound molecules are well known. Examples of such systems include flow cytometers, scanning cytometers, imaging cytometers, fluorescence microscopes, and confocal fluorescent microscopes.

In some embodiments, flow cytometry is used to detect fluorescence. A number of devices suitable for this use are available and known to those skilled in the art. Examples include BCI Navios, Gallios, Aquios, and CytoFLEX flow cytometers.

In other embodiments, an assay is used. The assay can be an immunoassay. Examples of immunoassays useful in the disclosure include, but are not limited to, fluoroluminescence assay (FLA), and the like. The assays can also be carried out on protein arrays. When the binding partners are antibodies, antibody or multiple antibody sandwich assays can also be used. A sandwich assay refers to the use of successive recognition events to build up layers of various binding partners and reporting elements to signal the presence of a particular analyte. Examples of sandwich assays are disclosed in U.S. Pat. No. 4,486,530 and in the references noted therein.

### (IX) Kits

The disclosure provides a kit comprising at least one DHP-cyanine or DHP-squaraine compound or polymer, labeled specific binding partner, or tandem dye according to the present disclosure. Aspects of the invention further include kits for use in practicing the subject methods and compositions. The compositions of the invention can be included as reagents in kits either as starting materials or provided for use in, for example, the methodologies described above.

A kit can include a DHP-cyanine or DHP-squaraine compound or polymer, labeled specific binding partner, or tandem dye as described herein and a container. Any convenient containers can be utilized, such as tubes, bottles, or wells in a multi-well strip or plate, a box, a bag, an insulated container, and the like. In some instances, the subject kits can include one or more components selected from a DHP-cyanine or DHP-squaraine compound or polymer, labeled specific binding partner, or tandem dye according to the present disclosure, a fluorophore, a chromophore, a specific binding member, a specific binding member conjugate, a support bound specific binding member, a cell, a support, a biocompatible aqueous elution buffer, and/or instructions for use. In some embodiments of the kit, the DHP-cyanine or DHP-squaraine compound or polymer, or tandem dye according to the present disclosure is covalently linked to a specific binding partner.

In some instances, the subject kits can be a “labeling kit” that include DHP-cyanine or DHP-squaraine compound or polymer, or tandem dye according to the present disclosure comprising a sidechain chemoselective functional group (also referred to as a “conjugation tag”) such as, for example, a NHS ester of a DHP-cyanine or DHP-squaraine compound or polymer and the like, to which any convenient target moiety of interest (e.g., a donor or acceptor dye, fluorophore, chromophore, a specific binding partner, a support) can be conjugated. The chemoselective functional group may include a reactive group (e.g., biotin) that targets specific functional groups on biomolecules (e.g., proteins or antibodies), such as, for example, primary amines, sulfhydryls, carboxyls, or carbohydrates. The chemoselective functional group can be one used in “click chemistry” reactions.

In certain instances, the conjugation tag includes a maleimide functional group and the target moiety includes a thiol functional group, or vice versa. In some instances, the conjugation tag includes an alkyne (e.g., a cyclooctyne group) functional group and the target moiety includes an azide functional group, or vice versa, which can be conjugated via Click chemistry. In certain instances, the conjugation tag includes an alkene (e.g., a cyclooctene group) functional group and the target moiety includes a tetrazine functional group, or vice versa, which can be conjugated via inverse-demand Diels-Alder cycloaddition reaction. In some instances, the conjugation tag includes an amine-reactive chemical group, such as, for example, a NHS ester (N-hydroxysuccinimide esters) or imidoester functional group and the target moiety includes a NH₂ functional group, or vice versa. In some instances, the conjugation tag includes a biotin-binding protein (e.g., Avidin, Streptavidin, or NeutrAvidin) and the target moiety includes a biotin molecule, or vice versa, which can non-covalently interact.

### EXAMPLES

### Example 1. General Procedures to Synthesize DHP Indole-Cyanine Compounds 1-5

Synthetic procedures for synthesis of DHP indole-cyanine compounds are illustrated in FIG. 2, Scheme 2. Synthetic procedures for preparation of Compounds 1-5, as shown in Table 1, are shown below.

### Synthesis of DHP-diol Intermediate Z; 3-bromo-9,10-dihydrophenanthrene-9,10-diol

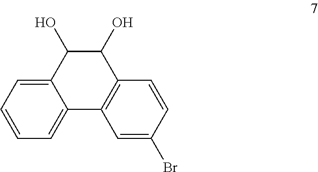 [SMILES: OC1c2ccccc2-c2cc(Br)ccc2C1O]

Commercially available 3-bromo DHP-dione was reduced using NaBH₄ according to previously published procedure in 85% yield (U.S. Pat. No. 11,208,527B2).

### Synthesis of DHP-OMe Intermediate 8; 3-bromo-9,10-dimethoxy-9,10-dihydrophenanthrene

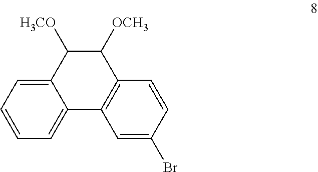 [SMILES: COC1c2ccccc2-c2cc(Br)ccc2C1OC]

To a nitrogen purged THE solution of DHP-diol (1 mmol) in ice, NaH (4 mmol) was added. After 5 minutes, methyl iodide solution was added dropwise. Reaction allowed to stir at room temperature for overnight. Next day reaction quenched using a few drops of water. After evaporating off the THF reaction mixture was extracted using chloroform. Pure compound was obtained after column chromatography in 80% yield.

### Synthesis of DHP-Hydrazone Intermediate 9; 1-(9,10-dimethoxy-9,10-dihydrophenanthren-3-yl)-2-(diphenylmethylene)hydrazine

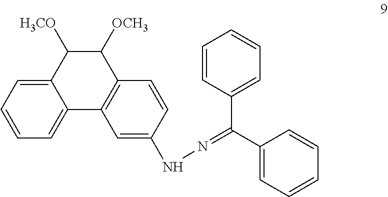 [SMILES: COC1c2ccccc2-c2cc(NN=C(c3ccccc3)c3ccccc3)ccc2C1OC]

Benzophenone hydrazone (1.50 mmol), Pd(OAc)2 (0.015 mmol), and (+)-BINAP (12.7 mg, 0.020 mmol; (2,2′-bis(diphenylphosphino)-1,1′-binaphthyl)) were suspended in toluene (2 mL). The reaction vessel was purged briefly with argon, stirred for 3 min. To the resulting purple solution was added DHP derivative (1.36 mmol) NaOt-Bu (1.90 mmol), and toluene (0.4 mL). The reaction vessel was heated at 100° C. for overnight. Later the reaction mixture was cooled to 25° C., filtered through a short pad celite. The clear filtrate was concentrated and the residue was purified by flash column Chromatography Yield 60%

### Synthesis of DHP-Indole Intermediate 10; 5,6-dimethoxy-8,8,9-trimethyl-6,8-dihydro-5H-naphtho[2,1-f]indole

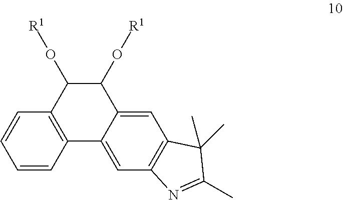

DHP-hydrazone (1.35 mmol), isopropyl methyl ketone (2.03 mmol), and TsOH*H2O (6.75 mmol) were heated for 15 h in ethanol (20 mL) at reflux. Purification of the crude product by flash column chromatography afforded the DHP-indole derivative in 25% yield.

### Synthesis of DHP-Indole Sulfonate Intermediate 11; 3-(5,6-dimethoxy-8,8,9-trimethyl-6,8-dihydro-5H-10l4-naphtho[2,1-f]indol-10-yl) propane-1-sulfonate

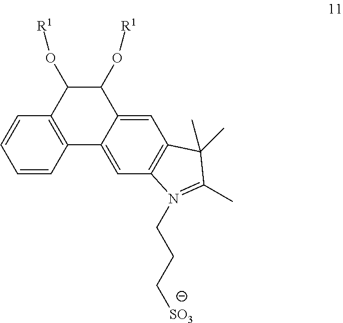

DHP-indole intermediate (1 mmol) 10, is heated with 1,3-propane sultone at 120 deg Celsius for 2h. Later the reaction mixture is cooled and washed with ether and dried to provide sulfonated DHP-indole intermediate compound 11.

### General Procedure to Synthesize Compounds 1-3

A solution of sulfonated DHP indole (1 mmol) 11 in MeCN was added to a refluxing mixture of the corresponding aldehyde dianiline hydrochloride (1.20 mmol), Ac₂O (120 mL), DIEA (600 mL) and NaOAc (78 mg) in a mixed solvent of MeCN/DCM. The resultant mixture was stirred for additional 2 h before adding excess of the sulfonated DHP indole (1.50 mmol). The mixture was again refluxed for 2 h. The reaction was quenched by water and solvent was removed by a rotary evaporator. The residue was washed sequentially with EtOAc, MeCN, EtOAc and water before purifying by column chromatography to obtain compounds 1-3 as shown in Table 1.

### General Procedure to Synthesize Compounds 4-5

A solution of sulfonated DHP indole (1 mmol) 11 in MeCN was added to a refluxing mixture of the corresponding aldehyde dianiline hydrochloride (1.20 mmol), Ac₂O (120 μL), DIEA (600 μL) and NaOAc (78 mg) in a mixed solvent of MeCN/DCM. The resultant mixture was stirred for additional 2 h before adding excess of the 1-(4-Sulfobutyl)-2,3,3-trimethyl-4,5-benzindolium, inner salt (1.50 mmol). The mixture was again refluxed for 2 h. The reaction was quenched by water and solvent was removed by a rotary evaporator. The residue was washed sequentially with EtOAc, MeCN, EtOAc and water before purifying by column chromatography to obtain compounds 4 or 5.

Compounds 1-5 may be derivatized with sulfonamide PEG side groups to increase water solubility. For example, a [symbol] unds 1-5 may be treated with thionyl chloride in DMF to form a propane sulfonyl chloride intermediate, then treated with NH₂PEG₅₅₀OMe, TEA, in CH₂Cl₂ to form sulfonamide PEG side groups.

### Example 2. Synthesis of DHP-Indole Propyl Sulfonate Intermediate 21

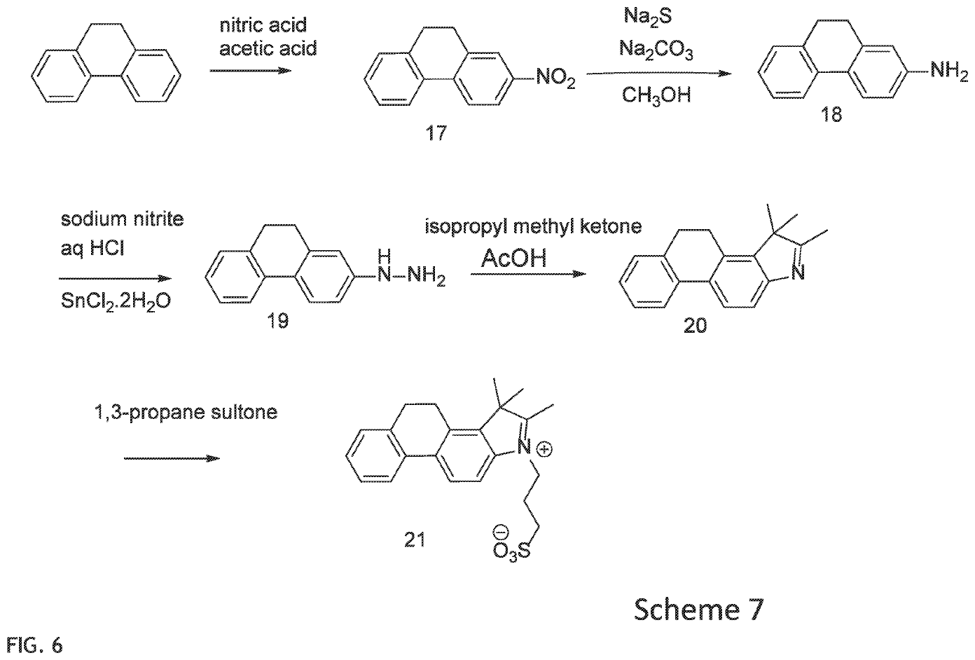

FIG. 6 shows synthetic scheme 7 for preparing N-propyl sulfonate DHP-indole compound 21 from dihydrophenanthrene.

### Synthesis of 2-Nitro DHP Intermediate 17; 2-nitro-9,10-dihydrophenanthrene

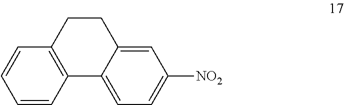 [SMILES: O=[N+]([O-])c1ccc2c(c1)CCc1ccccc1-2]

To 1 mmol of 9,10-dihydrophenanthrene 8 mL acetic acid and 2 mL of fuming nitric acid (20%) was added. Mixture was stirred keeping the temperature at 30 deg C. After 2 hours the red solution was poured into crushed ice. Allowed to complete the precipitation and decanted the aqueous layer. It was washed with more cold water (500 mL). Dried and obtained 2- and 4-nitro 9,10-dihydrophenanthrene 17 was used for the next step without further purification.

### Synthesis of 9,10-dihydrophenanthren-2-amine Intermediate 18

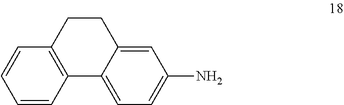 [SMILES: Nc1ccc2c(c1)CCc1ccccc1-2]

Na₂S (3 mmol) was dissolved in minimum amount of hot water and mixed with 240 mg of Na₂CO₃. This was added to 2.2 mmols of nitro-9,10-dihydrophenanthrene 17 in methanol. Mixture was heated overnight. Next day extracted using chloroform and dried. Carried out chromatography to separated out 2- and 4-amino-9,10-dihydrophenanthrene 18.

### Synthesis of (9,10-dihydrophenanthren-2-yl)hydrazine Intermediate 19

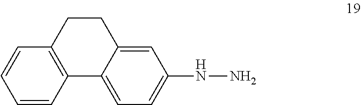 [SMILES: NNc1ccc2c(c1)CCc1ccccc1-2]

To an ice cold suspension of 2-amino-9,10-dihydrophenanthrene 18 (1 mmol) in 20% HCl (2 mL) sodium nitrite (100 mg in 250 uL) was added in dropwise. Stirring continued at 0 deg C. for 1h. A small amount of urea was added to decompose excess of nitrous acid. Temperature was kept at −5 deg C. To the [symbol] salt solution, SnCl₂·2H₂O (250 mg) in minimum amount of Conc HCl was added and stirred for 2h. Later the precipitate was filtered and dried. Later the precipitate is dissolved in chloroform and extracted using water containing 10% NaOH. Organic layer was dried followed by chromatography to obtain 19.

### Synthesis of 2,3,3-trimethyl-4,5-dihydro-3H-naphtho[2,1-e]indole Intermediate 20

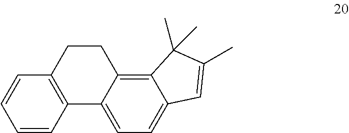 [SMILES: CC1=Cc2ccc3c(c2C1(C)C)CCc1ccccc1-3]

2-hydrazino-9,10-dihydrophenanthrene 19 (1 mmol) was dissolved using 5 mL glacial acetic acid. This was mixed with isopropyl methyl ketone (2 mmol) and the reaction carried out at 110 deg overnight. Next day extracted using ethylacetate and water followed by chromatography to obtain 20.

### Synthesis of DHP-Indole Propyl Sulfonate Intermediate 21; 3-(2,3,3-trimethyl-4,5-dihydro-3H-naphtho[2,1-e]indol-1-ium-1-yl) propane-1-sulfonate Intermediate 21

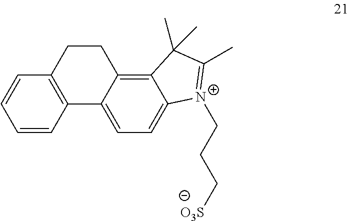

Dihydrophenanthrene indole derivative 20 (1 mmol) obtained from previous step was heated with 1,3-propanesultone at 120 deg Celsius for 2 h. After cooling the product was washed with ethyl acetate to remove any unreacted starting material to obtain 21.

### Example 3. Synthesis of Sulfoxide Bridged DHP-Indole Cyanine Antibody 25

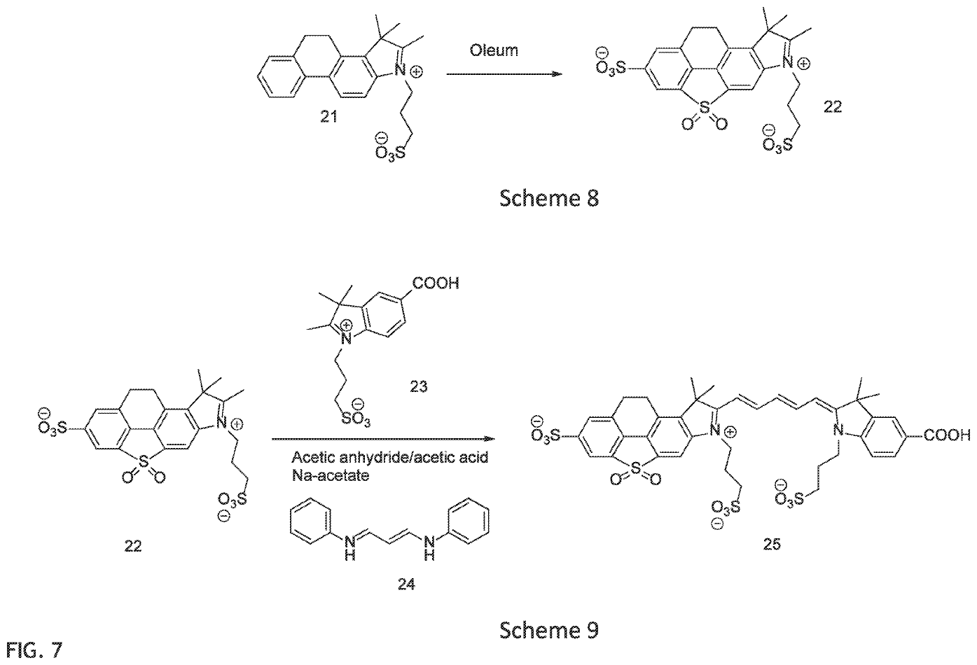

FIG. 7 shows synthetic schemes 8 and 9 for preparing sulfoxide bridged DHP indole cyanine dye 25 from -propyl sulfonate DHP-indole intermediate compound 21.

### Synthesis of Sulfoxide Bridged DHP-Indole Propyl Sulfonate Intermediate 22; 2-((l1-oxidaneyl)dioxo-l6-sulfaneyl)-6-(3-((l1-oxidaneyl)dioxo-l6-sulfaneyl)propyl)-7,8,8-trimethyl-9,10-dihydro-8H-6l4-thieno[2′,3′,4′,5′:4,5]phenanthro[2,1-b]pyrrole 4,4-dioxide 22

2-aminodihydrophenanthrene 18 was reacted using NaNO₂/HCl in an ice bath to convert to the corresponding diazonium salt. This upon reaction with SnCl₂/HCl at 0-5 deg C. formed the 2-hydrazinodihydrophenanthrene (19). This product was used without further purification for the next step. Later 19 was reacted with 3-methyl-2-butanone under Fisher indole synthesis conditions to produce isomers of DHP indole (20). The indole ring formation could happen at 1 and 2 positions as well as 2 and 3 positions of the DHP. Both isomers were isolated using flash chromatography. Indole formed at 1,2-position of the DHP was taken and reacted with 1,3-propane sultone to obtain intermediate DHP-indole compound 21 in 100% yield. This was further treated with 20% oleum that was precooled at −20 deg C. and the reaction continued overnight at room temperature. Crude product was precipitated using ether followed by a C18 reverse phase chromatography (100% water to 95% water 5% methanol) to obtain compound 22.

### Synthesis of Sulfoxide Bridged DHP-Indole Cyanine Dye 25; (Z)-2-((2E,4E)-5-(2-((l1-oxidaneyl)dioxo-l6-sulfaneyl)-6-(3-((l1-oxidaneyl)dioxo-l6-sulfaneyl)propyl)-8,8-dimethyl-4,4-dioxido-9,10-dihydro-8H-6l4-thieno[2′,3′,4′,5′:4,5]phenanthro[2,1-b]pyrrol-7-yl)penta-2,4-dien-1-ylidene)-1-(3-((l1-oxidaneyl)dioxo-l6-sulfaneyl)propyl)-3,3-dimethylindoline-5-carboxylic Acid 25

Sulfoxide bridged DHP indole (22) was treated with carboxylic acid functionalized sulfonated indole (23) and 24 in a mixture of acetic acid and acetic anhydride in presence of sodium acetate. After the reaction crude mixture was mixed with the cold ether and the precipitate was isolated. This was purified using C18 reverse phase chromatography (20% MeOH-80% water) to obtain DHP-sulfoxide Cy5 dye 25 which was found to exhibit Abs max 666 nm, emission max 686 nm, and extinction coefficient of 150450 M⁻¹ cm⁻¹.

### Example 4. Synthesis of Sulfoxide Bridged DHP-Indole Cyanine Antibody Conjugate 27

FIG. 8 shows synthetic scheme 10 for preparing sulfoxide bridged DHP indole cyanine dye antibody conjugate 27 from sulfoxide bridged DHP indole cyanine dye 25.

### Synthesis of Sulfoxide Bridged DHP-Indole Cyanine Dye Antibody Conjugate 27:

DHP-sulfoxide Cy5 dye (25) was converted to active NHS ester 26 using TSTU/DIPEA/DMSO/CH₃CN mixture. This was purified by precipitation techniques using diethyl ether. Active ester 26 was conjugated to the available amino groups in the antibody (Ab) at a pH ˜7.5-8. Unreacted excess dye was separated out using size exclusion spin column and the pure conjugate obtained (27) was used in flow cytometric studies.

### Example 5. Synthesis of DHP-Indole Intermediate 33; 1-(3-((λ1-oxidaneyl)dioxo-l6-sulfaneyl)propyl)-2,3,3,5,5-pentamethyl-4,5-dihydro-3H-1λ4-naphtho[2,1-e]indole 33

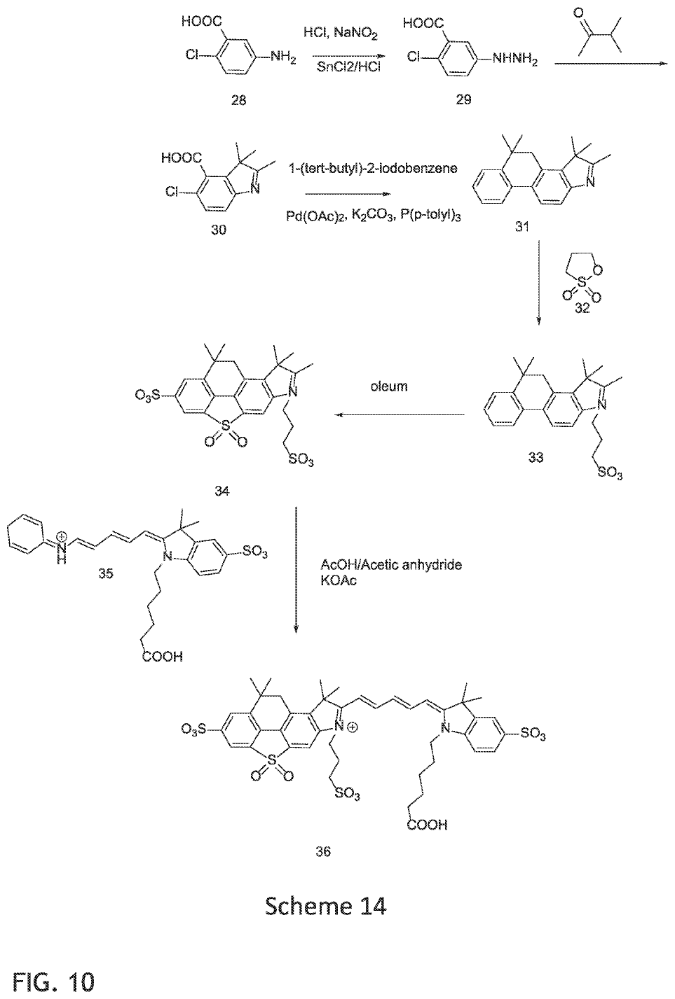

FIG. 10 shows exemplary synthetic scheme 14 for preparing N-propyl sulfonate DHP-indole intermediate compound 33 from chloroaminobenzoic acid 28. FIG. 10 also shows exemplary route for preparing sulfoxide bridged DHP indole cyanine dye 36 from N-propyl sulfonate DHP-indole intermediate compound 33.

### Preparation of 2-chloro-4-hydrazino Benzoic Acid 29

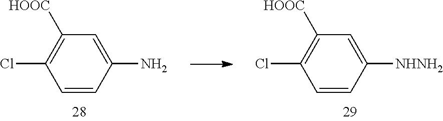 [SMILES: Nc1ccc(Cl)c(C(=O)O)c1 ; NNc1ccc(Cl)c(C(=O)O)c1]

Chloroaminobenzoic acid 28 was mixed with 20% HCl at 0 deg C. To this NaNO₂ (1 eq) in water was added and stirred for 1h. Later excess urea was added and stirred for 5 minutes. Reaction mixture was further cooled to about-5 deg C. To this, tin chloride (2.1 eq) was added in con HCl and stirred for another 2h. Later the reaction mixture was poured into ice cold water and filtered to obtain hydrazine compound 29.

### Preparation of 5-chloro-2,3,3-trimethyl-3H-indole-4-carboxylic Acid 30

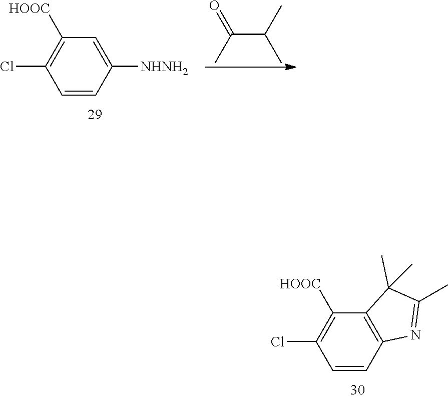

Hydrazino compound 29 was heated with 3-methyl-2-butanone at 120 deg C. for overnight. Next day the crude product was precipitated which upon repeated washings with EtOAc followed by silica chromatography gave the indole product 30 as a mixture of isomers.

### Preparation of 2,3,3,5,5-pentamethyl-4,5-dihydro-3H-naphtho[2,1-e]indole 31

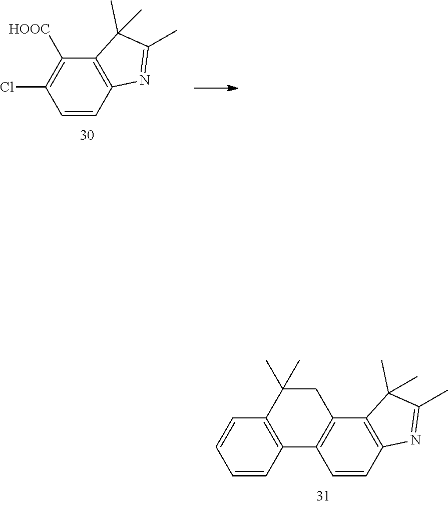 [SMILES: CC1=Nc2ccc(Cl)c(C(=O)O)c2C1(C)C ; CC1=Nc2ccc3c(c2C1(C)C)CC(C)(C)c1ccccc1-3]

Under Schlenk conditions, an overnight reaction was carried out between the indole derivative 30 along with 1-(tert-butyl)-2-iodobenzene in presence of Pd(OAc)₂, K₂CO₃, tri(o-tolyl)phosphine (P(p-tolyl)₃) and tetrabutyl ammonium bromide in DMF at 140 deg C. Next day crude product 31 was purified using column chromatography.

### Preparation of 1-(3-((l1-oxidaneyl)dioxo-l6-sulfaneyl)propyl)-2,3,3,5,5-pentamethyl-4,5-dihydro-3H-1l4-naphtho[2,1-e]indole 33

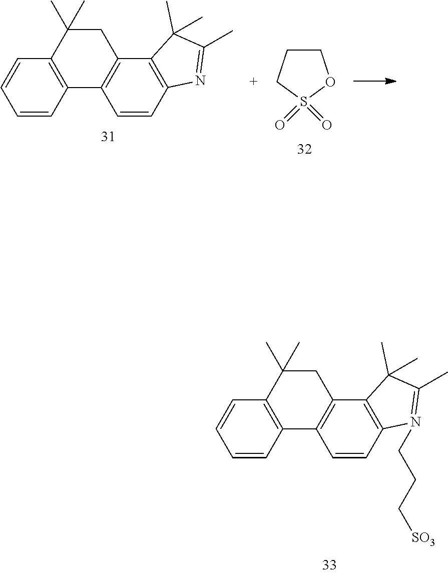

Compound 31 was mixed with 1 eqvt of 1,3-propane sultone 32 in THF at 120 deg C. for 90 min in a sealed tube. Later the crude product 33 was precipitated and washed using cold ether.

### Synthesis of Sulfoxide Bridged DHP-Indole Propyl Sulfonate Intermediate 34, 2-((l1-oxidaneyl)dioxo-l6-sulfaneyl)-6-(3-((l1-oxidaneyl)dioxo-l6-sulfaneyl)propyl)-7,8,8,10,10-pentamethyl-9,10-dihydro-8H-6l4-thieno[2′,3′,4′,5′:4,5]phenanthro[2,1-b]pyrrole 4,4-dioxide

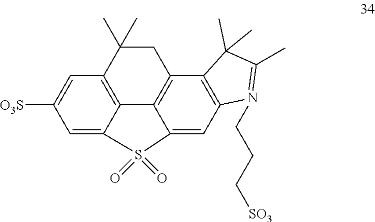

Sulfoxide bridged DHP-indole propyl sulfonate intermediate compound 34 was prepared from intermediate compound 33 in an analogous fashion as described for synthesis of compound 22, above.

### Synthesis of Sulfoxide Bridged DHP-Indole Cyanine dye 36, 2-((l1-oxidaneyl)dioxo-l6-sulfaneyl)-7-((1E,3E)-5-((Z)-5-((l1-oxidaneyl)dioxo-l6-sulfaneyl)-1-(5-carboxypentyl)-3,3-dimethylindolin-2-ylidene)penta-1,3-dien-1-yl)-6-(3-((l1-oxidaneyl)dioxo-l6-sulfaneyl)propyl)-8,8,10,10-tetramethyl-9,10-dihydro-8H-thieno[2′,3′,4′,5′:4,5]phenanthro[2,1-b]pyrrol-6-ium 4,4-dioxide

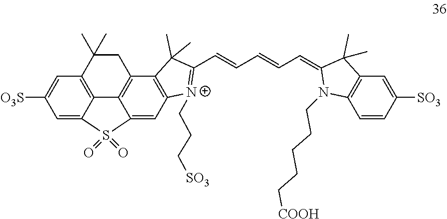

Sulfoxide bridged DHP indole (34) was treated with carboxylic acid functionalized sulfonated indole (35) in a mixture of acetic acid and acetic anhydride in presence of potassium acetate. After the reaction crude mixture was mixed with the cold ether and the precipitate was isolated. This was purified using C18 reverse phase chromatography to obtain DHP-sulfoxide Cy5 dye 36 which was found to exhibit Abs max 663 nm, emission max 683 nm. The above specification, examples and data provide a complete description of the manufacture and use of the composition of the invention. Since many embodiments of the invention can be made without departing from the spirit and scope of the invention, the invention resides in the claims hereinafter appended.

1. A fluorescent compound comprising a structure according to Formula (II): 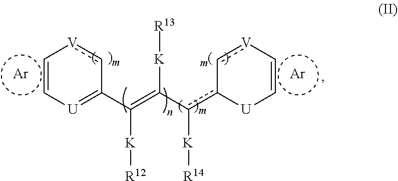
wherein
each 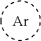
is independently selected from the group consisting of a substituted or unsubstituted benzene, benzene derivative, monocyclic aryl group, polycyclic aryl group, monocyclic heteroaryl group, and polycyclic heteroaryl group, wherein at least one 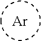
comprises 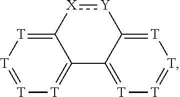
or a derivative thereof,
wherein the [symbol] in 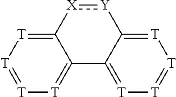
is a single bond, and
wherein the derivative optionally comprises an additional aryl or heteroaryl group fused at any available 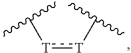
optionally wherein the derivative is 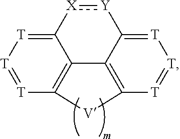
wherein
each V′ is independently SO₂, SO, S, NR¹¹, CR¹¹, C(R¹¹)₂, O, Si(R¹¹)₂, >C═O, >Se═O, —CH═CH—, or —N═CH—;
each T is independently selected from the group consisting of C, C(R¹), N, N(R¹), P, O, S, and Si(R¹);
each U is independently selected from the group consisting of NR¹⁰, O, P, Se, Te, and S;
each V is independently selected from the group consisting of NR¹¹, CR¹¹, C(R¹¹)₂, S, SO₂, O, Se, Te, and Si(R¹¹)₂;
each X is independently CR¹R² or SiR¹R²;
each Y is independently CR⁸R⁹ or SiR⁸R⁹;
wherein
each R¹, R², R⁸, and R⁹ is independently selected from the group consisting of a water-solubilizing moiety, a linked water-solubilizing moiety, a linker moiety, a linked E, a reactive group, a linked reactive group, binding partner, linked binding partner, a functional group, hydrogen, hydroxy, halogen, alkoxy, alkyl, alkenyl, alkynyl, cycloalkyl, haloalkyl, aryl, heteroaryl, (hetero)aryloxy, (hetero)arylamino, sulfonamide-PEG, phosphoramide-PEG, ammonium alkyl salt, ammonium alkyloxy salt, ammonium oligoether salt, carbonyl, acyl, sulfonate alkyl salt, sulfonate alkoxy salt, sulfonate oligoether salt, sulfonamido oligoether, sulfonamide, sulfinamide, phosphonamidate, phosphinamide, alkoxy sulfonamide PEG, alkoxy sulfonate, alkyl sulfonate, alkyl sulfonate salt, 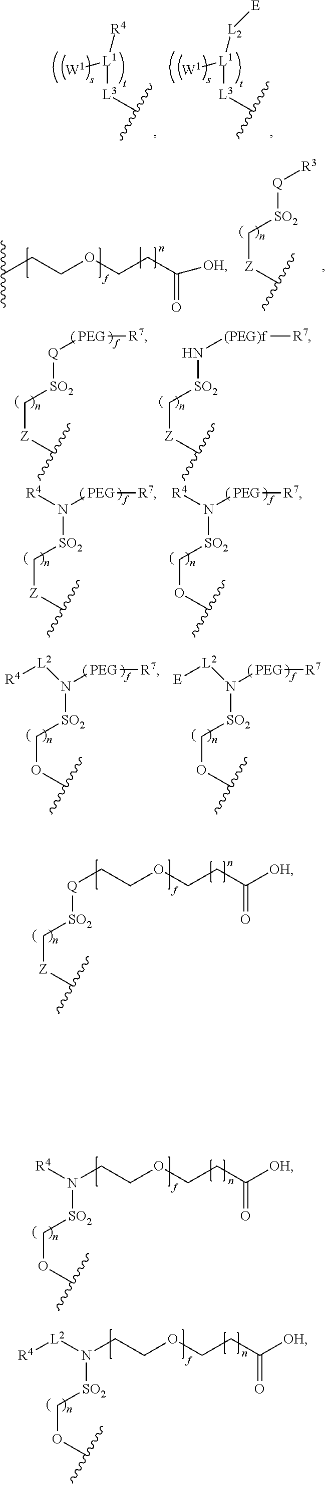 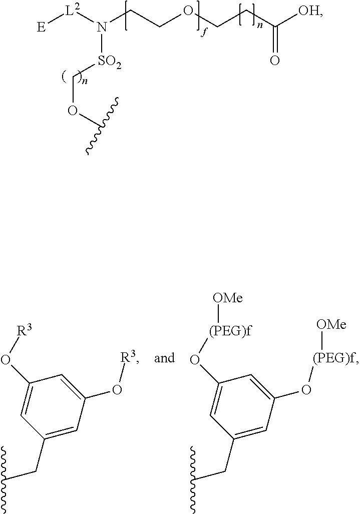
optionally wherein R¹ and R⁸ together form an unsubstituted or substituted cycloalkyl, cycloalkenyl, heterocycloalkyl, heterocycloalkenyl, cycloalkoxy, aryl, or heteroaryl having 3 to 9 ring members;
each R³ is independently selected from the group consisting of H, alkyl, alkene, alkyne, cycloalkyl, haloalkyl, alkoxy, (hetero)aryloxy, aryl, (hetero)arylamino, a water-solubilizing moiety, a chromophore, functional moiety, binding partner, and a PEG group;
each Q is independently a bond, NH, NR⁴, C₁-C₁₂ alkylene, CHR⁴, or CH₂;
each Z is independently CH₂, CHR⁴, O, NR⁴, or NH;
each W¹ is independently a water-solubilizing moiety;
L¹, L², and L³ are each independently selected linker moieties;
each E is independently selected from the group consisting of a chromophore, a functional moiety, a substrate, and a binding partner;
each R⁴ is independently selected from the group consisting of H, alkyl, PEG, a water-solubilizing moiety, a linker moiety, a chromophore, a functional group, a conjugation tag, carboxylic amine, amine, carbamate, carboxylic acid, carboxylate ester, maleimide, activated ester, N-hydroxysuccinimidyl, hydrazine, hydrazide, hydrazone, azide, alkyne, alkene, tetrazine, aldehyde, and thiol, or protected groups thereof;
each R⁷ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester, and C₁-C₁₂ alkoxy, a functional group, a chemoselective functional group, conjugation tag, linked conjugation tag, a linker, sulfonic acid, sulfonate, C₁-C₁₂ alkyl sulfonate, sulfonamide;
each R¹⁰ is independently selected from the group consisting of hydrogen, a linker moiety, a linked reactive group, a conjugation tag, a linked conjugation tag, a linked ionic group, a linked chromophore, a linked water-solubilizing moiety, a water-solubilizing moiety, a chromophore, a binding partner, a linked binding partner;
each R¹¹ is independently selected from the group consisting of hydrogen, a linker moiety, a linked reactive group, a linked ionic group, a linked chromophore, a linked water-solubilizing moiety, a chromophore, a binding partner, and a linked binding partner;
each R¹², R¹³, and R¹⁴ is independently selected from the group consisting of hydrogen, halogen, substituted or unsubstituted C₁-C₆ alkyl, substituted or unsubstituted C₁-C₆ heteroalkyl, substituted or unsubstituted C₁-C₆ alkene, substituted or unsubstituted C₃-C₁₀ cycloalkyl, substituted or unsubstituted heterocycloalkyl, substituted or unsubstituted aryl, and substituted or unsubstituted heteroaryl, CO₂R¹, CONR¹R², —O—CH₂CH₂-PEG-R⁷, —S—CH₂CH₂-PEG-R⁷, —N—CH₂CH₂-PEG-R⁷, O-aryl, S-aryl, N-aryl, —O-alkyl, S-alkyl, N-alkyl, wherein each alkyl or aryl can optionally be substituted with one or more R⁷, PEG, PEG-R⁷, or linking groups, optionally wherein each R¹², R¹³ and R¹⁴ is independently substituted with a R⁷ group; or at least two of R¹⁰, R¹¹, R¹², R¹³, and R¹⁴ together form an unsubstituted or substituted unsaturated or partially unsaturated C3-C10 cycloalkyl group, unsubstituted or substituted unsaturated or partially unsaturated C3-C10 heterocycloalkyl optionally substituted with O, an unsubstituted or substituted unsaturated or partially unsaturated cycloalkyl group having 3 to 8 ring members, or substituted or unsubstituted heterocycloalkyl having 3 to 8 ring members optionally substituted with O;
each K is independently a covalent bond, O, S, P, NR¹, Se, Te, CR¹R², or CH₂;
each f is independently an integer from 0 to 50;
each m is independently 0 or 1;
each n is independently 0, 1, 2, 3, or 4;
each s is independently 1 or 2; and
each t is independently 0, 1, 2, or 3,
optionally wherein at least one of R¹, R², R³, R⁴, R⁸, R⁹, R¹⁰, and R¹¹ comprises a water-solubilizing moiety or a linked water-solubilizing moiety, further optionally wherein the fluorescent compound is water-soluble.

2. The compound of claim 1, comprising a structure selected from the group consisting of Formula (IIa), (IIb), (IIc), (IId), (IIe), (IIf), (IIg), (IIk), and (IIl):

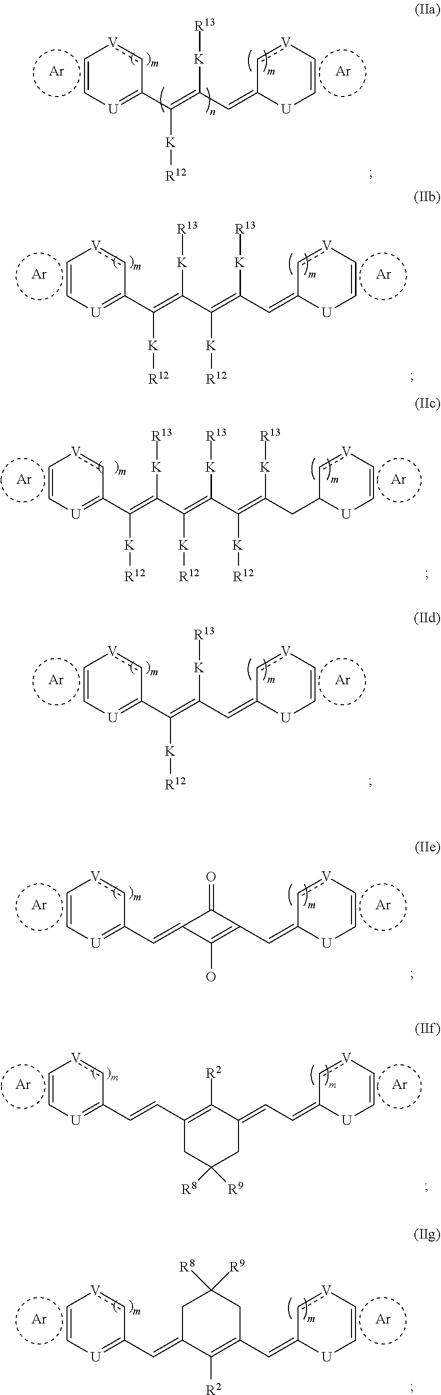

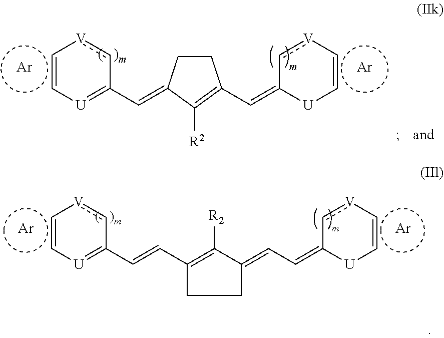

3. The compound of claim 1, wherein the 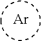
groups comprise the same 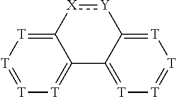
group or derivative thereof, optionally wherein the derivative of 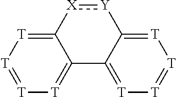
comprises an additional aryl or heteroaryl group fused at any available 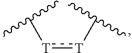
optionally

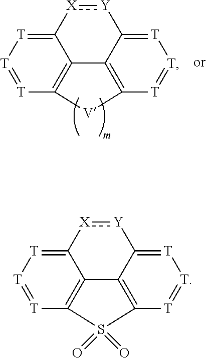

4. The compound of claim 1, wherein the 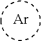
groups comprise different 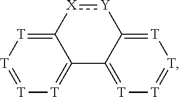
groups or derivatives thereof, optionally wherein the derivative of 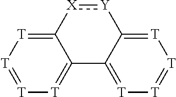
comprises an additional aryl or heteroaryl group fused at any available any available 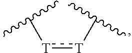
optionally

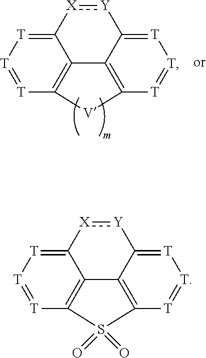

5. The compound of claim 1, wherein one 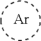
is an unsubstituted or substituted benzene or unsubstituted or substituted naphthalene;
U is N; and
V is CR¹¹ or C(R¹¹)₂, N, O, or S.

6. The compound of claim 1, wherein the at least one 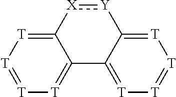
group or derivative thereof is selected from the group consisting of: 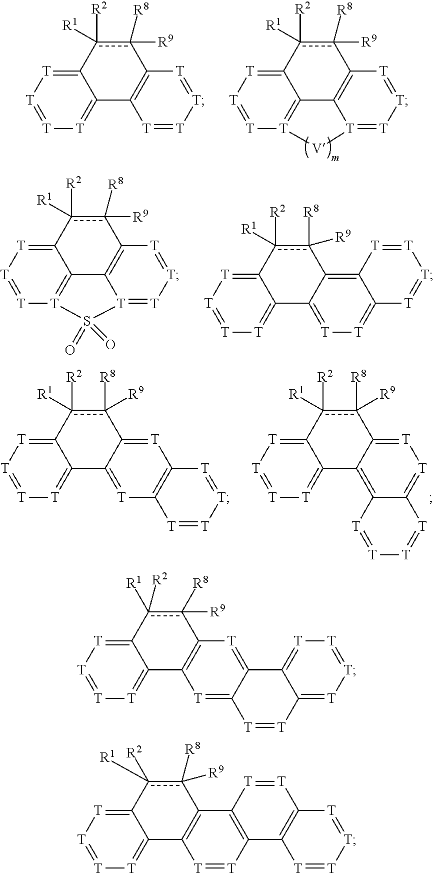 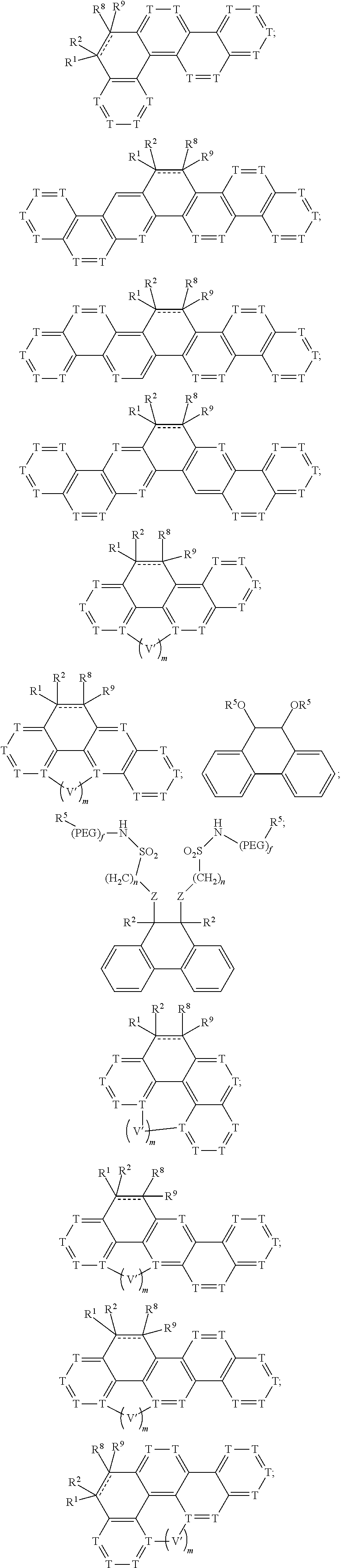 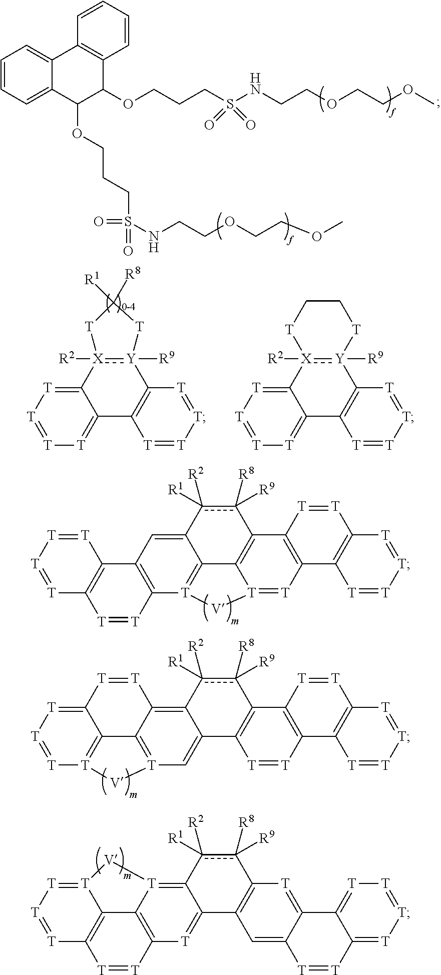 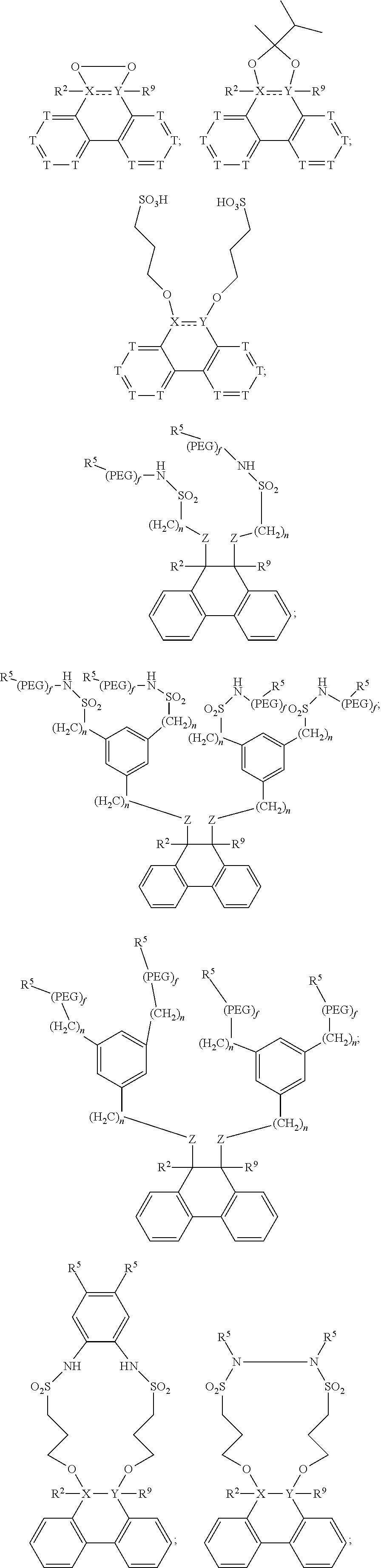 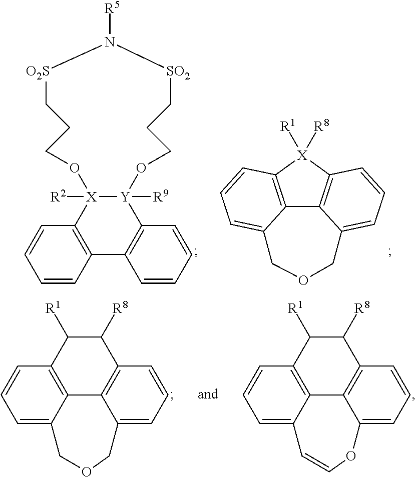
wherein each R⁵ is independently selected from the group consisting of halogen, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, a C₂-C₁₈ (hetero)aryl group, C₂-C₁₈ (hetero)aryloxy, C₂-C₁₈ (hetero)arylamino, carboxylic acid, carboxylate ester, (CH₂)_{x′}(OCH₂—CH₂)_{y′}OCH₃, and (CH₂)_{x′}(OCH₂—CH₂)_{y′}OCF₃, where each x′ is independently an integer from 0-20, and each y′ is independently an integer from 0-50.

7. The compound of claim 1, comprising a structure selected from the group consisting of Formula (IIIa), (IIIb), (IIIc), (IIId), (IIIe), (IIIf), (IIIy), (IIIbb), (IIIcc), (IIIdd), and (IIIee):

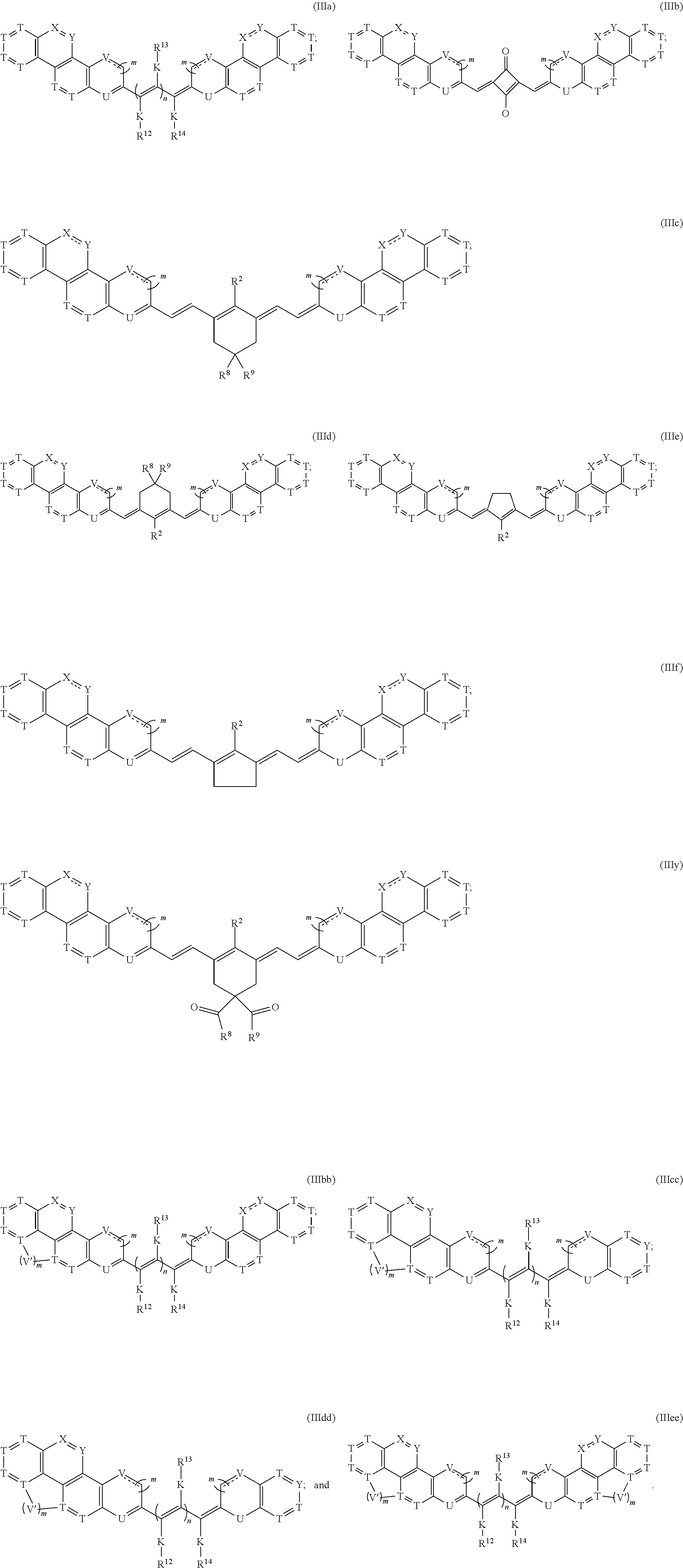

8. The compound of claim 1, wherein each R¹⁰ and each R¹¹ are independently selected from the group consisting of a water-solubilizing moiety, a linker moiety, a linked water-solubilizing moiety, hydrogen, alkyl, alkene, alkyne, cycloalkyl, haloalkyl, (hetero)aryloxy, aryl, heteroaryl, (hetero)arylamino, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide, alkoxy sulfonamide PEG, alkoxy sulfonate, alkyl sulfonate, alkyl sulfonate salt,

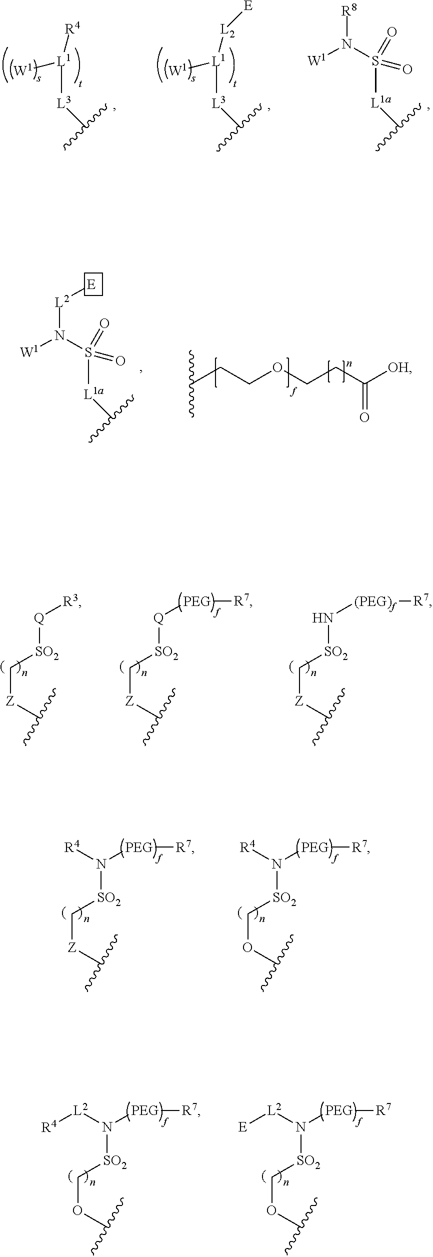

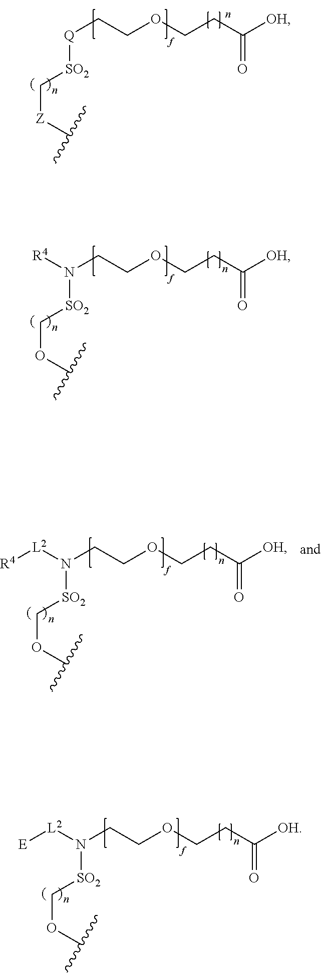

9. The compound of claim 1, comprising a structure according to any one of

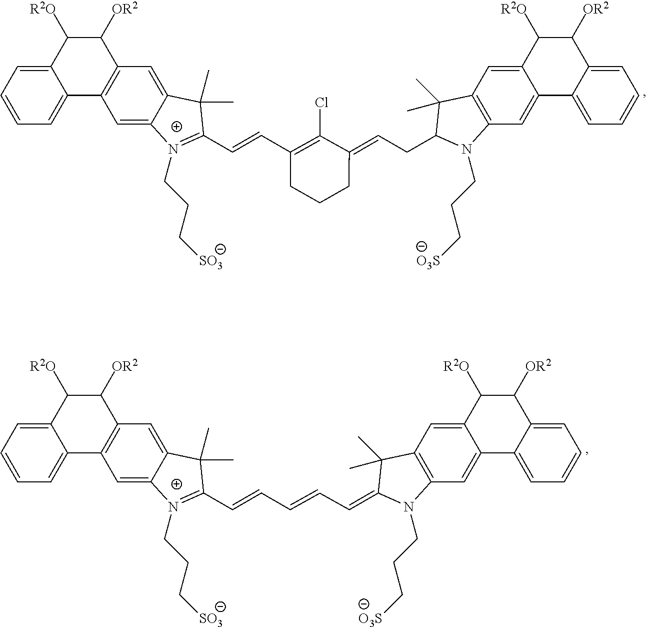

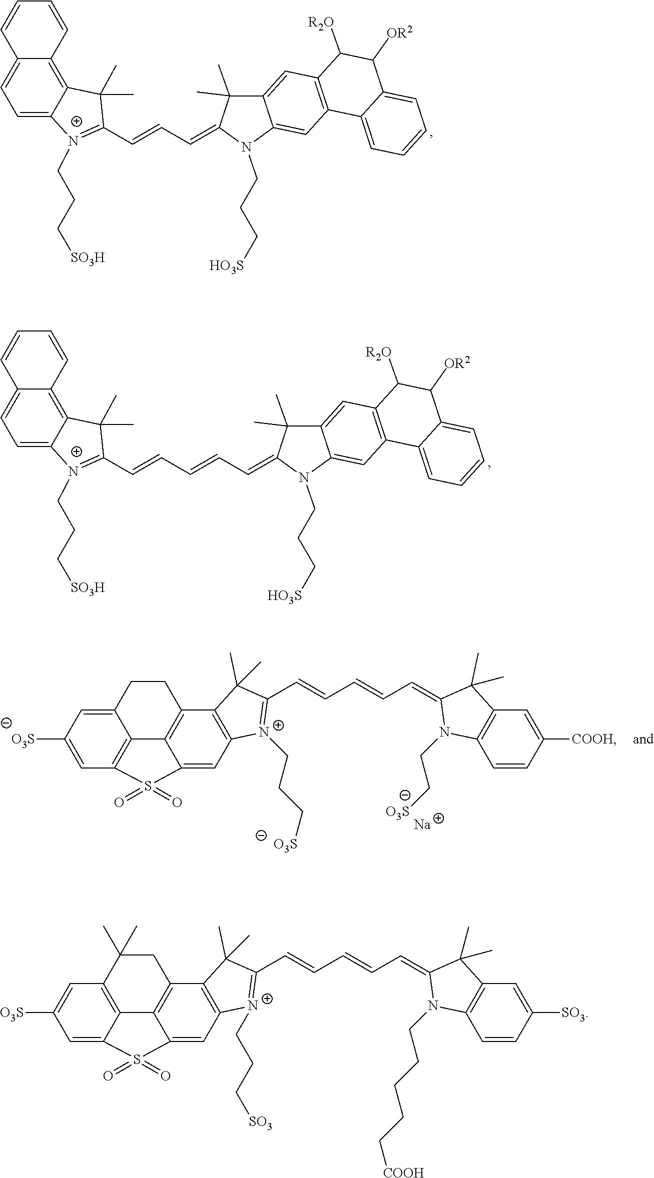

10. (canceled)

11. A polymer dye comprising a monomer comprising a structure according to Formula (IV) or (V): 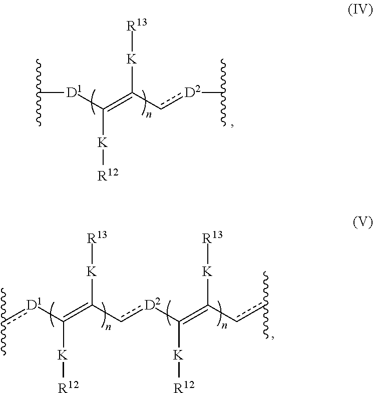
wherein
each [symbol] in (IV) or (V) is a point of attachment to the polymer dye backbone;
each D¹ and D² is independently selected from the group consisting of an aryl group, heteroaryl group, and 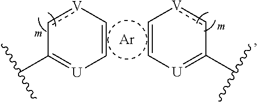
wherein
each 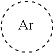
is independently selected from the group consisting of a substituted or unsubstituted benzene, benzene derivative, monocyclic aryl group, polycyclic aryl group, monocyclic heteroaryl group, and polycyclic heteroaryl group, wherein at least one of D¹ or D² is 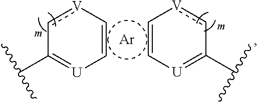
wherein 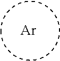
is 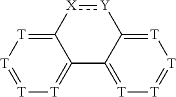
or a derivative thereof comprising an additional aryl or heteroaryl group fused at any available 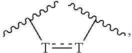
optionally wherein the derivative thereof comprises 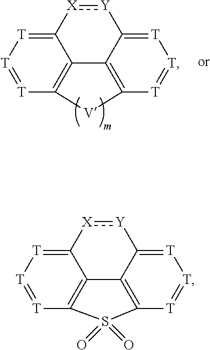
and wherein the 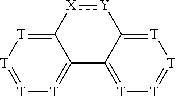
group or derivative thereof is fused to 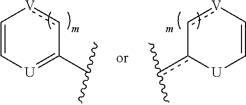
at any available 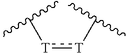
position on the 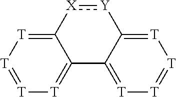
group or derivative thereof;
each V′ is independently SO₂, SO, S, NR¹¹, CR¹¹, C(R¹¹)₂, O, Si(R¹¹)₂, >C═O, >Se═O, —CH═CH—, or —N═CH—;
each T is independently selected from the group consisting of C, C(R¹), N, P, O, S, and Si(R¹);
each U is independently selected from the group consisting of NR¹⁰, O, P, Se, Te, and S;
each V is independently selected from the group consisting of NR¹¹, CR¹¹, C(R¹¹)₂, S, SO₂, O, Se, Te, and Si(R¹¹)₂;
each X is independently CR¹R² or SiR¹R²;
each Y is independently CR⁸R⁹ or SiR⁸R⁹;
wherein
each R¹, R², R⁸, and R⁹ is independently selected from the group consisting of a water-solubilizing moiety, a linked water-solubilizing moiety, a linker moiety, a linked E, a reactive group, a linked reactive group, binding partner, linked binding partner, a functional group, hydrogen, hydroxy, halogen, alkoxy, alkyl, alkenyl, alkynyl, cycloalkyl, haloalkyl, aryl, heteroaryl, (hetero)aryloxy, (hetero)arylamino, sulfonamide-PEG, phosphoramide-PEG, ammonium alkyl salt, ammonium alkyloxy salt, ammonium oligoether salt, carbonyl, acyl, sulfonate alkyl salt, sulfonate alkoxy salt, sulfonate oligoether salt, sulfonamido oligoether, sulfonamide, sulfinamide, phosphonamidate, phosphinamide, alkoxy sulfonamide PEG, alkoxy sulfonate, alkyl sulfonate, alkyl sulfonate salt, 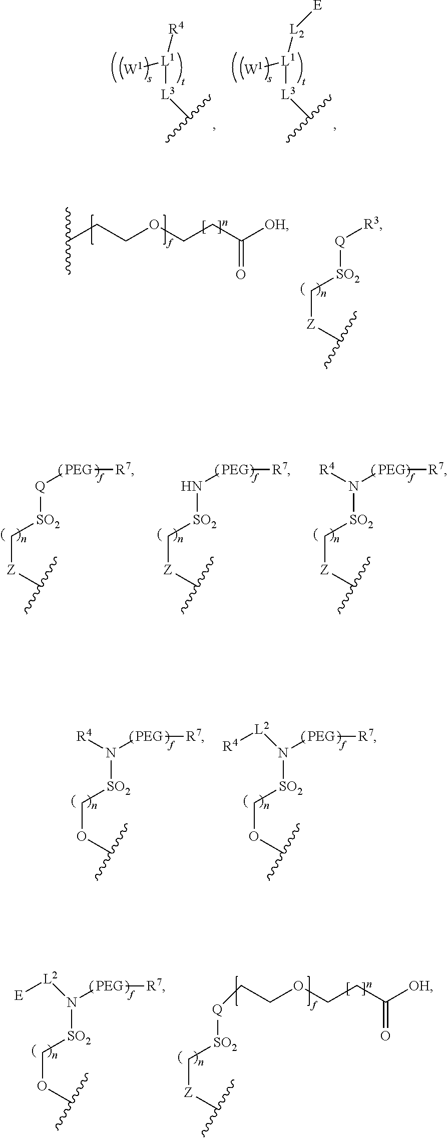 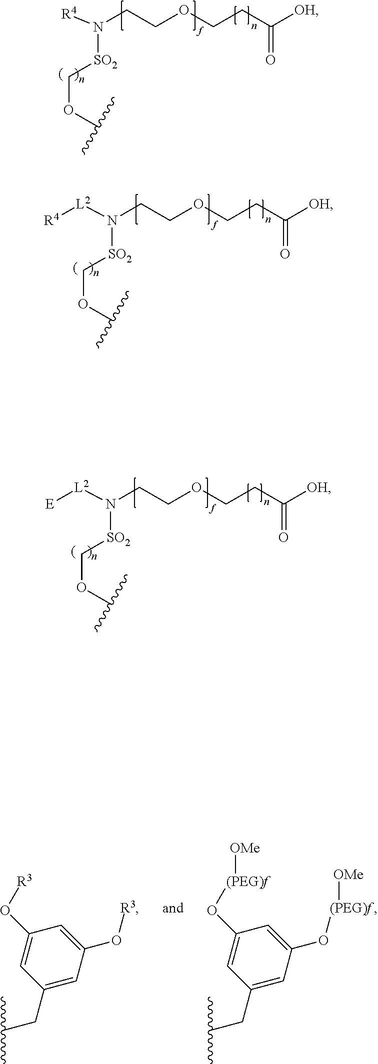
optionally wherein R¹ and R⁸ together form an unsubstituted or substituted cycloalkyl, cycloalkenyl, heterocycloalkyl, heterocycloalkenyl, cycloalkoxy, aryl, or heteroaryl having 3 to 9 ring members;
each R³ is independently selected from the group consisting of H, alkyl, alkene, alkyne, cycloalkyl, haloalkyl, alkoxy, (hetero)aryloxy, aryl, (hetero)arylamino, a water-solubilizing moiety, a chromophore, functional moiety, binding partner, and a PEG group;
each Q is independently a bond, NH, NR⁴, C₁-C₁₂ alkylene, CHR⁴, or CH₂;
each Z is independently CH₂, CHR⁴, O, NR⁴, or NH;
each W¹ is independently a water-solubilizing moiety;
L¹, L², and L³ are each independently selected linker moieties;
each E is independently selected from the group consisting of a chromophore, a functional moiety, a substrate, and a binding partner;
each R⁴ is independently selected from the group consisting of H, alkyl, PEG, a water-solubilizing moiety, a linker moiety, a chromophore, a functional group, a conjugation tag, carboxylic amine, amine, carbamate, carboxylic acid, carboxylate ester, maleimide, activated ester, N-hydroxysuccinimidyl, hydrazine, hydrazide, hydrazone, azide, alkyne, alkene, tetrazine, aldehyde, and thiol, or protected groups thereof;
each R⁷ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester, and C₁-C₁₂ alkoxy, a water-solubilizing moiety, a PEG moiety, a functional group, a chemoselective functional group, conjugation tag, linked conjugation tag, a linker, sulfonic acid, sulfonate, C₁-C₁₂ alkyl sulfonate, sulfonamide;
each R¹⁰ is independently selected from the group consisting of hydrogen, a linker moiety, a linked reactive group, a linked ionic group, a linked chromophore, a linked water-solubilizing moiety, a water-solubilizing moiety, a chromophore, a binding partner, a linked binding partner;
each R¹¹ is independently selected from the group consisting of hydrogen, a linker moiety, a linked reactive group, a linked ionic group, a linked chromophore, a linked water-solubilizing moiety, a chromophore, a binding partner, and a linked binding partner;
each R¹² and R¹³ is independently selected from the group consisting of hydrogen, halogen, substituted or unsubstituted C₁-C₆ alkyl, substituted or unsubstituted C₁-C₆ heteroalkyl, substituted or unsubstituted C₁-C₆ alkene, substituted or unsubstituted C₃-C₁₀ cycloalkyl, substituted or unsubstituted heterocycloalkyl, substituted or unsubstituted aryl, and substituted or unsubstituted heteroaryl; or R¹² and R¹³ together form an unsubstituted or substituted unsaturated cycloalkyl group having 3 to 8 ring members or substituted or unsubstituted heterocycloalkyl having 3 to 8 ring members optionally substituted with O;
each K is independently a covalent bond, O, S, P, NR¹, Se, Te, CR¹R², or CH₂;
each f is independently an integer from 0 to 50;
each m is independently 0 or 1;
each n is independently 0, 1, 2, 3, or 4;
each s is independently 1 or 2; and
each t is independently 0, 1, 2, or 3,
optionally wherein at least one of R¹, R², R³, R⁴, R⁸, R⁹, R¹⁰, and R¹¹ comprises a water-solubilizing moiety or a linked water-solubilizing moiety, further optionally wherein the polymer is water-soluble.

12. The polymer dye of claim 11, wherein the monomer of Formula (IV) or (V) comprises a structure selected from the group consisting of Formula (A1), (A2), (A3), (A4), (A5), (A6), (A7), (A8), (A9), (A10), (A11), (A12), (A13), (A14), (A15), (A16), (A17), (A18), and (A19):

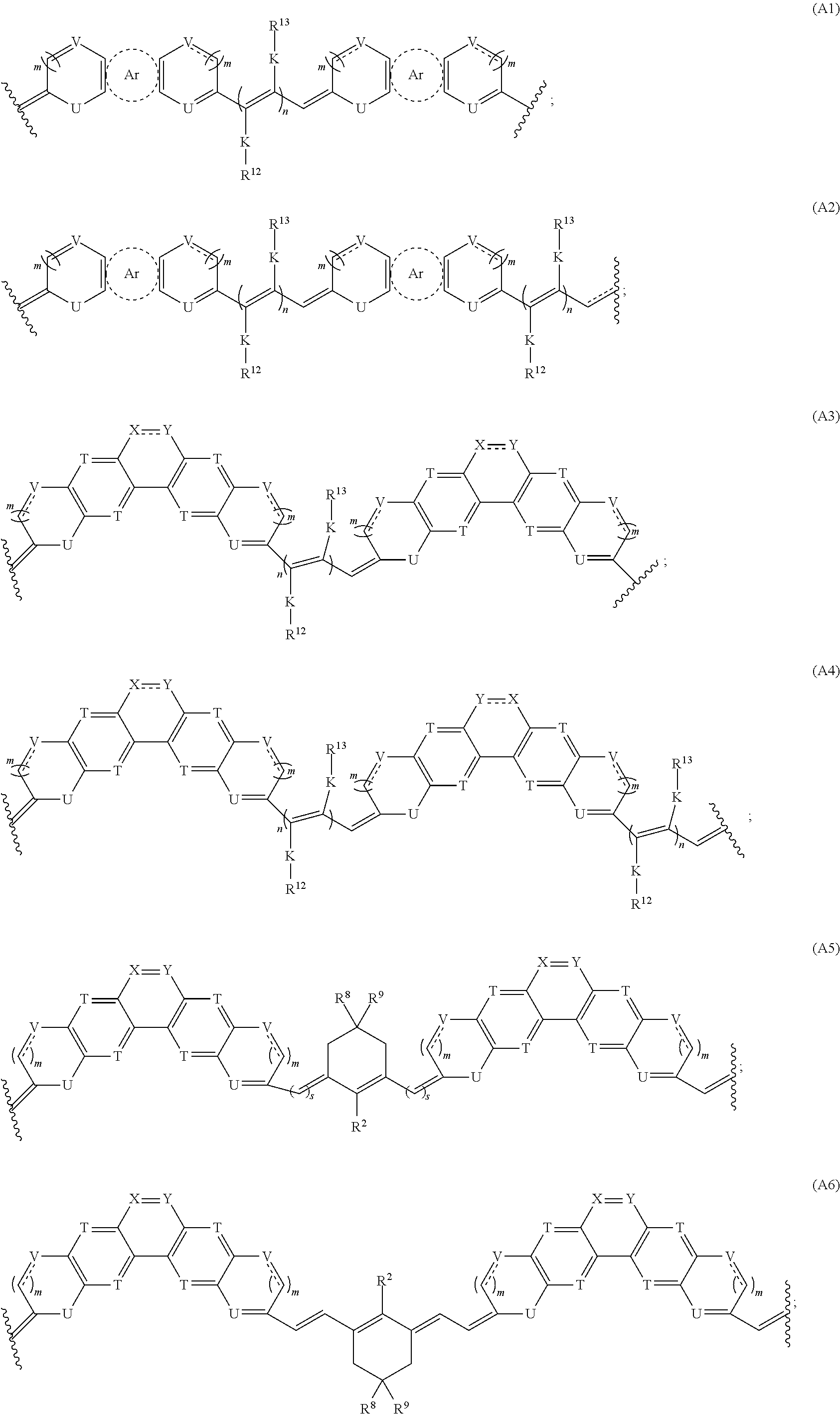

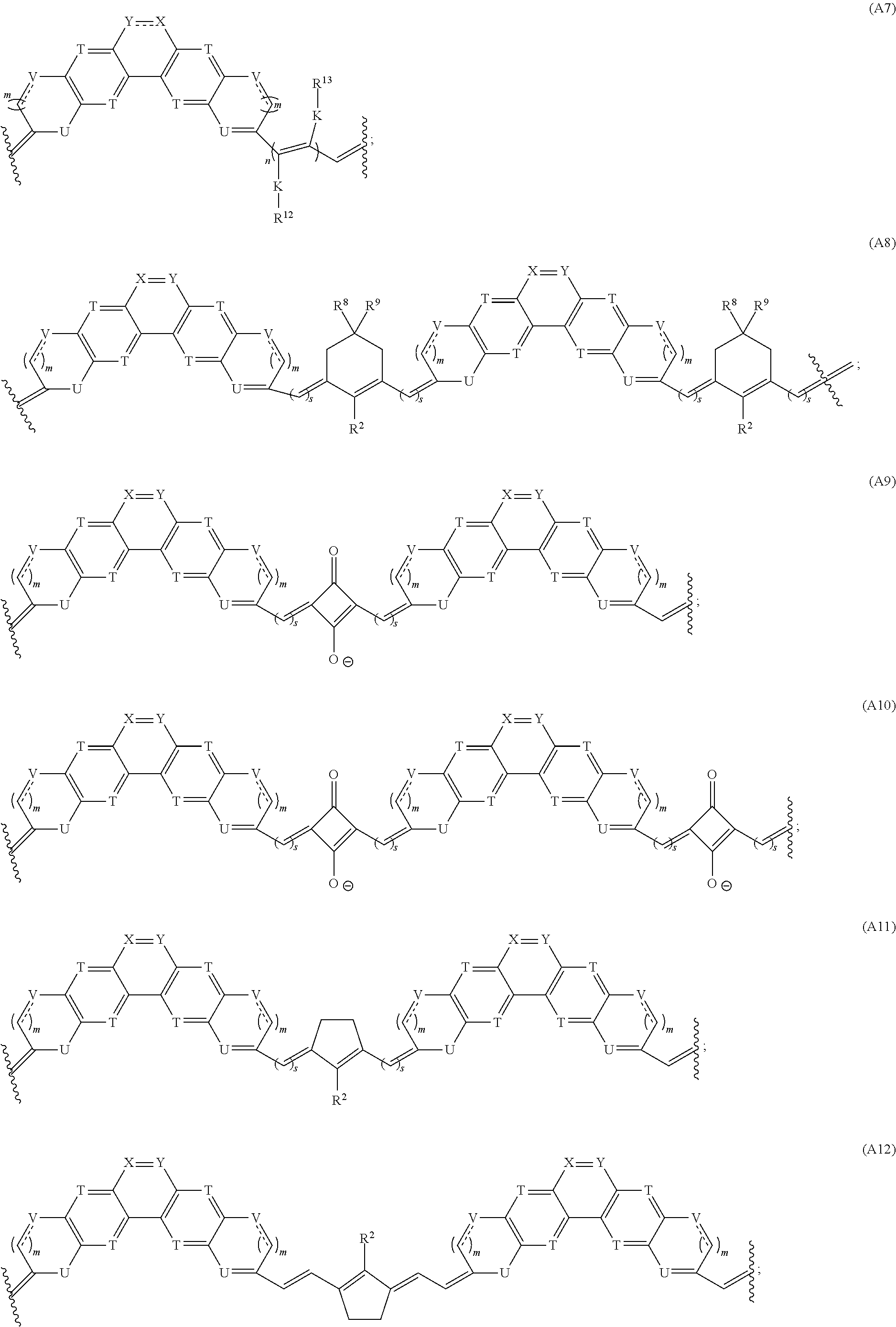

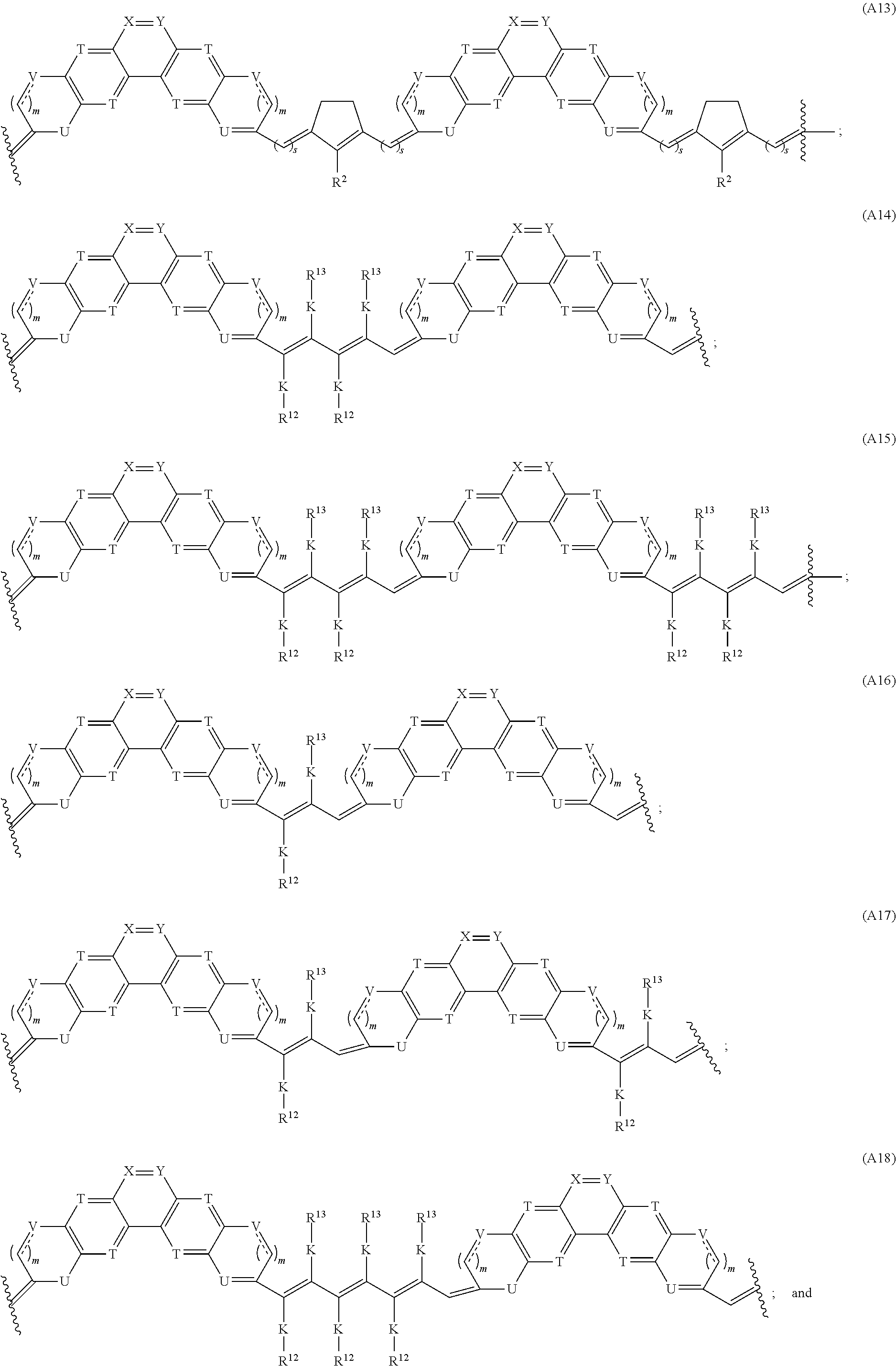

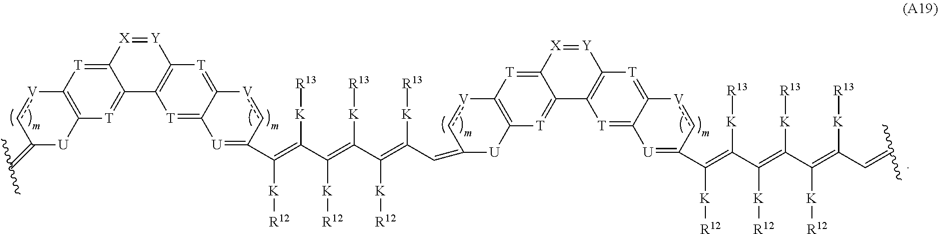

13. A labeled specific binding partner, comprising:
the fluorescent compound according to claim 1; and
a specific binding partner covalently linked to the fluorescent compound or polymer, optionally wherein the specific binding partner is selected from the group consisting of a protein, peptide, affinity ligand, antibody, antibody fragment, carbohydrate, lipid, nucleic acid, and an aptamer, further optionally wherein the specific binding partner is an antibody.

14.-15. (canceled)

16. A tandem dye, comprising:
the fluorescent compound according to claim 1; and
an acceptor chromophore or a donor chromophore covalently linked to the fluorescent compound, polymer, or labeled specific binding partner.

17.-18. (canceled)

19. A method for detecting a target analyte in a sample comprising:
providing a sample that is suspected of containing the analyte; and
contacting the sample with a specific binding partner conjugated to a fluorescent compound as defined in claim 1, wherein the specific binding partner is capable of interacting with the target analyte, optionally wherein the binding partner is a protein, peptide, affinity ligand, antibody, antibody fragment, carbohydrate, lipid, nucleic acid or an aptamer, further optionally wherein the binding partner is an antibody, and
a. the method is configured for flow cytometry;
b. the fluorescent compound is bound to a substrate;
c. the analyte is a protein expressed on a cell surface;
d. the method is configured as an immunoassay; or
e. the method further comprises providing additional binding partners for detecting additional analytes simultaneously.

20.-21. (canceled)

22. A kit comprising at least one fluorescent compound according to claim 1.

23. The polymer dye of claim 11,
wherein the [symbol] in 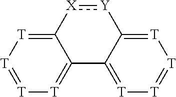
is a single bond.

24. A labeled specific binding partner, comprising:
the polymer dye according to claim 11; and
a specific binding partner covalently linked to the polymer, optionally wherein the specific binding partner is selected from the group consisting of a protein, peptide, affinity ligand, antibody, antibody fragment, carbohydrate, lipid, nucleic acid, and an aptamer, further optionally wherein the specific binding partner is an antibody.

25. A tandem dye, comprising:
the polymer dye according to claim 11; and
an acceptor chromophore or a donor chromophore covalently linked to the fluorescent compound, polymer, or labeled specific binding partner.

26. A method for detecting a target analyte in a sample comprising:
providing a sample that is suspected of containing the analyte; and
contacting the sample with a specific binding partner conjugated to a polymer dye as defined in claim 11, wherein the specific binding partner is capable of interacting with the target analyte, optionally wherein the binding partner is a protein, peptide, affinity ligand, antibody, antibody fragment, carbohydrate, lipid, nucleic acid or an aptamer, further optionally wherein the binding partner is an antibody, and:
a. the method is configured for flow cytometry;
b. the water-soluble fluorescent polymer is bound to a substrate;
c. the analyte is a protein expressed on a cell surface;
d. the method is configured as an immunoassay; or
e. the method further comprises providing additional binding partners for detecting additional analytes simultaneously.

27. A kit comprising at least one polymer dye according to claim 11.
